(19) United States
(12) Patent Application Publication
(10) Pub. No.: US 2004/0235939 A1
(43) Pub. Date: Nov. 25, 2004

(54) Synthesis of peloruside a and analogs thereof for use as antitumor agents

(76) Inventors: Jef De Brabander, Lewisville, TX (US); Xibin Liao, Dallas, TX (US)

Correspondence Address: BAKER & BOTTS, 30 ROCKEFELLER PLAZA, NEW YORK, NY, 10112

(21) Appl. No.: 10/783,848

(22) Filed: Feb. 20, 2004

Related U.S. Application Data
(60) Provisional application No. 60/448,851, filed on Feb. 20, 2003.

(51) Int. Cl. A61K 31/365; C07D 493/04

(52) U.S. Cl. 514/450; 549/267

(57) ABSTRACT

The present invention provides (+)-Peloruside A and analogs thereof having microtubule-stabilizing activity, and a process for synthesizing these compounds. Also provided are intermediates and a process for synthesizing thereof. These compounds exhibit antiproliferative activity and are useful for treating cancer.

### FIELD OF THE INVENTION

The present invention relates to the organic synthesis of a novel structural class of antimitotic compounds and anti-cancer pharmaceuticals, particularly Peloruside A and analogs thereof, having anti-proliferative activity and microtubule-stabilizing activity.

### BACKGROUND OF THE INVENTION

A number of biological metabolites isolated from natural sources such as sponges, tunicates, plants and bacteria have been found to have anti-cancer activity. A novel secondary sponge metabolite, Peloruside A, was recently isolated from the marine sponge Mycale sp. and shown to have cytotoxic effects on mammalian cells in culture. Hood et al., 2001, Anticancer Drug Design, 16:155-166 (“Hood 2001”). Hood 2001 disclose that Peloruside A is a polyoxygenated 16-membered ring macrolide containing a pyranose ring adjacent to a gem-dimethyl moiety and that it has similarity to bryostatins, which have larger rings but which also contain a pyranose ring adjacent to a gem-dimethyl. Hood 2001 further disclose that Peloruside A has a different activity than brysotatin-1, namely, Peloruside A is not protein kinase C-dependent.

In another publication, Hood et al. disclose that Peloruside A has microtubule-stabilizing activity. Hood et al., 2002, Cancer Research 62:3356-3360 (“Hood 2002”). Microtubule-stabilizing activity is a desired characteristic in an anti-cancer pharmaceutical, because drugs which interfere with mitosis have proven effective in the treatment of cancer. Hood 2002 show that Peloruside affects microtubule dynamics in a manner similar to paclitaxel (Taxol®). The structure described by Hood of Peloruside A is also shown in U.S. patent Publication No. US 2002/0193423 and PCT Publication No. WO 01/10869.

While Peloruside A has been previously described, there is no known means of synthesizing Peloruside A. Because large amounts of Peloruside A would be needed for pharmaceutical applications, a synthetic Peloruside A is desirable. The present invention describes the first synthesis of both enantiomeric forms of Peloruside A, i.e. (−)-Peloruside A (FIG. 1) and (+)-Peloruside A (FIG. 2) and assigns the absolute configuration of the natural product (+)-Peloruside as 2S, 3R, 5R, 7R, 8R, 9R, 11S, 13S, 15S, 18R (Peloruside numbering) The absolute configuration of natural, biologically active (+)-Peloruside A has not been assigned previously. Peloruside A has a unique architecture of a macrolactone with a very dense functionalization.

### SUMMARY OF THE INVENTION

The invention includes a method of synthesis of a novel structural class of microtubule-stabilizing compounds and anti-cancer pharmaceuticals, particularly Peloruside A and analogs thereof having anti-cancer activity and microtubule-stabilizing activity similar to that of paclitaxel (Taxol®). The synthetic Pelorusides described herein have a unique architecture of a macrolactone having very dense functionalization. The present invention also documents the first case of a configuration dependent mechanistic switch for a Mitsunobu lactonization and uses a unique approach of advancing highly complex intermediates with minimal use of protecting groups. Included in the invention are the compounds, compositions containing the compounds, methods of synthesis, and methods of treatment.

An embodiment of the invention is a synthetic compound having the ¹³C and ¹H NMR signatures of FIG. 4 and FIG. 5, wherein the compound is dextrarotary, and wherein the compound has microtubule-stabilizing activity.

Another embodiment of the invention is a compound of Formula I and compositions comprising a compound of Formula I:

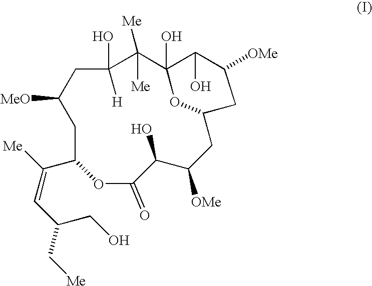

A further embodiment of the invention is a compound of the Formula II and compositions comprising a compound of Formula II:

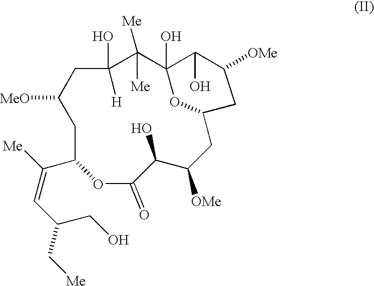

Another embodiment of the invention is a compound of the Formula III and compositions comprising a compound of Formula III:

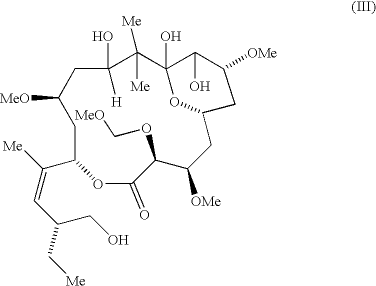

Still another embodiment of the invention is a compound of Formula IV and compositions comprising a compound of Formula IV:

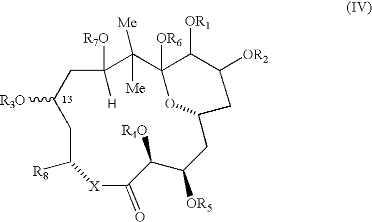

where R₁, R₂, R₃, R₄, R₅ and R₆ can be the same or different and include: H, Me, alkyl, funtionalized alkyl (functionalization may include, inter alia, heteroatoms, halides, aryl, heteroaryl), and where R₈═H, Me, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl). The configuration at the carbon bearing the OR₁, OR₂, and OR₃ substituents can have the R— or S-configuration.

In still a further embodiment, the invention provides a compound of Formula V and compositions comprising a compound of Formula V:

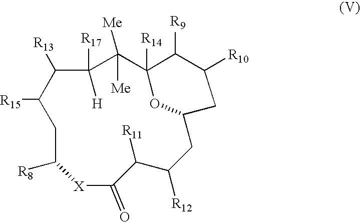

where R₁₃═H or Me, where R₁₄, R₁₇ can be the same or different and include H, OH, or OR, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈═H, Me, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅ and OR₁₅ substituents can be of the R— or S-configuration.

The invention also relates to a compound of Formula VI and to compositions comprising a compound of Formula VI:

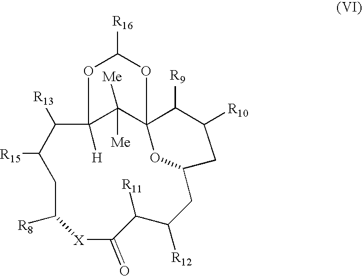

where R₁₃═H or Me, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅, R₁₆ and OR₅ substituents can be of the R— or S-configuration.

The invention also relates to a compound of Formula VII and to compositions comprising a compound of Formula VII:

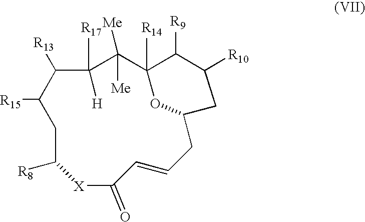

where R₁₃═H or Me, where R₁₄, R₁₇ can be the same or different and include H, OH, or OR, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅ substituents can be of the R— or S-configuration.

The invention also relates to a compound of Formula VIII and to compositions comprising a compound of Formula VIII:

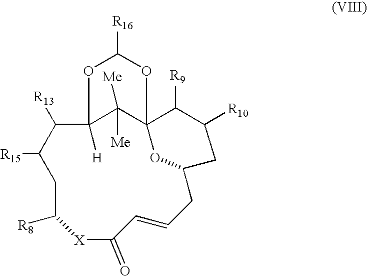

where R₁₃═H or Me, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅, R₁₆ substituents can be of the R— or S-configuration.

The invention also relates to a compound of Formula IX and to compositions comprising a compound of Formula IX:

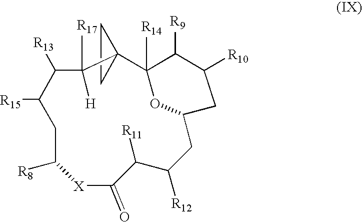

where R₁₃═H or Me, where R₁₄, R₁₇ can be the same or different and include H, OH, or OR, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅ and OR₅ substituents can be of the R— or S-configuration.

The invention also relates to a compound of Formula X and to compositions comprising a compound of Formula X:

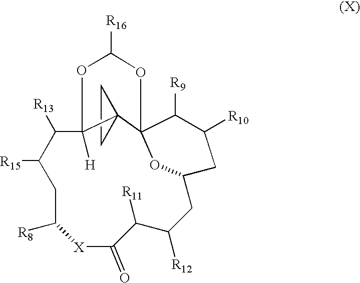

where R₁₃═H or Me, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅, R₁₆ and OR₅ substituents can be of the R— or S-configuration.

The invention also relates to a compound of Formula XI and to compositions comprising a compound of Formula XI:

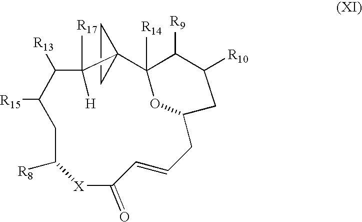

where R₁₃═H or Me, where R₁₄, R₁₇ can be the same or different and include H, OH, or OR, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅ substituents can be of the R— or S-configuration.

The invention also relates to a compound of Formula XII and to compositions comprising a compound of Formula XII:

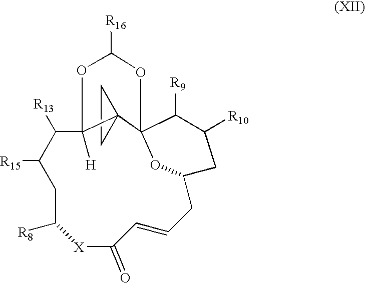

where R₁₃═H or Me, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅, R₁₆ substituents can be of the R— or S-configuration.

Yet another embodiment of the invention is a process for preparing a peloruside comprising the steps of a) synthesizing a pyran ring containing a first substituent having a carboxylic acid group and a second substituent having a hydroxyl group; and b) reacting the carboxylic acid group with the hydroxyl group to form a lactone.

The invention also provides a method of treating or preventing cancer, comprising the step of administering to a patient a therapeutically effective amount of one or more macrolactone Peloruside compounds of Formulas I through XII.

More specifically, the method for treating cancer comprises the step of contacting a tumor cell within a subject with a macrolactone peloruside of the present invention under conditions permitting the uptake of said peloruside by said tumor cell. The tumor cell may be derived from a tissue selected from the group consisting of breast, brain, lung, liver, spleen, kidney, lymph node, small intestine, blood, pancreas, colon, stomach, endometrium, prostate, testicle, ovary, skin, head, and neck, esophagus, and bone marrow. In one embodiment, the tumor cell is derived from breast and is resistant to Taxol®. In a further embodiment, the subject is human. The compounds of the present invention are also useful for suppressing the growth of tumor cells.

The invention also provides a method for stabilizing microtubule formation. The method comprises contacting microtubules with the compounds of the present invention in an amount sufficient to stabilize microtubule formation. The microtubule-stabilizing activity of the compounds of the present invention is useful, inter alia, for the treatment and prevention of cancer.

The invention further provides a method of regulating cell growth and proliferation in normal and malignant cells, comprising the step of administering to the cells an effective amount of a compound of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 shows the absolute configuration of (−)-Peloruside A, the biologically inactive enantiomer according to the present invention;

FIG. 2 shows the absolute configuration of (+)-Peloruside A, the biologically active enantiomer according to the present invention;

FIG. 3 shows the strategy for synthesizing Peloruside A;

FIG. 4 is a ¹³C NMR spectrum of synthetic Peloruside A, 75 MHz, CDCl₃;

FIG. 5 is a ¹H NMR spectrum of synthetic Peloruside A, 400 MHz, CDCl₃, with inserts showing magnified views of the regions from about 4.3 to about 3.6 ppm and 2.1 ppm;

FIG. 6 shows the strategy for the preparation of a C1-C13 fragment;

FIG. 7 shows the problematic glycal-epoxide solvolysis of the C1-C13 fragment;

FIG. 8 shows the solution for the problematic glycal-epoxide solvolysis for the C1-C13 fragment by elimination of the C11 sterogenic centrum;

FIG. 9 is the ¹H NMR signature of Ketone 6, 400 MHz, CDCl₃;

FIG. 10 is the ¹³C NMR signature of Ketone 6, 75 MHz, CDCl₃;

FIG. 11 is the ¹H NMR signature of compound 9, 400 MHz, CDCl₃;

FIG. 12 is the ¹³C NMR signature of compound 9, 75 MHz, CDCl₃;

FIG. 13 is the ¹H NMR signature of compound 11, 400 MHz, CDCl₃;

FIG. 14 is the ¹³C NMR signature of compound 11, 75 MHz, CDCl₃;

FIG. 15 is the ¹H NMR signature of compound 12, 400 MHz, CDCl₃;

FIG. 16 is the ¹³C NMR signature of compound 12, 75 MHz, CDCl₃;

FIG. 17 is the ¹H NMR signature of compound 15, 400 MHz, CDCl₃;

FIG. 18 is the ¹³C NMR signature of compound 15, 75 MHz, CDCl₃;

FIG. 19 is the ¹H NMR signature of compound 16, 400 MHz, CDCl₃;

FIG. 20 is the ¹³C NMR signature of compound 16, 75 MHz, CDCl₃;

FIG. 21 is the ¹H NMR signature of compound 17, 400 MHz, CDCl₃;

FIG. 22 is the ¹³C NMR signature of compound 17, 75 MHz, CDCl₃;

FIG. 23 is the ¹H NMR signature of compound 19, 400 MHz, CDCl₃;

FIG. 24 is the ¹³C NMR signature of compound 19, 75 MHz, CDCl₃;

FIG. 25 is the ¹H NMR signature of compound 20, 400 MHz, CDCl₃;

FIG. 26 is the ¹³C NMR signature of compound 20, 75 MHz, CDCl₃;

FIG. 27 is the ¹H NMR signature of compound 21, 400 MHz, CDCl₃;

FIG. 28 is the ¹³C NMR signature of compound 21, 75 MHz, CDCl₃;

FIG. 29 is the ¹H NMR signature of compound 22, 400 MHz, CDCl₃;

FIG. 30 is the ¹³C NMR signature of compound 22, 75 MHz, CDCl₃;

FIG. 31 is the ¹H NMR signature of compound 23, 400 MHz, CDCl₃;

FIG. 32 is the ¹³C NMR signature of compound 23, 75 MHz, CDCl₃;

FIG. 33 is the ¹H NMR signature of compound 24, 400 MHz, CDCl₃;

FIG. 34 is the ¹³C NMR signature of compound 24, 75 MHz, CDCl₃;

FIG. 35 is the 1D NOE IR signature of compound 24 at 5.87 ppm, 400 MHz, CDCl₃;

FIG. 36 is the 1D NOE IR signature of compound 24 at 1.22 ppm, 400 MHz, CDCl₃;

FIG. 37 is the ¹H NMR signature of compound 29, 400 MHz, CDCl₃;

FIG. 38 is the 1D NOE IR signature of compound 29 at 5.53 ppm, 400 MHz, CDCl₃;

FIG. 39 is the 1D NOE IR signature of compound 29 at 4.54 ppm, 400 MHz, CDCl₃;

FIG. 40 is the 2D NOE IR signature of compound 29, 400 MHz, CDCl₃;

FIG. 41 is the ¹³C NMR signature of compound 29, 75 MHz, CDCl₃;

FIG. 42 is the ¹H NMR signature of compound 30, 400 MHz, CDCl₃;

FIG. 43 is the ¹³C NMR signature of compound 30, 75 MHz, CDCl₃;

FIG. 44 is the ¹H NMR signature of compound 31, 400 MHz, CDCl₃;

FIG. 45 is the ¹³C NMR signature of compound 31, 75 MHz, CDCl₃;

FIG. 46 is the ¹H NMR signature of compound 32a, 400 MHz, CDCl₃;

FIG. 47 is the ¹³C NMR signature of compound 32a, 75 MHz, CDCl₃;

FIG. 48 is the ¹H NMR signature of compound 33a, 400 MHz, CDCl₃;

FIG. 49 is the ¹³C NMR signature of compound 33a, 75 MHz, CDCl₃;

FIG. 50 is the ¹H NMR signature of compound 34a, 400 MHz, CDCl₃;

FIG. 51 is the ¹³C NMR signature of compound 34a, 75 MHz, CDCl₃;

FIG. 52 is the ¹H NMR signature of compound 36, 400 MHz, C₆D₆;

FIG. 53 is the 1D NOE IR signature of compound 36 at 5.74 ppm, 400 MHz, C₆D₆;

FIG. 54 is the ¹³C NMR signature of compound 36, 75,MHz, CDCl₃;

FIG. 55 is the ¹H NMR signature of compound 37, 400 MHz, CDCl₃;

FIG. 56 is the ¹H NMR signature of compound 38, 400 MHz, C₆D₆;

FIG. 57 is the ¹H NMR signature of compound 38, 400 MHz, CDCl₃;

FIG. 58 is the 1D NOE IR signature of compound 38 at 6.16 ppm, 400 MHz, C₆D₆;

FIG. 59 is the ¹³C NMR signature of compound 38, 75 MHz, CDCl₃;

FIG. 60 is the ¹H NMR signature of compound 39, 400 MHz, CDCl₃;

FIG. 61 is the ¹³C NMR signature of compound 39, 75 MHz, CDCl₃;

FIG. 62 is the ¹H NMR signature of compound Formula II (claim 2), 400 MHz, CDCl₃;

FIG. 63 is the ¹H NMR signature of compound Formula III (claim 3), 400 MHz, CDCl₃;

FIG. 64 is the ¹³C NMR signature of compound Formula III (claim 3), 75 MHz, CDCl₃;

FIG. 65 is the ¹H NMR signature of compound ent-40 (+)-Peloruside A, 400 MHz, CDCl₃;

FIG. 66 shows growth curves demonstrating the effect of Peloruside A treatment on the proliferation of tumor cell lines, MDA-MB-231, BT-549, PC-3, DU-145, LoVo, and Capan-1;

FIG. 67 shows growth curves demonstrating the effect of Peloruside A treatment on the proliferation of tumor cell lines, NCI-H460, NCI-H23, NCI-H1395, and NCI-H2887;

FIG. 68 shows growth curves demonstrating the effect of Peloruside A treatment on the proliferation of tumor cell lines, Hep-G2, SK-HEP-1, HCT-1 16, HCT-15, SK-MEL-28, and SK-MEL-5;

FIG. 69 shows growth curves demonstrating the effect of Peloruside A treatment on the proliferation of tumor cell lines, MiaPaCa-2. SK-OV-3, CAKI-1, and A498;

FIG. 70 shows growth curves demonstrating the effect of Taxol® treatment on the proliferation of tumor cell lines, MDA-MB-231, BT-549, PC-3, DU-145, LoVo, and Capan-1;

FIG. 71 shows growth curves demonstrating the effect of Taxol® treatment on the proliferation of tumor cell lines, NCI-H460, NCI-H23, NCI-H1395, and NCI-H2887;

FIG. 72 shows growth curves demonstrating the effect of Taxol® treatment on the proliferation of tumor cell lines, Hep-G2, SK-HEP-1, HCT-116, HCT-15, SK-MEL-28, and SK-MEL-5;

FIG. 73 shows growth curves demonstrating the effect of Taxol® treatment on the proliferation of tumor cell lines, MiaPaCa-2. SK-OV-3, CAKI-1, and A498;

FIG. 74 shows the effect of 5 μM and 10 μM Taxol® on tubulin polymerization;

FIG. 75 shows the effect of 5 μM and 10 μM Peloruside A on tubulin polymerization;

FIG. 76A shows a bar graph of Peloruside A analog, LX3111, treatment on the viability of HeLa cells at 24, 48, 72 and 110 hours of treatment at various concentrations;

FIG. 76B a bar graph of Peloruside A analog, LX3111, treatment on the viability of HeLa cells at 26, 48, 73 and 106 hours of treatment at various concentrations;

FIG. 77 shows bar graph of Peloruside A analog, LX3111, treatment on the viability of HeLa cells at 48 hours of drug treatment as measured by luminescence;

FIG. 78A shows a bar graph of Peloruside A analog, LX3111, treatment on the viability of SK-MEK-5 cells at 24, 48, 72 and 110 hours of treatment at various concentrations;

FIG. 78B shows a bar graph of Peloruside A analog, LX3111, treatment on the viability of SK-MEK-5 cells at 26, 48, 73 and 106 hours of treatment at various concentrations;

FIG. 79 shows a bar graph of Peloruside A analog, LX3136, treatment on the viability of SK-MEK-5 cells at 24, 49, 70 and 107 hours of treatment at various concentrations;

FIG. 80 shows bar graphs of the cytotoxic effect of Taxol® treatment on 1A9, ptx10 and ptx22 cell growth;

FIG. 81 shows bar graphs of the cytotoxic effect of Peloruside A treatment on 1A9, ptx10 and ptx22 cell growth;

FIG. 82 shows bar graphs of the cytotoxic effect of Taxol® and Peloruside A treatment on 1A9, ptx10 and ptx22 cell growth at 20, 50 and 100 nM;

FIG. 83 shows several schemes for the synthesis of various intermediates of the present invention. Compound numbers refer to schemes presented in

FIGS. 84-96;

FIG. 84 shows the schemes for the synthesis of intermediates 62E and 62G;

FIG. 85 shows the scheme for the synthesis of intermediate 62A;

FIG. 86 shows the scheme for the synthesis of intermediate 62C;

FIG. 87 shows the scheme for the synthesis of intermediate 62D;

FIG. 88 shows the scheme for the synthesis of intermediate 62I;

FIG. 89 shows the scheme for the synthesis of intermediate 62J;

FIG. 90 shows the schemes for the synthesis of intermediates 62F and 62H;

FIG. 91 shows the scheme for the synthesis of intermediate 62B;

FIG. 92 shows the structure of intermediates 61A, 61B, 61E, 61F, 61G, 61H;

FIG. 93 shows the structure of intermediates 62A-62J;

FIG. 94 shows the structure of intermediates 63A-63J;

FIG. 95 shows the structure of intermediates 64A-64J;

FIG. 96 shows the structure of intermediates 65A-65J;

FIG. 97 shows immunofluorescent staining of tubulin in BSC-1 (Monkey Kidney epithelial) cells treated with 200 nM of Taxol® or 50 nM of Peloruside A.

### DETAILED DESCRIPTION OF THE INVENTION

Natural products that elicit a specific and unique biological response in mammalian cells represent valuable tools for new pharmaceuticals for the treatment for various disease states. In this context, the recent isolation of the macrolactone, Peloruside A, from the marine sponge Mycale sp. is noteworthy. Peloruside A represents a novel structural class of microtubule-stabilizing agents. Microtubule-stabilizing compounds can be divided into three groups (1) the terpenoids, which include taxanes, paclitaxel (Taxol®) and taxotere, (2) the macrolides, including epothilones and laulimalides, and (3) the polyhydroxylated alkatetraene lactones, including discodermolide. Peloruside is similar to epitholones in that it is a macrolide containing a 16-membered ring.

The present invention is directed to the first synthesis of both enantiomeric forms of a compound, Peloruside A. The present invention is based on the observation that the levarotatory enantiomeric form, (−)-Peloruside A, having the 2R, 3S, 5S, 7S, 8S, 9S, 11R, 13R, 15R, 18S absolute configuration, is biologically inactive, with regard to cytotoxicity and microtubule-stabilizing activity. The present invention also establishes for the first time that the biologically active dextrarotatory enantiomeric form, (+)-Peloruside A, has the 2S, 3R, 5R, 7R, 8R, 9R, 11S, 13S, 15S, 18R absolute configuration. Through the synthesis of both enantiomeric forms of Peloruside A, the present invention discloses the absolute configuration of the naturally occurring and biologically active (+)-Peloruside A as 2S, 3R, 5R, 7R, 8R, 9R, 11S, 13S, 15S, 18R. The naturally isolated Peloruside A and both enantiomeric forms of Peloruside A synthesized by the present invention have identical NMR and Mass-spectra signatures. However, only the synthetic enantiomeric form with the 2S, 3R, 5R, 7R, 8R, 9R, 11S, 13S, 15S, 18R absolute configuration is dextrarotatory (rotates the plane of polarized light, NaD-line, in a clockwise manner) and biologically active. Accordingly, the present invention discloses a method of making synthetic (+)-Peloruside A with assigned absolute configuration, and provides the first description of the absolute configuration of the biologically active dextrarotatory enantiomer. The (−) form of Peloruside A was inactive even at 10 μM concentrations when tested against the human tumor cell lines SK-MEL-5 and HeLa, whereas the natural Peloruside A is active at concentrations from about 4 nM to about 15 nM (see Hood et al., 2001, Anticancer Drug Design 16:155-166).

The molecular formula, relative configuration and spectroscopic data of Peloruside A were disclosed in Northcote et al., (WO 01/10869 and U.S. patent Publication No. 2002/0193423), (+)-Peloruside A. The absolute configuration of natural (+)-Peloruside A, however, was not assigned in Northcote et al., (WO 01/10869 and U.S. patent Publication No. 2002/0193423).

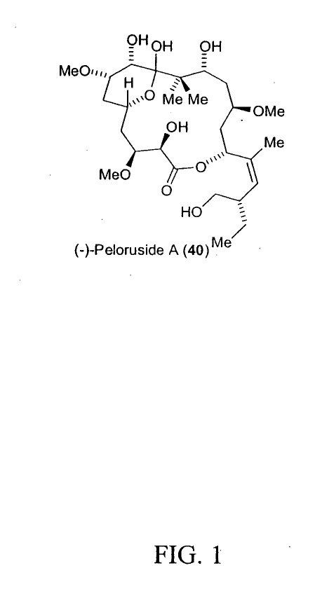

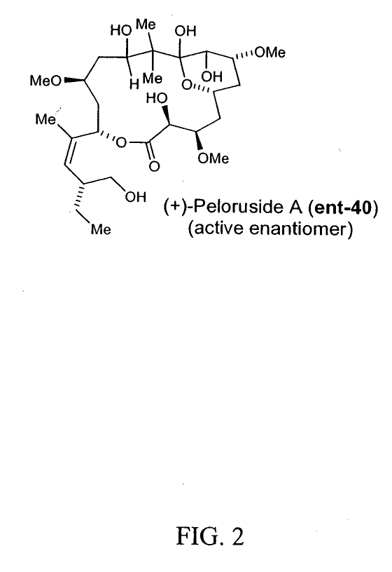

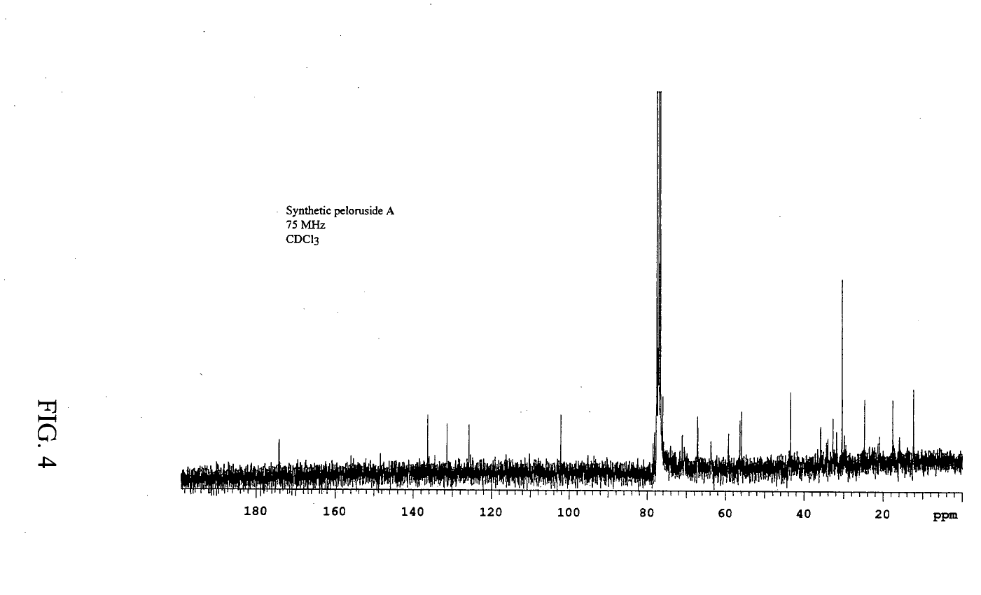

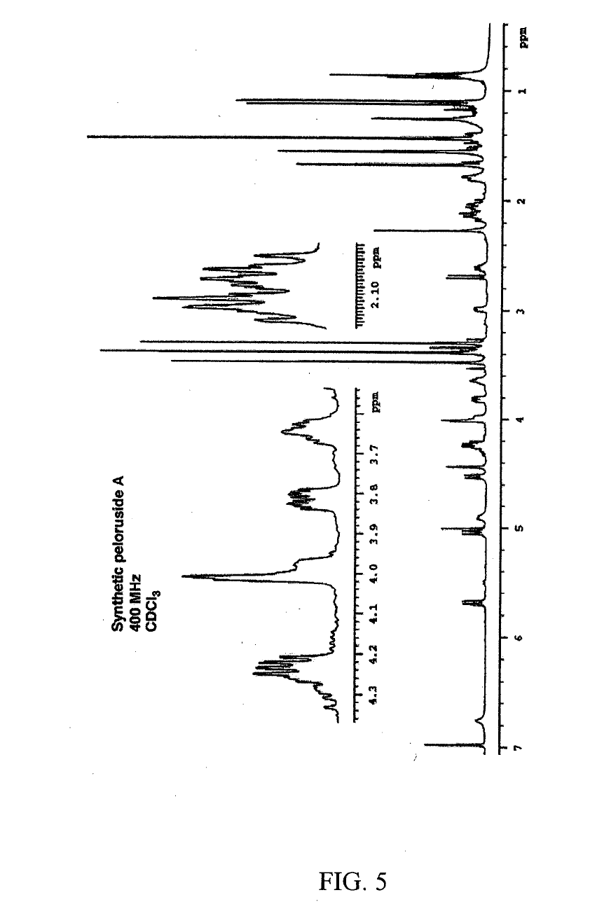

The synthetically produced Peloruside A with the 2R, 3S, 5S, 7S, 8S, 9S, 11R, 13R, 15R, 18S absolute configuration of the present invention had spectroscopic properties (¹H and ¹³C NMR shown in FIGS. 4 and 5; IR, HRMS) identical to the natural isolate (see West, Northcote and Battershill, 2000, J. Org. Chem. 65:445-449 for the natural isolate). However, the optical rotation of this enantiomeric form of Peloruside A with 2R, 3S, 5S, 7S, 8S, 9S, 11R, 13R, 15R, 18S absolute configuration shown in FIG. 1 was of opposite sign ([α]²³_{D}=−16; c=0.12 in CH₂Cl₂) to the one reported for the natural product ([α]²³_{D}=+16; c=0.3 in CH₂Cl₂; see West, Northcote and Battershill, 2000, J. Org. Chem. 65:445-449). The synthetically produced Peloruside A with the 2S, 3R, 5R, 7R, 8R, 9R, 11S, 13S, 15S, 18R absolute configuration of the present invention had also spectroscopic properties (¹H and ¹³C NMR shown in FIGS. 4 and 5; IR, HRMS) identical to the natural isolate (see West, Northcote and Battershill, 2000, J. Org. Chem. 65:445-449 for the natural isolate). This time, however, the optical rotation of this enantiomeric form of Peloruside A with 2S, 3R, 5R, 7R, 8R, 9R, 11S, 13S, 15S, 18R absolute configuration shown in FIG. 2 was of identical sign ([α]²³_{D}=+15.5; c=0.2 in CH₂Cl₂) to the one reported for the natural product ([α]²³_{D}=+16; c=0.3 in CH₂Cl₂; see West, Northcote and Battershill, 2000, J. Org. Chem. 65:445-449), which established the absolute configuration of the dextrarotatory (+)-Peloruside A as 2S, 3R, 5R, 7R, 8R, 9R, 11S, 13S, 15S, 18R.

The present invention has determined the absolute configuration of (+)-Peloruside A as 2S, 3R, 5R, 7R, 8R, 9R, 11S, 13S, 15S, 18R as shown in FIG. 2. The synthesis of (−)-and (+)-Peloruside A as described in Examples 1 and 2 respectively.

An embodiment of the invention is a synthetic compound having the NMR signatures of FIG. 4 and 5, wherein the compound is dextrarotary and wherein the compound has microtubule-stabilizing activity.

Another embodiment of the invention is a compound of Formula I and compositions comprising a compound of Formula I:

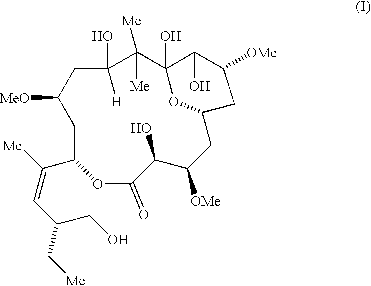

A further embodiment of the invention is a compound of the Formula II and compositions comprising a compound of Formula II:

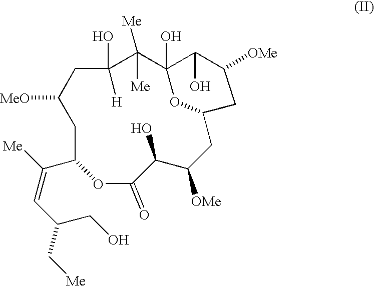

Another embodiment of the invention is a compound of the Formula III and compositions comprising a compound of Formula III:

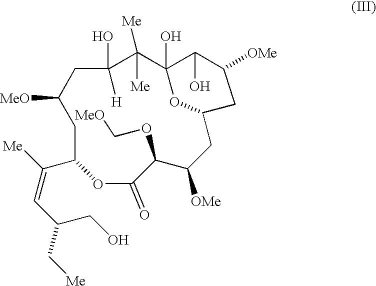

Still another embodiment of the invention is a compound of Formula IV and compositions comprising a compound of Formula IV:

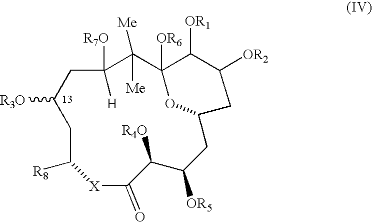

where R₁, R₂, R₃, R₄, R₅ and R₆ can be the same or different and include: H, Me, alkyl, funtionalized alkyl (functionalization may include, inter alia, heteroatoms, halides, aryl, heteroaryl), and where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl). The configuration at the carbon bearing the OR₁, OR₂, and OR₃ substituents can have the R— or S-configuration.

In still a further embodiment, the invention provides a compound of Formula V and compositions comprising a compound of Formula V:

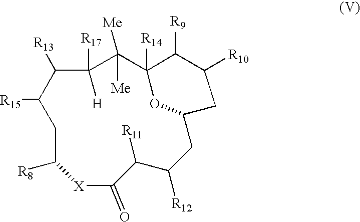

where R₁₃═H or Me, where R₁₄, R₁₇ can be the same or different and include H, OH, or OR, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅ and OR₅ substituents can be of the R— or S-configuration.

The invention also relates to a compound of Formula VI and to compositions comprising a compound of Formula VI:

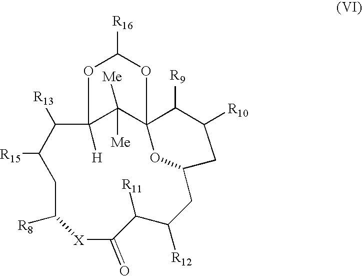

where R₃═H or Me, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₅, R₁₆ and OR₅ substituents can be of the R— or S-configuration.

The invention also relates to a compound of Formula VII and to compositions comprising a compound of Formula VII:

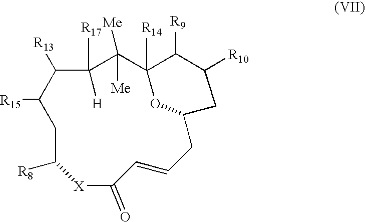

where R₁₃═H or Me, where R₁₄, R₁₇ can be the same or different and include H, OH, or OR, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅ substituents can be of the R— or S-configuration.

The invention also relates to a compound of Formula VIII and to compositions comprising a compound of Formula VIII:

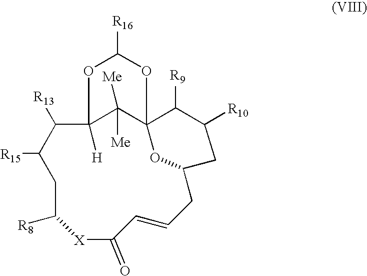

where R₁₃═H or Me, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅, R₁₆ substituents can be of the R— or S-configuration.

The invention also relates to a compound of Formula IX and to compositions comprising a compound of Formula IX:

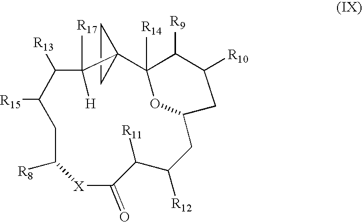

where R₁₃═H or Me, where R₁₄, R₁₇ can be the same or different and include H, OH, or OR, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅ and OR₅ substituents can be of the R— or S-configuration.

The invention also relates to a compound of Formula X and to compositions comprising a compound of Formula X:

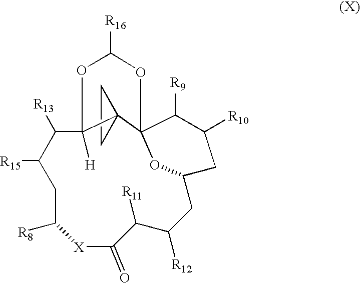

where R₁₃═H or Me, where R₉, R₁₀, R₁₁, R₁₅ can be the same or different and include H, Me, OR, where R and R₅ can be the same or different and includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₁, R₁₃, R₁₅, R₁₆ and OR₅ substituents can be of the R— or S-configuration.

The invention also relates to a compound of Formula XI and to compositions comprising a compound of Formula XI:

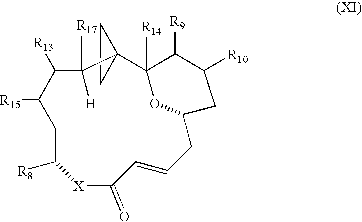

where R₁₃═H or Me, where R₁₄, R₁₇ can be the same or different and include H, OH, or OR, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈═H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅ substituents can be of the R— or S-configuration.

The invention also relates to a compound of Formula XII and to compositions comprising a compound of Formula XII:

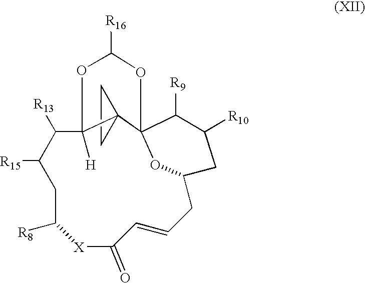

where R₁₃═H or Me, where R₉, R₁₀, R₁₅ can be the same or different and include H, Me, OR, where R includes H, Me, alkyl, or functionalized alkyl (functionalization may include, inter alia, heteroatoms, halides, aryl, heteroaryl), where R₈, R₁₆ can be the same or different and include H, aryl, heteroaryl, alkyl, functionalized alkyl, alkenyl, functionalized alkenyl, alkynyl, functionalized alkynyl (functionalization may include, inter alia, heteroatoms, halides, aryl, heteroaryl), and where X═O or NH. The configuration at the carbons bearing the R₉, R₁₀, R₁₃, R₁₅, R₁₆ substituents can be of the R— or S-configuration.

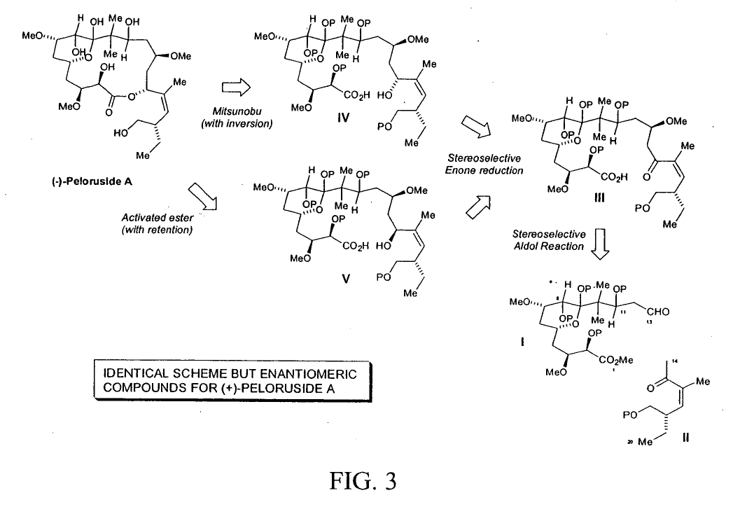

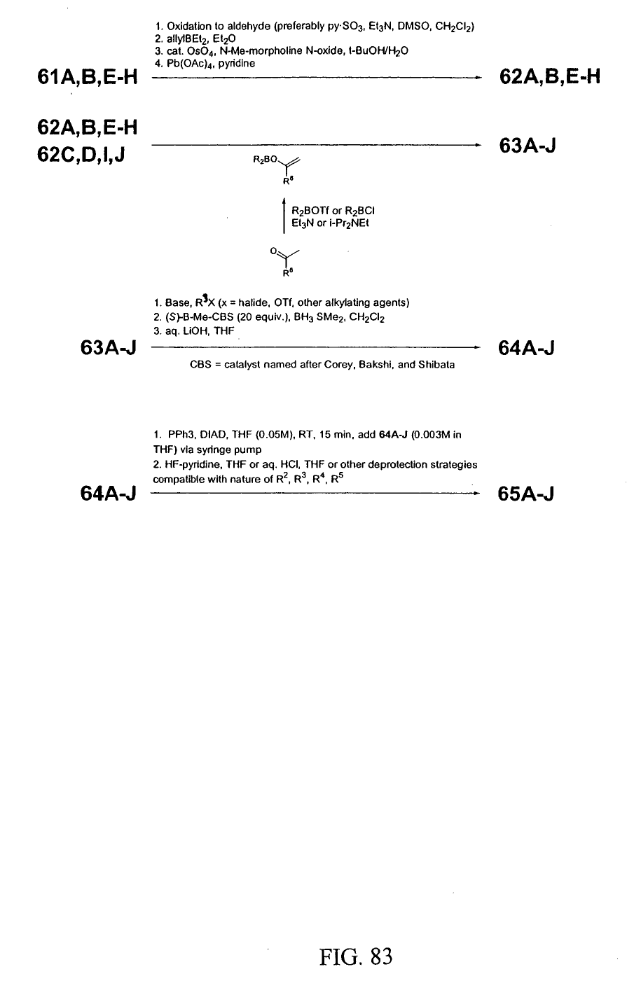

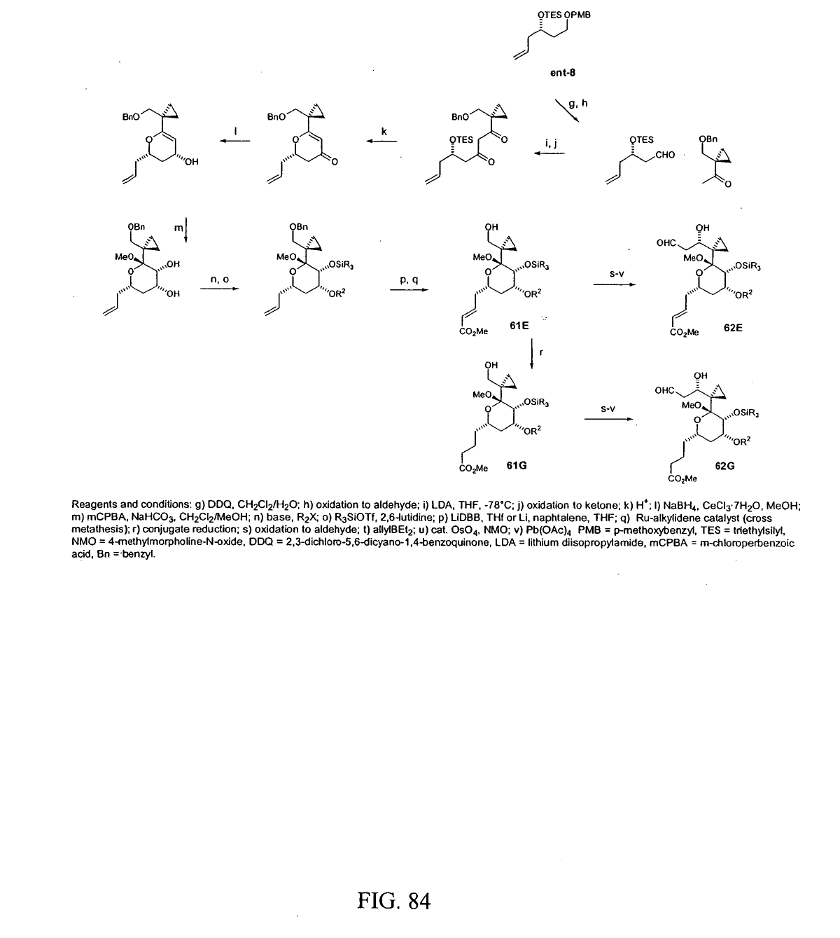

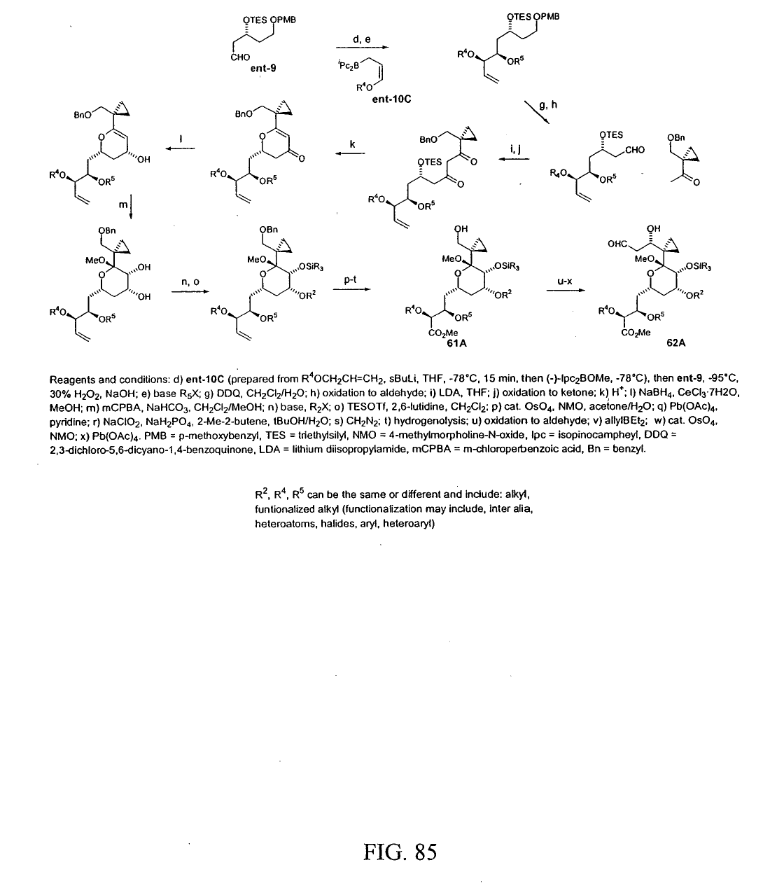

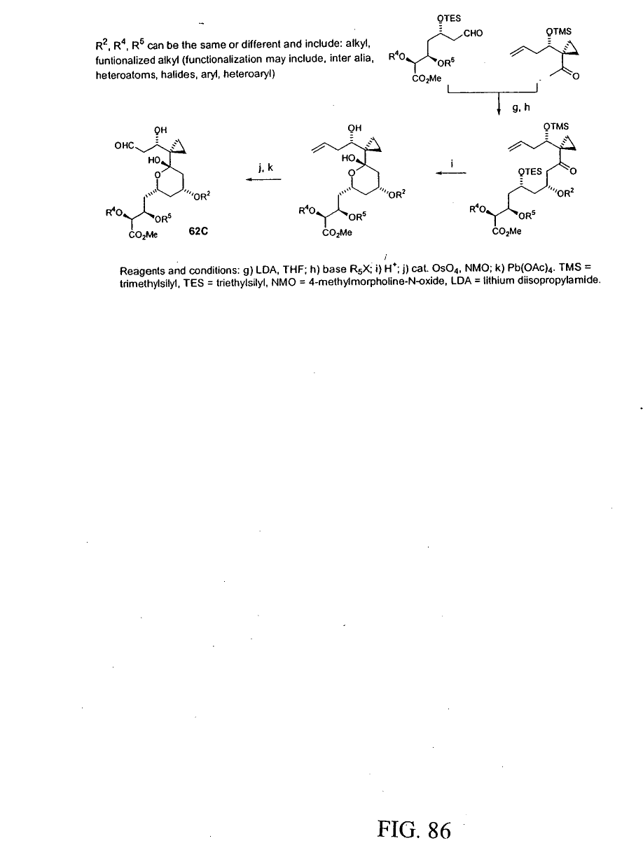

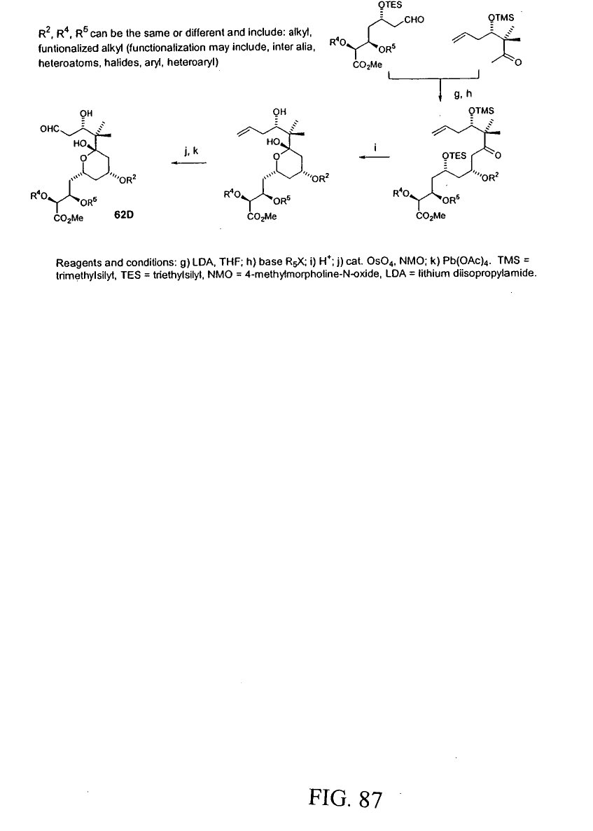

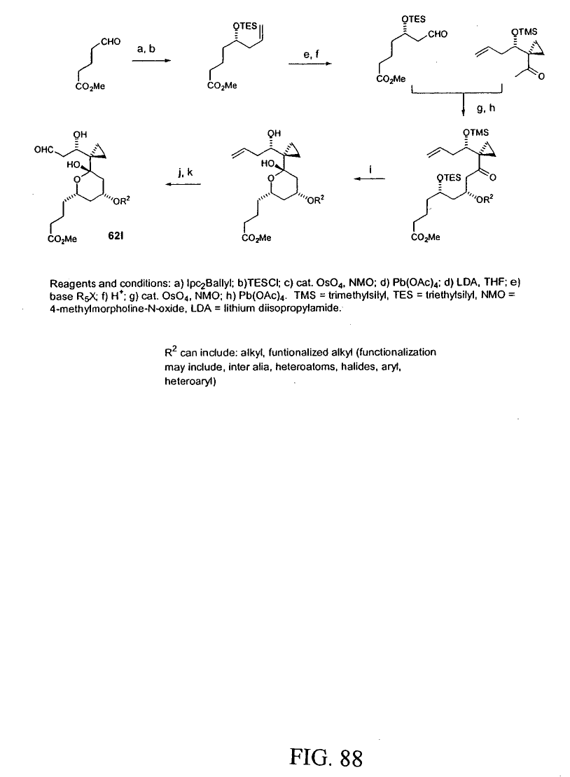

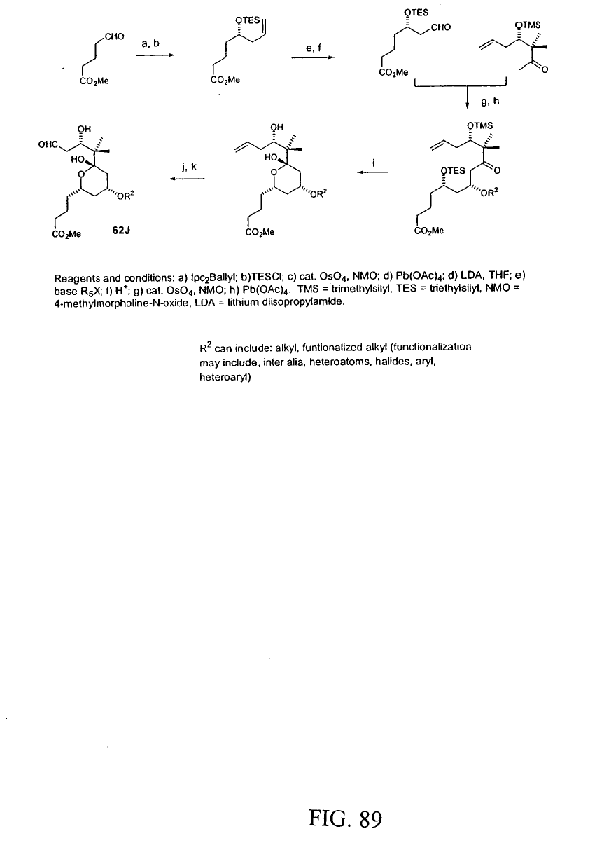

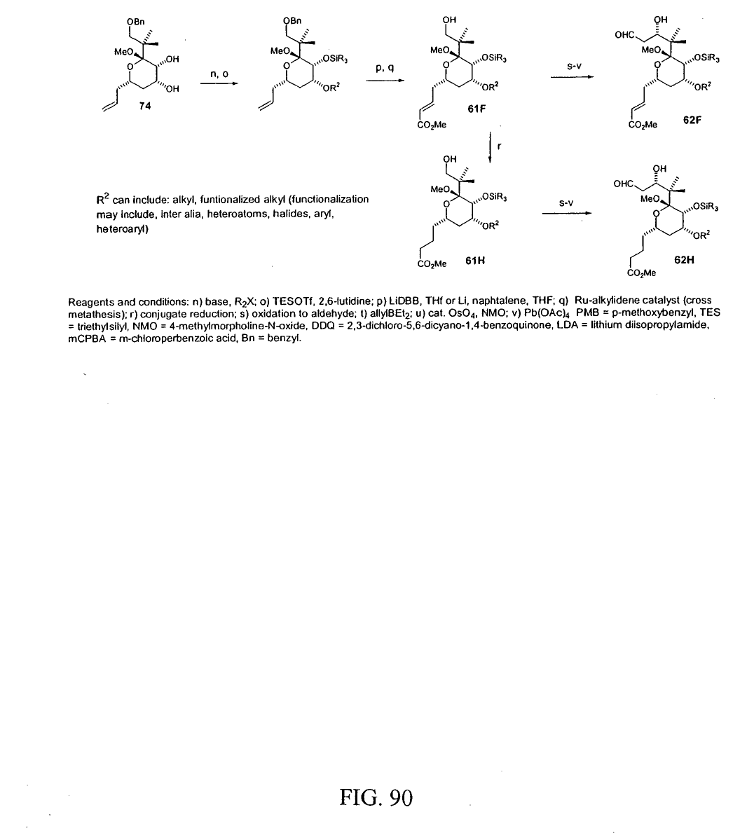

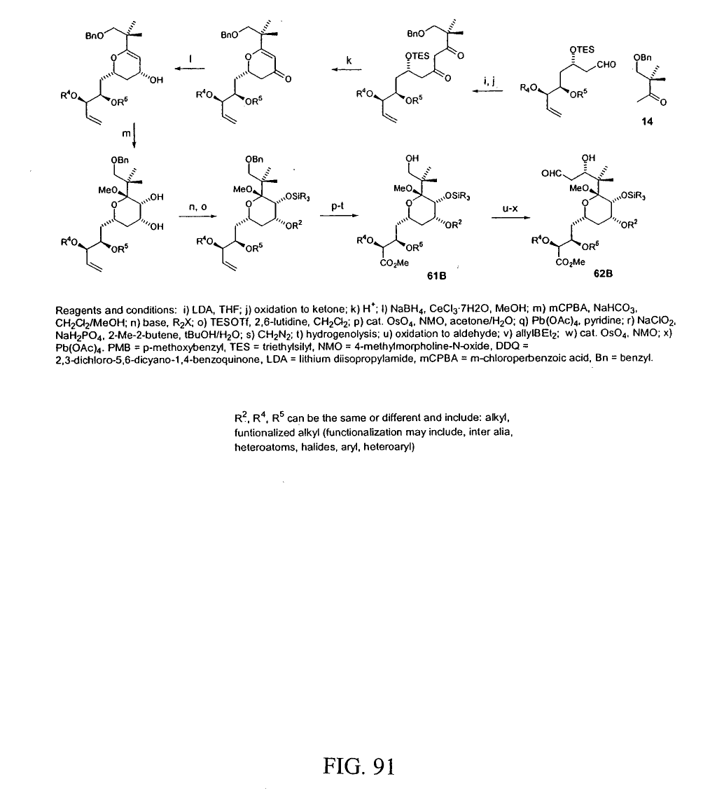

The present invention provides a process for preparing a macrolactone Peloruside comprising the steps of a) synthesizing a pyran ring containing a first substituent having a carboxylic acid group and a second substituent having a hydroxyl group; and b) reacting the carboxylic acid group with the hydroxyl group to form a macrolactone. The scheme for synthesizing the compounds of the present invention are shown in FIGS. 3 and 83.

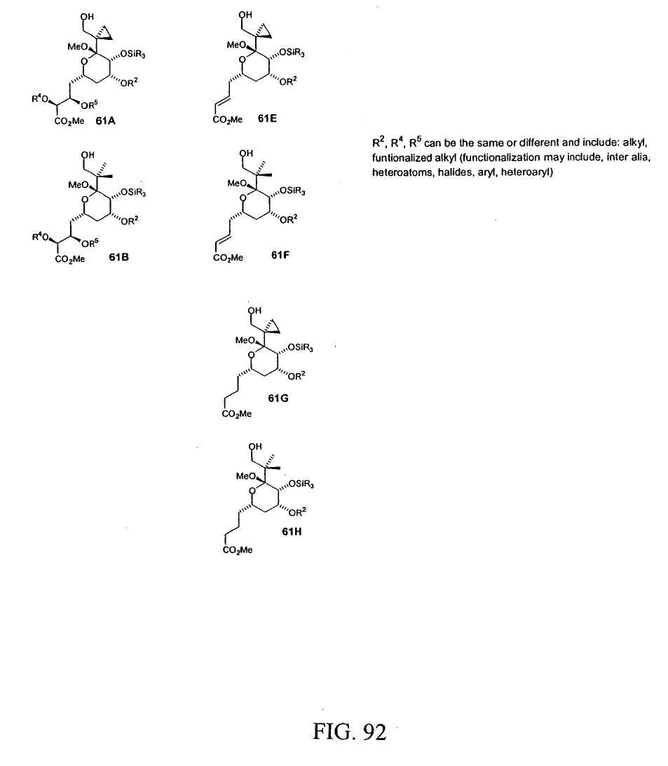

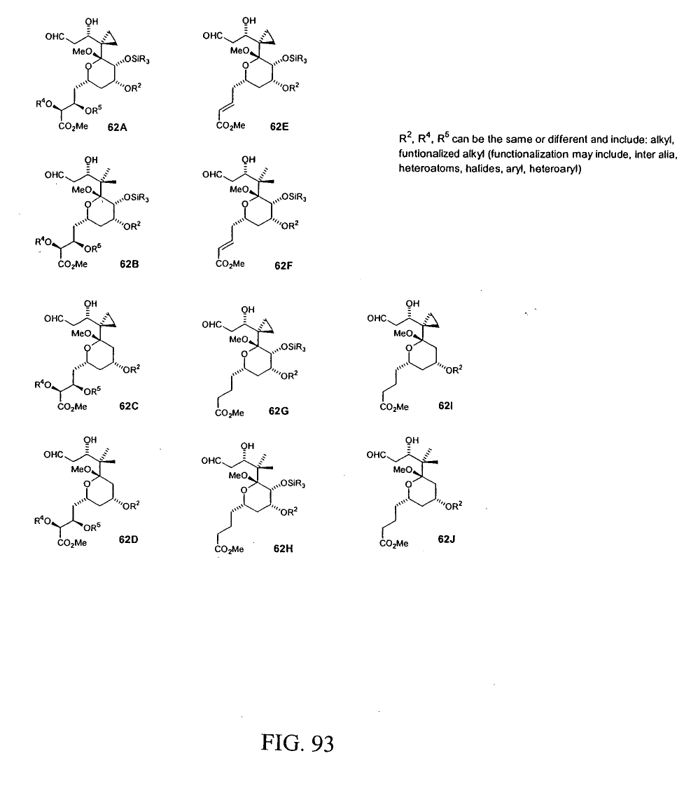

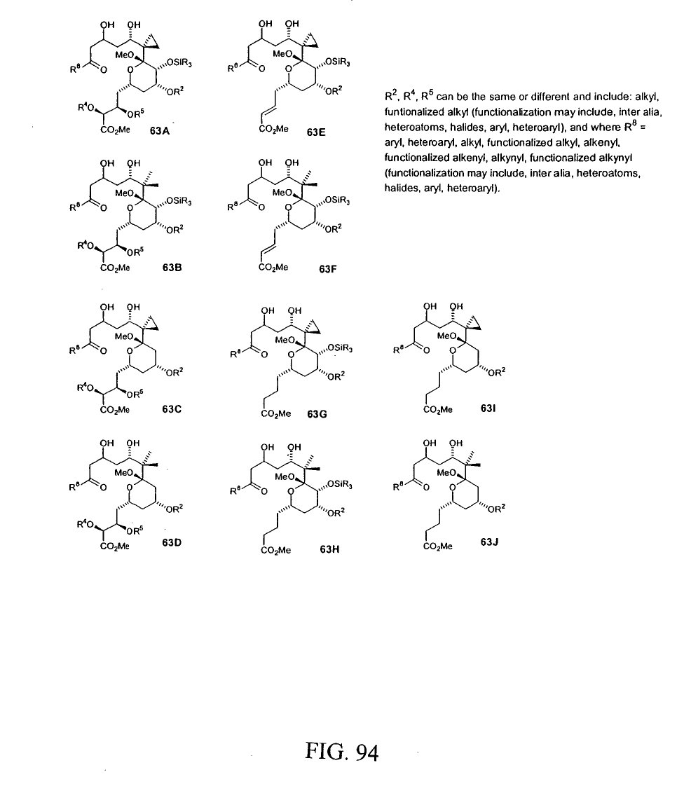

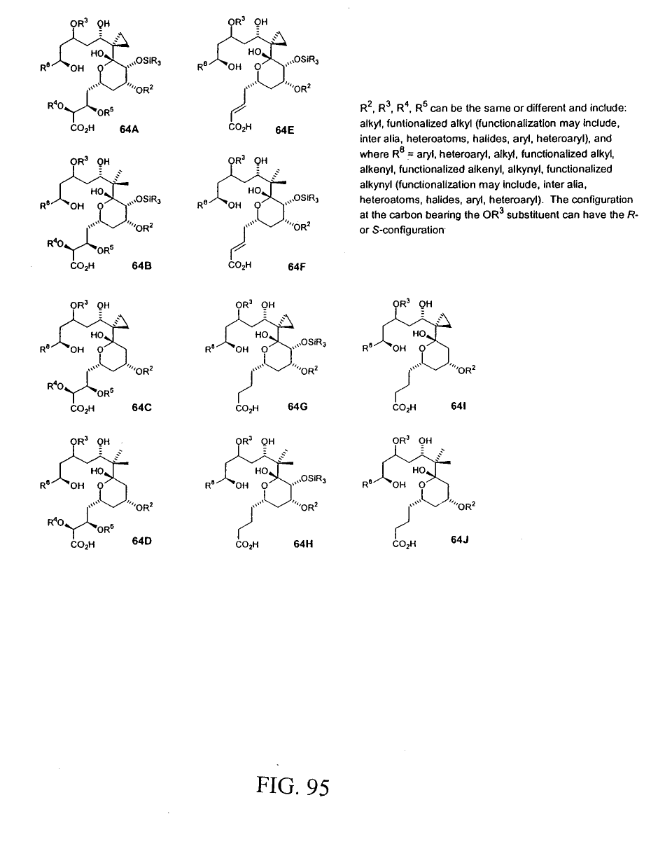

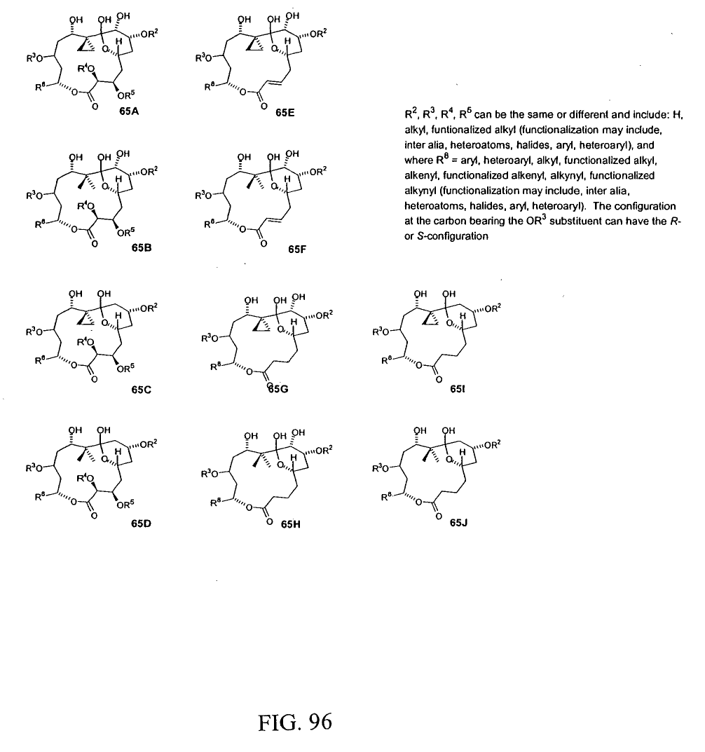

The present invention also provides for compounds identified as intermediates in the synthesis of Peloruside A. They include the compounds 23, 31, 32a, 32b, 33a, 33b, 34a, and 34b, as discussed in Example 2. In addition, the present invention relates to the identification of compounds 61A, 61B, 61E, 61F, 61G, 61H, 62A-62J, 63A-63J, 64A-64J, and 65A-65J, as shown in FIGS. 92, 93, 94, 95 and 96, respectively.

The compounds of the present invention are useful for stabilizing microtubule formation. They are cytostatic and cytotoxic and can inhibit the growth of proliferating cells, and preferably tumor cells. Therefore, the compounds of the present invention are useful for inhibiting the growth of tumor cells for treating cancer. The compounds of the present invention possess microtubule-stabilizing activity similar to that of Taxol® and may therefore also be useful for inhibiting the growth of tumor cells which have become resistant to Taxol®.

Compounds may be tested for activity in the yeast Saccharomyces cervisiae. Yeast-based screening methods are fast and would allow for the rapid identification of macrolactones capable of stabilizing microtubule formation. To alleviate potential problems of drug resistance, Δerg6 mutant strains, displaying reduced multi-drug resistance and more permeability to drugs due to more fluid membranes, can be used.

In a preferred embodiment, the temperature used for synthesis is, except where stated to be different, in a range from about −78° C. to about 125° C., preferably 0° C. to 90° C.

The present invention provides for pharmaceutical compositions comprising the compounds of the present invention. Aqueous pharmaceutical compositions of the present invention comprise an effective amount of a macrolactone Peloruside of the present invention or pharmaceutically acceptable salt thereof, dissolved and/or dispersed in a pharmaceutically acceptable carrier and/or aqueous medium.

As used herein, “physiologically and/or pharmaceutically acceptable carrier” includes any and/or all solvents, dispersion media, coatings, antibacterial and/or antifungal agents, isotonic and/or absorption delaying agents and/or the like. The use of such media and/or agents for pharmaceutically active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of aqueous compositions that contain a therapeutically effective amount of the macrolactone Pelorusides of the invention or pharmaceutically acceptable salts thereof as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

Pelorusides of the present invention can be formulated into a composition in a neutral and/or salt form. Pharmaceutically acceptable salts, include the acid addition salts and/or which are formed with inorganic acids such as, for example, hydrochloric and/or phosphoric acids, and/or such organic acids as acetic, oxalic, tartaric, mandelic, and/or the like.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

The macrolactone pelorusides of the present invention may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered. It is believed that dosages may be similar to those used for Taxol®.

The present invention also provides kits comprising the pelorusides of the present invention or pharmaceutically acceptable salts thereof. Such kits will generally contain, in suitable container means, a pharmaceutically acceptable formulation of the Pelorusides of the present invention in a pharmaceutically acceptable formulation.

The container means will generally include at least one vial, test tube, flask, bottle, syringe and/or other container means, into which the macrocyclic lactones of the present invention formulation are placed, preferably, suitably allocated. The kits may also comprise a second container means for containing a sterile, pharmaceutically acceptable buffer and/or other diluent.

In order to increase the effectiveness of the macrolactones of the present invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the Pelorusides of the present invention and other agent(s) at the same time.

Cancer therapies may include a variety of combination therapies with both chemical and radiation based treatments. The compounds may also be used together with immunotherapy. The compounds of the present invention may also be combined with gene therapy in which a therapeutic polynucleotide is administered before, after, or at the same time as the Peloruside of the present invention. Delivery of a vector encoding one of the following gene products will have a combined anti-hyperproliferative effect on target tissues. The compounds of the present invention may further be used in combination with surgery.

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers.

Other objects, features and advantages of the present invention will become apparent from the following specific examples. It should be understood, however, that the detailed description and the specific examples, while indicating preferred embodiments of the invention, are given by way of illustration only, since various changes and modifications within the spirit and scope of the invention will become apparent to those skilled in the art.

### EXAMPLES

### Example 1

### Synthesis of (−)-Peloruside A

Strategically, a late stage aldol coupling between a fully functionalized C1-C13 aldehyde I with a C14-C20 methyl ketone II was chosen. See FIG. 3. This approach was chosen because it was believed that access to diastereomeric seco-acids IV and V, via reduction of a common C15 ketone III, increases the odds for a successful macrocyclization by accessing two mechanistically distinct pathways, namely acylative or invertive macrolactonization. A concise, enantioselective synthesis of fragment II is outlined in Scheme 1 below. The known homoallylic alcohol 2, prepared in enantiopure form according to the procedure described by Xu et al. (J. Am. Chem. Soc. 119:10302-10316 (1997)) was acylated with methacryloyl chloride followed by ring-closing olefin metathesis with Grubb's second generation catalyst 4. Chatterjee et al., 2000, J. Am. Chem. Soc. 383-3784. The resulting lactone 5 then provides a valuable entry to the (Z)-trisubstituted enone 6 by treatment with methyllithium and silylation of the primary alcohol.

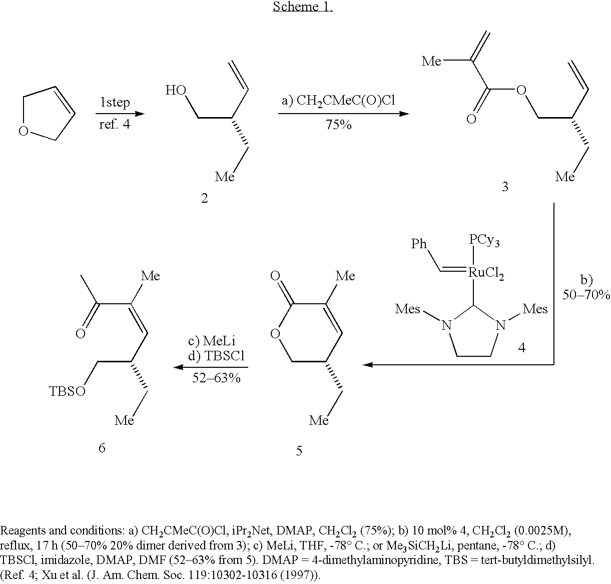

Scheme 2 below shows the approach towards the densely functionalized tetrahydropyranyl containing C1-C13 fragment. Low temperature addition of aldehyde 9 (from 7 as shown; Smith et al., 2001, J. Am. Chem. Soc., 123:10942-10953) to (Z)-alkoxyallylborane 10 produced the desired homoallyl alcohol diastereomer 11 (>10:1). See Brown et al., 1988, J. Am Chem. Soc. 110:1535-1538; and Smith et al., 1993, J. Am. Chem. Soc. 115:7612-7624. The relative 1,3-syn stereochemical relationship was demonstrated by ¹³C NMR analysis of the acetonide derivative 13 (isopropylidene carbon resonances at 20.0, 30.3, and 98.8 ppm). See Rychnovsky, Rogers & Yang, 1993, J. Org. Chem. 58:3511-3515. Compound 11 was advanced to aldehyde 15 via methylation (→12), oxidative debenzylation (DDQ), and oxidation of the resulting primary alcohol. Addition of aldehyde 15 to a cold solution of the lithium enolate derived from 14 and Dess-Martin oxidation of the resulting aldol product fashioned β-diketone 16. Transformation to dihydropyranone 17 ensued smoothly by stirring 16 in an acidic toluene solution (TES-deprotection/cyclodehydration). It proved critical to perform the subsequent Luche (Luche, Rodriguez-Hahn & Crabbé, 1978, J. Chem. Soc., Chem. Commun. 601-602) reduction at −30° C. to prevent decomposition (→18). Most satisfactorily, hydroxy-directed epoxidation of crude 18 followed by in situ methanolysis of the incipient glycal-epoxide produced one single glycoside 19 in high yield (72% from 17). See, e.g., Wender et al., 1998, J. Am. Chem. Soc., 120:4534-4535; and Evans et al., 1999, J. Am. Chem. Soc. 121:7540-7552. Sequential methylation (eq. OH)/silylation (ax. OH) of the α-diol (→20), oxidative transformation of the double bond to a C1 carboxylic acid, and diazomethane treatment furnished methyl ester 21. Access to the C11 aldehyde 22 by hydrogenolytic removal of the benzyl ether and oxidation set the stage for progression via C11-C12 carbon bond formation. Concerns related to the feasibility of this critical bond-forming event (see e.g., Wender et al., 1998, J. Am. Chem. Soc., 120:4534-4535; Evans et al., 1999, J. Am. Chem. Soc. 121:7540-7552; and Kageyama et al., 1990, J. Am. Chem. Soc. 112:7407-7408) seemed justified when various attempts to add carbon-nucleophiles to the sterically demanding aldehyde 22 met with failure. That is allylmagnesium bromide reacted competitively with the methyl ester. Lewis-acid catalyzed allyl-transfer (allylSiMe₃, TiCl₄ or BF₃Et₂O) or acetate aldol reactions (lithium or boron enloates) resulted in recovery of starting material. Ultimately, a highly efficient allyl-transfer was achieved with allyldimethylborane. Remarkably, a single alcohol diastereomer 23 was produced with C11 configuration ascertained by NOE analysis of spirocyclic ketal 24 and advancement of 23 to Peloruside A (vide infra). This fortunate sterochemical outcome was not predicted a priori.

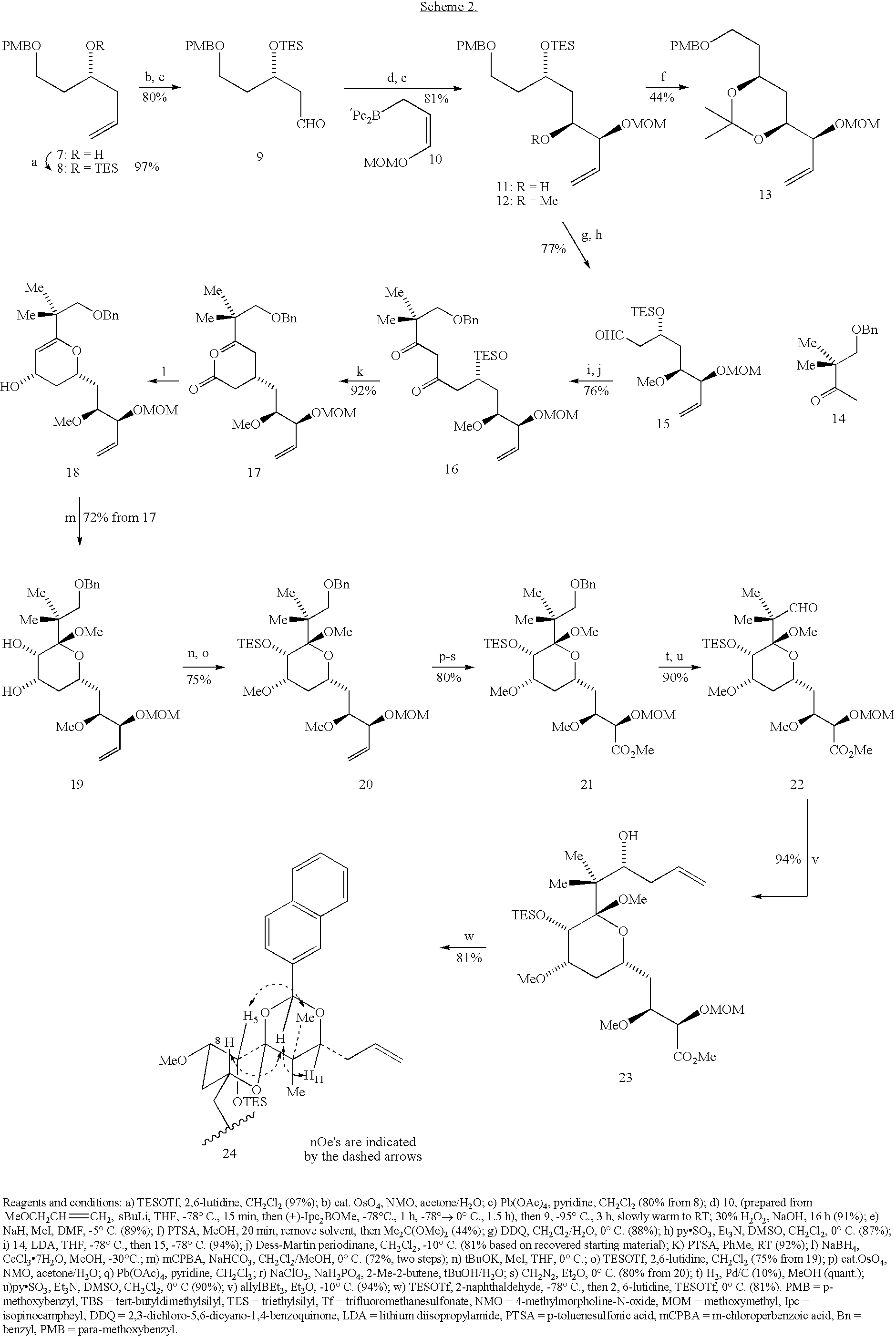

As shown in Scheme 3 below, β-hydroxy aldehyde 31 (from 23 as shown) was a viable partner for coupling with the enolborinate derived from 6 (Et₂BOTf, iPr₂NEt, −78° C., CH₂Cl₂). Most gratifyingly, a separable 2:1 mixture of C13 epimers 32a,b was generated in 87% yield. At this stage, the stereochemical assignment was left unresolved, and both isomers 32a/32b were advanced individually. Surprisingly, methylation of these compounds occurred with concomitant hydrolysis to the C9 hemiketals 33a,b. The minor isomer 33b was separated from the major isomer 33a and both were advanced individually to macrocyclic lactones. Asymmetric reduction of enone 33b using (S)—or (R)—B-Me-CBS-oxazaborolidine and H₃B.SMe₂ (80-94% yield, ratio's >12:1) afforded allylic alcohols 35b and 34b respectively. See, e.g., Corey & Helal, 1998, Angew. Chem. Int. Ed. 37:1986-2012; and Corey & Helal, 1998, Angew. Chem. Int. Ed. 110:2092-2118). Note that no concomitant reduction of the C9 hemiketal was observed under these conditions. The corresponding carboxylic acids 34b/35b were liberated quantitatively by saponification. Re-esterification with CH₂N₂ demonstrated that no epimerization had occurred. The (Z)-configuration of the double bond was maintained throughout the synthesis and verified by NOE.

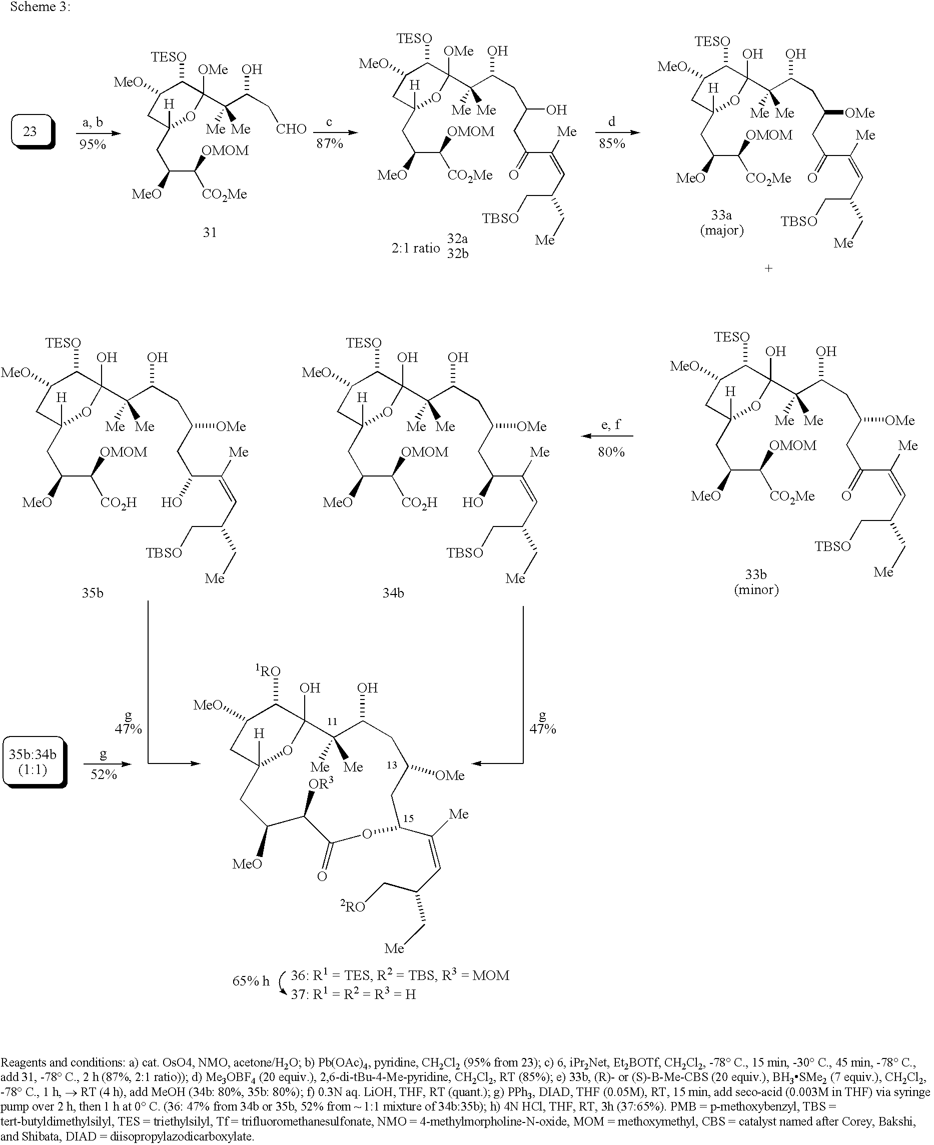

Both epimers 34b and 35b were explored as substrates for a Mitsunobu-type macrolactonization. Remarkably, the same lactone 36 was produced from either 34b, 35b, or an equimolar mixture of both. No diasteromeric or transposed (allylic) macrolactones were detected by NMR analysis of the non-purified mixture. This lactone was shown to have a C13 epimeric relationship to Peloruside A (vide infra). Clues about the sterochemistry of 36 were provided at this point by virtue of mutual NOE enhancements between the H11-H13, and H13-H15 proton pairs. This is believed to represent the first documented case of a configuration-dependent mechanistic switch for a Mitsunobu lactonization. For selected examples of Mitsunobu esterifications leading to retention of configuration when hindered alcohols are involved, see, Ahn & DeShong, 2002, J. Or. Chem. 67:1754-1759; and Smith, Safanov, & Corbett, 2002, J. Am. Chem. Soc. 124:11102-11113.

The structure of 36 was assigned retrospectively, i.e., after the completion of (−)-Peloruside A from precursor 35a (vide infra). Initial evidence for a C13 epimeric configuration surfaced however, when the ¹H NMR data of a globally deprotected lactone (i.e., 37), attained through the action of aqueous HCl in THF, did not match those reported for the natural product. By inference, it was realized that Peloruside A would fortuitously derive from the major aldol diastereomer 33a (Scheme 4).

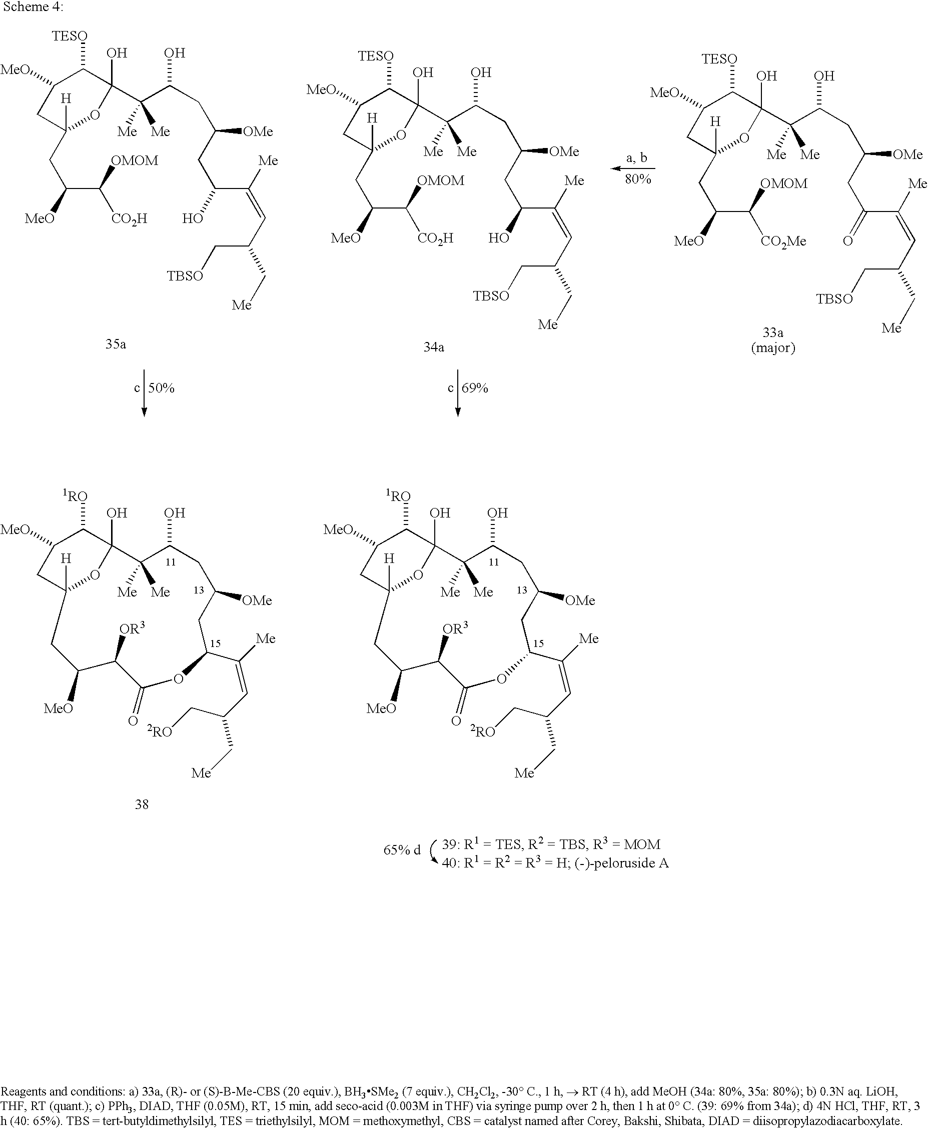

Asymmetric reduction of enone 33a using (S)—or (R)—B-Me-CBS-oxazaborolidine and H₃B.SMe₂ (80-94% yield, ratio's >12:1) afforded allylic alcohols 35a and 34a respectively. See, e.g., Corey & Helal, 1998, Angew. Chem. Int. Ed. 37:1986-2012; and Corey & Helal, 1998, Angew. Chem. Int. Ed. 110:2092-2118). Note that no concomitant reduction of the C9 hemiketal was observed under these conditions. The corresponding carboxylic acids 34a/35a were liberated quantitatively by saponification. Re-esterification with CH₂N₂ demonstrated that no epimerization had occurred. The (Z)-configuration of the double bond was maintained throughout the synthesis and verified by NOE. In the event, Mitsunobu lactonization of 34a (69%) followed by simultaneous cleavage of the MOM and silyl protecting groups in lactone 39 (65%), did produce a pure compound (40) with spectroscopic properties (¹H and ¹³C NMR, IR, HRMS) identical to the natural isolate. However, the optical rotation of synthetic Peloruside A (40) was of the opposite sign ([α]²³_{D}=−16; c=0.12 in CH₂Cl₂) to the one reported for the natural product ([α]²⁰η=−16; c=0.12 in CH₂Cl₂). Also, synthetic 40 (up to 10 μM) had no effect on the growth of cultured human tumor cell lines (SK-MEL-5, HeLa) and did not polymerize tubulin dimers into microtubule polymers. Based on these results, the absolute configuration of synthetic (−)-Peloruside A (compound 40) was assigned as 2R, 3S, 5S, 7S, 9S, 11R, 13R, 15R, 18S. By inference, natural (+)-Peloruside A was assigned the 2S, 3R, 5R, 7R, 8R, 9R, 11S, 13S, 15S, 18R absolute configuration. This was confirmed by the synthesis of (+)-Peloruside A (see example 2 below).

### Example 2

### Synthesis of (+)-Peloruside A

The synthesis method described above was useful for preparing (+)-Peloruside A. The synthesis utilizes mirror image starting materials and reagents, more specifically, the mirror image of compounds 2, 7 and 10, i.e. ent-2, ent-7 and ent-10. This synthesis was reduced to practice.

For producing (+)-Peloruside A, the homoallylic alcohol ent-2, prepared in enantiopure form according to the procedure described by Xu et al. (J. Am. Chem. Soc. 119:10302-10316 (1997)) was acylated with methacryloyl chloride followed by ring-closing olefin metathesis with Grubb's second generation catalyst 4 (Scheme 5). Chatterjee et al., 2000, J. Am. Chem. Soc. 383-3784. The resulting lactone ent-5 then provides a valuable entry to the (Z)-trisubstituted enone ent-6 by treatment with methyllithium and silylation of the primary alcohol.

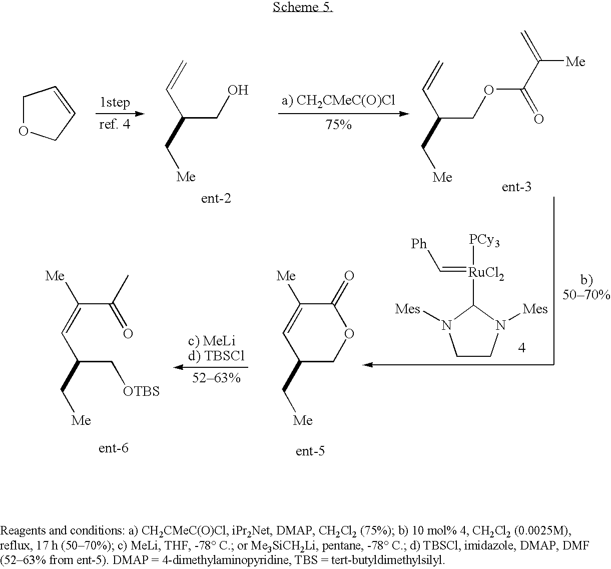

Scheme 6 below shows the approach towards producing the densely functionalized tetrahydropyranyl containing C1-C13 fragment. Low temperature addition of aldehyde ent-9 (from ent-7 as shown; Smith et al., 2001, J. Am. Chem. Soc., 123:10942-10953) to (Z)-alkoxyallylborane ent-10 produced the desired homoallyl alcohol diastereomer ent-11 (>10:1). See Brown et al., 1988, J. Am. Chem. Soc. 110:1535-1538; and Smith et al., 1993, J. Am. Chem. Soc. 115:7612-7624. Compound ent-11 was advanced to aldehyde ent-15 via methylation (→ent-12), oxidative debenzylation (DDQ), and oxidation of the resulting primary alcohol. Addition of aldehyde ent-15 to a cold solution of the lithium enolate derived from 14 and Dess-Martin oxidation of the resulting aldol product fashioned β-diketone ent-16. Transformation to dihydropyranone ent-17 ensued smoothly by stirring ent-16 in an acidic toluene solution (TES-deprotection/cyclodehydration). It proved critical to perform the subsequent Luche (Luche, Rodriguez-Hahn & Crabbé, 1978, J. Chem. Soc., Chem. Commun. 601-602) reduction at −30° C. to prevent decomposition (→ent-18). Most satisfactorily, hydroxy-directed epoxidation of crude ent-18 followed by in situ methanolysis of the incipient glycal-epoxide produced one single glycoside ent-19 in high yield (72% from ent-17). See, e.g., Wender et al., 1998, J. Am. Chem. Soc., 120:4534-4535; and Evans et al., 1999, J. Am. Chem. Soc. 121:7540-7552. Sequential methylation (eq. OH)/silylation (ax. OH) of the α-diol (→ent-20), oxidative transformation of the double bond to a C1 carboxylic acid, and diazomethane treatment furnished methyl ester ent-21. Access to the C11 aldehyde ent-22 by hydrogenolytic removal of the benzyl ether and oxidation set the stage for progression via C11-C12 carbon bond formation. A highly efficient allyl-transfer was achieved with allyldimethylborane. A single alcohol diastereomer ent-23 was produced.

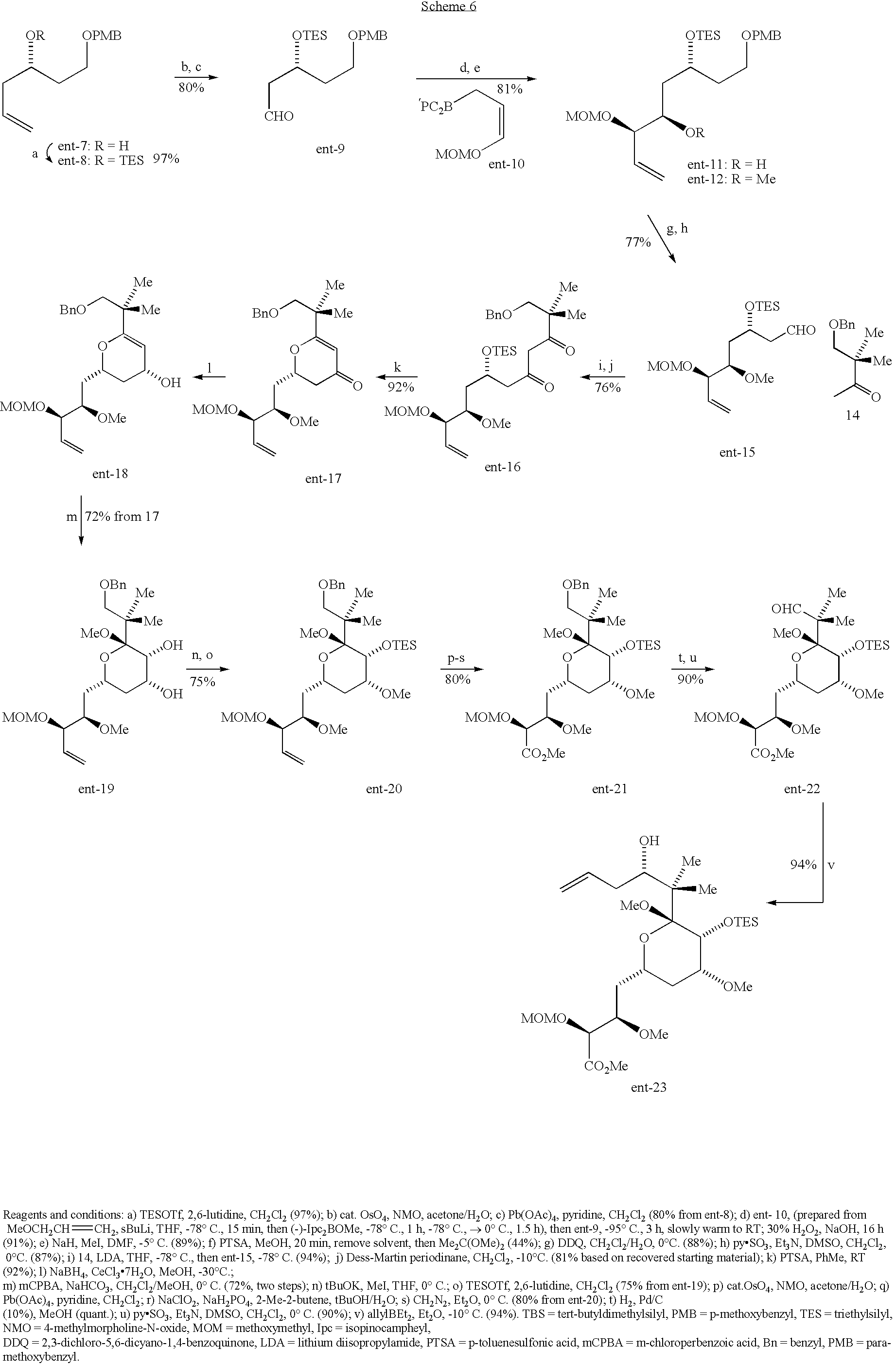

An alternative synthesis of compound ent-20 is shown in Scheme 6b below. The experimental procedures for the synthesis and characterization of compounds 70-77 are provided in the experimentals section.

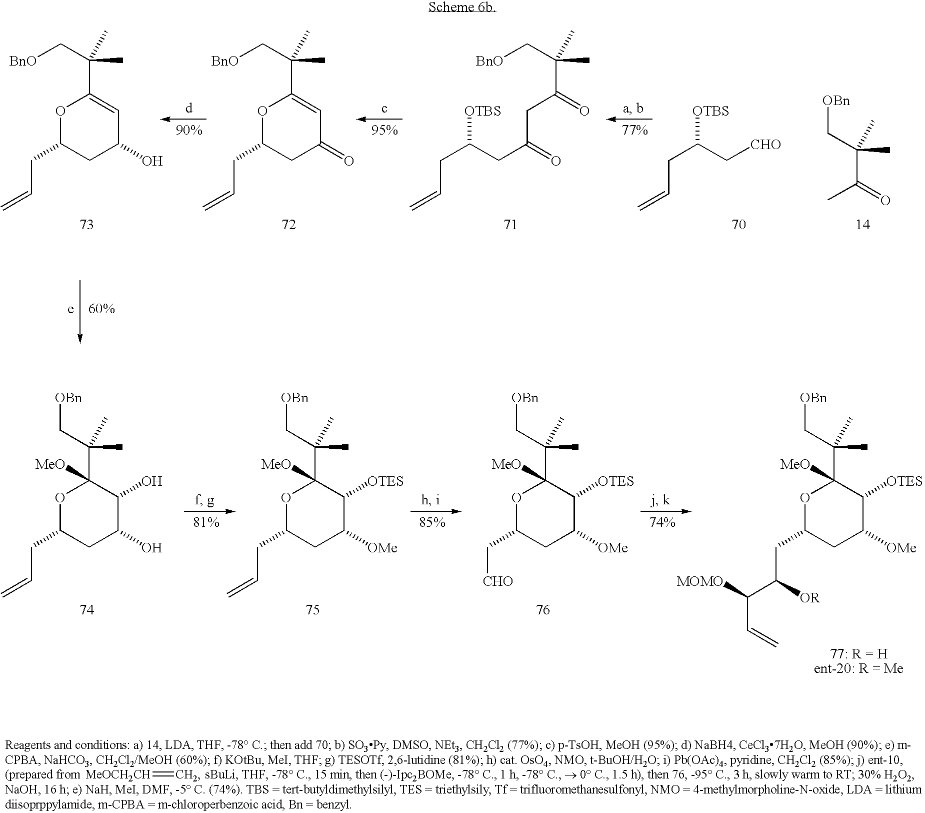

As shown in Scheme 7 below, β-hydroxy aldehyde ent-31 (from ent-23 as shown) was a viable partner for coupling with the enolborinate derived from ent-6 (Et₂BOTf, iPr₂NEt, −78° C., CH₂Cl₂). Most gratifyingly, a separable 2:1 mixture of C13 epimers ent-32a,b was generated in 87% yield. Methylation of these compounds occurred with concomitant hydrolysis to the C9 hemiketals ent-33a,b. The minor isomer ent-33b was separated from the major isomer ent-33a and both were advanced individually to macrocyclic lactones. Asymmetric reduction of enone ent-33b using (S)—or (R)—B-Me-CBS-oxazaborolidine and H₃B.SMe₂ (80-94% yield, ratio's >12:1) afforded allylic alcohols ent-34b and ent-35b respectively. See, e.g., Corey & Helal, 1998, Angew. Chem. Int. Ed. 37:1986-2012; and Corey & Helal, 1998, Angew. Chem. Int. Ed. 110:2092-2118). Note that no concomitant reduction of the C9 hemiketal was observed under these conditions. The corresponding carboxylic acids ent-34b/ent-35b were liberated quantitatively by saponification. Re-esterification with CH₂N₂ demonstrated that no epimerization had occurred. The (Z)-configuration of the double bond was maintained throughout the synthesis and verified by NOE. Both epimers ent-34b and ent-35b were explored as substrates for a Mitsunobu-type macrolactonization. The same lactone ent-36 was produced from either ent-34b, ent-35b, or an equimolar mixture of both. Global deprotection of lactone ent-36 (aq. HCl, THF) produced Peloruside analog ent-37. Asymmetric reduction of enone ent-33a using (R)—or (S)-B-Me-CBS-oxazaborolidine and H₃B.SMe₂ (80-94% yield, ratio's >12:1) afforded allylic alcohols ent-35a and ent-34a respectively. See, e.g., Corey & Helal, 1998, Angew. Chem. Int. Ed. 37:1986-2012; and Corey & Helal, 1998, Angew. Chem. Int. Ed. 110:2092-2118). Note that no concomitant reduction of the C9 hemiketal was observed under these conditions. The corresponding carboxylic acids ent-34a/ent-35a were liberated quantitatively by saponification. Re-esterification with CH₂N₂ demonstrated that no epimerization had occurred. The (Z)-configuration of the double bond was maintained throughout the synthesis and verified by NOE. In the event, Mitsunobu lactonization of ent-34a (69%) followed by simultaneous cleavage of the MOM and silyl protecting groups in lactone ent-39 (65%), did produce a pure compound (ent-40) with spectroscopic properties (¹H and ¹³C NMR, IR, HRMS) identical to the natural isolate. In addition, the optical rotation of synthetic Peloruside A (ent-40) was of the same sign ([α]²³_{D}=+15.5; c=0.2 in CH₂Cl₂) to the one reported for the natural product ([α]²⁰_{D}=+16; c0.30 in CH₂Cl₂).

Also, synthetic ent-40 did inhibit the growth of various cultured human tumor cell lines and did polymerize tubulin dimers into microtubule polymers. Based on these results, the absolute configuration of synthetic (+)-Peloruside A (compound ent-40) was assigned as 2S, 3R, 5R, 7R, 8R, 9R, 11S, 13S, 15S, 18R, fully confirming the absolute configuration of biologically active (+)-Peloruside A (ent-40).

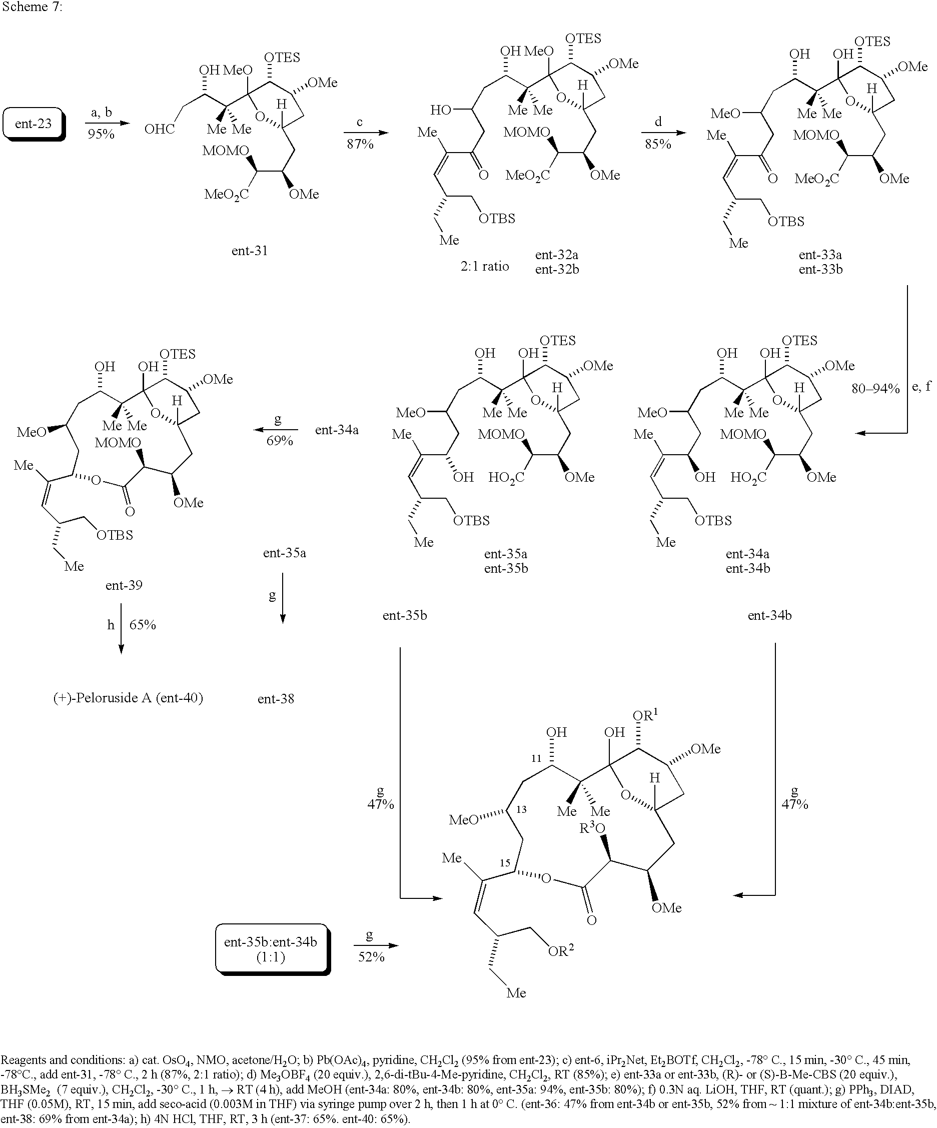

In summary, the first total synthesis of (+)-Peloruside A was accomplished. As shown above, this confirmed the absolute configuration of the dextrorotatory natural product.

### Example 3

### Synthesis of Peloruside Analogs

Various modifications of the synthetic routes described above in examples 1 and 2 can be readily adapted to synthesize peloruside analogs. It should be evident to skilled practicioners of the art that the syntheses of both enantiomeric series of the analogs described below are identical except for the provision that enantiomeric forms of homochiral starting materials, intermediates, reagents and catalysts are to be used for enantiomeric forms of Peloruside analogs.

An example for the synthesis of analogs with C9-C11 hydroxyls protected as a cyclic acetal is provided below. The biologically active forms of these analogs will have an enantiomeric relationship to the ones drawn in the schemes below. Union of fragments 6 and 25 and completion of a Peloruside analog is shown in Scheme 8 below. Mukayama-type aldol reaction of aldehyde 25 with the enolsilane derived from methyl ketone 6 (BF₃.Et₂O, CH₂Cl₂, −78° C.) afforded almost exclusively (14:1) the 1,3-syn aldol product. This stereochemical outcome is contrary to the outcome expected from extensive literature precedents. Evans et al., 1996, J. Am. Chem. Soc. 118:4322-4343. Evidence provided below however, indicates that aldehyde 25 entered this reaction with an unprecedented bias for the formation of the 1,3-syn β-hydroxy ketone 26 (80% yield). Hydroxy ketone 26 was protected as C13 methyl ether 27, followed by CBS reduction (see, e.g., Corey & Helal, 1998, Angew. Chem. Int. Ed. 37:1986-2012; and Corey & Helal, 1998, Angew. Chem. Int. Ed. 110:2092-2118) and ester hydrolysis to reach seco-acid 28. Slow addition of this compound to a premixed solution of PPh₃ and diisopropylazodicarboxylate instigated formation of macrolactone 29 in 40-50% yield (see, e.g., Mitsunobu, 1981, Sythesis 1-28). The stereochemistry of 29 was deduced based on a series of NOE correlations that locate H11, H13 and H15 on the same upper side of the macrolactone ring in agreement with the assigned C13(S) configuration. The synthesis of 29 provides an example of Peloruside spiroacetal analogs, i.e. Peloruside macrocycles that have the C9 and C11 hydroxyl groups incorporated into an acetal ring, and can be prepared according to the general outline of Scheme 8. Note that enantiomeric 29, i.e. ent-29, and analogs will be biologically active.

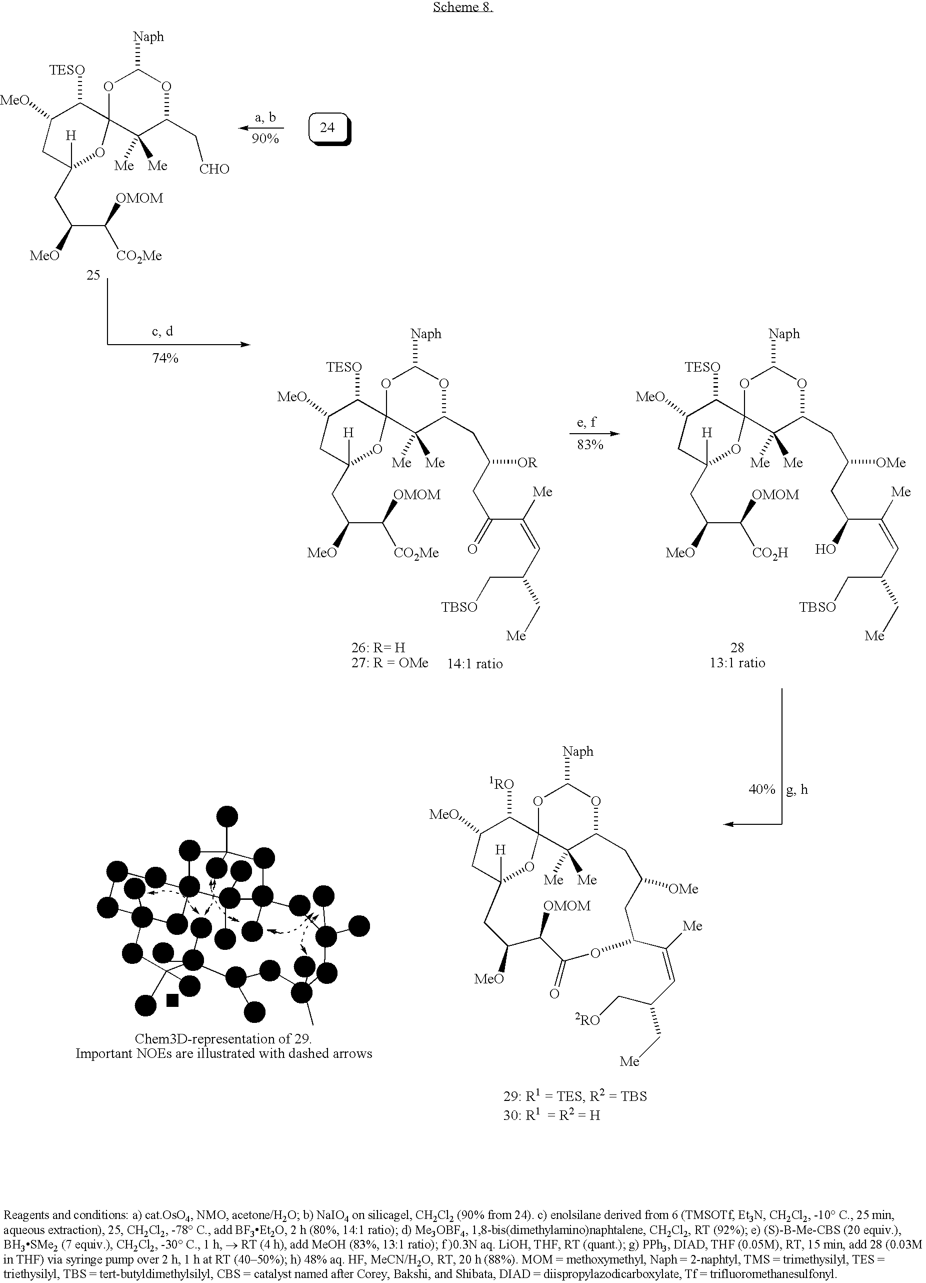

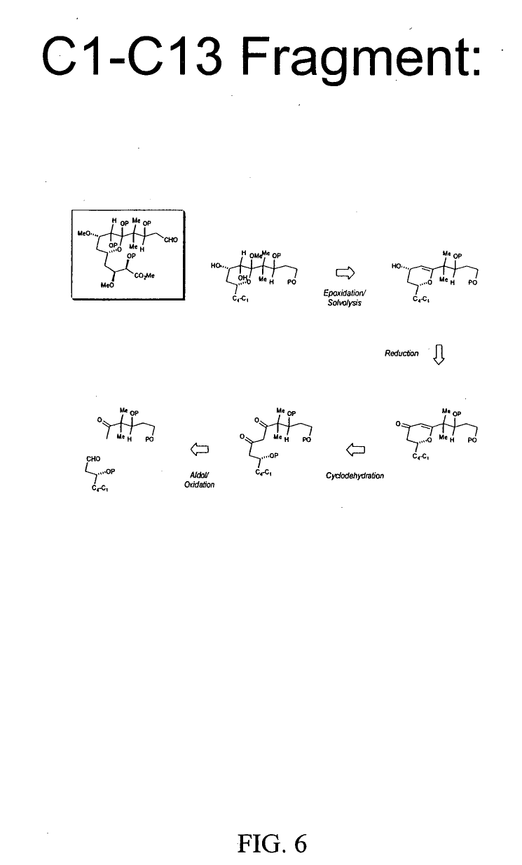

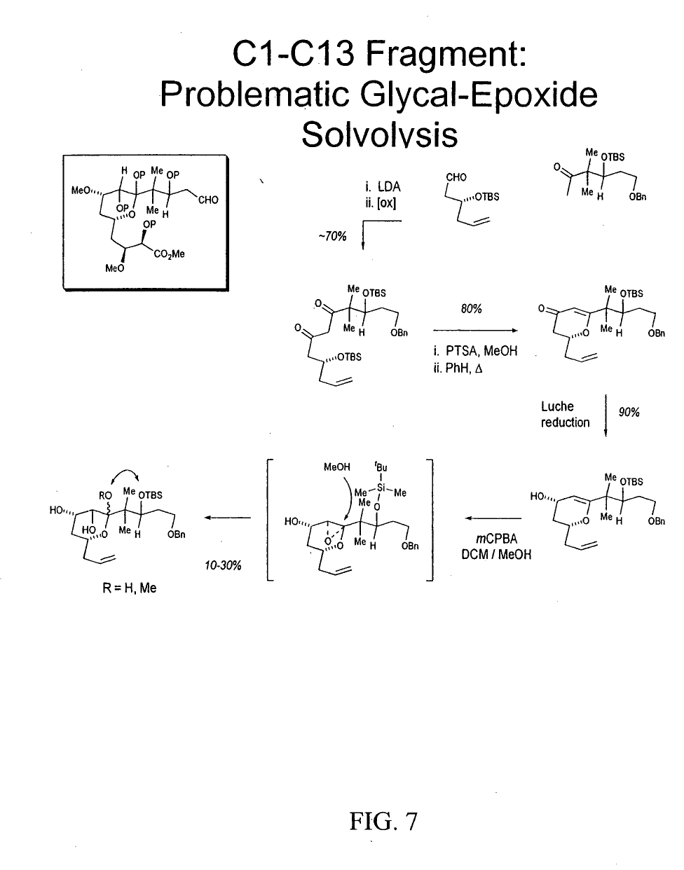

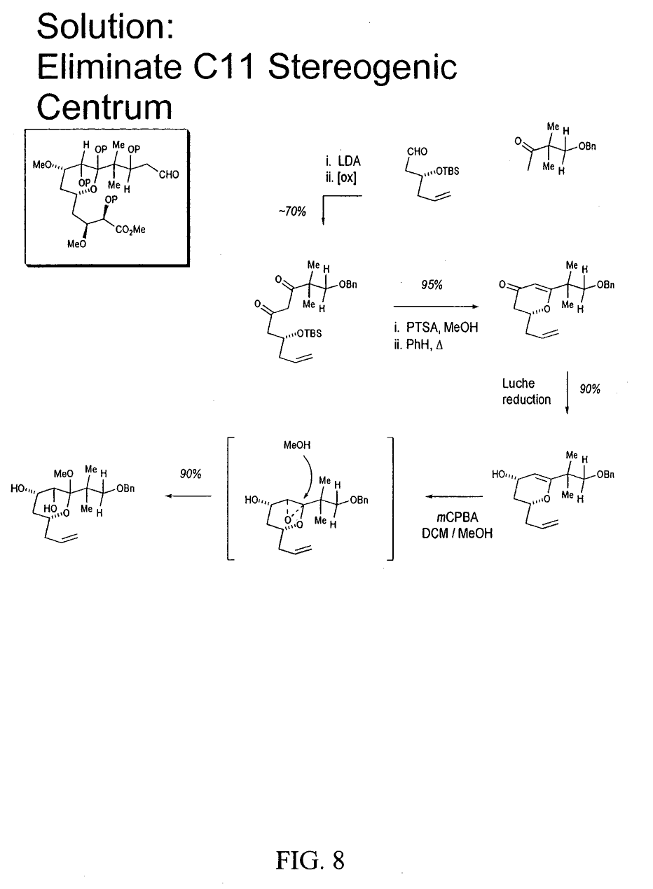

Accordingly, the present invention has accomplished the first total synthesis of both enantiomeric forms of Peloruside A and documented the absolute configuration of the dextrorotatory natural product. The advancement of minimally protected intermediates was key to the successful synthesis of Peloruside A, for example, 23, 31, 32a,b, 33a,b, 34a,b. FIGS. 3 and 6-8 further depicts the synthetic strategy of the present invention.

A. General Techniques

Unless otherwise noted, commercially available materials were used without further purification. All solvents used were of HPLC- or ACS-grade. Solvents used for moisture sensitive operations were distilled from drying agents under a nitrogen atmosphere: Et₂O and THF from sodium benzophenone ketyl; benzene and toluene from sodium; CH₂Cl₂, CH₃CN, NEt₃ and pyridine from CaH₂.

All moisture sensitive reactions were carried out under a nitrogen atmosphere with magnetic stirring. Flash chromatography (FC) was performed using E Merck silicagel 60 (240-400 mesh) according to the protocol of Still, Kahn, and Mitra (J. Org. Chem. 1978, 43, 2923). Thin Layer chromatography was performed using precoated plates purchased from E. Merck (silicagel 60 PF254, 0.25 mm) that were visualized using a KMnO₄ or Ce (IV) stain.

Nuclear magnetic resonance (NMR) spectra were recorded in CDCl₃, unless otherwise specified, on either a Varian Inova-400 or Mercury-300 spectrometer at operating frequencies of 400/300 MHz (¹H NMR) or 100/60 MHz (¹³C NMR). Chemical shifts (δ) are given in ppm relative to residual solvent (usually chloroform; δ 7.27 for ¹H NMR or δ 77.25 for proton decoupled ¹³C NMR), and coupling constants (J) in Hz. Multiplicity is tabulated as s for singlet, d for doublet, t for triplet, q for quadruplet, and m for multiplet, whereby the prefix app is applied in cases where the true multiplicity is unresolved, and br when the signal in question is broadened.

Infrared spectra were recorded on a Perkin-Elmer 1000 series FTIR with wavenumbers expressed in cm⁻¹ using samples prepared as thin films between salt plates. High-resolution mass spectra (HRMS) were recorded at the NIH regional mass spectrometry facility at the University of Washington, St. Louis, Mo. Optical rotations were measured on a Perkin-Elmer 241 MC polarimeter.

B. Experimentals

Note that all the experimental procedures and spectroscopic data for compounds with an enantiomeric relationship to the ones provided below are identical, except for the optical rotations of optically active compounds, which is of opposite sign.

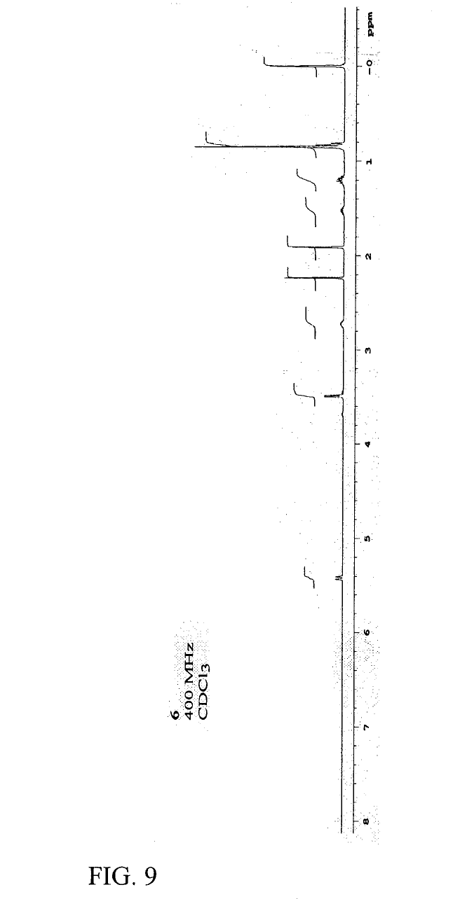

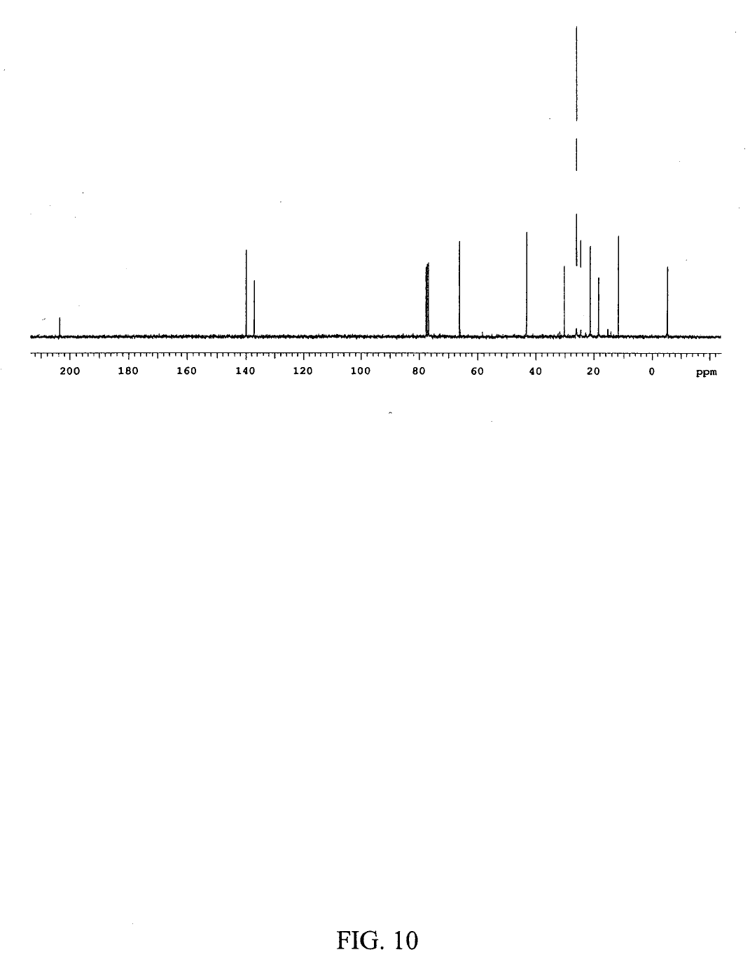

Ketone 6:

[α]_{D}²³=+18 (c 0.1, CHCl₃); IR 2925, 1690, 1457, 1253, 1100, 836 cm⁻¹; ¹H NMR (400 MHz [FIG. 9], CDCl₃) δ 5.45 (1H, dq, J=1.6, 10.8 Hz), 3.52 (2H, d, J=6.0 Hz), 2.76 (1H, m), 2.27 (3H, s), 1.94 (3H, d, J=1.6 Hz), 1.56 (1H, m), 1.23 (1H, m), 0.88 (9H, s), 0.86 (3H, t, J=7.6 Hz), 0.03 (3H, s), 0.02 (3H, s) ppm; ¹³C NMR (75 MHz [FIG. 10], CDCl₃) δ 203.6, 139.9, 137.1, 66.3, 43.4, 30.3, 26.0 (3C), 24.6, 21.2, 18.4, 11.8, −5.2, −5.3 ppm; HRMS Calcd for C₁₅H₃₀O₂SiLi ([M+Li]⁺): 277.2175. Found: 277.2183.

Compound 9:

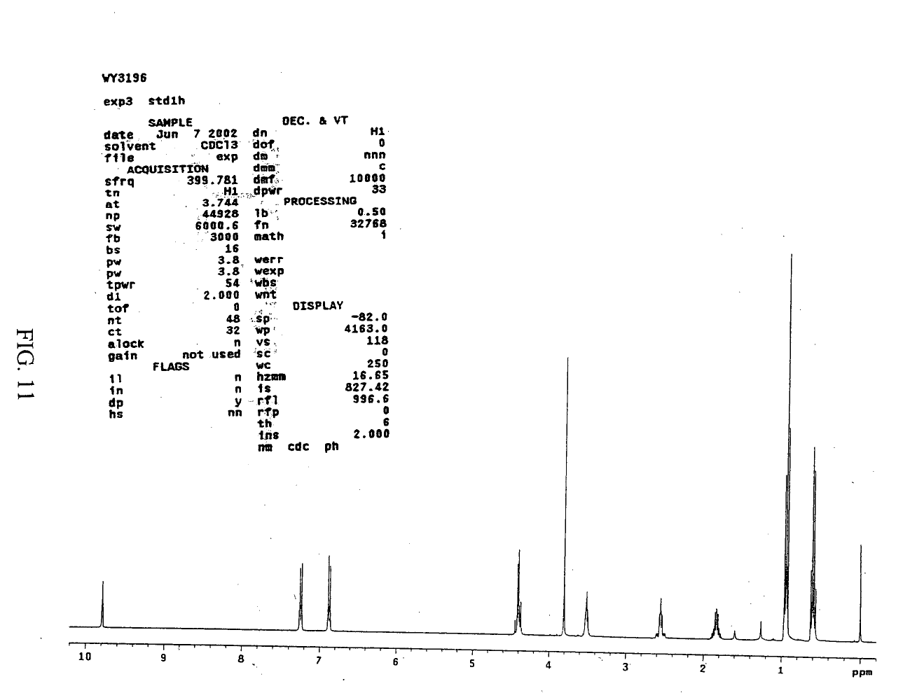

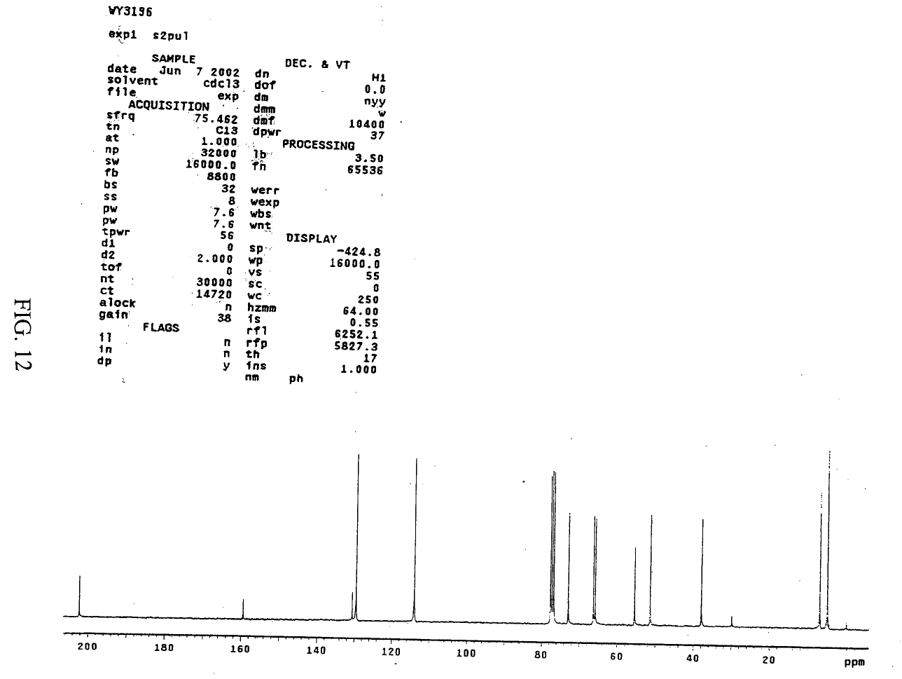

To a solution of7 (9.5 g, 40.2 mmol) in CH₂Cl₂ (100 mL) was added 2,6-lutidine (7.1 mL, 60.3 mmol) at −65° C., followed by the drop-wise addition of TESOTf (11 mL, 48.24 mmol). The reaction mixture was slowly warmed to −45° C. over 1 h, and brine (200 mL) was added. The organic layer was separated and the aq. phase was extracted with Et₂O (50 mL×3), dried over Na₂SO₄ and concentrated. The crude intermediate 8 (15.0 g) was used for the next step without further purification. To a solution of the crude intermediate 8 (15.0 g, 40.2 mmol) in acetone (125 mL) at 0° C. was added N-methyl-morpholine-N-oxide (12.5 mL, 60.3 mmol, 50% in water), followed by addition of OsO₄ (80.0 mL, 0.8 mmol, 0.1 M in BuOH). The mixture was stirred at RT for 25 h; EtOAc (50 mL) and brine (50 mL) were added, followed by Na₂SO₃ (4.00 g). After stirring for 20 min, the layers were separated and the aq. phase was extracted with EtOAc (150 mL×3). The combined organic layers were dried over Na₂SO₄ and concentrated. The crude diol (17.0 g) was used for the next step. To a solution of diol (16 g, 38.0 mmol) in CH₂Cl₂ (160 mL) at 0° C. was added pyridine (18.8 mL, 228 mmol), followed by addition of Pb(OAc)₄. The resultant mixture was stirred at 0° C. for 2 h. The precipitate was filtered and washed with CH₂Cl₂. The filtrate was washed with brine (200 mL), and the aq. phase was back-extracted with Et₂O (80 mL×3). The combined organic layers were dried over Na₂SO₄ and concentrated. The residue was separated by FC (10/90/0.2 EtOAc/hexane/Et₃N) to give 8.2 g product 9 in 61% yield (three steps). 9: [α]_{D}²³=−13.7 (c 0.7, CHCl₃); IR: 2955, 1726, 1614, 1514, 1248, 1095, 1037, 922, 744 cm⁻¹; ¹H NMR (400 MHz [FIG. 11], CDCl₃): 9.80 (1H, t, J=2.2 Hz), 7.24 (2H, d, J=8.6 Hz), 6.87 (2H, d, J=8.6 Hz), 4.42 (1H, d, J =11.6 Hz), 4.38 (1H, d, J=11.6 Hz), 5.52 (1H, dd, J=6.0, 12.0 Hz), 3.81 (3H, s), 3.51 (2H, m), 2.58 (1H, ddd, J=2.0, 5.4, 16.0 Hz), 5.52 (1H, ddd, J=2.0, 6.0, 16.0 Hz), 1.84 (2H, m), 0.94 (9H, t, J=8.0 Hz), 0.60 (6H, q, J=8.0 Hz) ppm; ¹³C NMR (75 MHz [FIG. 12], CDCl₃): 202.3, 159.7, 130.6, 129.5 (2C), 114.0 (2C), 72.9, 66.3, 65.8, 55.5, 51.4, 38.0, 7.0 (2C), 5.1 (2C) ppm.

Compound 11:

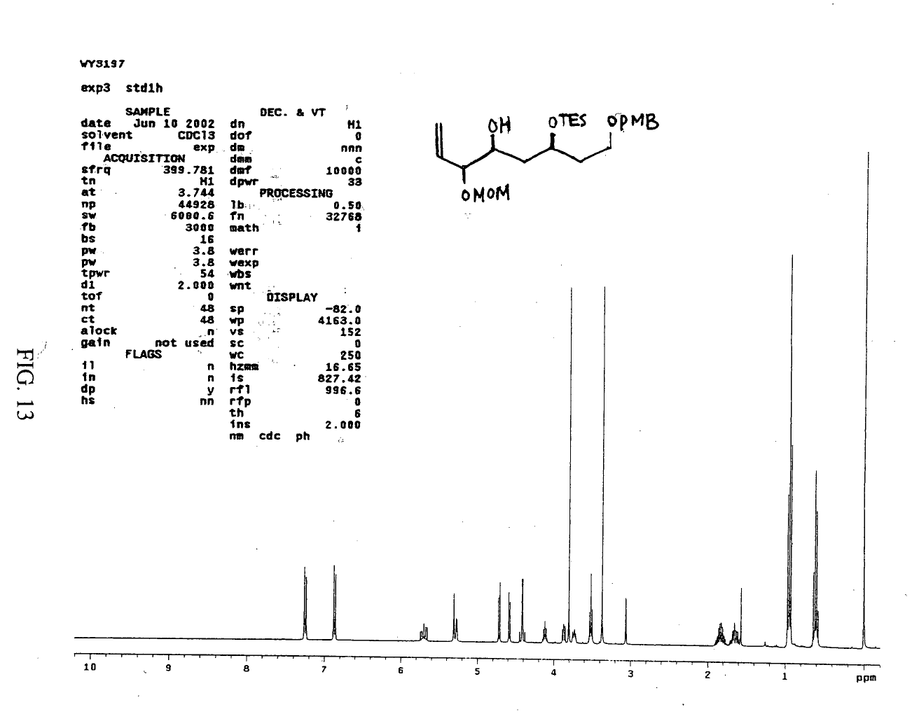

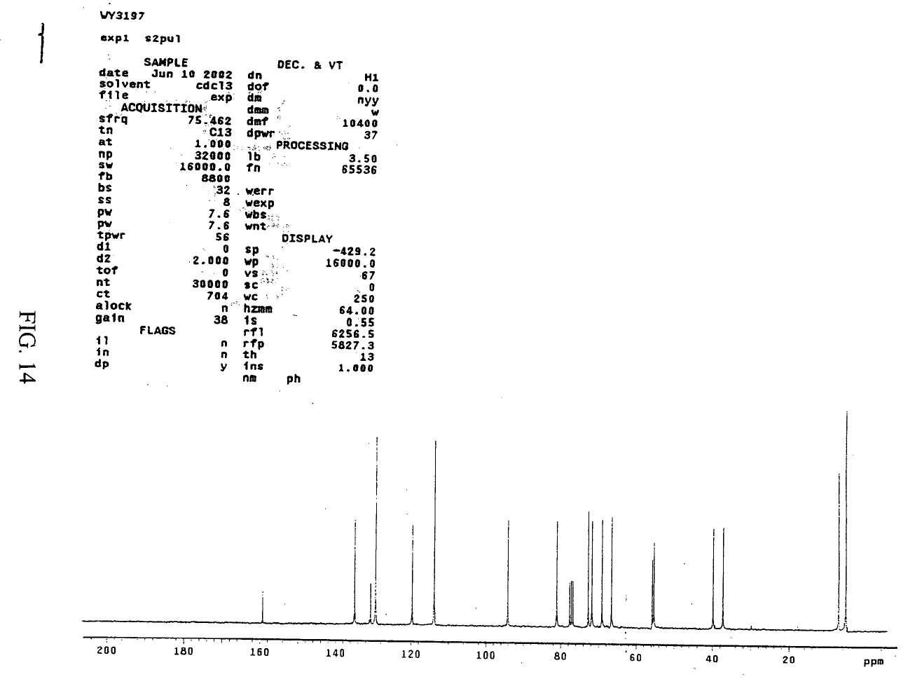

To a solution of 1-(methoxymethyl)oxy-2-propene (37.2 g, 36.5 mmol) in THF (60 mL) at −78° C. was added sec-BuLi (27 mL, 33.9 mmol, 1.3 M). The resulting orange yellow solution was stirred at −78° C. for 25 min, and (+)-Ipc₂BOMe (11.60 g, 36.533 mmol) in THF (60 mL) was added over 10 min. After the addition was completed, the color disappeared. The mixture was stirred at −78° C. for 20 min, and the cooling bath was removed. The mixture was further stirred at RT for 1.5 h and THF (40 mL) was added. The solution was cooled to −100° C., followed by addition of aldehyde 9 (8.1 g, 23 mmol) in THF (40 mL) over 15 min by double-tipped needle. The resultant mixture was stirred at −100 to −90° C. for 3 h, and warmed to RT overnight. THF was removed and Et₂O (200 mL) was added. The mixture was cooled to 0° C., and 2N aq. NaOH (70 mL) was added, followed by 30% aq. H₂O₂ (40 mL) drop-wise. The mixture was extracted with Et₂O (100 mL×3), and the organic layers were dried over Na₂SO₄ and concentrated. The residue was purified by FC (hexane, then 15% to 20% EtOAc in hexane, 0.2% Et₃N included) to afford 9.46 g product 11 in 90.6% yield. 11: [α:]_{D}²³=+36.9 (c 1.3, CHCl₃); IR: 3333, 2954, 1614, 1514, 1248, 1096, 1036, 922, 743 cm⁻¹; ¹H NMR (400 MHz [FIG. 13], CDCl₃): 7.25 (2H, d, J=9.0 Hz), 6.87 (2H, d, J=9.0 Hz), 5.70 (1H, ddd, J=7.6, 10.0, 16.6 Hz), 5.30 (1H, d, J=11.0 Hz), 5.29 (1H, d, J=16.6 Hz), 4.72 (1H, d, J=6.8 Hz), 4.58 (1H, d, J=6.8 Hz), 4.43 (1H, d, J=11.6 Hz), 4.40 (1H, d, J=11.6Hz), 4.12 (1H, ddd, J=6.0, 6.0, 12.0 Hz), 3.87 (1H, dd, J=5.6, 7.6 Hz), 3.81 (3H, s), 3.73 (1H, dddd, J=2.8, 2.8, 6.0, 8.8 Hz), 3.52 (2H, t, J=6.6 Hz), 3.37 (3H, s), 3.06 (1H, d, J=2.4 Hz), 1.86 (1H, ddd, J=6.4, 6.4, 20.0 Hz), 1.80 (1H, ddd, J=7.2, 7.2, 20.0 Hz), 1.69 (1H, dddd, J=2.4, 6.4, 14.4, 20.0 Hz), 1.62 (1H, dddd, J=2.8, 6.4, 14.4, 20.0 Hz), 0.95 (9H, t, J=6.0 Hz), 0.61 (6H, q, J=6.0 Hz) ppm; ¹³C NMR (75 MHz [FIG. 14], CDCl₃): 159.3, 134.9, 130.7, 129.4 (2C), 119.7, 113.8 (2C), 94.1, 81.1, 72.8, 71.8, 69.1, 66.6, 55.8, 55.4, 39.8, 37.2, 7.0 (3C), 5.1 (3C) ppm; HRMS Calcd for C₂₄H₄₂O₆SiLi ([M+Li]⁺): 461.2911. Found: 461.2905.

Compound 12:

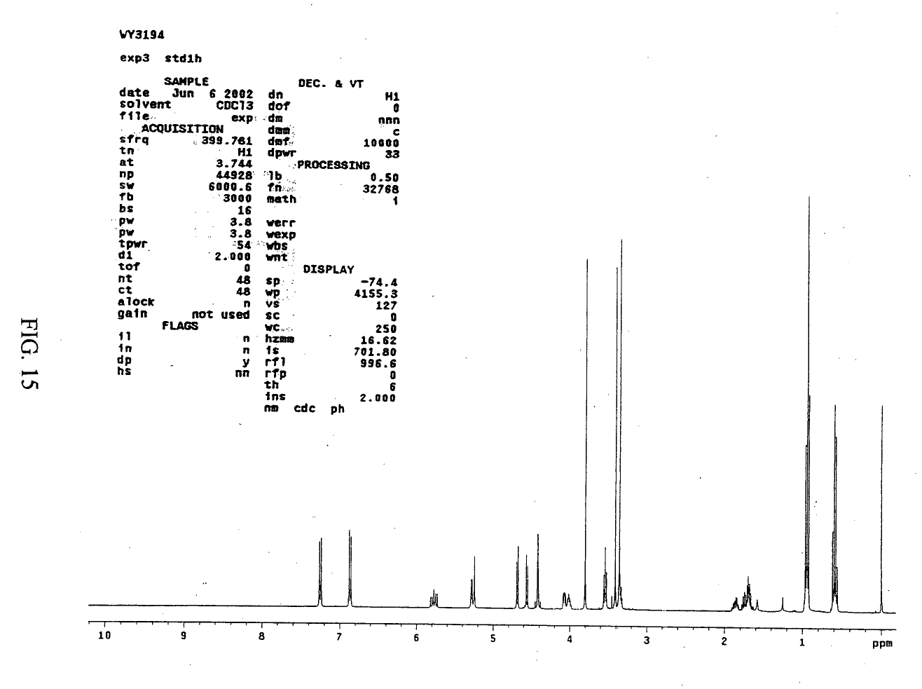

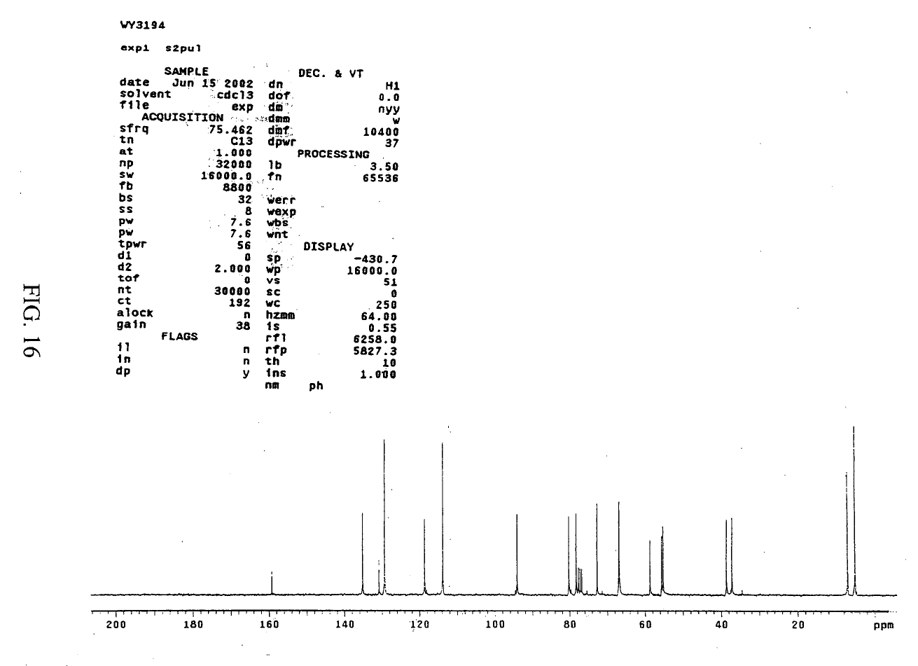

To a solution of 11 (3.70 g, 8.14 mmol) in DMF (50 mL) at −78° C. was added MeI (4.10 mL, 65.1 mmol), followed by the addition of NaH (651 mg, 16.275 mmol, 60% suspension in mineral oil) The resulting suspension was stirred at −5° C. for 2 h. The mixture was poured into brine (150 mL), extracted with Et₂O (50 mL×3), and the combined organic layers were dried over Na₂SO₄ and concentrated. The residue was purified by FC (hexane, then 5% to 10% EtOAc in hexane, 0.2% Et₃N included) to give 3.4 g product 12 in 98% yield. 12: [α]_{D}²³=+15.7 (c 2.0, CHCl₃); IR: 2954, 1614, 1514, 1248, 1099, 1038, 920, 743 cm⁻¹. ¹H NMR (400 MHz [FIG. 15], CDCl₃): 7.25 (2H, d, J=8.4 Hz), 6.87 (2H, d, J=8.4 Hz), 5.78 (1H, ddd, J=7.2, 10.5, 17.0 Hz), 5.27 (1H, d, J=17.0 Hz), 5.25 (1H, d, J=10.5 Hz), 4.68 (1H, d, J=7.0 Hz), 4.56 (1H, d, J=7.0 Hz), 4.43 (1H, d, J=11.6 Hz), 4.40 (1H, d, J=11.6 Hz), 4.07 (1H, dd, J=4.4, 7.2 Hz), 4.02 (1H, m), 3.80 (3H, s), 3.54 (2H, t, J=6.4 Hz), 3.41 (3H, s), 3.36 (3H, s), 3.35 (1H, m), 1.86(1H, dddd, J=4.4, 6.8, 6.8, 13.8 Hz), 1.66-1.78 (3H, m), 0.95 (9H, t, J=8.0 Hz), 0.58 (6H, q, J=8.0 Hz) ppm; ¹³C NMR (75 MHz [FIG. 16], CDCl₃): 159.2, 135.2, 130.8, 129.3 (2C), 118.6, 113.8 (2C), 94.1, 80.2, 78.3, 72.7, 67.0, 66.9, 58.7, 55.7, 55.3, 38.7, 37.2, 7.0 (3C), 5.1 (3C) ppm; HRMS Calcd for C₂₅H₄₄O₆SiLi ([M+Li]⁺): 475.3067. Found: 475.3056.

Compound 15:

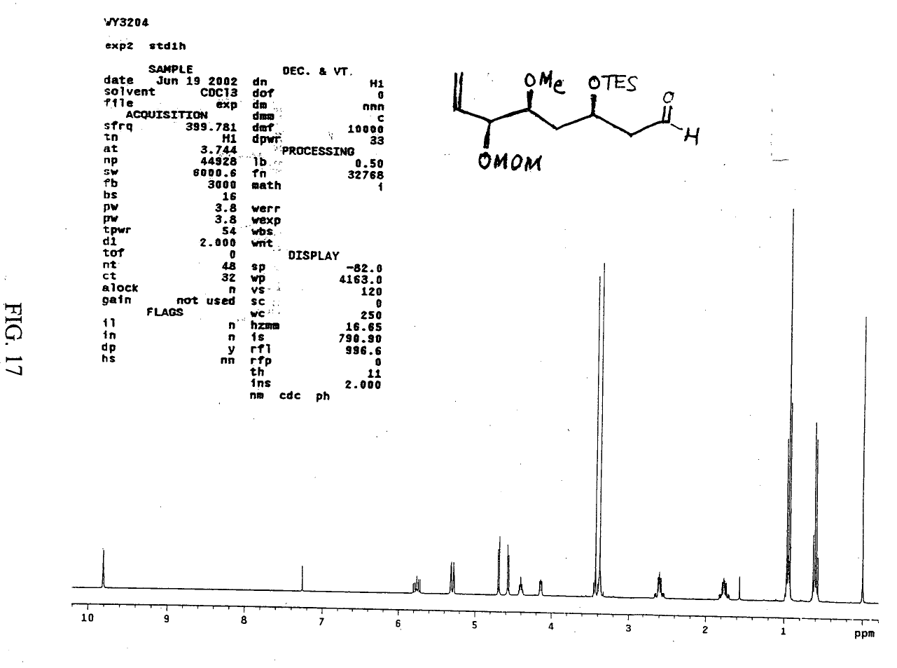

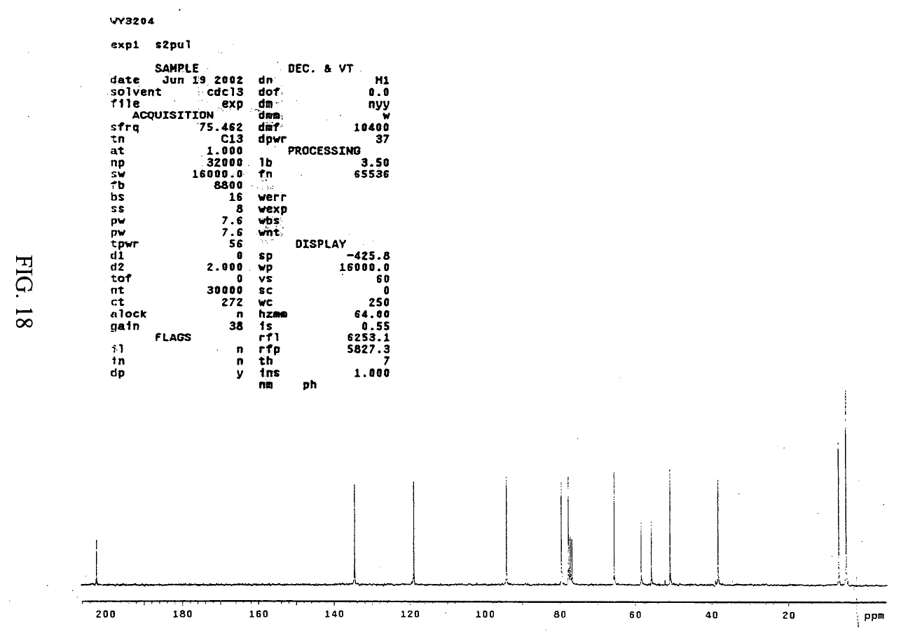

To a solution of 12 (6.9 g, 14.72 mmol) in CH₂Cl₂ (160 mL) at 0° C. was added H₂O (8.9 mL), followed by addition of DDQ (4.1 g, 17.7 mmol) in one portion. The resulting mixture was stirred at 0° C. for 2 h, followed by the sequential addition of Et₃N (2.0 mL), brine (200 mL) and aq. sat. NaHCO₃ (5 mL). The mixture was passed through celite. The aq. phase was extracted with Et₂O (100 mL×3), and the combined organic layers were dried over MgSO₄ and concentrated. The residue was purified by FC (20/80/0.2 EtOAc/Hexane/Et₃N) to afford 4.5 g of product intermediate i in 88% yield. i: [α]_{D}²³=+11 (c 1.4, CHCl₃); IR: 3336, 2954, 2880, 1463, 1153, 1100, 1034, 923, 743 cm⁻¹; ¹H NMR (400 MHz, CDCl₃): 5.77 (1H, ddd, J=7.2, 11.0, 17.0 Hz), 5.30 (1H, d, J=17.0 Hz), 5.29 (1H, d, J=11.0 Hz), 4.69 (1H, d, J=7.0 Hz), 4.58 (1H, d, J=7.0 Hz), 4.12 (2H, m), 3.81 (1H, m), 3.76 (1H, m), 3.44 (3H, s), 3.38 (3H, s), 3.31 (1H, ddd, J=4.0, 4.0, 8.8 Hz), 2.65 (1H, s), 1.68-1.93 (4H, m), 0.97 (9H, t, J=8.0 Hz), 0.62 (6H, q, J=8.0 Hz) ppm. ¹³C NMR (75 MHz, CDCl₃): 134.7, 118.8, 94.2, 80.1, 78.1, 69.0, 60.1, 58.6, 55.6, 38.1, 37.8, 6.9 (3C), 5.1 (3C) ppm; HRMS Calcd for C₁₇H₃₆O₅SiLi ([M+Li]⁺): 355.2492. Found: 355.2477. To a solution of intermediate i (4.5 g, 12.91 mmol) in DMSO (30 mL) and CH₂Cl₂ (30 mL) at 0° C. was added Et₃N (5.7 mL, 40.61 mmol), followed by addition of Py.SO₃ (4.30 g, 27.07 mmol) in one portion. The mixture was stirred at 0° C. for 1.5 h, and poured into Et₂O (100 mL) and brine (150 ml). The organic layer was separated and the aq. phase was extracted with Et₂O (50 mL×3). The combined organic extracts were dried over MgSO₄ and concentrated. The residue was purified by FC (12/88/0.2 EtOAc/Hexane/Et₃N) to give 3.9 g product 15 in 87% yield. 15: [α]_{D}²³=+17 (c 0.7, CHCl₃); IR: 2954, 2880 1726, 1097, 1035, 920, 743 cm⁻¹; ¹H NMR (400 MHz [FIG. 17], CDCl₃): 9.81 (1H, t, J=2.8 Hz), 5.76 (1H, ddd, J=7.6, 11.0, 16.0 Hz), 5.30 (1H, d, J=16.0 Hz), 5.29 (1H, d, J=11.0 Hz), 4.69 (1H, d, J=7.2 Hz), 4.57 (1H, d, J=7.2 Hz), 4.41 (1H, ddd, J=4.6, 6.8, 11.6 Hz), 4.14 (1H, dd, J=4.6, 7.4 Hz), 3.43 (3H, s), 3.38 (3H, s), 3.38 (1H, m), 2.63 (1H, ddd, J=2.4, 5.2, 16.0 Hz), 2.57 (1H, ddd, J=3.2, 7.2, 16.0 Hz), 1.80 (1H, ddd, J=4.0, 7.2, 14.4 Hz), 1.74 (1H, ddd, J=4.6, 8.8, 14.4 Hz), 0.95 (9H, t, J=8.0 Hz), 0.60 (6H, q, J=8.0 Hz) ppm; ¹³C NMR (75 MHz [FIG. 18], CDCl₃): 202.3, 134.7, 1119.0, 94.3, 79.6, 77.8, 65.6, 58.5, 55.8, 50.8, 38.4, 7.0 (3C), 5.0 (3C) ppm.

Compound 16:

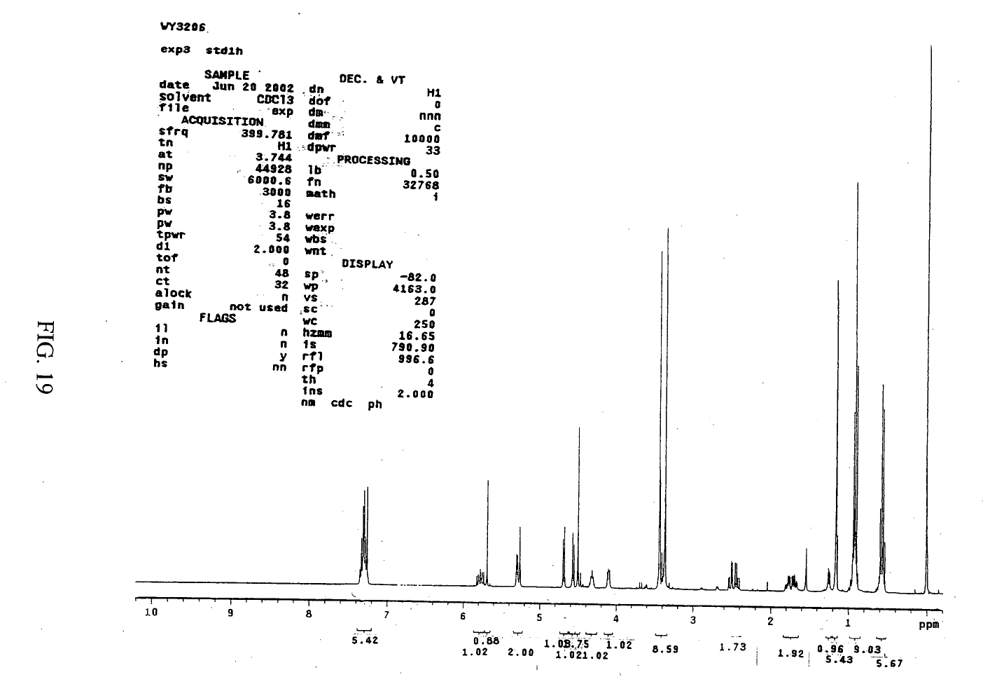

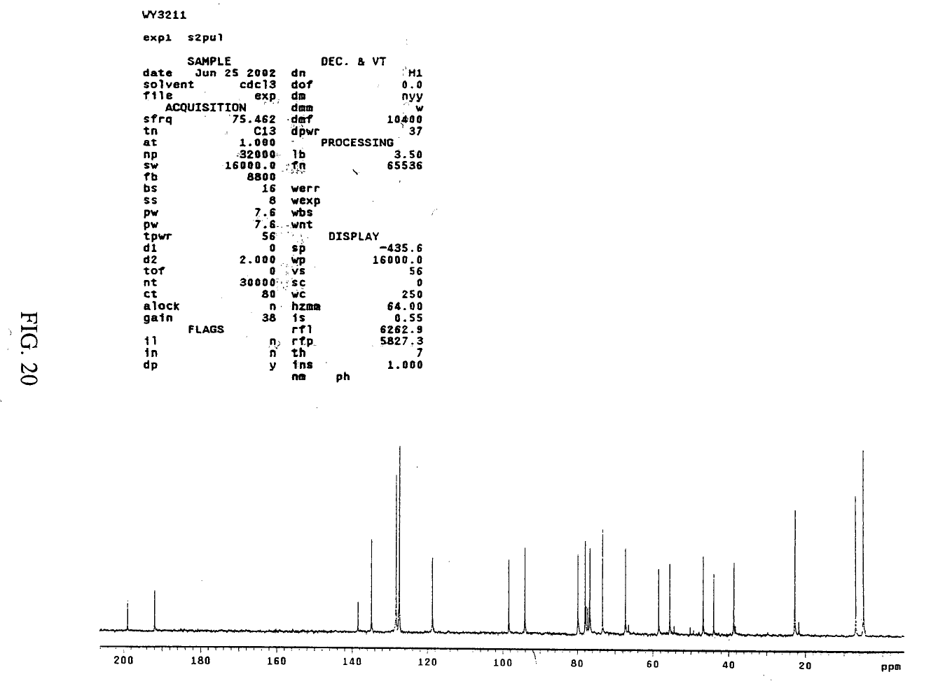

To a solution of diisopropylamine (2.65 mL, 18.89 mmol) in THF (30 mL) at 0° C. was added n-BuLi (7.1 mL, 17.705 mmol, 2.5M in hexane). The resulting solution was stirred at 0° C. for 1 h and cooled to −78° C., followed by the addition of a solution of ketone 14 (3.2 g, 15.345 mmol) in THF (15 mL) over 5 min. The resulting mixture was stirred at −78° C. for 20 min, and warmed to −40° C. over 35 min. After the solution was re-cooled to −78° C., a solution of aldehyde 15 (3.9 g, 11.254 mmol) in THF (15 mL) was added drop-wise over 10 min. The mixture was stirred at −78° C. for 2.5 h and quenched by addition of a solution of acetic acid (1. 1 mL, 18.9 mmol) in Et₂O (10 mL). Stirring was continued for 10 min, followed by pouring into brine (150 mL), extraction with Et₂O (80 mL×3), and drying of the combined organic extracts over MgSO₄. After concentration, the residue was purified by FC (20/80/0.2 EtOAc/Hex/Et₃N) to give 6.0 g of product intermediate ii in 96% yield (mixture of two diastereoisomers). To a solution of intermediate ii (2.0 g, 3.62 mmol) in CH₂Cl₂ (35 mL) at −7° C. was added Dess-Martin periodinane (DMP) (2.4 g, 5.43 mmol) in one portion. The resulting mixture was stirred at −7° C. for 1.3 h, followed by the addition of a solution of Na₂SO₃ (5 g) in H₂O (20 mL) and sat. aq. NaHCO₃ (3 ml). After stirring for 10 min, the organic phase was separated and the aq. phase was extracted with Et₂O (30 mL×3). The combined organic layers were dried over Na₂SO₄ and concentrated The residue was isolated by FC (10/90/0.2 EtOAc/Hexane/Et₃N), providing 1.0 g of product 16 and 760 mg of recovered starting material (ii) (81% yield based on recovered starting material). 16: [α]_{D}²³=−5.9 (c 1.5, CHCl₃); IR: 2955, 1603, 1460, 1098, 1031, 927, 739 cm¹; ¹H NMR (400 MHz [FIG. 19], CDCl₃) 7.24-7.36 (5H, m), 5.78 (1H, ddd, J=7.4, 10.5, 17.5 Hz), 5.69 (1H, s), 5.28 (1H, d, J=17.5 Hz), 5.27 (1H, d, J=10.5 Hz), 4.69 (1H, d, J=6.8 Hz), 4.56 (1H, d, J=6.8 Hz), 4.51 (2H, s), 4.32 (1H, dddd, J=4.8, 4.8, 4.8, 12.0 Hz), 4.10 (1H, dd, J=4.8, 7.4 Hz), 3.45 (3H, s), 3.44 (2H, m), 3.40 (1H, m), 3.37 (3H, s), 2.52 (1H, dd, J=4.8, 14.0 Hz), 2.43 (1H, dd, J=7.6, 14.0 Hz), 1.78(1H, ddd, J=4.0, 7.2, 14.4 Hz), 1.70 (1H, ddd, J=4.0, 8.4, 14.4 Hz), 1.17 (6H, s), 0.92 (9H, t, J=8.0 Hz), 0.56 (6H, q, J=8.0 Hz) ppm; ¹³C NMR (75 MHz [FIG. 20], CDCl₃): 199.0, 191.9, 138.4, 134.9, 128.3 (2C), 127.5 (3C), 118.7, 98.4, 94.1, 79.8, 77.9, 76.6, 73.3, 67.3, 66.4, 58.5, 55.6, 46.8, 44.0, 38.7, 22.7, 6.9 (3C), 4.9 (3C) ppm; HRMS Calcd for C₃₀H₅₀O₇SiLi ([M+Li]⁺): 557.3486. Found: 557.3510.

Compound 17:

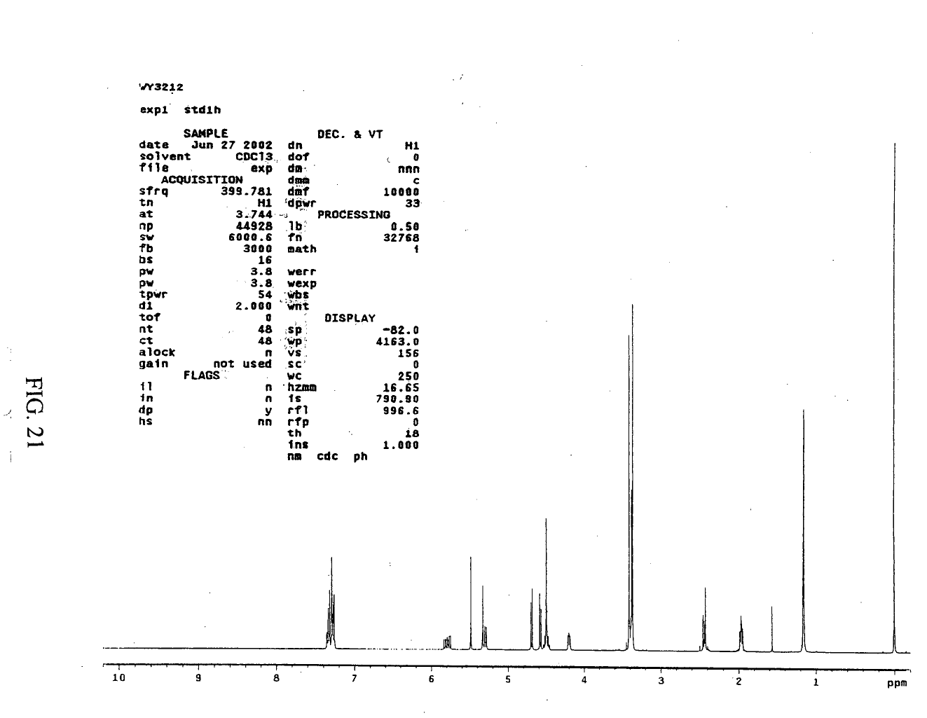

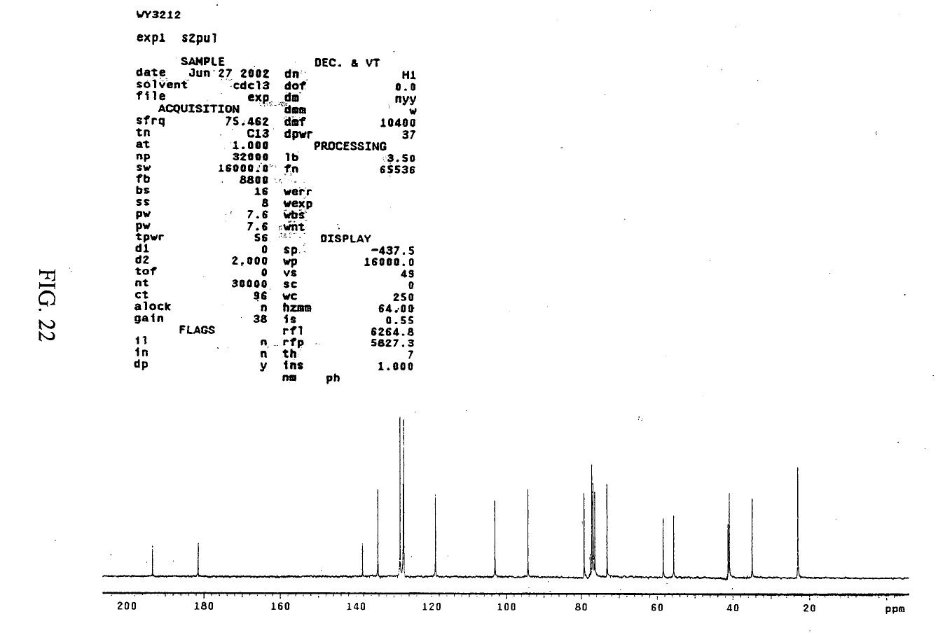

To a solution of 16 (4.5 g, 8.17 mmol) in toluene (130 mL) at RT was added para-toluenesulfonic acid (233 mg, 1.225 mmol). The solution was stirred at RT for 18 h. The reaction mixture was directly transferred to column for separation by FC (35/65 EtOAc/Hexane), which afforded 3.13 g product 17 in 91% yield. 17: [α]_{D}²³=+99.4 (c 0.65, CHCl₃); IR: 2889, 1667, 1598, 1457, 1338, 1098, 1033, 923, 740, 699 cm⁻¹; ¹H NMR (400 MHz [FIG. 21], CDCl₃): 7.26-7.36 (5H, m), 5.79 (1H, ddd, J=7.2, 10.8, 17.0 Hz), 5.49 (1H, s), 5.32 (1H, d, J=10.6 Hz), 5.31 (1H, d, J=17.0 Hz), 4.70 (1H, d, J=6.6 Hz), 4.58 (1H, d, J=6.6 Hz), 4.50 (2H, s), 4.50 (1H, m), 4.20 (1H, dd, J=4.8, 6.8 Hz), 3.41 (3H, s), 3.34-3.41 (3H, m), 2.45 (1H, d, J=11.6 Hz), 2.44 (1H, d, J=5.6 Hz), 1.97 (2H, ddd, J=2.8, 6.8, 6.8 Hz), 1.16 (3H, s), 1.15 (3H, s) ppm; ¹³C NMR (75 MHz [FIG. 22], CDCI₃): 193.4, 181.5, 138.3, 134.3, 128.4 (2C), 127.5, 127.4 (2C), 119.0, 103.1, 94.3, 79.3, 77.2, 76.9, 76.4, 73.2, 58.4, 55.6, 41.3, 41.0, 35.0, 23.0 (2C) ppm; HRMS Calcd for C₂₄H₃₄O₆Li ([M+Li]⁺): 425.2515. Found: 425.2512.

Compound 19:

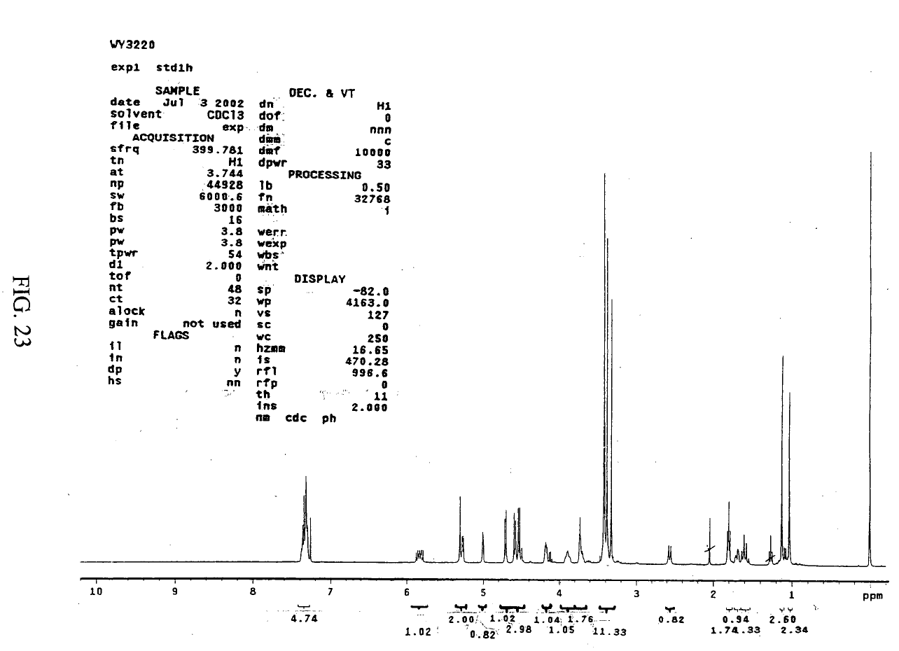

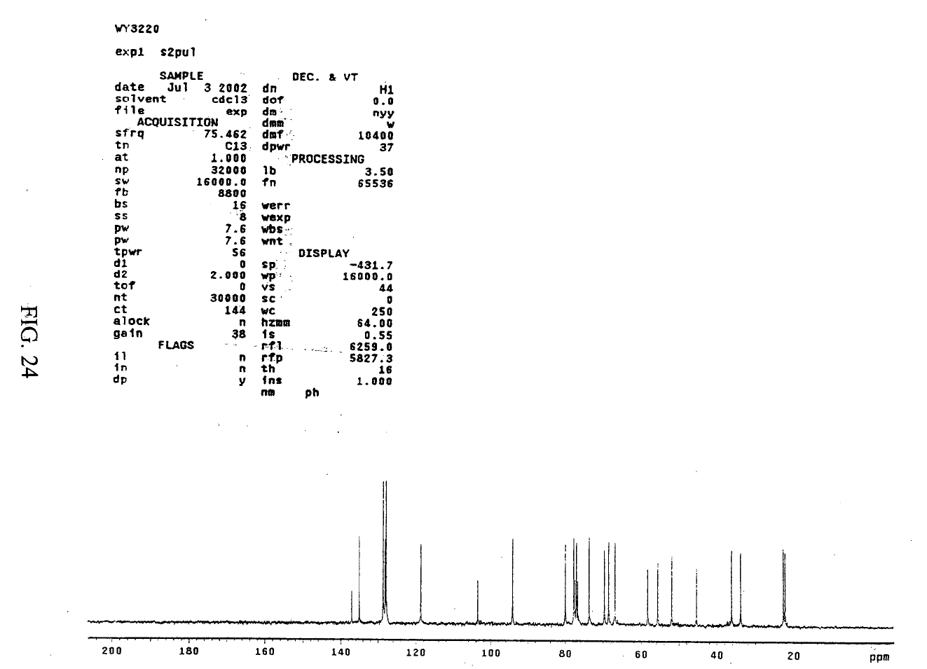

To a solution of 17 (2.3 g, 5.496 mmol) in MeOH (100 mL) was added CeCl₃.7H₂O. After cooling the solution to −30° C., NaBH₄ (180 mg) was added, followed by the addition of a second portion of NaBH₄ (120 mg) after 10 min. The mixture was stirred at −30° C. for 30 min and poured into brine (100 mL) and EtOAc (50 mL). The organic phase was separated and the aq. phase was extracted with EtOAc (50 mL×3). The combined organic layers were dried over Na₂SO₄ and concentrated. The residue 18 (1.95 g) was used for the next step. To a solution of intermediate 18 (1.95 g, 4.64 mmol) in CH₂Cl₂ (100 mL) and MeOH (50 mL) at 0° C. was added NaHCO₃, followed by the addition of m-CPBA (920 mg, 5.33 mmol) in three portions every 5 min. The mixture was stirred at 0° C. for 20 min and Et₃N (24 mL) was added. After 20 min, the mixture was poured into 20 mL of Et₂O and stirring was continued for 20 min. After passing the mixture through celite, the filtrate was concentrated and the residue was purified by FC (40/60/0.4 EtOAc/Hexane/Et₃N) to give 1.57 g product 19 in 72% yield (two steps). 19: [α]_{D}²³=+54.9 (c 0.65, CHCl₃); IR: 3447, 2930, 1457, 1100, 1067, 1036, 926, 737, 697 cm⁻¹; ¹HNMR (400 MHz [FIG. 23], CDCl₃): 7.29-7.38 (5H, m), 5.83 (1H, ddd, J=7.2, 11.2, 16.0 Hz), 5.38 (1H, d, J=16.0 Hz), 5.37 (1H, d, J=11.2 Hz), 5.00 (1H, d, J=3.2 Hz), 4.71 (1H, d, J=6.6 Hz), 4.58 (1H, d, J=6.6 Hz), 4.55 (1H, d, J=11.6 Hz), 4.51 (1H, d, J=11.6 Hz), 4.17 (1H, dd, J=4.4, 7.24 Hz), 3.89 (1H, m), 3.73 (1H, d, J=3.2 Hz), 3.72 (1H, d, J=3.2 Hz), 3.70 (1H, dddd, J=3.2, 6.4, 9.0 Hz), 3.38-3.44 (2H, m), 3.42 (3H, s), 3.38 (3H, s), 3.33 (3H, s), 2.58 (1H, d, J=10.8 Hz), 2.43 (1H, dd, J=7.6, 14.0 Hz), 1.80 (2H, t, J=6.0 Hz), 1.69 (1H, ddd, J=3.0, 4.4, 12.0 Hz), 1.60 (1H, m), 1.12 (3H, s), 1.02 (3H, s) ppm; ¹³C NMR (75 MHz [FIG. 24], CDCl₃): 137.1, 135.1, 128.7 (2C), 128.1, 127.9 (2C), 118.7, 103.6, 94.2, 80.1, 77.8, 77.1, 73.8, 69.3, 68.6, 67.0, 58.4, 55.7, 52.0, 45.4, 36.3, 33.9, 22.8, 22.3 ppm; HRMS Calcd for C₂₅H₄₀O₈Li ([M+Li]⁺): 475.2883. Found: 475.2881.

Compound 20:

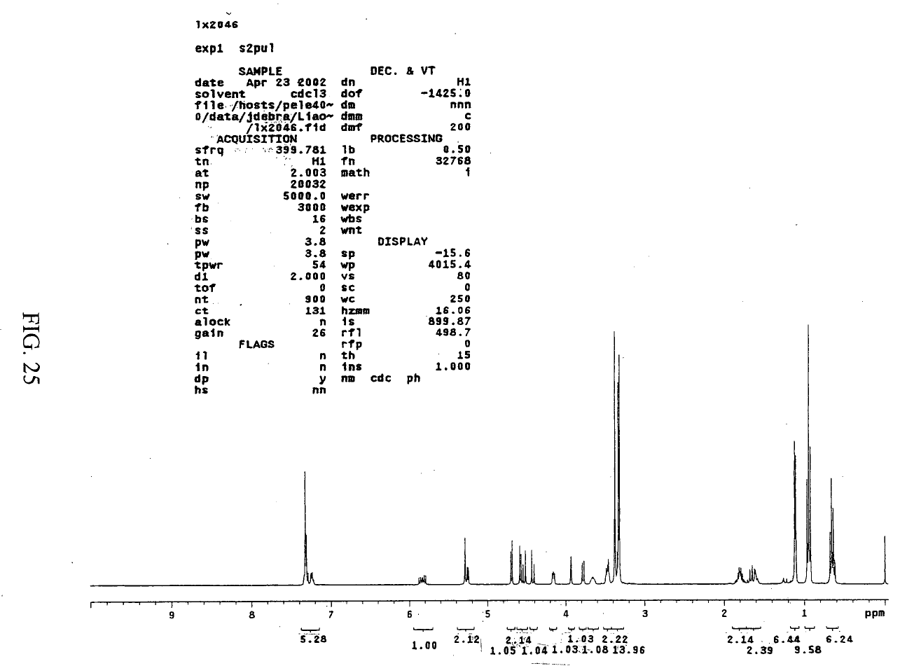

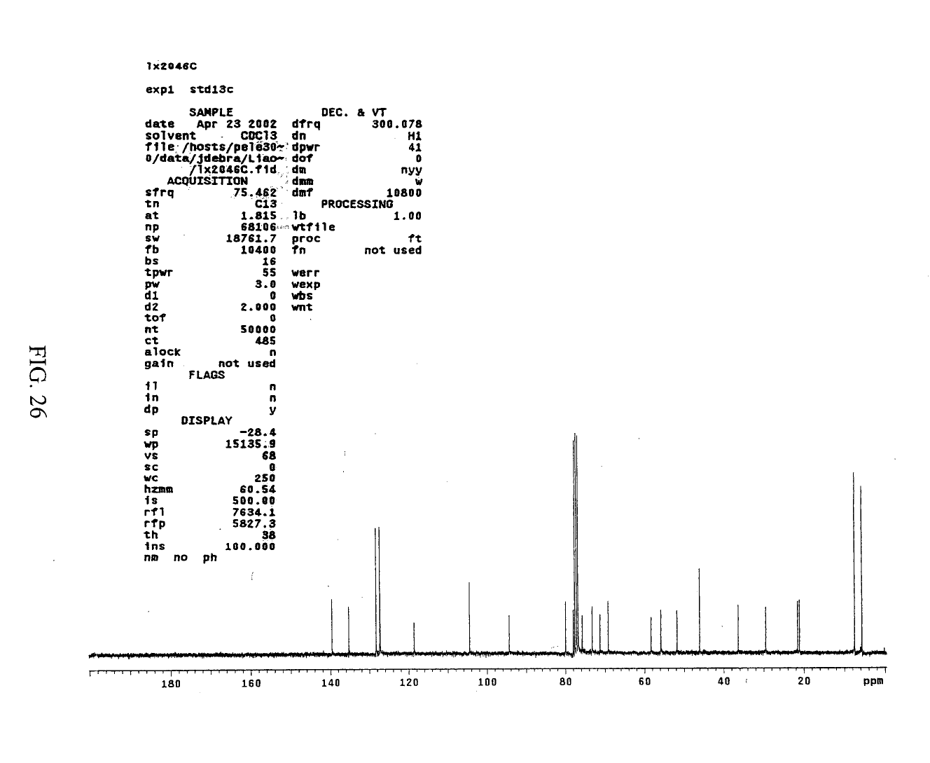

To a solution of 19 (1.28 g, 1.28 mmol) in THF (17 mL) at 0° C. was added KOBu^{t}(182 mg, 1.54 mmol) in one portion. The resulting orange solution was stirred at 0° C. for 7 min and a solution of MeI (1.61 mL, 2.561 mmol) in THF (84 mL) was added drop-wise. The resulting suspension was stirred at 0° C. for 30 min, and 0.5 mL Et₃N was added followed by brine (50 mL) and Et₂O (30 mL). The aq. phase was extracted with EtOAc (30 mL×3), and the combined organic layers were dried over Na₂SO₄ and concentrated. The residue was isolated by FC (35/65/0.3 to 45/55/0.2 EtOAc/Hexane/Et₃N) to afford 325 mg product (intermediate iii) and 180 mg recovered starting material 19 in 75% yield (based on recovered starting material). Intermediate iii: [α]_{D}²³=+42.8 (c 0.8, CHCl₃); IR: 3480, 2930, 1457, 1100, 1067, 927, 740, 700 cm⁻¹; ¹H NMR (400 MHz, CDCl₃): 7.27-7.35 (5H, m), 5.83 (1H, ddd, J=7.4, 11.2, 16.4 Hz), 5.28 (1H, d, J=16.4 Hz), 5.27 (1H, d, J=11.2 Hz), 4.70 (1H, d, J=7.2 Hz), 4.58 (1H, d, J=7.2 Hz), 4.53 (2H, s), 4.20 (1H, d, J=2.0 Hz), 4.17 (1H, dd, J=4.8, 6.8 Hz), 3.93 (1H, t, J=2.0 Hz), 3.72 (1H, dddd, J=2.8, 2.8, 6.0, 8.8 Hz), 3.58 (2H, m), 3.44 (1H, m), 3.40 (3H, s), 3.39 (3H, s), 3.38 (3H, s), 3.34 (3H, s), 3.33 (1H, m), 1.82 (1H, t, J=6.0 Hz), 1.76 (1H, q, J=12.0 Hz), 1.66 (1H, td, J=3.2, 12.0 Hz), 1.11 (3H, s), 1.08 (3H, s) ppm; ¹³C NMR (75 MHz, CDCl₃): 137.9, 135.2, 128.6 (2C), 127.9 (3C), 118.8, 104.1, 94.3, 80.1, 77.9, 76.7, 76.6, 73.7, 68.9, 66.9, 58.4, 55.8, 55.7, 52.0, 45.8, 36.3, 30.2, 22.7, 21.9 ppm; HRMS Calcd for C₂₆H₄₂O₈Li ([M+Li]⁺): 489.3040. Found: 489.3024. To a solution of the alcohol intermediate iii (1.2 g, 2.487 mmol) in dry THF at −78° C. was added 2,6-lutidine (731 RL, 6.22 mmol), followed by TESOTf (859 μL, 3.73 mmol). The solution was stirred at the same temperature for 40 min, followed by dilution with Et₂O (300 mL) and sat. aq. NH₄Cl (100 mL). The organic layer was separated and washed with 100 mL sat. aq. NH₄Cl, dried over Na₂SO₄ and concentrated. The residue was purified by FC (EtOAc/hexane 12/88) to provide the product 20 (1.2 g, 81%). [α]_{D}²³=+32 (c 0.5, CHCl₃) {ent-20: [α]_{D}²³=−24 (c 0.5, CHCl₃)}; IR 2947, 1459, 1099, 1039, 740 cm⁻¹; ¹H NMR (400 MHz [FIG. 25], CDCl₃) δ 7.31 (5H, m), 5.83 (1H, dddd, J=7.2, 7.6, 10.0, 17.2 Hz), 5.26 (2H, m), 4.70 (1H, d, J=6.4 Hz), 4.58 (1H, d, J=6.4 Hz), 4.53 ( 1H, d, J=12.4 Hz), 4.42 (1H, d, J=12.4 Hz), 4.16 (1H. dd, J=4.4, 7.2 Hz), 3.93 (1H, d, J=1.6 Hz), 3.78 (1H, d, J=8.8 Hz), 3.67 (1H, m), 3.47 (1H, m), 3.38 (3H, s), 3.37 (3H, s), 3.34 (1H, d, J=8.8 Hz), 3.33 (3H, s), 3.32 (3H, s), 1.80 (2H, m), 1.65 (2H, m), 1.12 (3H, s), 1.11 (3H, s), 0.94 (9H, t, J=8.0 Hz), 0.66 (6H, q, J=8.0 Hz) ppm; ¹³C NMR (75 MHz [FIG. 26], CDCl₃) δ 6 139.7, 135.3, 128.3, 128.0, 127.4, 118.6, 104.5, 94.4, 80.0, 78.0, 77.2, 75.7, 73.2, 71.2, 69.2, 58.3, 55.9, 55.8, 51.7, 46.0, 36.3, 29.4, 21.5, 21.1, 7.4 (3C), 5.6 (3C) ppm; HRMS Calcd for C₃₂H₅₆O₈SiLi ([M+Li]⁺): 603.3904. Found: 603.3900.

Compound ent-21:

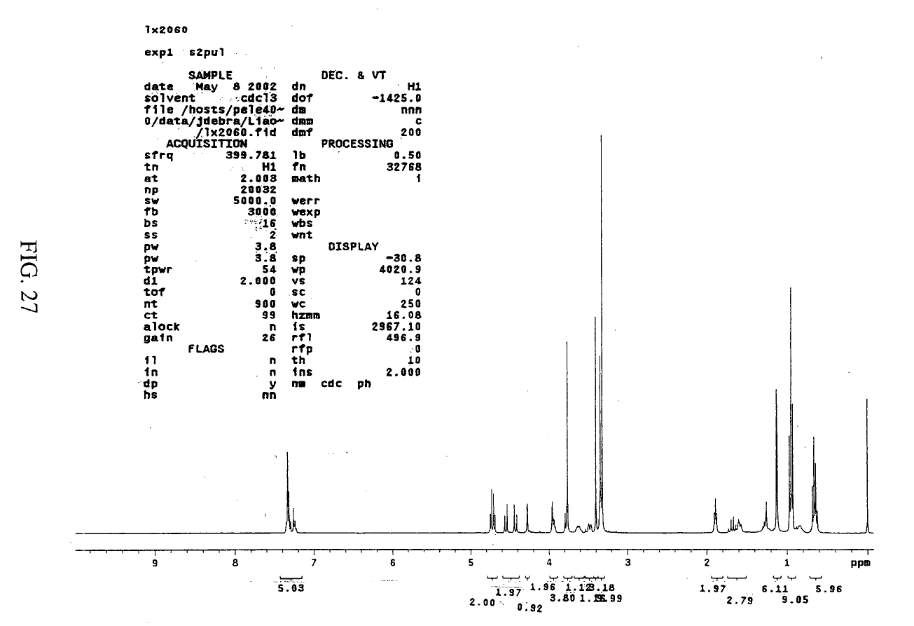

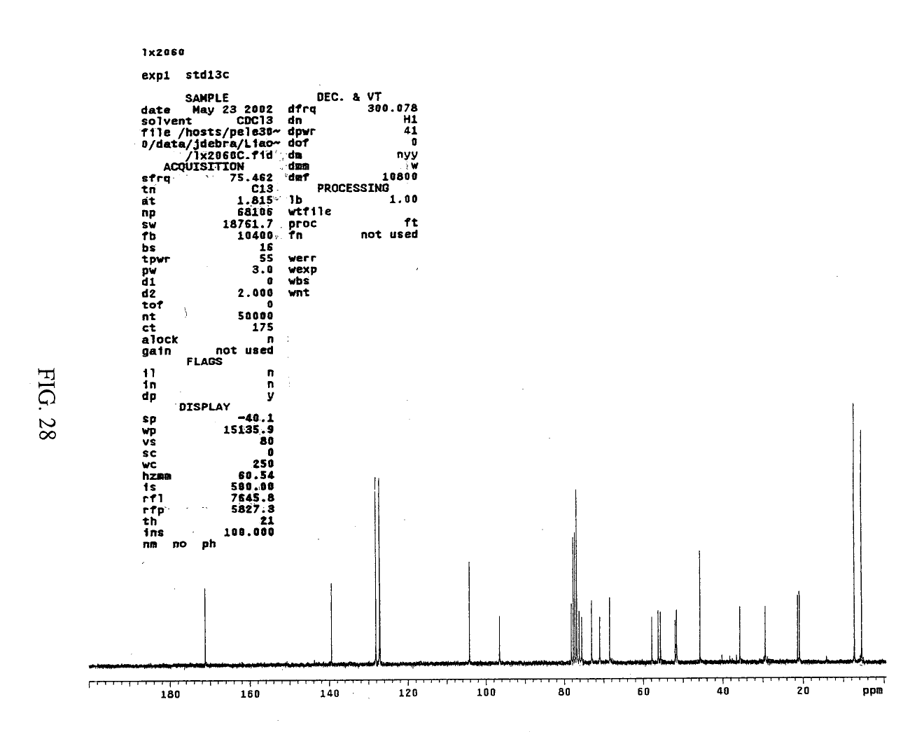

To a solution of olefin ent-20 (20 mg, 0.033 mmol) and N-methylmorpholine-N-oxide (12 μL) in 1 mL acetone at 0° C. was added OsO₄ (10 μL, 0.1 M solution in t-BuOH). The reaction was allowed to warm to RT and stirred overnight. After completion of the reaction, Na₂SO₃ solution was added at 0° C., and stirring was continued for another 1 h. After addition of EtOAc, the organic phase was separated, and the aq. phase was extracted with EtOAc. The combined organic extracts were dried (Na₂SO₄) and concentrated to provide 23 mg of crude intermediate iv, which was used in the next step without further purification. The crude intermediate iv was dissolved in CH₂Cl₂ and cooled to 0° C. Pyridine (10 μL, 0.126 mmol) and Pb(OAc)₄ (20 mg, 0.0432 mmol) were then added, and the mixture was vigorously stirred for 25 min at 0° C. After completion of the reaction, the mixture was filtered through silica gel and washed with ether. The filtrate was washed with brine, dried over Na₂SO₄, and concentrated. The residue was purified by FC (EtOAc/hexane 1/2) to provide aldehyde intermediate v (18 mg, 90% for two steps). Intermediate v: [α]_{D}²³=−54 (c 0.5, CHCl₃); IR 2953, 2873, 1733, 1453, 1136, 1106, 1038, 736 cm⁻¹; ¹H NMR (400 MHz, CDCl₃) δ 9.72 (1H, d, J=1.6 Hz), 7.30 (5H, m), 4.78 (1H, d, J=7.2 Hz), 4.72 (1H, d, J=7.2 Hz), 4.55 (1H, d, J=12.0 Hz), 4.42 (1H, d, J=12.0 Hz), 4.03 (1H, dd, J=1.6, 3.2 Hz), 3.92 (1H, d, J=1.6 Hz), 3.91 (1H, ddd, J=2.8, 2.8, 6.4 Hz), 3.75 (1H, d, J=8.8 Hz), 3.60 (1H, dddd, J=2.8, 6.4, 9.2, 11.6 Hz), 3.47 (1H, ddd, J=2.4, 4.0, 12.0 Hz), 3.43 (3H, s), 3.33 (3H, s), 3.32 (3H, s), 3.31 (1H, d, J=8.8 Hz), 3.29 (3H, s), 1.89 (2H, t, J=6.4 Hz), 1.68 (1H, dd, J=11.6, 23.6 Hz), 1.59 (1H, m), 1.13 (3H, s), 1.11 (3H, s), 0.95 (9H, t, J=8.4 Hz), 0.67 (6H, q, J=8.4Hz) ppm; ¹³C NMR (75 MHz, CDCl₃) δ 203.1, 139.5, 128.3, 127.5, 127.3, 104.5, 97.5, 82.8, 78.2, 75.8, 73.2, 71.0, 68.4, 58.0, 56.4, 55.9, 51.8, 45.8, 35.8, 31.1, 29.5, 21.5, 21.2, 7.4 (3C), 5.6 3C) ppm. MS (ES) m/z (%): 621.30 ([M+Na]⁺, 50). Aldehyde intermediate v (1.5 g, 2.5 mmol), ^{t}BuOH (43 mL), NaClO₂ (849 mg, 7.5 mmol), NaH₂PO₄ (517 mg, 3.75 mmol), 2-methyl-2-butene (43 mL, 2M in THF) and H₂O (8.6 mL) were combined and the mixture was stirred at RT for 1 h. The solvent was removed and the residue was diluted with EtOAc and washed with brine. The organic layers were dried over Na₂SO₄ and concentrated to afford 1.7 g of crude carboxylic acid intermediate vi which was used in the next step without further purification. To a solution of the crude acid intermediate vi in ether at 0° C. was added a CH₂N₂ solution in ether. After stirring at the same temperature for 1 h, the solvent was removed and the residue was purified was by FC (EtOAc/hexane 1/5) to provide the product methylester ent-21 (1.095 g, 70% for four steps from ent-20). Ent-21: [α]_{D}²³=−26 (c 1.0, CHCl₃); IR 2951, 1753, 1460, 1206, 1136, 1106, 1038, 735 cm⁻¹; ¹H NMR (400 MHz [FIG. 27], CDCl₃) δ 7.31 (5H, m), 4.74 (1H, d, J=7.2 Hz), 4.70 (1H, d, J=7.2 Hz), 4.55 (1H, d, J=12.0 Hz), 4.43 (1H, d, J=12.0 Hz), 4.28 (1H, d, J=3.2 Hz), 3.96 (1H, d, J=2.0 Hz), 3.93 (1H, m), 3.78 (1H, d, J=8.8 Hz), 3.76 (3H, s), 3.62 ( 1H, m), 3.47 (1H, ddd, J=2.4, 4.0, 11.6 Hz), 3.40 (3H, s), 3.35(1H, d, J=8.8 Hz), 3.34 (3H, s), 3.32 (6H, s), 1.89 (2H, t, J=6.0, 6.4 Hz), 1.69 (1H, dd, J=12.0, 24.0 Hz), 1.59 (1H, ddd, J=3.2, 3.2, 12.0 Hz), 1.13 (3H, s), 1.12 (3H, s), 0.95 (9H, t, J=8.4 Hz), 0.67 (6H, q, J=8.0 Hz) ppm; ¹³C NMR (75 MHz [FIG. 28], CDCl₃) δ 171.2, 139.4, 128.1, 127.2, 127.1, 104.2, 96.5, 78.2, 76.1, 75.5, 73.0, 71.0, 68.4, 57.8, 56.2, 55.6, 51.9, 51.6, 45.7, 35.7, 29.3, 21.3, 20.9, 7.2 (3C), 5.4 (3C) ppm; HRMS Calcd for C₃₂H₅₆O₁₀SiLi ([M+Li]⁺): 635.3803. Found: 635.3811.

Compound ent-22:

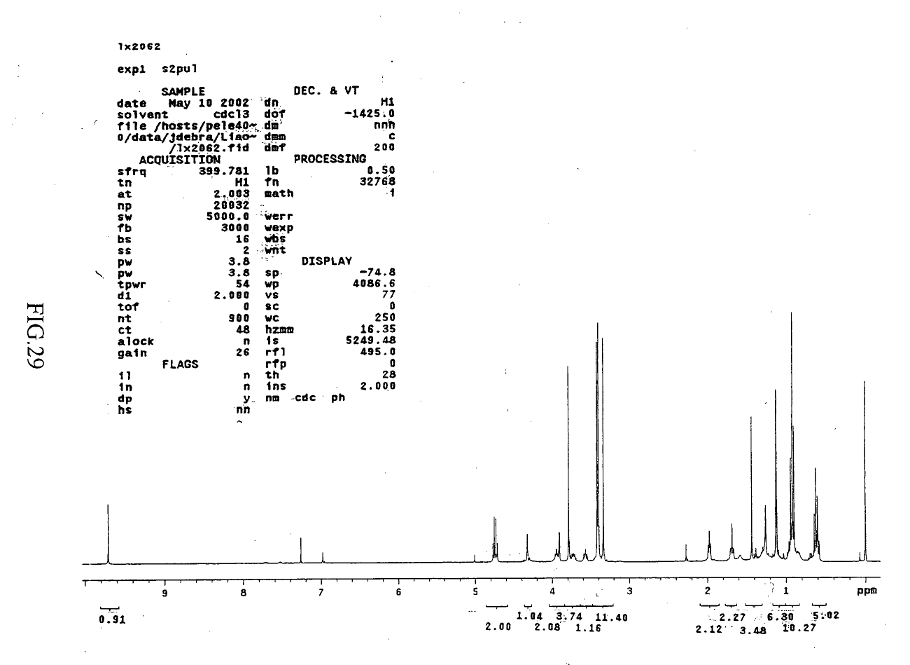

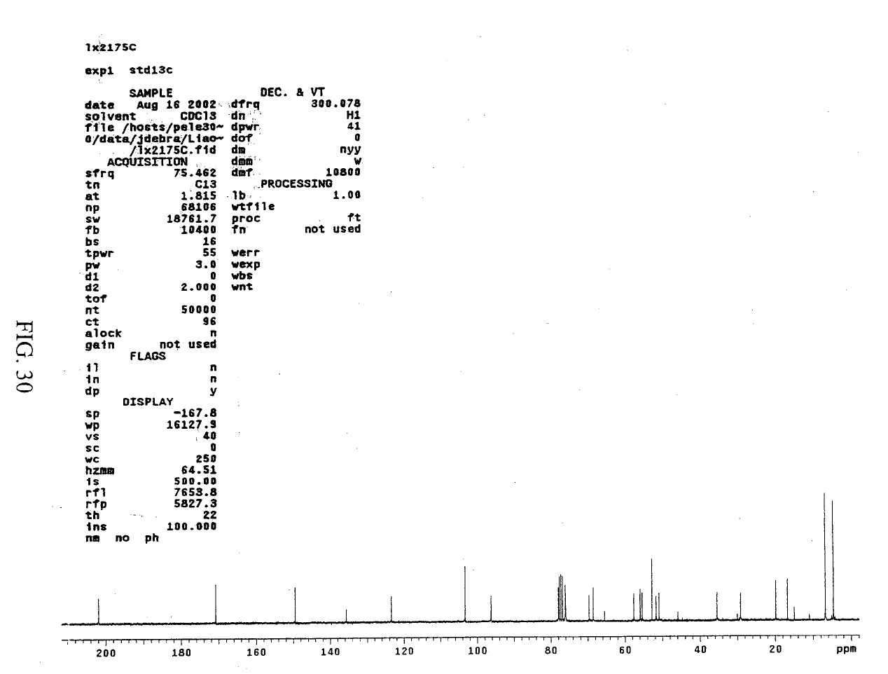

To a solution of ester ent-21 (217mg, 0.345 mmol) in MeOH (22 ml) was added 10% Pd/C (87 mg). After stirring the reaction mixture under an atmosphere of H₂ (balloon pressure) for 5 h, the reaction mixture was filtered through a plug of Celite, washed with ether, and concentrated to afford 185 mg of product intermediate vii (98%). [α]_{D}²³=−50 (c 0.8, CHCl₃); IR 3513, 2952, 1751, 1206, 1156, 1106, 1064, 736 cm⁻¹; ¹HNMR (400 MHz, CDCl₃) δ 4.74 (1H, d, J=7.2 Hz), 4.70 (1H, d, J=6.4 Hz), 4.30 ( 1H, d, J=3.6 Hz), 3.98 (1H, d, J=2.0 Hz), 3.91 (1H, dd, J=1.6, 9.2 Hz), 3.86 (1H, ddd, J=3.6, 6.4, 6.4 Hz), 3.78 (3H, s), 3.71 (1H, m), 3.52 (1H, ddd, J=2.4, 4.8, 11.6 Hz), 3.41 (3H, s), 3.40 (3H, s), 3.39 (3H, s), 3.33 (3H, s), 3.26 (1H, dd, J=7.2, 10.8 Hz), 3.04(1H, m), 1.91 (2H, t, J=6.0, 6.4 Hz), 1.68 (2H, m), 1.10 (3H, s), 1.03 (3H, s), 0.96 (9H, t, J=8.4 Hz), 0.68 (6H, q, J=8.0 Hz) ppm; ¹³C NMR (75 MHz, CDCl₃) δ 171.0, 105.5, 96.5, 78.6, 76.5, 75.8, 70.9, 70.5, 69.6, 57.9, 56.2, 55.6, 51.9, 51.6, 44.9, 36.0, 29.2, 22.3, 21.6, 7.1 (3C), 5.3 (3C) ppm. To a solution of alcohol intermediate vii (185 mg, 0.3455 mmol) and triethylamine (240 μL, 1.73 mmol) in DMSO/CH₂Cl₂ (1/1, 14 mL) was added Py.SO₃ at 0° C. After 45 min, the reaction mixture was allowed to warm to RT for 75 min, followed by dilution with ether. After washing with water and brine, drying over Na₂SO₄, and concentration, 175 mg of crude aldehyde ent-22 was obtained and used in the next step without further purification. Ent-22: [α]_{D}²³=−56 (c 1.0, CHCl₃); IR 2952, 1753, 1720, 1393, 1210, 1153, 1042, 733 cm⁻¹; ¹H NMR (400 MHz [FIG. 29], CDCl₃) δ 4.74 (1H, d, J=7.2 Hz), 4.70 (1H, d, J=6.4 Hz), 4.30 ( 1H, d, J=3.6 Hz), 3.98 (1H, d, J=2.0 Hz), 3.91 (1H, dd, J=1.6, 9.2 Hz), 3.86 (1H, ddd, J=3.6, 6.4, 6.4 Hz), 3.78 (3H, s), 3.71 (1H, m), 3.52 (1H, ddd, J=2.4, 4.8, 11.6 Hz), 3.41 (3H, s), 3.40 (3H, s), 3.39 (3H, s), 3.33 (3H, s), 3.26 (1H, dd, J=7.2, 10.8 Hz), 3.04(1H, m), 1.91 (2H, t, J=6.0, 6.4 Hz), 1.68 (2H, m), 1.10 (3H, s), 1.03 (3H, s), 0.96 (9H, t, J=8.4 Hz), 0.68 (6H, q, J=8.0 Hz) ppm; ¹³C NMR (75 MHz [FIG. 30], CDCl₃) δ 202.4, 171.1, 103.5, 96.5, 78.2, 76.4, 76.2, 69.8, 68.8, 57.9, 56.2, 55.7, 53.0, 52.0, 51.2, 40.9, 35.7, 29.4, 20.1, 17.0, 7.02 (3C), 4.89 (3C) ppm; MS (ES) m/z (%: 559.36 ([M+Na]⁺, 20).

Compound ent-23:

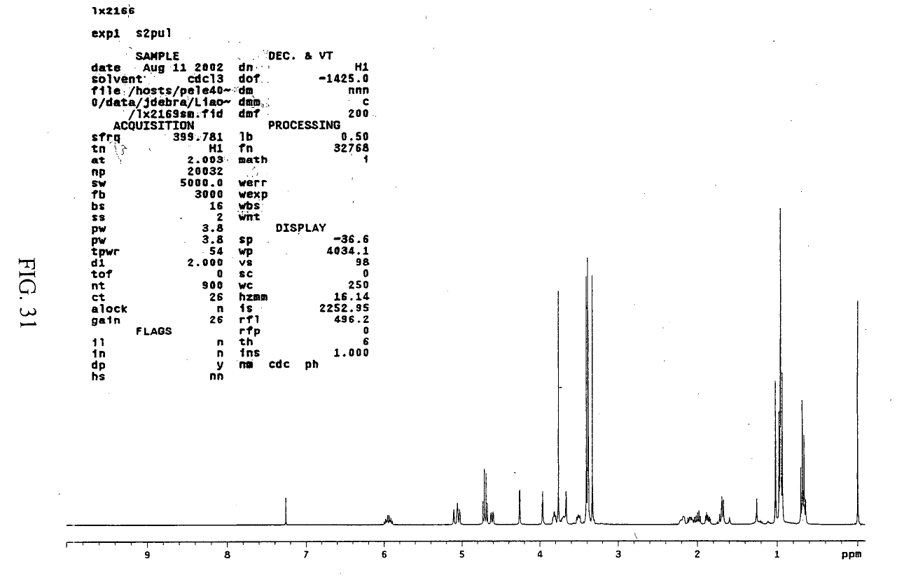

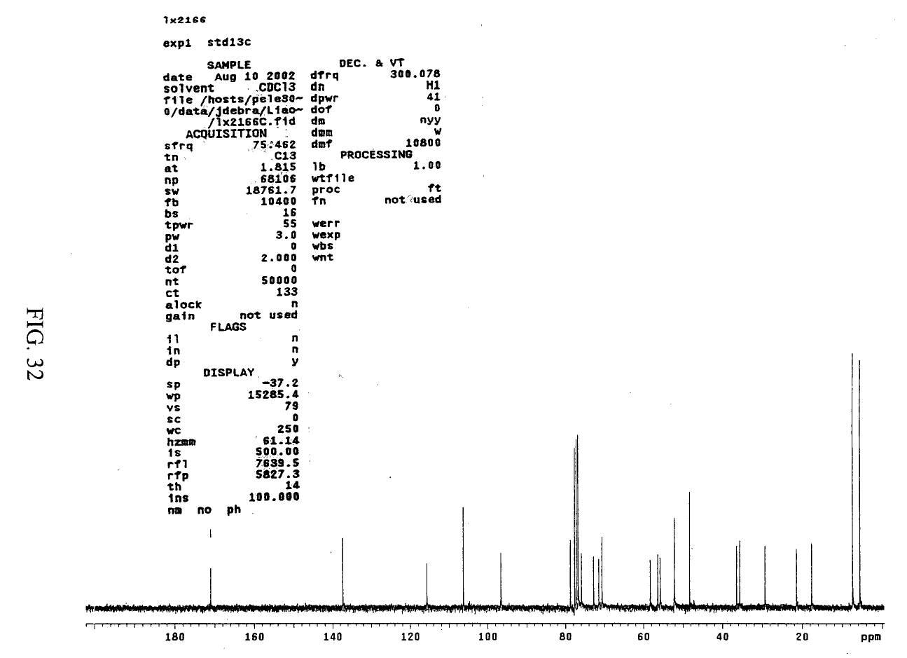

To a solution of diethylmethoxyborane (361 μL, 2.5 mmol) in ether (2.14 mL) was added allylmagnesium bromide (1.0 M in ether, 2.5 mL) drop-wise at 0° C. The white precipitous mixture was stirred at 0° C. for 40 min and then allowed to stand for 6 min. A 1.3 mL aliquot of this solution was added drop-wise to a cold (−10° C.) solution of aldehyde ent-22 (175 mg, 0.326 mmol) in 8 mL of ether. After stirring for 25 min at the same temperature, 5 mL aq. sat. NH₄Cl and ether were added. The organic layer was washed with brine, dried over Na₂SO₄ and concentrated. The residue was purified by FC (EtOAc/hexane 5/8) to provide the product ent-23 (170 mg, 85% for three steps). Ent-23: [α]_{D}²³=−52 (c 1.0, CHCl₃); IR 3533, 2940, 1750, 1210 1157, 1107, 1053, 740 cm⁻¹; ¹H NMR (400 MHz [FIG. 31], CDCl₃) δ 5.95 (1H, dddd, J=6.8, 6.8, 10.0, 17.2 Hz), 5.06 (2H, m), 4.72 (1H, d, J=7.2 Hz), 4.68 (1H, d, J=7.2 Hz), 4.61 (1H, dd, J=1.6, 10.4 Hz), 4.26 (1H, d, J=3.6 Hz), 3.96 (1H, d, J=2.0 Hz), 3.82 (1H, ddd, J=4.0 6.4, 6.4 Hz), 3.76 (3H, s), 3.70 (1H, m), 3.66 (1H, s), 3.51 (1H, m), 3.41 (3H, s), 3.39 (3H, s), 3.38 (3H, s), 3.33 (3H, s), 2.17-2.22 (1H, m), 2.04-2.12 (1H, m), 1.98 (1H, dd, J=3.2, 3.6 Hz), 1.86 (1H, ddd, J=5.2, 6.0, 14.4 Hz), 1.69 (2H, m), 1.02 (3H, s), 0.95 (3H, s), 0.95 (9H, t, J=8.0 Hz), 0.68 (6H, q, J=8.0 Hz) ppm; ¹³C NMR (75 MHz [FIG. 32], CDCl₃) δ 171.0, 137.3, 115.7, 106.3, 96.6, 78.8, 76.8, 75.9, 72.8, 71.4, 70.6, 58.3, 56.4, 55.7, 52.1, 48.3, 36.4, 35.6, 29.2, 21.3, 17.6, 7.3 (3C), 5.5 (3C) ppm. HRMS Calcd for C₂₈H₅₄O₁₀SiLi ([M+Li]⁺): 585.3646. Found: 585.3658.

Compound 24:

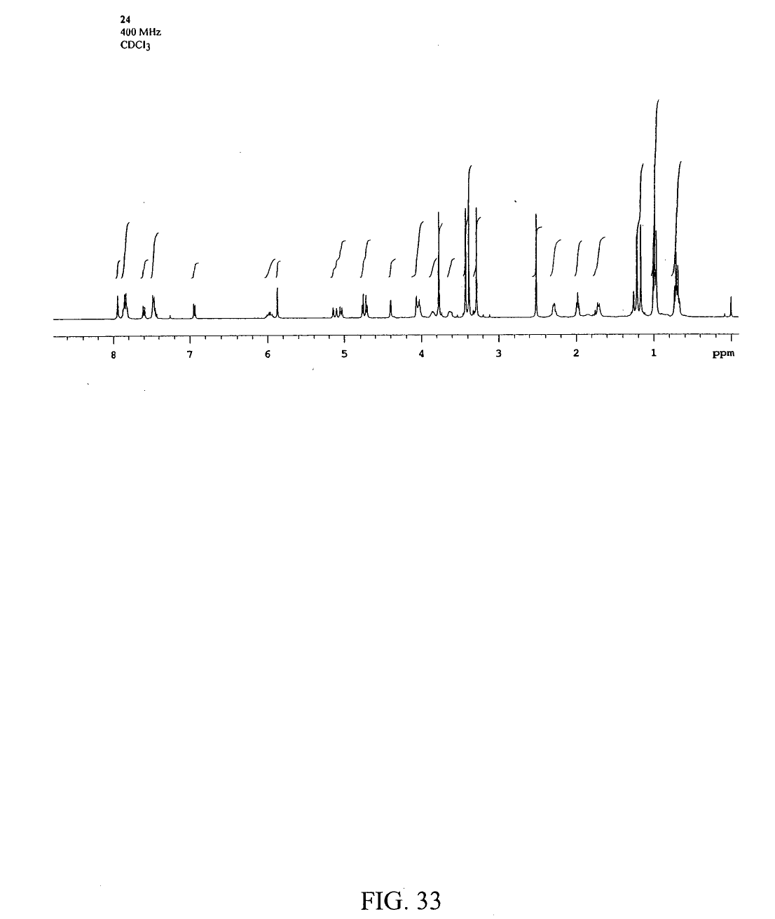

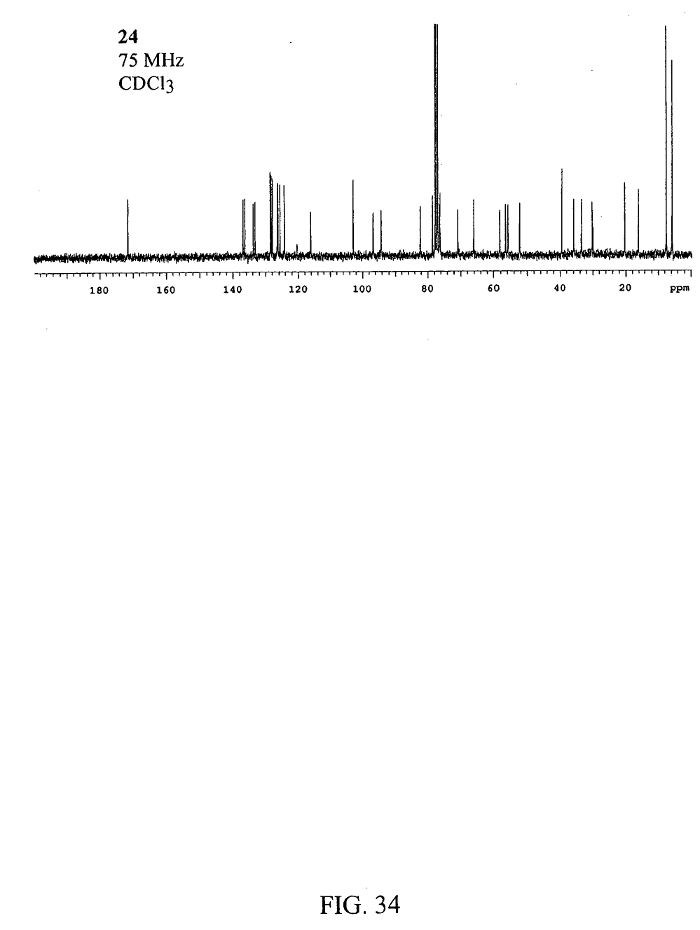

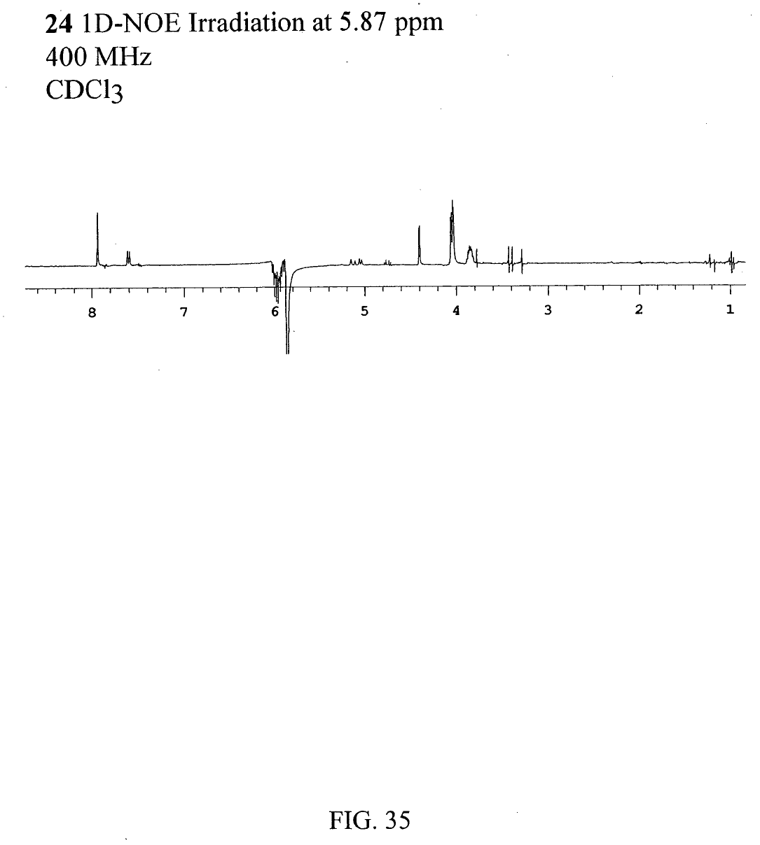

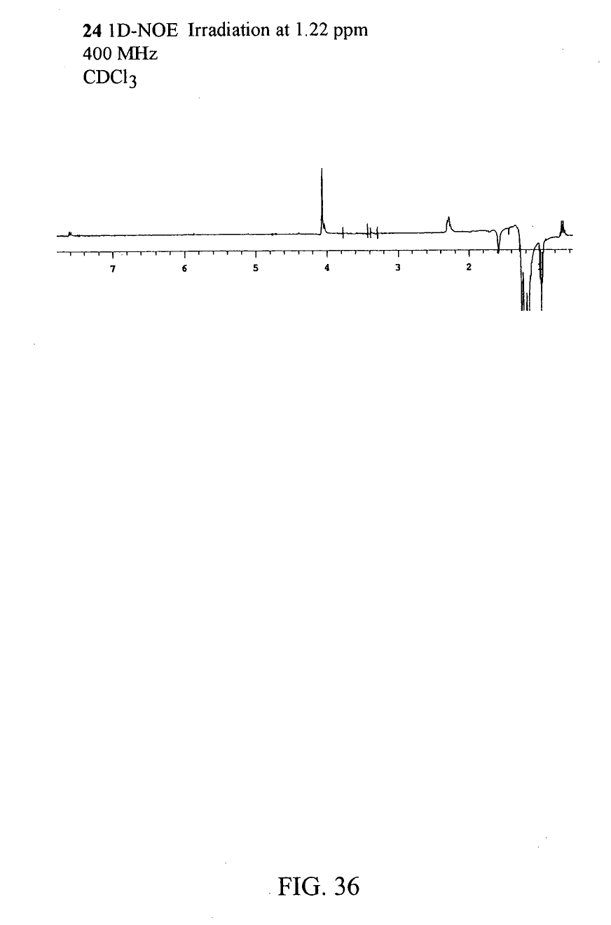

To a solution of alcohol 23 (50 mg, 0.0865 mmol) and 2-naphthaldehyde (67 mg, 0.432 mmol, 5 eq) in CH₂Cl₂ (5 mL) was added TESOTf (10 μL, 0.0433 mmol, 0.5 eq) dropwise at −78° C. After stirring for 15 min at −78° C., 2,6-lutidine (81 μl, 0.692 mmol, 8 eq) was added followed by the addition of TESOTf (100 μL, 0.4325 mmol, 5 eq). The reaction mixture was allowed to warm to 0° C. over 1 hour, after which no starting alcohol was detected by TLC. Ether (50 mL) and aqueous NaHCO₃ (10 mL) were added, the organic phase was separated, and the aqueous phase was extracted with ether (2×20 mL). The combined organic solution was dried (Na₂SO₄) and filtered and the solvents were removed under reduced pressure. Flash column chromatography (1:9 EtOAc/Hexanes) provided pure product 24 (47 mg, 81%). [α]_{D}²³=+97 (c 0.25, CHCl₃); IR 2953, 1750, 1383, 1153, 1130, 1103, 1010, 816, 736 cm⁻; ¹H NMR (400 MHz [FIG. 33], CDCl₃) δ 7.93 (1H, s), 7.80-7.90 (3H, m), 7.60 (1H, dd, J=1.6, 8.4 Hz), 7.45-7.50 (2H, m), 5.97 (1H, dddd, J=6.8, 6.8, 10.4, 17.6 Hz), 5.86 (1H, s), 5.13 (1H, dd, J=1.6, 17.6 Hz), 5.04 (1H, dd, J=1.2, 10.4 Hz), 4.77 (1H, d, J=6.8 Hz), 4.71 (1H, d, J=6.8 Hz), 4.39 (1H, d, J=3.2 Hz), 4.06 (1H, d, J=2.4 Hz), 4.00-4.08 (2H, m), 3.82-3.90 (1H, m), 3.78 (3H, s), 3.63 (1H, ddd, J=2.4, 5.6, 10.4 Hz), 3.43 (3H, s), 3.39 (3H, s), 3.28 (3H, s), 2.22-2.34 (2H, m), 1.98 (2H, app.t, J=6.0 Hz), 1.68-1.78 (2H, m), 1.22 (3H, s), 1.17 (3H, s), 0.99 (9H, t, J=8.0 Hz), 0.70 (6H, q, J=8.0 Hz) ppm; ¹³C NMR (75 MHz [FIG. 34], CDCl₃) δ 171.4, 136.9, 136.3, 133.8, 133.2, 128.5, 128.2, 127.8, 126.4, 126.2, 125.6, 124.2, 116.0, 102.8, 96.7, 94.2, 82.2, 78.5, 76.3, 76.1, 70.8, 66.1, 58.2, 56.4, 55.7, 52.2, 39.4, 35.8, 33.4, 30.2, 20.3, 16.1, 7.4 (3C), 5.7 (3C) ppm. HRMS Calcd for C₃₈H₅₉O₁₀SiLi ([M+Li]⁺): 709.3959. Found: 709.3943.

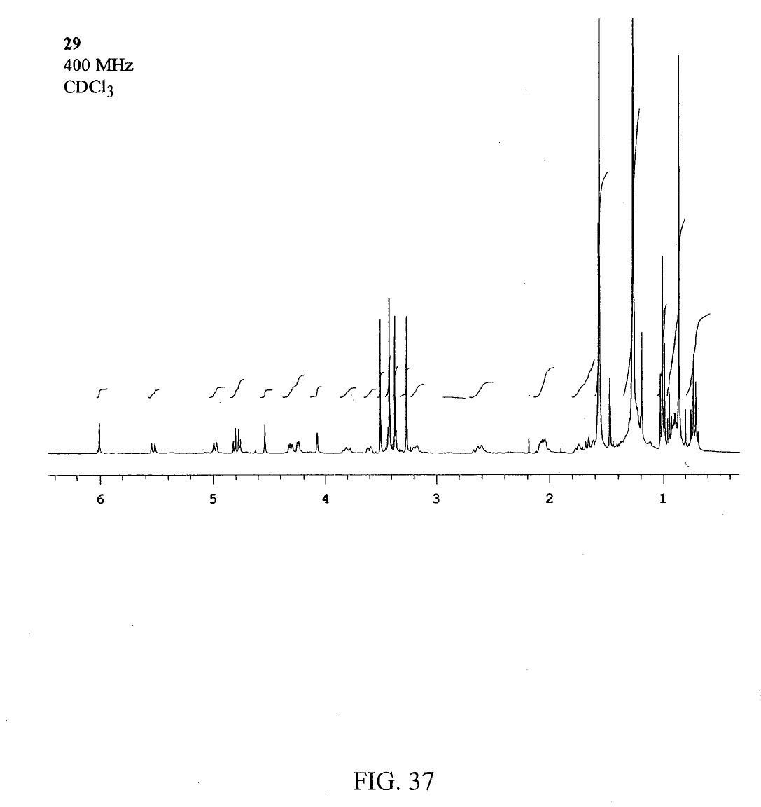

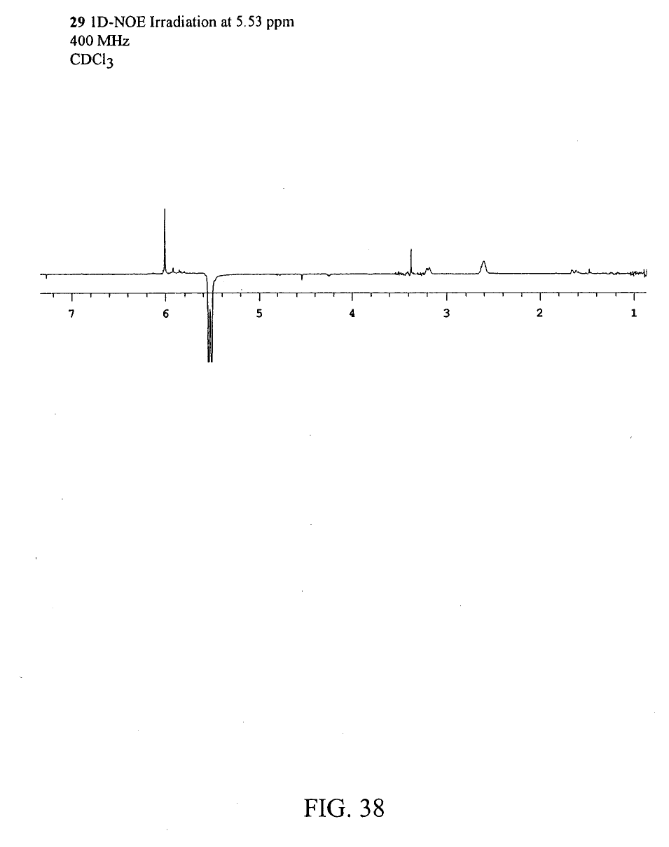

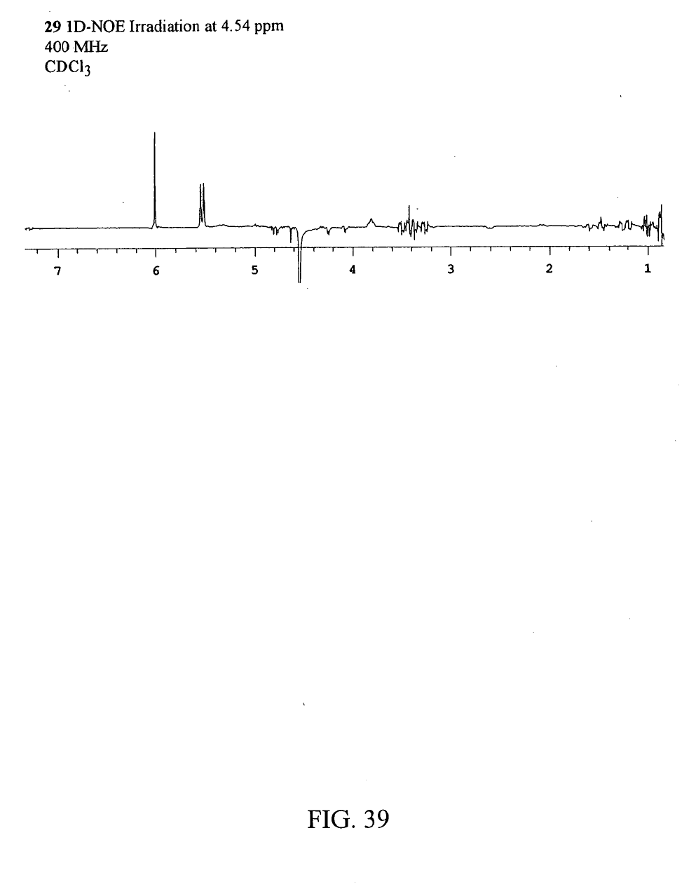

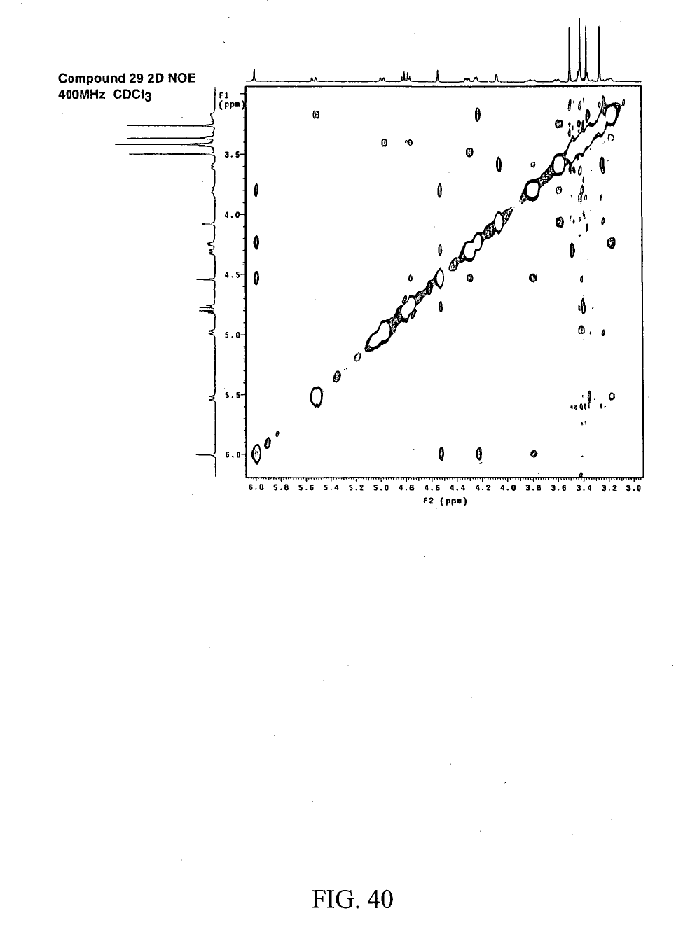

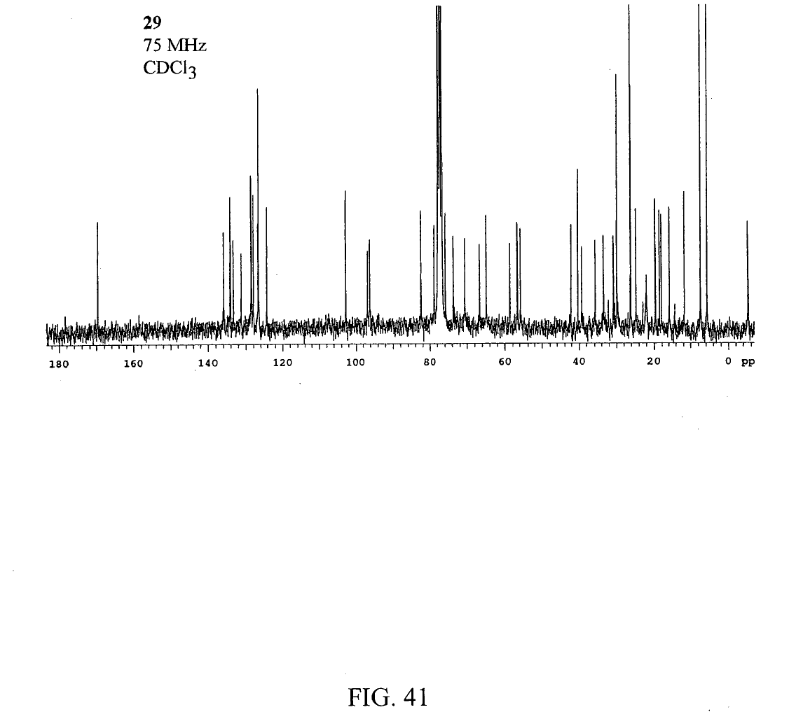

Compound 29:

[α]_{D}²³=+55 (c 0.15, CHCl₃); IR 2928, 1756, 1613, 1467, 1379, 1301, 1258, 1152, 1052, 834 cm⁻¹; ¹H NMR (400 MHz, CDCl₃ [FIG. 37]) δ 7.94 (1H, s), 7.82-7.90 (3H, m), 7.65 (1H, dd, J=1.2, 8.4 Hz), 7.48-7.53 (2H, m), 6.00 (1H, s), 5.53 (1H, d, J=12.0 Hz), 4.98 (1H, d, J=10.0 Hz), 4.81 (1H, d, J=7.2 Hz), 4.76 (1H, d, J=7.2 Hz), 4.54 (1H, s), 4.31 (1H, dd, J=4.0, 10.0 Hz), 4.24 (1H, d, J=5.2 Hz), 4.08 (1H, d, J=2.4 Hz), 3.76-3.86 (1H, m), 3.60 (1H, ddd, J=2.4, 4.0, 12.0 Hz), 3.50 (3H, s), 3.42 (3H, s), 3.40 (2H, m), 3.37 (3H, s), 3.26 (3H, s), 3.14-3.22 (1H, m), 2.56-2.68 (2H, m), 1.99-2.12 (3H, m), 1.54-1.80 (4H, m), 1.47 (3H, s), 1.21-1.42 (2H, m), 1.26 (3H, s), 1.19 (3H, s), 1.00 (9H, t, J=8.0 Hz), 0.94 (3H, t, J=7.6 Hz), 0.86 (9H, s), 0.73 (6H, q, J=8.0 Hz), 0.00 (3H, s), −0.01 (3H, s) ppm; ¹³C NMR (75 MHz, CDCl₃ [FIG. 41]) δ 169.7, 135.8, 134.1, 134.0, 133.2, 131.2, 128.5, 128.4, 127.9, 126.6, 126.4, 124.2, 102.8, 96.7, 96.1, 82.4, 78.8, 77.9, 77.4, 75.8, 73.7, 70.6, 66.6, 64.9, 58.7, 56.6, 56.5, 55.8, 42.2, 40.4, 39.3, 35.8, 34.5, 30.9, 29.9, 26.2 (3C), 24.8, 19.7, 18.6, 18.1, 15.7, 11.7, 7.4 (3C), 5.7 (3C), −5.2, −5.3 ppm; HRMS Calcd for C₅₂H₈₆O₁₂Si₂Li ([M+Li]⁺): 965.5818. Found: 965.5820.

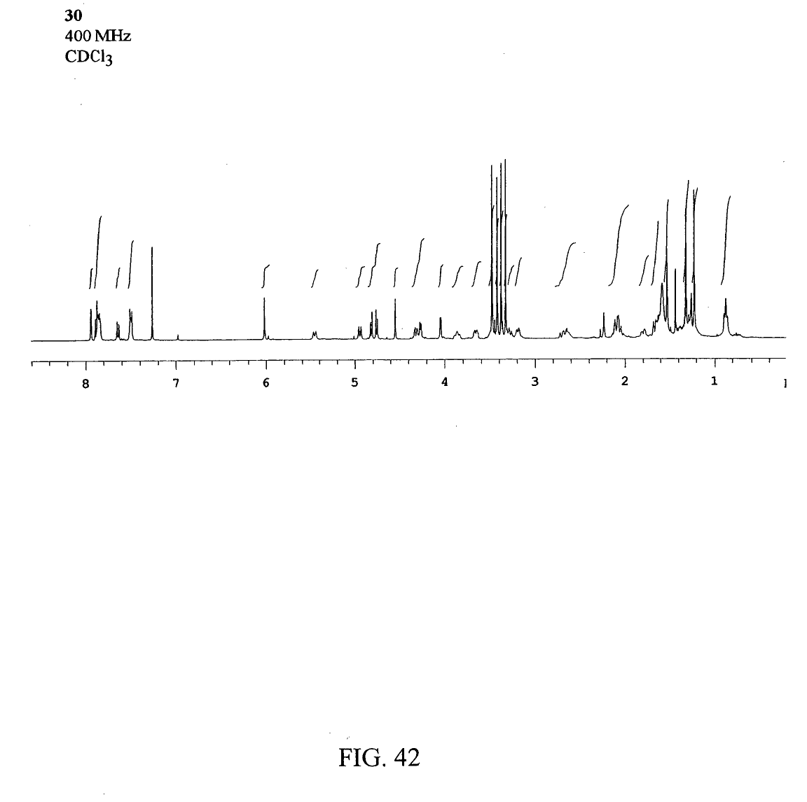

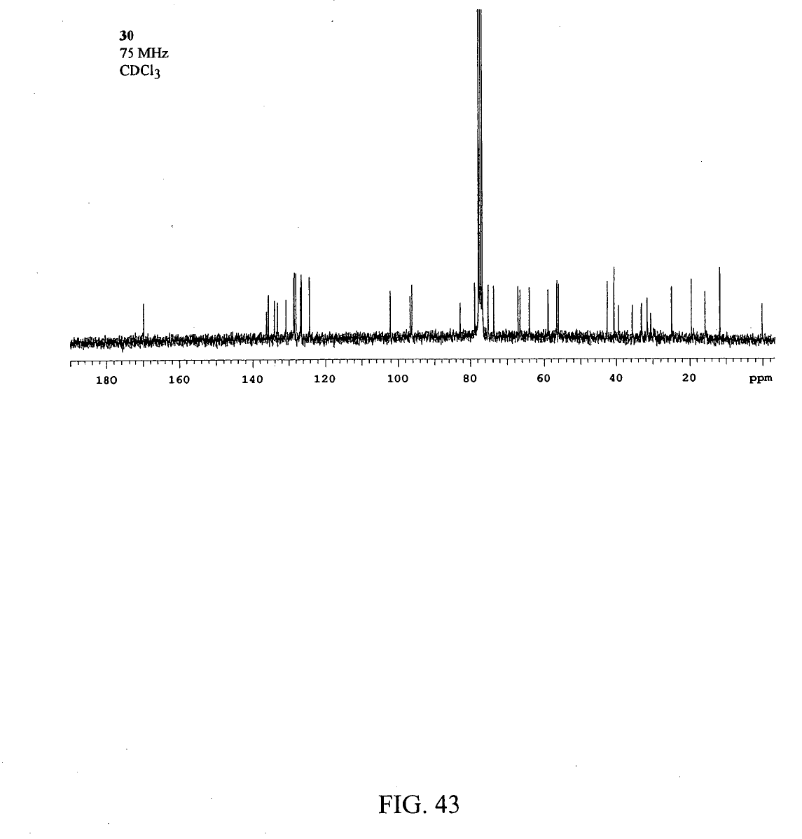

Compound 30:

[α]_{D}²³=+50 (c 0.2, CHCl3); IR 3453, 2928, 1757, 1460, 1383, 1150, 1102, 1050, 951, 750 cm⁻¹; ¹H NMR (400 MHz, CDCl₃ [FIG. 42]) δ 7.94 (1H, s), 7.82-7.90 (3H, m), 7.64 (1H, dd, J=1.2, 8.8 Hz), 7.50 (2H, m), 6.03 (1H, s), 5.46 (1H, d, J=10.8 Hz), 4.95 (1H, d, J=10.4 Hz), 4.83 (1H, d, J=6.8 Hz), 4.76 (1H, d, J=6.8 Hz), 4.56 (1H, s), 4.33 (1H, dd, J=4.0, 9.6 Hz), 4.28 (1H, d, J=5.6 Hz), 4.05 (1H, d, J=2.8 Hz), 3.88 (1H, m), 3.66 (1H, ddd, J=3.2, 4.8, 11.6 Hz), 3.48 (3H, s), 3.48 (1H, m), 3.43 (3H, s), 3.38 (3H, s), 3.32 (3H, s), 3.29 (1H, m), 3.20 (1H, m), 2.60-2.72 (2H, m), 1.98-2.18 (3H, m), 1.80 (1H, m), 1.50-1.70 (3H, m), 1.56 (3H, s), 1.20-1.44 (2H, m), 1.32 (3H, s), 1.23 (3H, s), 0.87 (3H, t, J=6.8 Hz) ppm; ¹³C NMR (75 MHz, CDCl₃ [FIG. 43]) δ 169.8, 136.2, 135.6, 134.0, 133.2, 130.8, 128.6, 128.4, 128.0, 126.7, 126.5, 126.4, 124.1, 102.1, 96.7, 96.2, 82.7, 78.7, 77.8, 77.4, 75.2, 73.7, 67.0, 66.4, 64.0, 58.8, 56.6, 56.5, 56.0, 42.6, 40.6, 39.4, 35.7, 33.0, 31.6, 30.5, 24.9, 19.5, 15.8, 11.7 ppm; MS (ES) m/z (%): 753.20 ([M+Na]⁺, 100).

Compound ent-31:

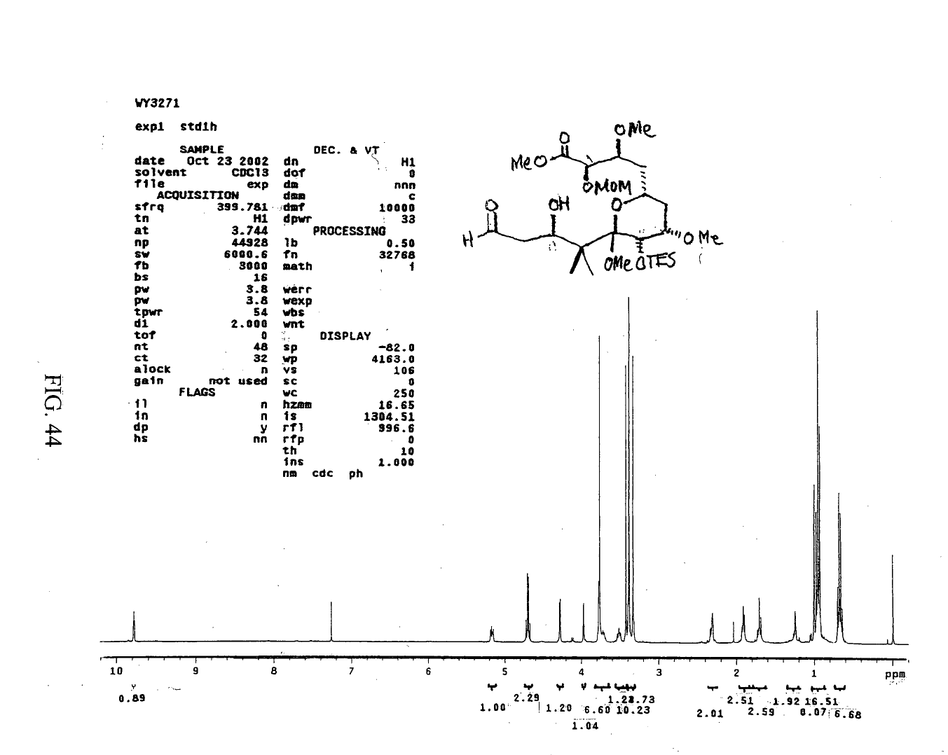

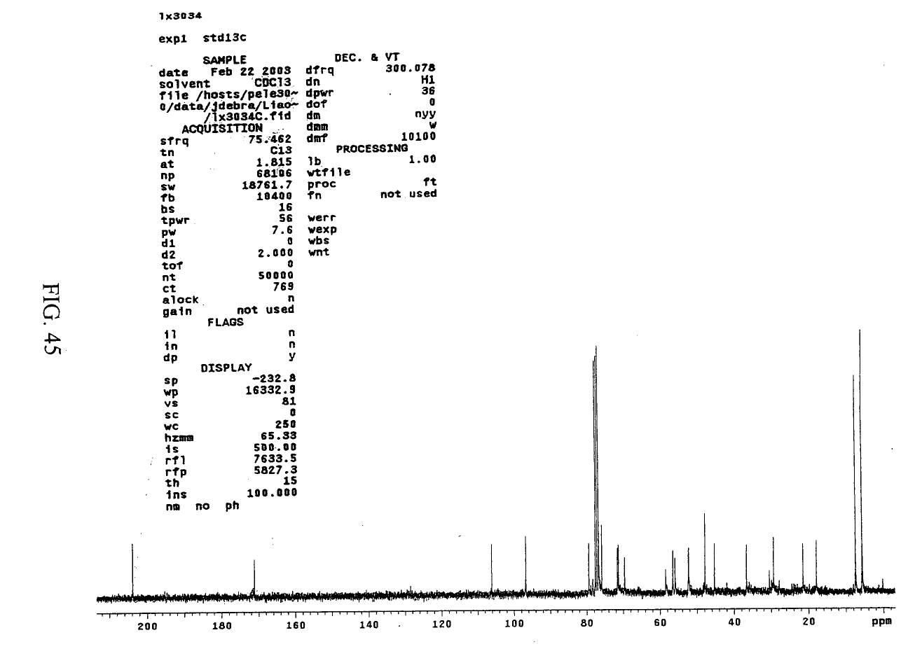

To a solution of olefin ent-23 (82 mg, 0.1417 mmol) and N-methylmorpholine-N-oxide (110 μL) in 4.0 mL acetone at 0° C. was added OSO₄ (80 μL, 0.1 M solution in t-BuOH). The mixture was allowed to warm to RT and stirred overnight. After completion of the reaction, Na₂SO₃ solution was added at 0° C., and stirring was continued for another 1 h, followed by the addition of EtOAc. The organic phase was separated, and the aq. phase was extracted with EtOAc. The combined organic extracts were dried (Na₂SO₄) and concentrated to provide 92 mg of crude diol intermediate viii which was used in the next step without further purification. The diol intermediate viii was dissolved in CH₂Cl₂ and cooled to 0° C. Pyridine (69 μL, 0.85 mmol) and Pb(OAc)₄ (132 mg, 0.283 mmol) were then added, and the mixture was vigorously stirred for 1.5 min at 0° C. After completion of the reaction, the mixture was filtered through silica gel and washed with ether. The filtrate was washed with brine, dried over Na₂SO₄, and concentrated. The residue was purified by FC (EtOAc/hexane 1/2) to give aldehyde ent-31 (73 mg, 95% for two steps). Ent-31: [α]_{D}²³=−30 (c 0.8, CHCl₃); IR 3510, 2953, 1749, 1461, 1385, 1209, 1155, 1102, 1042, 808, 736 cm⁻¹; ¹H NMR (400 MHz [FIG. 44], CDCl₃) δ 9.78 (1H, dd, J=1.6, 3.2 Hz), 5.17 (1H, dd, J=4.0, 9.8 Hz), 4.71 (1H, d, J=6.8 Hz), 4.68 (1H, d, J=6.8 Hz), 4.28 (1H, d, J=4.0 Hz), 3.97 (1H, d, J=2.0 Hz), 3.78 (3H, s), 3.69-3.79 (2H, m), 3.51 (1H, ddd, J=2.4, 8.4, 8.4 Hz), 3.43 (3H, s), 3.39 (3H, s), 3.34 (3H, s), 2.29-2.34 (2H, m), 1.89-1.94 (2H, m), 1.69-1.73 (2H, m), 1.00 (3H, s), 0.96 (9H, t, J=8.0 Hz); 0.93 (3H,s), 0.68 (6H, q, J=8.0 Hz) ppm: ¹³C NMR (75 MHz [FIG. 45], CDCl₃) δ 203.8, 171.1, 106.1, 96.8, 79.4, 76.7, 75.9, 71.6, 71.3, 69.6, 58.4, 56.5, 55.9, 52.3, 52.3, 47.9, 45.3, 36.7, 29.4, 21.4, 17.9, 7.4 (3C), 5.7 (3C) ppm; MS (ES) m/z (%): 603.20 ([M+Na]⁺, 100).

Compounds ent-32a and ent-32b:

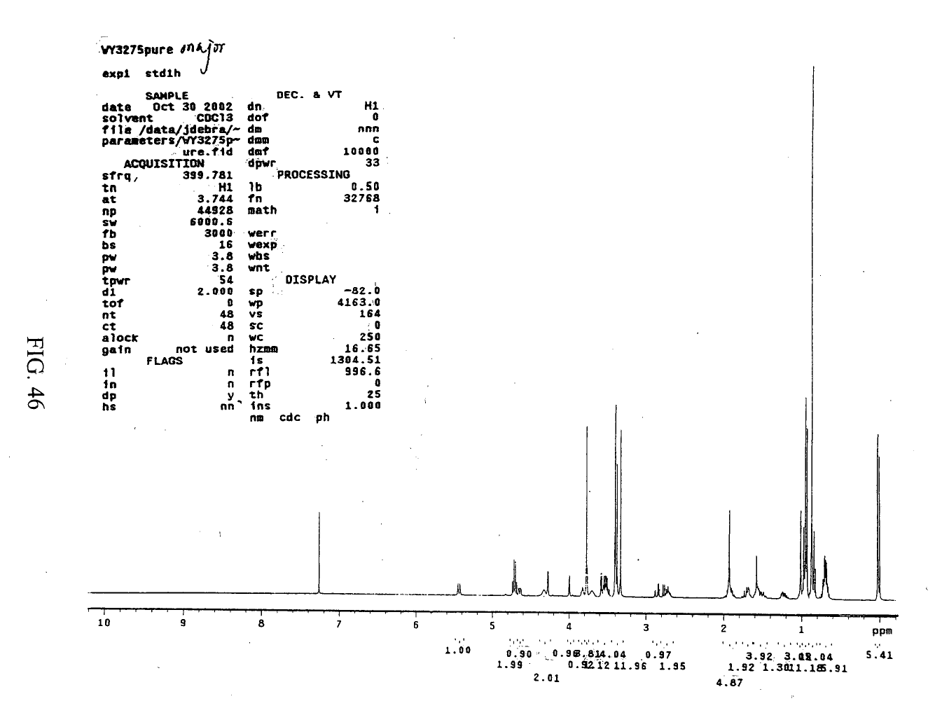

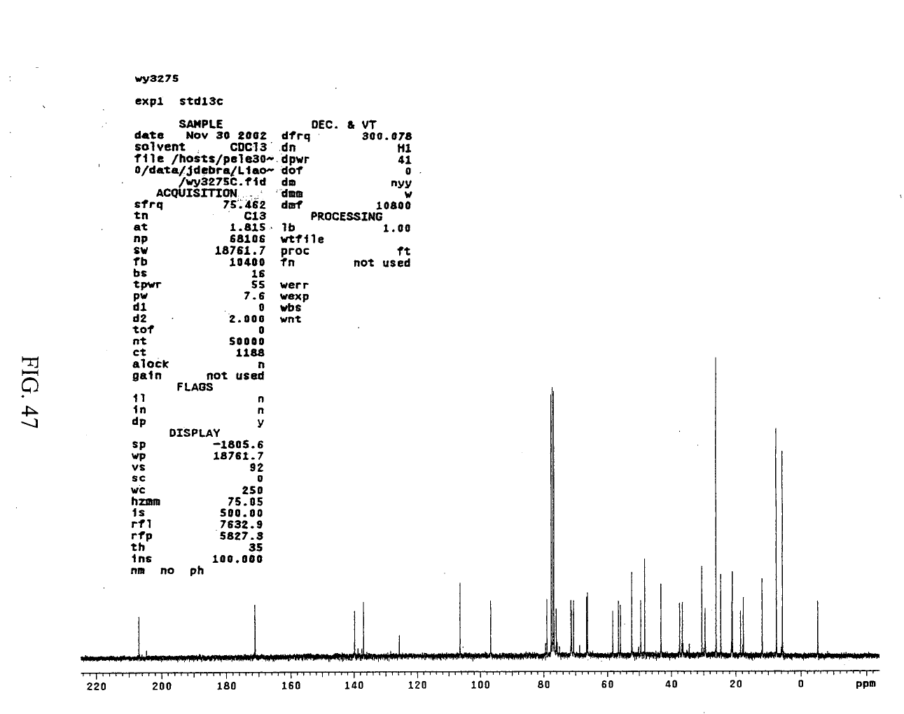

To a solution of ketone ent-6 (90 mg, 0.333 mmol) in ether (1.5 mL) at −78° C. was added diisopropylethylamine (83 μL, 0.473 mmol), followed by addition of diethylboron trifluoromethanesulfonate (324 μL, 0.324 mmol). After stirring for 15 min at −78° C. and 45 min at −30° C., the opaque white solution was cooled to −78° C. followed by the drop-wise addition of a solution of aldehyde ent-23 in ether (0.5 mL, 0.25 mL rinse). The mixture was stirred for 2 h at this temperature and quenched by the addition of 1.6 mL MeOH. After 5 min, 330 μl pH 7.0 phosphate buffer was added and the mixture was allowed to warm to RT. After a mixture of MeOH and 30% aq. H₂O₂ (2 mL, 2:1) was added and stirring continued for 1 h, the aq. layer was extracted with ether (50 mL×3). The combined organic layers were washed with aq. sat. NaHCO₃ and brine, dried and concentrated. The residue was purified by FC to afford 95 mg of a diastereomeric mixture of aldol products ent-32a and ent-32b (2:1 ratio as determined by H NMR, yield 87%). Ent-32a: [α]_{D}²³=−40 (c 0.25, CHCl₃); IR 3520, 2954, 1747, 1687, 1460, 1383, 1210,1101, 1053, 833 cm⁻¹; ¹H NMR (400 MHz [FIG. 46], CDCl₃) δ 5.44 (1H, dd, J=1.6, 10.0 Hz), 4.73 (1H, d, J=7.2 Hz), 4.69 (1H, d, J=7.6 Hz), 4.64 (1H, dd, J=2.0, 10.4 Hz); 4.33 (1H, m), 4.28 (1H, d, J=3.6 Hz), 3.94 (1H, d, J=1.6 Hz), 3.83 (1H, ddd, J=3.6, 6.0, 6.0 Hz), 3.77 (3H, s), 3.70 (1H, m), 3.58 (1H, d, J=4.4 Hz), 3.54 (1H, dd, J=5.6, 10.0 Hz), 3.53 (1H, m), 3.50 (1H, dd, J=5.6, 9.6 Hz), 3.40 (6H, s), 3.38 (3H, s), 3.33 (3H, s), 2.86 (1H, dd, J=3.2, 17.2 Hz), 2.75 (1H, dd, J=8.8, 17.6 Hz), 2.71 (1H, m), 1.93 (3H, d, J=1.2 Hz), 1.85-2.00 (2H, m), 1.64-1.78 (2H, m), 1.45-1.62 (2H, m), 1.17-1.29 (1H, m), 1.03 (1H, m), 1.02 (3H, s), 0.96 (9H, t, J=8.4 Hz); 0.94 (3H,s), 0.88 (9H, s), 0.85 (3H, t, J=8.0 Hz), 0.70 (6H, dq, J=4.4, 7.2 Hz); 0.03 (3H, s); 0.02 (3H, s); ¹³C NMR (75 MHz [FIG. 47], CDCl₃) δ 207.0, 171.2, 139.8, 137.0, 106.4, 96.8, 79.0, 76.9, 76.1, 71.5, 71.3, 70.6, 66.5, 66.2, 58.3, 56.5, 55.9, 52.3, 49.5, 48.3, 37.4, 36.6, 30.5, 29.5, 26.1 (3C), 24.6, 21.2, 18.5, 17.6, 11.8, 7.5 (3C), 5.6 (3C), −5.18, −5.23 ppm; HRMS Calcd for C₄₂H₈₂O₁₃Si₂Li ([M+Li]⁺): 857.5454. Found: 857.5452. Ent-32b: [α]_{D}^{≦}=−50 (c 0.6, CHCl₃ ); IR 3480, 2955, 1750, 1687, 1457, 1383, 1101, 837, 737cm⁻¹; ¹H NMR (400 MHz, CDCl₃) δ 5.39 (1H, dd, J=1.2, 10.4 Hz), 4.82 (1H, d, J=7.2 Hz), 4.71 (1H, d, J=7.2 Hz), 4.69 (1H, d, J=7.2 Hz); 4.34 (1H, m), 4.28 (1H, d, J=4.0 Hz), 4.22 (1H, s), 3.95 (1H, d, J=2.0 Hz), 3.78 (3H, s), 3.75 (1H, m), 3.70 (1H, m), 3.47-3.56 (3H, m), 3.39 (3H, s), 3.37 (3H, s), 3.33 (3H, s), 3.23 (1H, d, J=6.0 Hz), 2.82 (1H, dd, J=6.8, 16.4 Hz), 2.69 (1H, m), 2.56 (1H, dd, J=5.6, 16.0 Hz), 1.94 (3H, s), 1.87 -1.90 (2H, m), 1.68-1.73 (2H, m), 1.53-1.58 (2H, m), 1.12-1.29 (2H, m), 0.99 (3H, s), 0.97 (9H, t, J=8.0 Hz); 0.90 (3H,s), 0.87 (9H, s), 0.85 (3H, t, J=7.2 Hz), 0.70 (6H, q, J=8.0 Hz); 0.02 (3H, s); 0.01 (3H, s); ¹³C NMR (75 MHz, CDCl₃) δ 207.7, 171.2, 138.8, 137.5, 106.4, 96.8, 79.4, 75.8, 75.1, 71.5, 71.4, 68.7, 66.3, 58.3, 56.5, 55.9, 52.4, 52.3, 50.2, 48.3, 43.1, 36.7, 36.4, 30.5, 29.4, 26.1 (3C), 24.6, 21.3, 21.1, 18.5, 17.8, 11.8, 7.5 (3C), 5.7 (3C), −5.1, −5.2 ppm.

Compound ent-33a:

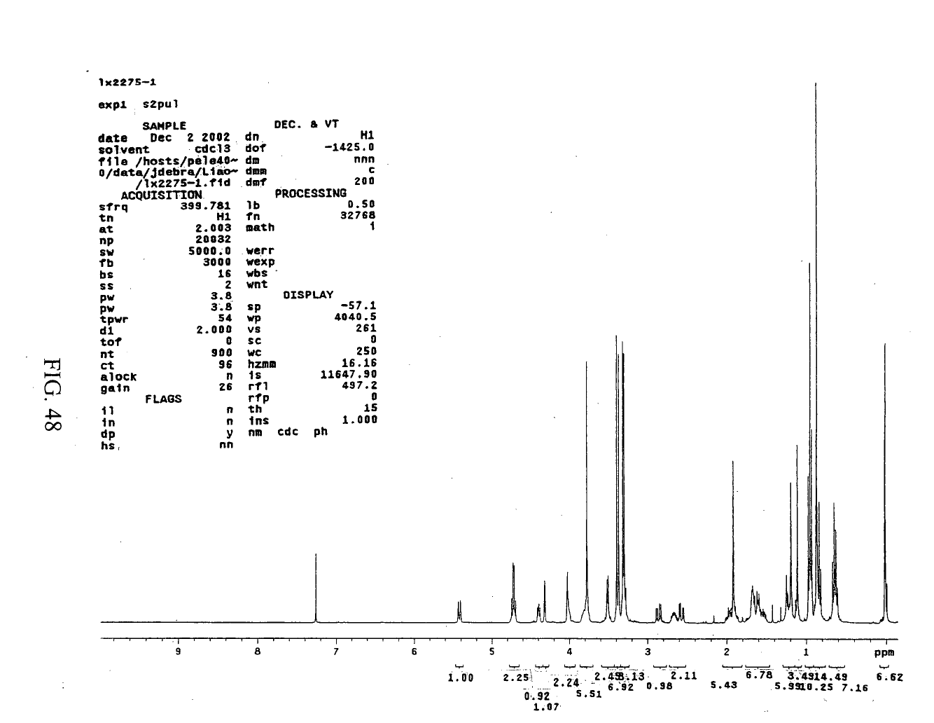

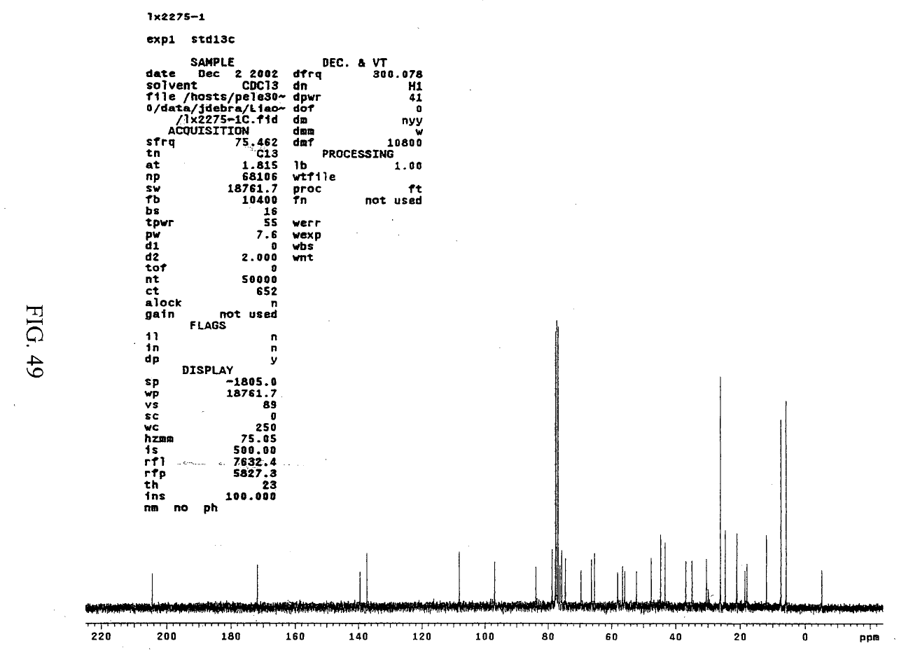

To a solution of the alcohol ent-32a (33 mg, 40.7 umol) in CH₂Cl₂ (4 mL) was added 2,6-tert-butyl-methyl-pyridine (256 mg, 1.221mmol) and Me₃OBF₄ (120 mg, 0.814 mmol) at RT. After stirring for 1 h, the mixture was diluted with ether and washed with sat. aq. NH₄Cl and brine. The organic phase was dried over Na₂SO₄ and concentrated. The residue was purified by FC (EtOAc/Hexanes 1/4) to afford 28 mg of product ent-33a (85%). Ent-33a: [α]_{D}²³=−45 (c 0.5, CH₂Cl₂); IR 2953.3, 2927, 1757, 1693, 1457, 1257, 1103 cm⁻¹; ¹H NMR (400 MHz [FIG. 48], CDCl₃) δ 5.42 (1H, dd, J=1.2, 12.0Hz), 4.74 (1H, d, J=7.2 Hz), 4.70 (1H, d, J=7.6 Hz), 4.40 (1H, dd, J=4.8, 7.6 Hz); 4.32 (1H, d, J=2.8 Hz), 4.03 (1H, d, J=2.0 Hz), 4.01 (1H, ddd, J=2.1, 5.2, 8.0 Hz), 3.82 (1H, m), 3.79 (1H, m), 3.78 (3H, s), 3.51 (2H, m), 3.40 (3H, s), 3.37 (3H, s), 3.32 (3H, s), 3.30 (3H, s), 2.86 (1H, dd, J=7.2, 16.8 Hz), 2.67 (1H, m), 2.57 (1H, dd, J=5.2, 16.8 Hz), 1.92 (3H, d, J=1.2 Hz), 1.86 -2.02 (2H, m), 1.49-1.76 (4H, m), 1.18-1.26 (2H, m), 1.19 (3H, s), 1.11 (3H, s), 0.95 ( 9H, t, J=8.0 Hz); 0.94 (3H,s), 0.87 (9H, s), 0.84 (3H, t, J=8.0 Hz), 0.64 (6H, q, J=8.0 Hz); 0.03 (3H, s); 0.02 (3H, s); ¹³C NMR (75 MHz [FIG. 49], CDCl₃) δ 204.0, 171.8, 139.4, 137.3, 108.2, 96.9, 83.9, 78.7, 76.2, 75.7, 74.5, 69.6, 66.3, 65.3, 58.1, 58.0, 56.5, 55.8, 52.1, 47.5, 44.6, 43.2, 36.8, 34.9, 26.1 (3C), 24.6, 21.0, 18.5, 17.9, 11.9, 7.4 (3C), 5.8 (3C), −5.20 (2C) ppm; HRMS Calcd for C₄₂H₈₀O₁₂Si₂Li ([M+Li—H₂O]⁺): 839.5348. Found: 839.5346.

Compound ent-34a:

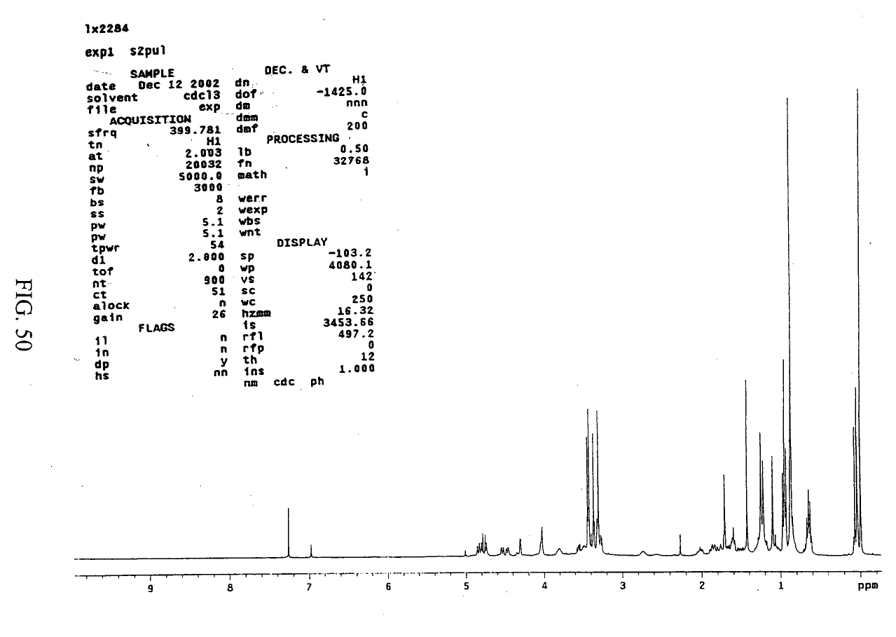

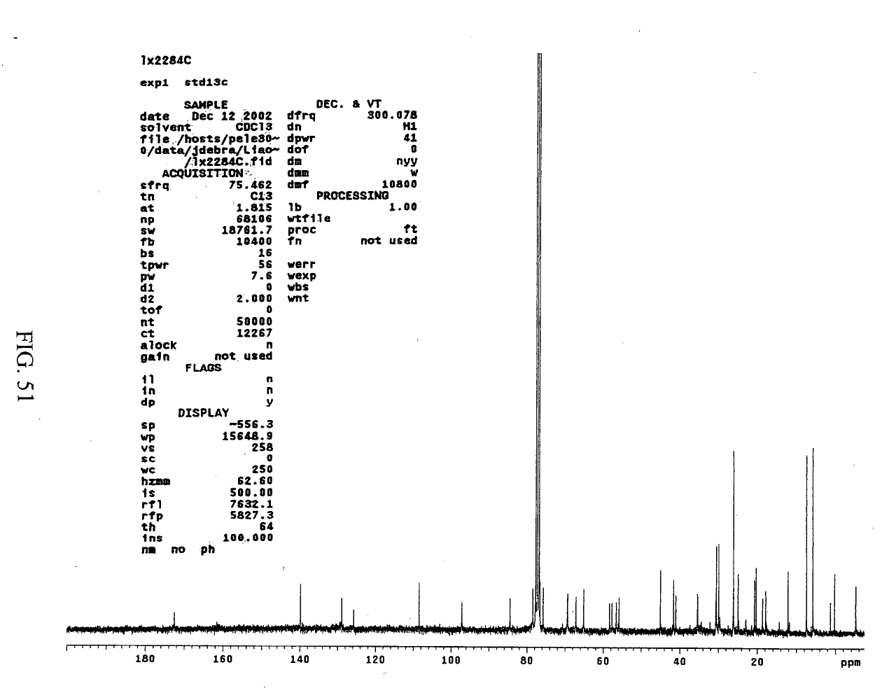

To a solution of ketone ent-33a (8 mg) (azeotropically dried with PhH) in 1.5 mL of CH₂Cl₂ was added a 1 M solution of (S)—B-Me-CBS reagent in toluene (200 μL, 0.2 mmol) at −30° C. followed by the addition of 1 M solution of BH₃.Me₂S (60 μL, 0.06 mmol) in CH₂Cl₂. After stirring at −30° C. for 1 h, the mixture was allowed to warm to RT and quenched with 1 mL of EtOH and ether. The solution was washed with water and brine, dried (Na₂SO₄) and concentrated. The residue was purified by FC (EtOAc/Hexanes 1/4 to 1/3) to provide pure alcohol intermediate ix (7.2 mg, 90%). Intermediate ix: [α]_{D}²³=−65 (c 0.5, CHCl₃); IR 3473, 2929, 1753, 1460, 1380, 1257, 1099, 836, 733 cm⁻¹; ¹H NMR (400 MHz, CDCl₃) δ 4.86 (1H, d, J=10.4 Hz), 4.75 (1H, d, J=7.2 Hz), 4.71 (1H, d, J=7.2 Hz), 4.57 (1H, d, J=6.4 Hz), 4.44 (1H, dd, J=3.6, 8.8 Hz); 4.33 (1H, d, J=2.8 Hz), 4.03 (1H, d, J=1.2 Hz), 4.01 (1H, m), 3.82 (1H, m), 3.78 (3H, s), 3.52 (1H, m), 3.49 (1H, dd, J=5.6, 9.6 Hz), 3.41 (3H, s), 3.38 (3H, s), 3.37 (3H, s), 3.33 (1H, m), 3.31 (3H, s), 3.16 (1H, s), 2.67 (1H, m), 2.00 (1H, ddd, J=5.6, 8.8, 14.0 Hz), 1.90 (1H, ddd, J=4.0, 5.6, 12.8 Hz), 1.81(1H, td, J=3.6, 14.0 Hz), 1.72 (3H, d, J=1.2 Hz), 1.43-1.78 (5H, m), 1.22 (3H, s), 1.13 (3H, s), 1.04-1.24 (2H, m), 0.96 (9H, t, J=7.6 Hz); 0.88 (9H, s), 0.88 (3H, t, J=7.2 Hz), 0.65 (6H, q, J=7.6 Hz); 0.03 (6H, s) ppm; ¹³C NMR (75 MHz, CDCl₃) δ 171.8, 139.9, 128.8, 108.2, 96.9, 84.2, 78.8, 76.2, 76.0, 75.7, 69.6, 68.8, 67.0, 65.3, 58.1, 56.5, 55.8, 52.2, 44.6, 41.8, 40.8, 36.5, 34.8, 30.5, 29.9, 26.1 (3C), 24.9, 21.0, 20.2, 18.6, 17.9, 12.0, 7.4 (3C), 5.8 (3C), −5.20 (2C) ppm; HRMS Calcd for C₄₂H₈₂O₁₂Si₂Li ([M+Li—H₂O]⁺): 841.5505. Found: 841.5507. To a stirred solution of alcohol intermediate ix (2 mg) in 0.5 mL THF at RT was added 0.5 ml of a 0.3 N aq. LiOH solution. The reaction mixture was stirred at RT overnight and diluted with EtOAc, washed with 3 mL of a 1M aq. solution of NaH₂PO₄ and brine. The organic extracts were dried over Na₂SO₄ and concentrated to provide 2 mg of crude carboxylic acid ent-34a, which was used in the next step without further purification. Ent-34a: IR 3440, 2927, 1732, 1463, 1257, 1104, 837; cm⁻¹; ¹H NMR (400 MHz [FIG. 50], CDCl₃) δ 4.84 (1H, d, J=10.0 Hz), 4.79 (1H, d, J=6.8 Hz), 4.71 (1H, d, J=6.8 Hz), 4.53 (1H, dd, J=2.0, 10.0 Hz), 4.47 (1H, dd, J=3.6, 9.2 Hz); 4.30 (1H, d, J=3.6 Hz), 4.04 (1H, m), 4.02 (1H, d, J=2.0 Hz), 3.80 (2H, m), 3.55 (1H, dd, J=6.0, 9.6 Hz), 3.49 (1H, m), 3.44 (3H, s), 3.42 (3H, s), 3.37 (3H, s), 3.32 (1H, m), 3.30 (3H, s), 3.27 (1H, m), 2.74 (1H, m), 2.02 (1H, ddd, J=4.0, 10.0, 14.0 Hz), 1.75-1.90 (4H, m), 1.71 (3H, s), 1.46-1.70 (3H, m), 1.22 (3H, s), 1.10 (3H, s), 1.12-1.28 (2H, m), 0.96 ( 9H, t, J=8.0 Hz); 0.88 (9H, s), 0.87 (3H, t, J=8.0 Hz), 0.64 (6H, q, J=8.0 Hz); 0.04 (6H, s); ¹³C NMR (75 MHz [FIG. 51], CDCl₃) δ172.5, 139.9, 128.9, 108.5, 97.1, 84.5, 78.4, 77.0, 76.6, 75.7, 69.4, 69.2, 67.1, 65.2, 58.3, 57.6, 56.5, 55.8, 45.0, 41.6, 41.0, 35.4, 35.3, 30.5, 29.9, 26.1 (3C), 24.9, 20.3, 17.8, 12.1, 7.4 (3C), 5.8 (3C), −5.20 (2C) ppm. MS (ES) m/z (%): 843.25 ([M+Na—H₂O]⁺, 100).

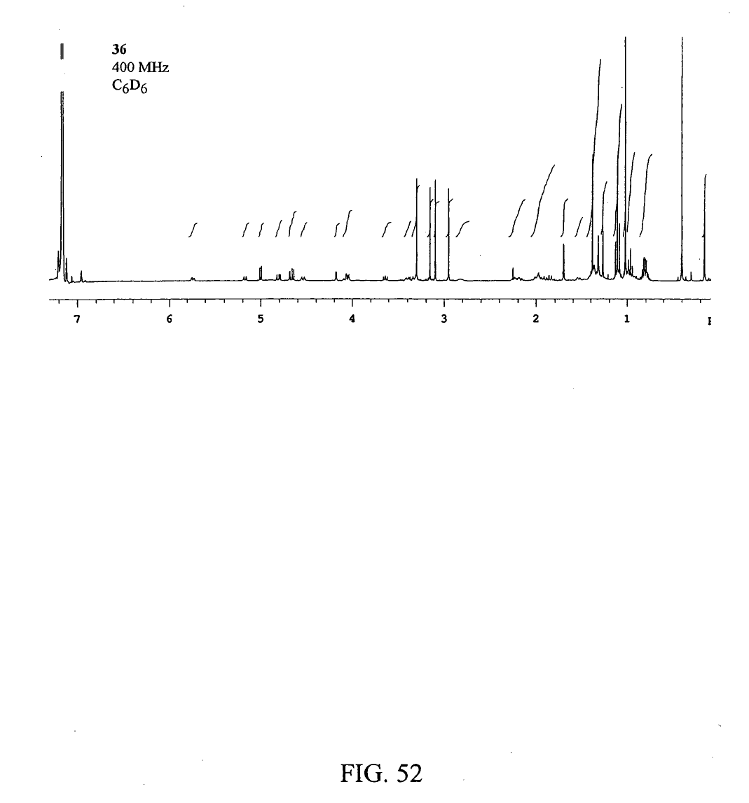

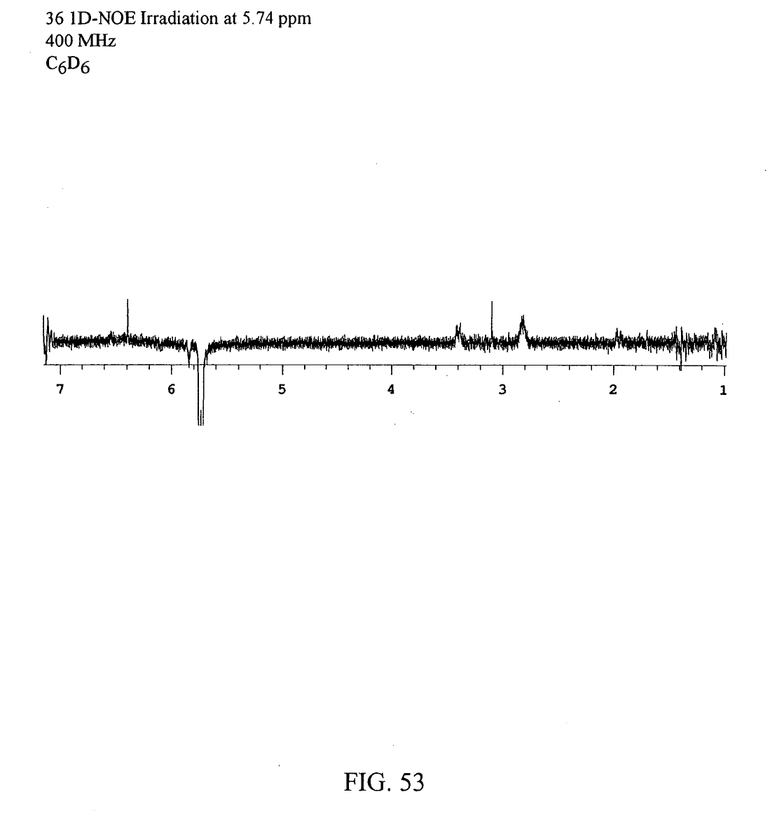

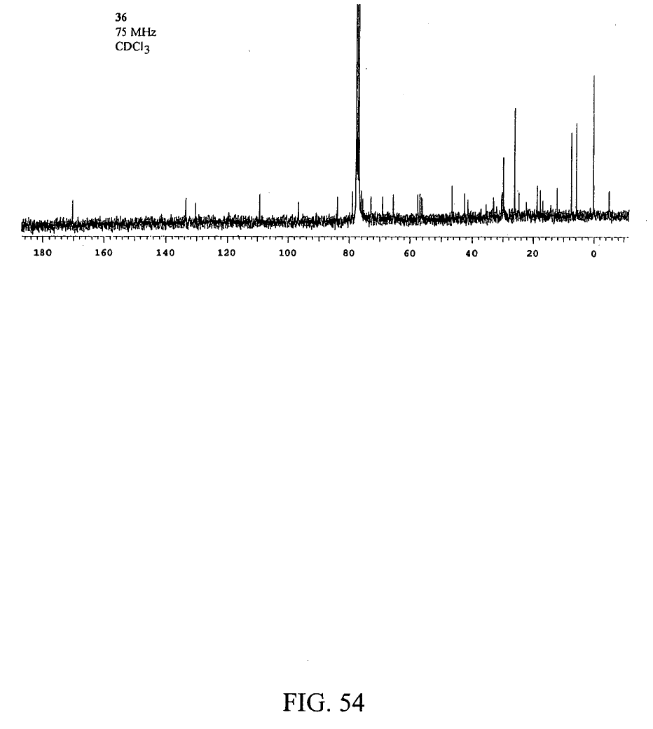

Compound 36:

[α]_{D}²³=+13 (c 0.1, CHCl₃); IR 2923, 2846, 1634, 1460, 1377, 1263, 1017, 797 cm⁻¹; NMR (400 MHz, C₆D₆ [FIG. 52]) δ 5.74 (1H, dd, J=3.6, 9.6 Hz), 5.17 (1H, d, J=7.2 Hz), 5.00 (1H, d, J=7.2 Hz), 4.81 (1H, d, J=10.0 Hz), 4.68 (1H, d, J=1.6Hz), 4.64 (1H, d, J=7.2 Hz), 4.53 (1H, td, J=1.6, 11.2 Hz), 4.18 (1H, d, J=2.4 Hz), 4.06 (1H, br.t, J=11.8 Hz), 4.05 (1H, dd, J=3.6, 9.6 Hz), 3.64 (1H, dd, J=7.6, 9.6 Hz), 3.3.6-3.42 (1H, m), 3.32 (1H, td, J=2.4, 10.8 Hz), 3.30 (3H, s), 3.16 (3H, s), 3.10 (3H, s), 2.95 (3H, s), 2.83 (1H, br.m), 2.15-2.26 (2H, m), 1.89-2.04 (4H, m), 1.84 (1H, app.q, J=11.2 Hz), 1.70 (3H, d, J=1.2 Hz), 1.53 (1H, dd, J=4.0, 11.6 Hz), 1.20-1.40 (2H, m), 1.38 (3H, s), 1.27 (3H, s), 1.10 (9H, t, J=8.4 Hz), 1.02 (9H, s), 0.96 (3H, t, J=7.6 Hz), 0.82 (6H, app.dq, J=2.8, 8.0 Hz); 0.15 (3H, s), 0.14 (3H, s) ppm; ¹³C NMR (75 MHz, CDCl₃ [FIG. 54]) δ 170.2, 133.5, 130.3, 109.2, 96.5, 83.9, 79.0, 77.8, 77.4, 76.0, 75.6, 72.8, 69.0, 65.5, 65.4, 57.6, 56.9, 56.0, 46.5, 42.5, 41.3, 33.2, 30.5, 30.3, 29.9, 26.2 (3C), 24.7, 18.6, 17.6, 16.8, 12.2, 7.4 (3C), 5.83 (3C), -5.1 (2C) ppm; MS (ES) m/z (%): 826.40 ([M+Na—H₂O]⁺, 60).

Compound ent-37:

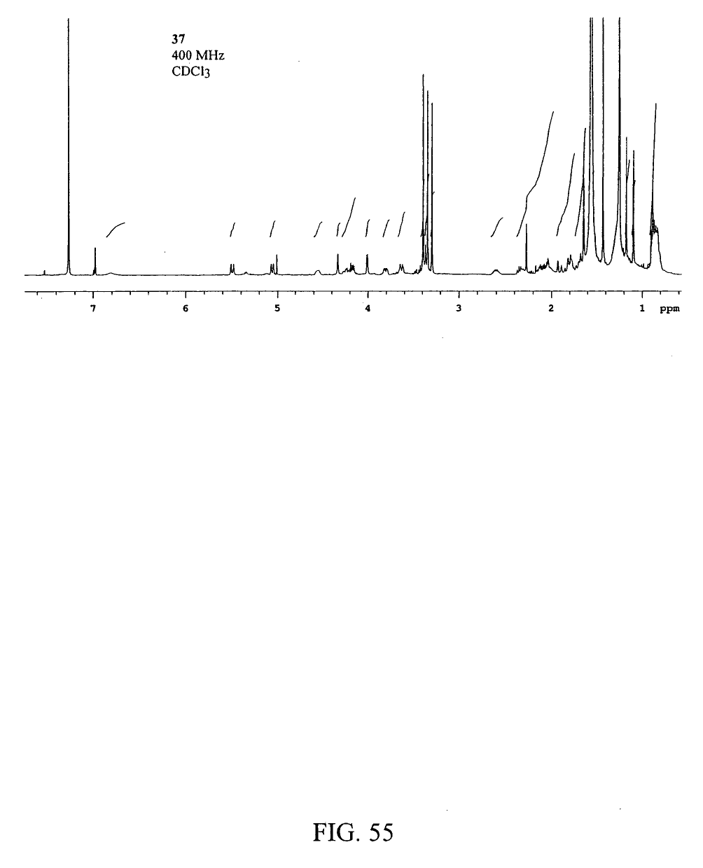

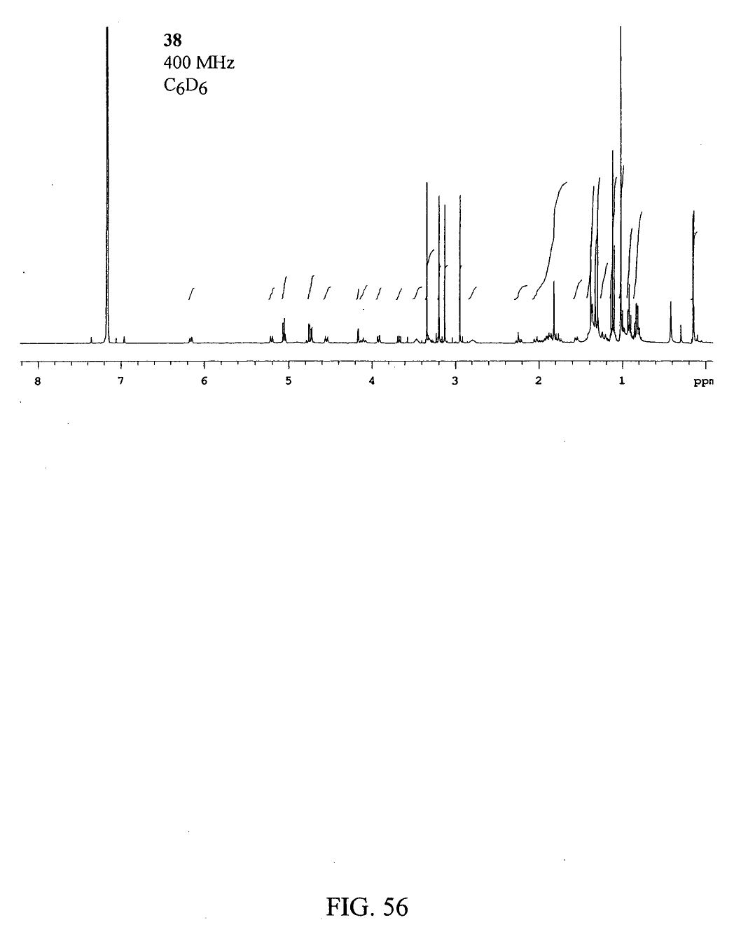

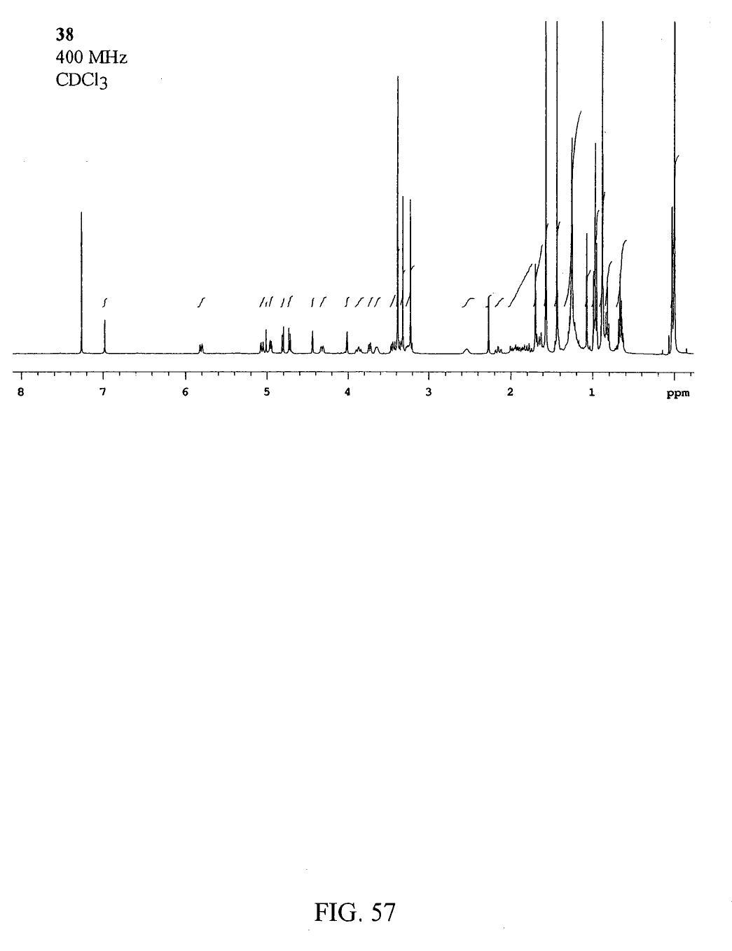

Compound ent-37 is obtained by stirring compound ent-36 according to the procedure described below for the preparation of 40. Ent-37 (compound formula II of claim 2): [α]_{D}²³=+16 (c 0.08, CHCl₃) {37: [α]_{D}²³=−20 (c 0.05, CHCl₃)}; IR 3440, 2924, 1737, 1667, 1460, 1260, 1091, 1017, 801 cm⁻; ¹H NMR (400 MHz [FIG. 55], CDCl₃) δ 6.81 (1H, br.s), 5.50 (1H, d, J=10.8 Hz), 5.05 (1H, d, J=10.4 Hz), 4.55 (1H, br.d, J=8.0 Hz), 4.33 (1H, s), 4.24 (1H, tdd, J=2.4, 4.4, 11.6 Hz), 4.14-4.22 (2H, m), 4.01 (1H, d, J=2.8 Hz), 3.80 (1H, ddd, J=3.2, 4.8, 11.6 Hz), 3.63 (1H, dd, J=3.6, 10.4 Hz), 3.39 (3H, s), 3.32-3.40 (2H, m), 3.34 (3H, s), 3.29 (3H, s), 2.55-2.65 (1H, m), 1.96-2.18 (4H, m), 1.15-1.90 (6H, m), 1.64 (3H, d, J=1.2 Hz), 1.17 (3H, s), 1.09 (3H, s), 0.88 (3H, t, J=7.6 Hz) ppm; MS (ES) m/z (%): 571.2 ([M+Na]⁺, 100).

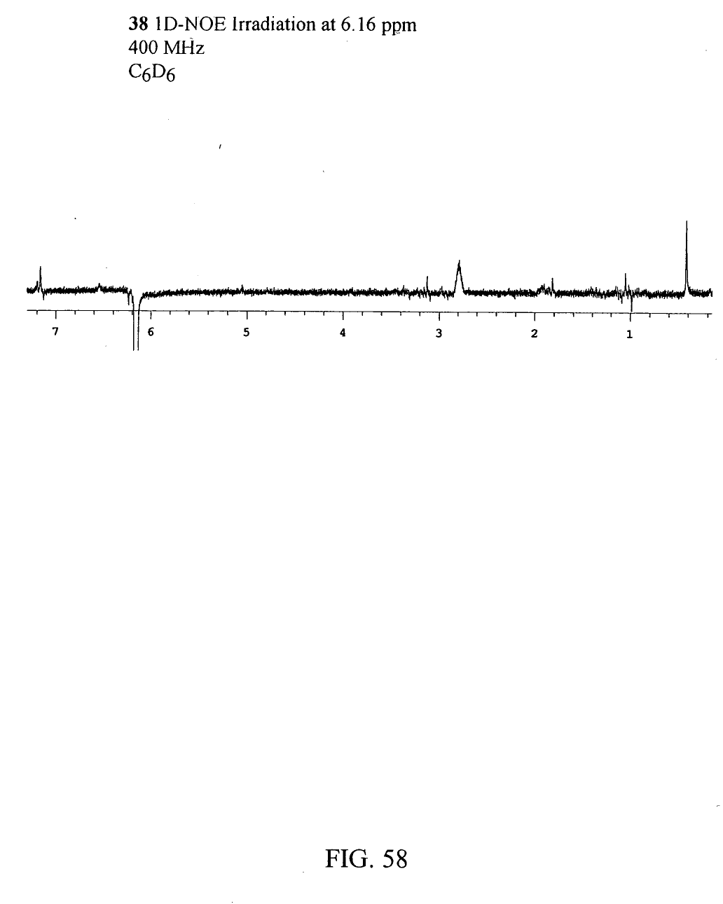

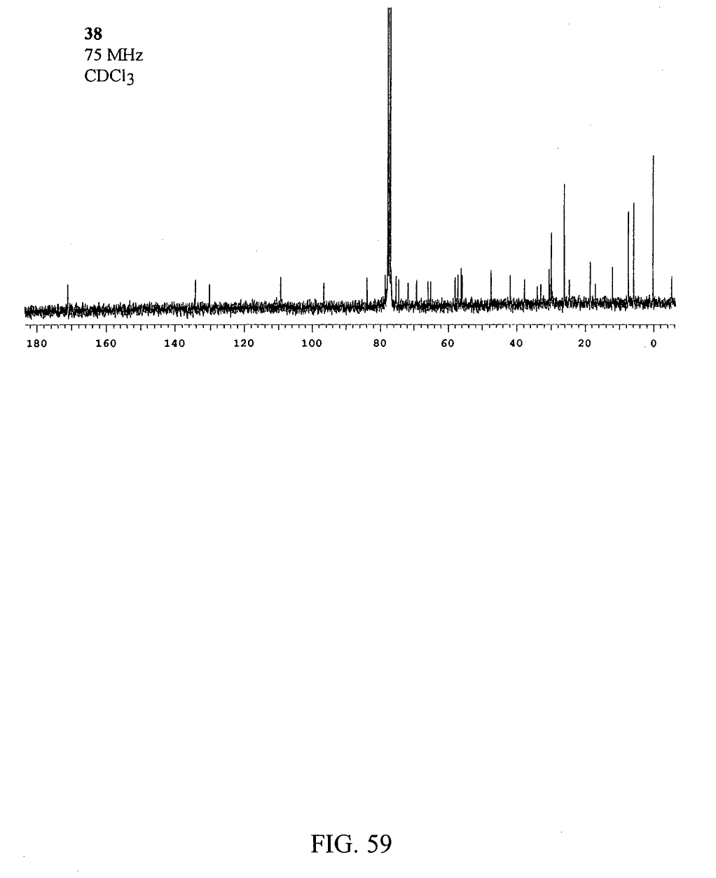

Compound 38:

[α]_{D}²³=+49 (c 0.3, CHCl₃); IR 2925, 2853, 1749, 1463, 1377, 1253, 1157, 1106, 1003, 833 cm⁻¹; ¹H NMR (400 MHz, CDCl₃)=[FIG. 58]) δ 5.80 (1H, dd, J=2.4, 10.0 Hz), 5.06 (1H, d, J=10.0 Hz), 4.95 (1H, d, J=5.2 Hz), 4.81 (1H, d, J=7.2 Hz), 4.72 (1H, d, J=7.2 Hz), 4.43 (1H, d, J=1.2 Hz), 4.32 (1H, br.d, J=9.6 Hz), 4.01 (1H, d, J=2.8 Hz), 3.87 (1H, br.t, J=11.2 Hz), 3.73 (1H, dd, J=4.0, 10.0 Hz), 3.65 (1H, m), 3.45 (1H, dd, J=4.0, 10.8 Hz), 3.38 (6H, s), 3.32 (3H, s), 3.26 (1H, ddd, J=2.4, 4.8, 10.0 Hz), 3.22 (3H, s), 2.50-2.58 (1H, m), 2.15 (1H, dt, J=3.6, 12.4 Hz), 1.96 (1H, td, J=6.0, 11.6 Hz), 1.92 (1H, ddd, J=2.4, 9.2, 15.2 Hz), 1.83 (1H, ddd, J=4.8, 10.8, 15.2 Hz), 1.77 (1H, dt, J=2.0, 12.4 Hz), 1.70 (3H, d, J=0.4 Hz), 1.58-1.68 (2H, m), 1.41-1.46 (1H, m), 1.25 (3H, s), 1.20-1.30 (2H, m), 1.07 (3H, s); 0.97 (9H, t, J=8.0 Hz), 0.88 (9H, s), 0.82 (3H, t, J=7.6 Hz), 0.66 (6H, q, J=7.8 Hz); 0.03 (3H, s); 0.02 (3H, s) ppm; ¹³C NMR (75 MHz, CDCl₃ [FIG. 59]) δ 171.0, 134.2, 130.2, 109.3, 96.6, 83.8, 78.5, 77.4, 75.2, 74.5, 71.7, 69.2, 65.8, 65.0, 57.9, 57.2, 56.2, 55.9, 47.4, 41.8, 37.7, 34.0, 33.0, 30.5, 29.9, 26.2 (3C), 24.6, 18.6, 18.5, 17.0, 12.1, 7.4 (3C), 5.8 (3C), −5.1, −5.2 ppm; HRMS. Calcd for C₄₁H₇₈O₁₁Si₂Li ([M+Li]⁺): 809.5242. Found: 809.5238.

Compound ent-39:

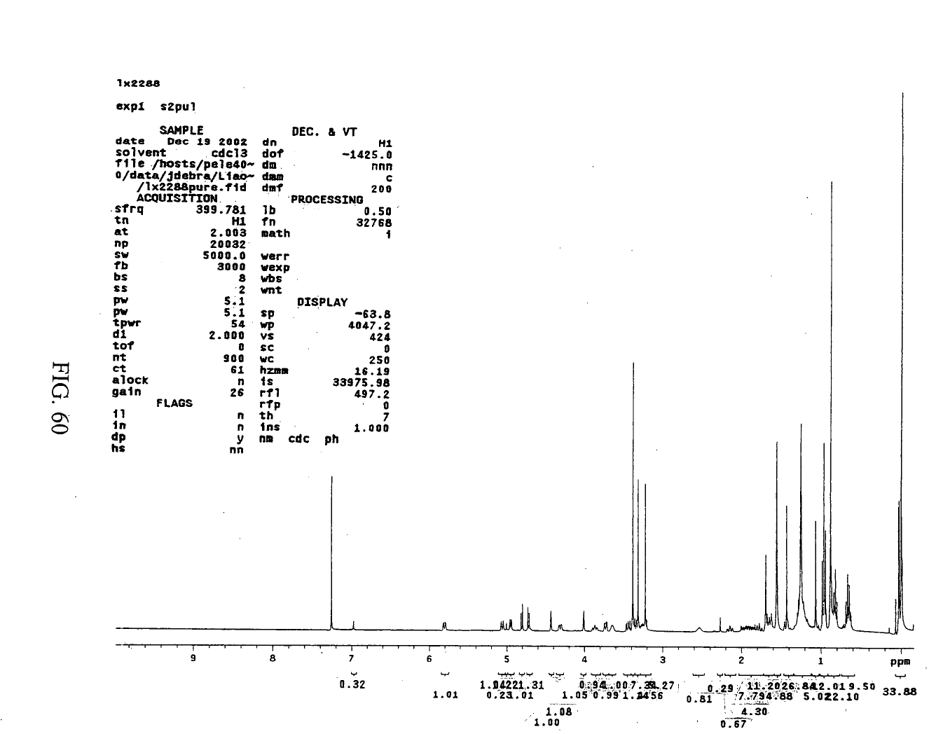

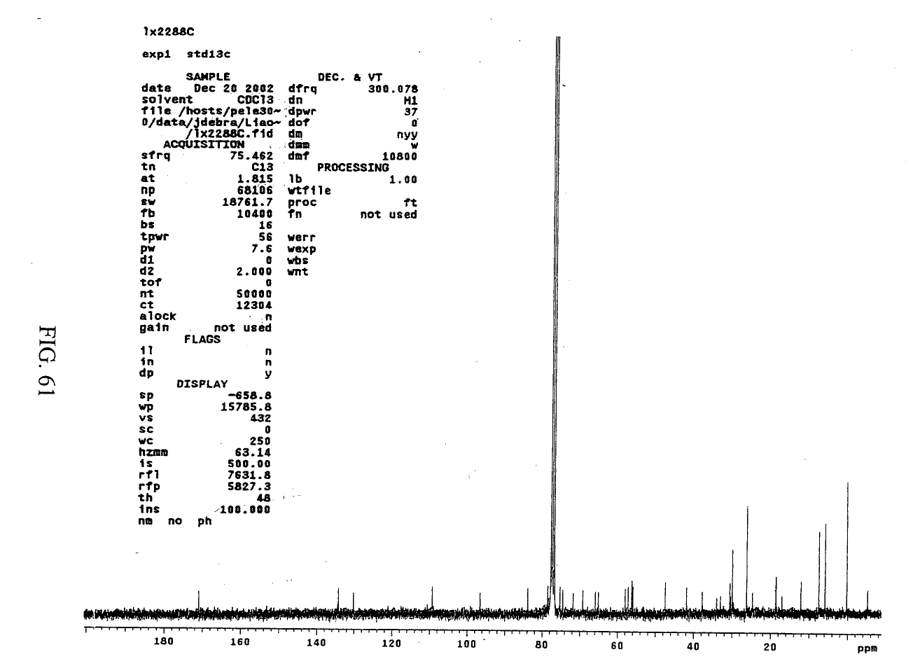

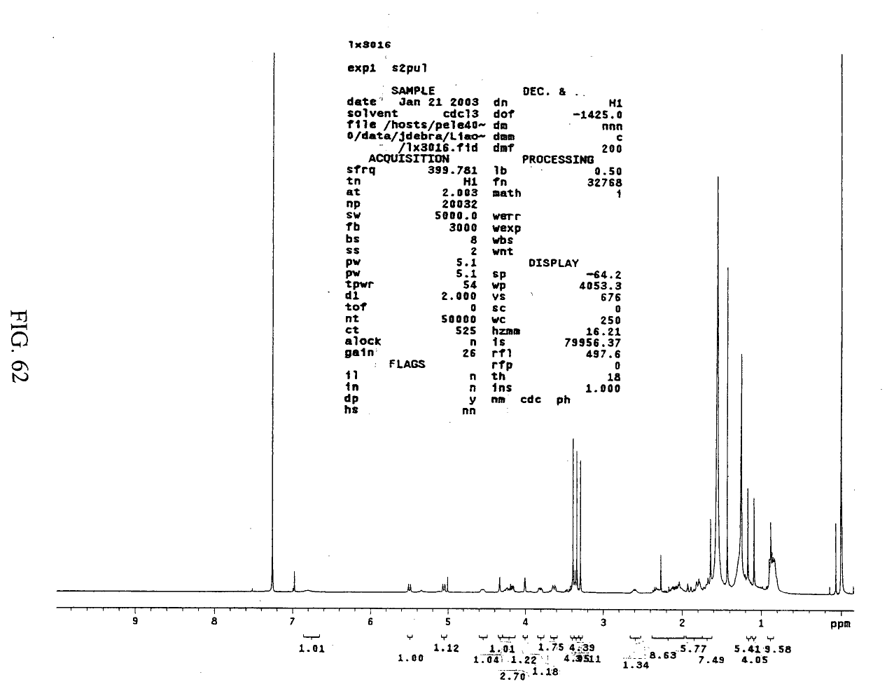

To a solution of PPh₃ (28.4 mg, 0.1073 mmol, azeotropically dried with benzene) in 2 mL of THF was added DIAD (22 μL, 0.1073 mmol) drop-wise at RT. The solution was stirred at RT for 20 min. To this solution was added a solution of crude acid ent-34a (7.5 mg, 0.00894 mmol) in 3 mL of THF at 0° C. over a period of 2 h via syringe pump. The resulting mixture was stirred for an additional h at this temperature and quenched with water. The aq. layer was extracted with EtOAc and the combined organic layers were washed with brine, dried over Na₂SO₄, and concentrated. The residue was purified by FC (EtOAc/Hexanes 1/4) to afford 5 mg of product ent-39 (69%). Ent-39: [α]_{D}²³=−60 (c 0.45, CHCl₃); IR 2925, 2853, 1749, 1463, 1377, 1253, 1157, 1106, 1003, 833 cm⁻¹; ¹H NMR (400 MHz [FIG. 60], CDCl₃) δ 5.81 (1H, dd, J=2.4, 10.0 Hz), 5.06 (1H, d, J=10.0 Hz), 4.95 (1H, d, J=5.2 Hz), 4.81 (1H, d, J=7.2 Hz), 4.72 (1H, d, J=7.2 Hz), 4.43 (1H, d, J=1.2 Hz), 4.32 (1H, d, J=9.6 Hz), 4.01 (1H, d, J=2.8 Hz), 3.87 (1H, t, J=12.0 Hz), 3.73 (1H, dd, J=4.0, 10.0 Hz), 3.65 (1H, m), 3.45 (1H, dd, J=4.0, 10.8 Hz), 3.38 (6H, s), 3.32 (3H, s), 3.26 (1H, ddd, J=2.4, 4.8, 10.0 Hz), 3.22 (3H, s), 2.54 (1H, m), 2.15 (1H, td, J=3.6, 12.8 Hz), 1.96 (1H, td, J=6.0, 10.0 Hz), 1.91 (1H, ddd, J=2.4, 9.2, 15.2 Hz), 1.83 (1H, ddd, J=4.8, 10.8, 15.2 Hz), 1.77 (1H, td, J=2.0, 12.4 Hz), 1.70 (3H, d, J=0.4 Hz), 1.20-1.68 (5H, m), 1.07 (3H, s); 0.97 (9H, t, J=8.0 Hz), 0.88 (12H, s), 0.82 (3H, t, J=7.6 Hz), 0.67 (6H, q, J=7.8 Hz); 0.03 (3H, s); 0.02 (3H, s) ppm; ¹³C NMR (75 MHz [FIG. 61], CDCl₃) δ 171.0, 134.2, 130.2, 109.3, 96.6, 83.8, 78.5, 77.4, 75.2, 74.5, 71.7, 69.2, 65.8, 65.0, 57.9, 57.2, 56.2, 55.9, 47.4, 41.8, 37.7, 34.0, 33.0, 30.5, 29.9, 26.2 (3C), 24.6, 18.6, 18.5, 17.0, 12.1, 7.4 (3C), 5.8 (3C), −5.1, −15.2 ppm; HRMS Calcd for C₄l H₇₈O₁₁Si₂Li ([M+Li]⁺): 809.5242. Found: 809.5238.

Compound ent-40 [(+)-Peloruside A]:

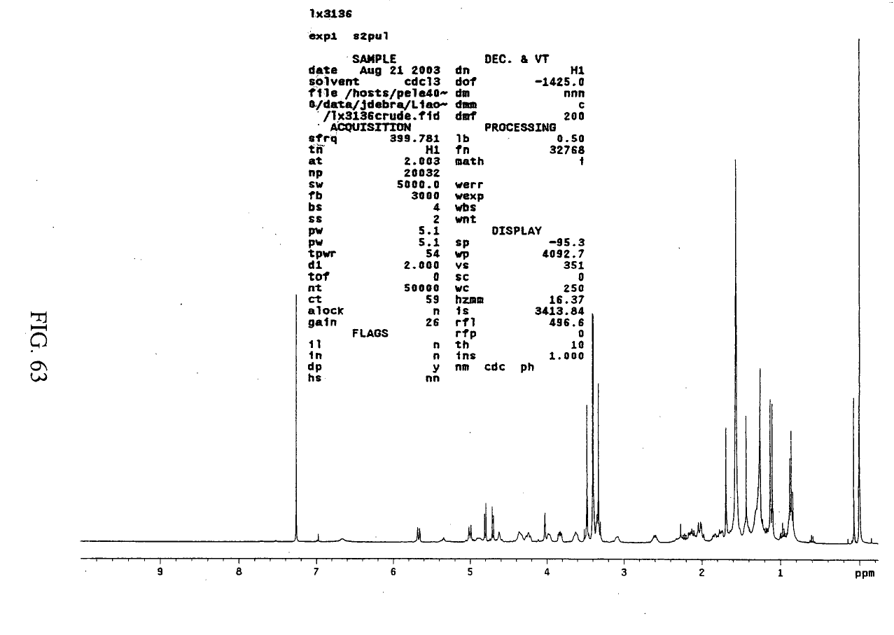

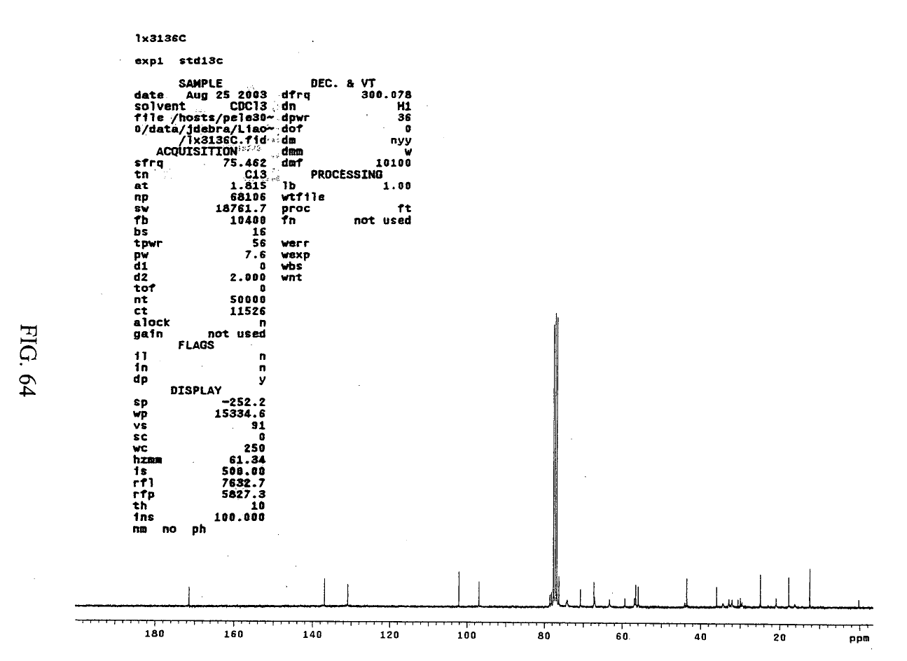

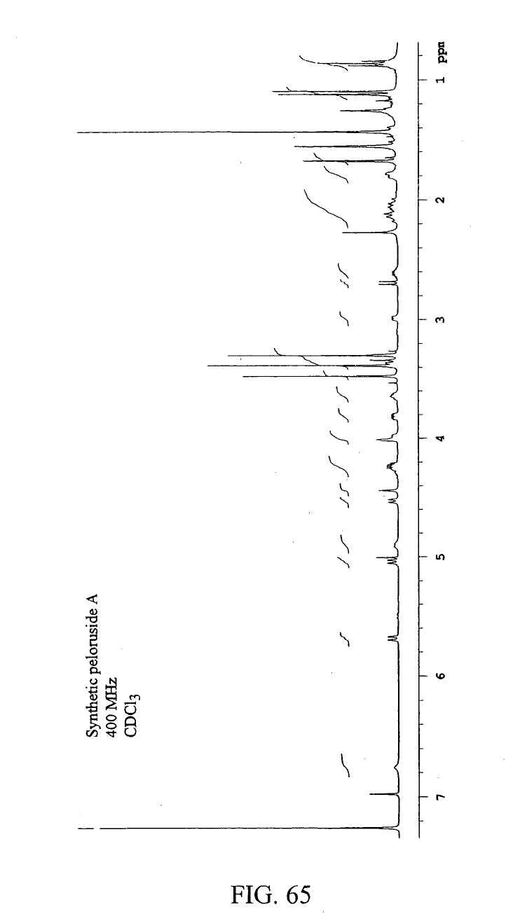

To a solution of ent-39 (2 mg) in CH₃CN/H₂O (9/1, 1 mL) at RT was added a 48% aq. HF solution (58 uL). The reaction mixture was stirred for 27 h and quenched by the addition of sat. aq. NaHCO₃ and EtOAc. The organic layer was washed with brine, dried over Na₂SO₄ and concentrated. FC of the residue (2% to 4% MeOH in CH₂Cl₂) provided compound intermediate x (i.e. the compound with formula III of claim 3 (1.2 mg, 83%). [α]_{D}²³=−15 (c 0.4, CHCl₃); IR 3435, 2929, 1738, 1455, 1382, 1216, 1154, 1084, 1026, 931 cm⁻¹; ¹H NMR (400 MHz [FIG. 63], CDCl₃) δ 6.67 (1H, s), 5.68 (1H, d, J=10.4 Hz), 5.01 (1H, d, J=10.4 Hz), 4.89 (1H, m), 4.81 (1H, d, J=7.2 Hz), 4.71 (1H, d, J=7.2 Hz), 4.63 (1H, s), 4.37 (1H, s), 4.35 (1H, m), 4.25 (1H, ddd, J=2.8, 4.0, 11.2 Hz), 4.04 (1H, d, J=2.8 Hz), 3.98 (1H, br. d, J=8.8 Hz), 3.84 (1H, ddd, J=2.8, 5.2, 11.6 Hz), 3.63 (1H, td, J=2.8, 10.0 Hz), 3.49 (3H, s), 3.41 (3H, s), 3.40 (3H, s), 3.36 (1H, m), 3.34 (3H, s), 3.10 (1H, d, J=7.6 Hz), 2.61 (1H, m), 1.98-2.21 (5H, m), 1.84 (1H, ddd, J=4.4, 11.2, 12.4 Hz), 1.77 (1H, ddd, J=2.0, 4.8, 12.4 Hz), 1.70 (3H, d, J=0.8 Hz), 1.20- 1.60 (3H, m), 1.14 (3H, s), 1.11 (3H, s), 0.87 (3H, t, J=7.6 Hz); ¹³C NMR (75 MHz [FIG. 64], CDCl₃) δ 171.4, 136.9, 130.8, 102.0, 96.9, 78.6, 78.1, 76.3, 74.1, 70.7, 67.3, 67.0, 63.3, 59.3, 56.9, 56.5, 55.9, 44.0, 43.5, 35.9, 34.3, 32.8, 32.0, 29.9, 24.9, 20.9, 17.7, 16.2, 12.4 ppm; MS (ES) m/z (%): 615.35 ([M+Na]⁺, 100). To a solution of intermediatex (4 mg, 0.00488 mmol) in THF (1.5 mL) was added 4N aq. HCl (1.5 mL). The resulting solution was stirred at RT for 3 h and quenched by the slow addition of sat. aq. NaHCO₃. EtOAc was added and the organic solution was washed with brine and dried over Na₂SO₄. Filtration and concentration followed by FC (4% to 6% MeOH in CH₂Cl₂) provided (+)-Peloruside A (ent-40) (1.8 mg, 68%). (+)-Peloruside A can also directly be obtained by stirring compound ent-39 in a THF/4N aq. HCl mixture as described here for deprotection of intermediate x. Ent-40 |(+)-Peloruside A] (compound formula I of claim 1): [α]_{D}²³=+15.5 (c 0.2, CH₂Cl₂); IR 3447, 2928, 2853, 1729, 1460, 1383, 1273, 1123, 1072, 740 cm⁻¹; ¹H NMR (400 MHz [FIGS. 5, 65], CDCl₃) δ 6.77 (1H, s), 5.70 (1H, d, J=10.4 Hz), 5.05 (1H, d, J=10.0 Hz), 4.90 (1H, br.d, J=10.4 Hz), 4.54 (1H, d, J=9.6 Hz), 4.44 (1H, s), 4.27 (1H, tdd, J=2.4, 4.4, 11.2 Hz), 4.24 (1H, dd, J=5.2, 10.4 Hz), 4.02 (1H, s), 3.99 (1H, m), 3.82 (1H, ddd, J=2.4, 5.2, 11.2 Hz), 3.65 (1H, ddd, J=4.0, 8.4, 10.4 Hz), 3.48 (3H, s), 3.39 (3H, s), 3.37 (1H, td, J=3.2, 10.4 Hz), 3.31 (3H, s), 3.00 (1H, dd, J=3.2, 8.4 Hz), 2.71 (1H, d, J=9.6 Hz), 2.61 (1H, m), 2.17 (1H, ddd, J=2.0, 10.0, 15.6 Hz), 2.15 (1H, m), 2.07 (1H, ddd, J=4.8, 11.6, 15.6 Hz), 2.03 (1H, ddd, J=4.8, 11.6, 15.6 Hz), 1.81 (1H, m), 1.80 (1H, ddd, J=2.4, 5.2, 12.4 Hz), 1.68 (3H, d, J=0.8 Hz), 1.19-1.60 (4H, m), 1.13 (3H, s), 1.10 (3H, s), 0.87 (3H, t, J=7.2 Hz) ppm; ¹³C NMR (75 MHz [FIG. 4], CDCl₃) δ174.2, 136.3, 131.4, 102.1, 78.5, 78.1, 76.1, 74.1, 71.1, 70.5, 67.2, 67.1, 63.7, 59.3, 56.3, 55.9, 43.8, 43.5, 35.9, 34.1, 32.8, 31.9, 24.8, 21.0, 17.7, 16.0, 12.5 ppm; HRMS Calcd for C₂₇H₄₈O₁₁Li ([M+Li]⁺): 555.3357. Found: 555.3338.

(−)-Peloruside A (40):

To a solution of lactone 38 (4 mg, 0.00488 mmol) in THF (1.5 mL) was added 4N HCl (1.5 mL). The resultant solution was stirred at room temperature for 3 h. Aqueous NaHCO₃ was added dropwise followed by extraction with EtOAc. The organic solution was washed with brine and dried over Na₂SO₄. Filtration and concentration followed by flash chromatography (4%→6% MeOH in CH₂Cl₂) provided (−)-Peloruside 40 (1.8 mg, 65%). [α]_{D}²³=−16 (c 0.15, CH₂Cl₂); IR 3447, 2928, 2853, 1729, 1460, 1383, 1273, 1123, 1072, 740 cm⁻¹; ¹H NMR (400 MHz, CDCl₃) δ 6.75 (1H (OH), br.s), 5.68 (1H, d, J=10.4 Hz), 5.05 (1H (OH), d, J=10.0 Hz), 4.89 (1H, br.d, J=10.4 Hz), 4.53 (1H, d, J=9.6 Hz), 4.43 (1H (OH), s), 4.25 (1H, tdd, J=2.4, 4.4, 11.2 Hz), 4.22 (1H, dd, J=5.2, 10.4 Hz), 4.02 (1H, br.s), 3.98-4.02 (1H, m), 3.82 (1H, ddd, J=2.4, 5.2, 11.2 Hz), 3.64 (1H, ddd, J=4.0, 8.0, 10.5 Hz), 3.48 (3H, s), 3.38 (3H, s), 3.36 (1H, dt, J=3.2, 10.5 Hz), 3.31 (3H, s), 3.00 (1H (OH), dd, J=3.2, 8.0 Hz), 2.70 (1H (OH), d, J=9.6 Hz), 2.57-2.66 (1H, m), 2.15 (1H, br.dd, J=10.4, 15.5 Hz), 2.12-2.16 (1H, m), 2.07 (1H, ddd, J=4.4, 11.2, 14.6 Hz), 2.02 (1H, br.dd, J=11.6, 15.5 Hz), 1.78 (1H, ddd, J=2.4, 5.2, 12.4 Hz), 1.78 (1H, m), 1.67 (3H, d, J=0.8 Hz), 1.53 (1H, q, J=12.0 Hz), 1.40-1.46 (1H, m), 1.40 (1H, br.d, J=14.6 Hz), 1.16-1.20 (1H, m), 1.12 (3H, s), 1.10 (3H, s), 0.86 (3H, t, J=7.4 Hz) ppm; ¹³C NMR (75 MHz, CDCl₃) δ 174.0, 136.1, 131.2, 101.9, 78.3, 77.9, 75.9, 73.9, 70.9, 70.3, 67.0, 66.9, 63.5, 59.1, 56.1, 55.7, 43.6, 43.4, 35.7, 33.9, 32.6, 31.7, 24.6, 20.8, 17.5, 15.8, 12.3 ppm; HRMS Calcd for C₂₇H₄₈O₁₁Li ([M+Li]⁺): 555.3357. Found: 555.3338.

To a solution of i-Pr₂NH (8.07 mL, 57.6 mmol) in 150 ml of THF at 0° C. was added n-BuLi (21.6 mL, 54 mmol, 2.5 M in hexanes) over 5 min, then the mixture was stirred for another 30 min. After cooling to −78° C., a solution of ketone 14 (8.6 g, 42 mmol) in 15 mL THF was added over 5 min. After stirring for 15 min at −78° C., the solution was allowed to warm to −40° C. over a period of 1 h. The reaction mixture was cooled to −78° C, and a solution of aldehyde 70 (8.3 g, 36 mmol) in THF (10 mL) was added dropwise over 15 min. The resulting mixture was stirred for 45 min at −78° C. and quenched by the addition of AcOH in ether (2 mL), and aq. saturated NaHCO₃. The aqueous layer was extracted with Et₂O, and the combined organic layers were washed with water and brine. The organic extracts were dried over MgSO₄ and concentrated and the residue was purified by FC (EtOAc/Hexane, 1:4) to afford 14 g (89%) of product.

To a solution of aldol product (13 g, 29.9 mmol), DMSO (50 mL) and Et₃N (10 mL, 71.6 mmol) in 50 mL CH₂Cl₂ at 0° C. was added Py.SO₃ (11.0 g, 69.0 mmol). After stirring at this temperature for one hour, a saturated aqueous NH₄Cl solution (300 mL) was added and the mixture was extracted with ether (150 mL×3). The organic layers were combined and washed with water and brine and dried over MgSO₄. Concentration provided a residue that was purified by FC (5% to 15% EtOAc in hexanes) to afford 10 g (77.4%) of diketone 71. 71: [α]_{D}²³=+41.0 (c 0.4, CHCl₃); IR 2929, 2857, 1603, 1472, 1255, 1095, 836, 776 cm⁻¹; ¹H NMR (400 MHz, CDCl₃) δ 7.30 (5H, m), 5.88 (1H, dddd, J=6.8, 6.8, 10.4, 18.0 Hz), 5.66 (1H, s), 5.07 (2H, m), 4.51 (2H, s), 4.20 (1H, m), 3.45 (1H, d, J=8.8 Hz), 3.42 (1H, d, J=8.8 Hz), 2.43 (1H, dd, J=4.8, 13.6 Hz), 2.36 (1H, dd, J=8.0, 13.6 Hz), 2.27(2H, m), 1.17 (3H, s), 1.16 (3H, s), 0.85 (9H, s), 0.04 (3H, s), −0.02 (3H, s) ppm; ¹³C NMR (75 MHz, CDCl₃) 6 199.4, 191.9, 138.6, 134.4, 128.5 (2C), 127.6 (3C), 117.9, 98.7, 76.8, 73.5, 69.6, 46.2, 44.2, 42.6, 26.0, 22.9 (3C), 22.8, 18.2, −4.42, −4.83 ppm. MS (ES) m/z (%):455.20 ([M+Na]⁺, 100).

To a solution of diketone 71 (9 g, 21 mmol) in 100 mL of MeOH was added p-TsOH (600 mg, 3.15 mmol) at 0° C. After stirring at this temperature for 0.5 h, the ice-bath was removed and the mixture was allowed to warm to RT and stirred for 4 h. The solvent was removed by vacuum and the residue was purified by FC (EtOAc/Hexane, 1:6 to 1:4) to provide the product 72 in 95% yield. 72: [α]_{D}²³=−10.0 (c 1.0, CHCl₃); IR 2973, 2872, 1668, 1599, 1454, 1336, 1242, 1095, 922, 739, 699 cm⁻¹; ¹H NMR (400 MHz, CDCl₃) δ 7.27-7.36 (5H, m), 5.77 (1H, dddd, J=6.8, 6.8, 10.4, 16.0 Hz), 5.47 (1H, s), 5.14 (1H, d, J=16.0 Hz), 5.13 (1H, d, J=10.4 Hz), 4.49 (2H, s), 4.32 (1 h, ddd, J=6.0, 6.0, 17.2 Hz), 3.40 (1H, d, J=8.8 Hz), 3.35 (1H, d, J=8.8 Hz), 2.43 (4H, m), 1.16 (3H, s), 1.15 (3H, s) ppm; ¹³C NMR (75 MHz, CDCl₃): 193.4, 181.4, 138.4, 132.5, 128.4, 127.6, 127.5, 118.6, 103.1, 78.4, 76.5, 73.3, 41.4, 40.5, 38.6, 23.1, 23.0 ppm.

To a solution of dihydropyranone 72 (0.05 mmol/mL) in MeOH at RT was added CeCl₃.H₂O (1.0 eq). The mixture was cooled to −30° C., followed by addition of NaBH₄ (1.5 eq). After 10 min, another portion NaBH₄ (0.5 eq) was added. The mixture was stirred at −30° C. for 30 min, followed by quenching with sat. aq. NaHCO₃ (3 mL) and brine (20 mL). The aqueous phase was extracted with Et₂O (30 mL×3), and the combined organic extracts were dried over MgSO₄. Concentration provided a residue that was purified by FC (EtOAc/Hexane, 1:4) to provide glycal 73 in 90% yield. 73: [α]_{D}²³=−12.0 (c 0.9, CHCl₃); IR 3387, 2927, 2860, 1663, 1457, 1277, 1100, 1073, 1030, 917, 733, 700 cm⁻¹; ¹H NMR (400 MHz, CDCl₃) δ 7.26-7.34 (5H, m), 5.80 (1H, dddd, J=6.8, 6.8, 10.0, 16.8 Hz), 5.10 (1H, d, J=16.8 Hz), 5.00 (1H, d, J=10.0 Hz), 4.67 (1H, dd, J=2.0, 2.0 Hz), 4.51 (2H, s), 4.45 (1H, dddd, J=2.0, 7.0, 9.2, 9.2 Hz), 3.90 (1H, dddd, J=2.0, 7.0, 7.0, 12.0 Hz), 3.35 (1H, d, J=8.8 Hz), 3.28 (1H, d, J=8.8 Hz), 2.36 (1H, ddd, J=6.8, 14.0, 14.0 Hz), 2.30 (1H, ddd, J=6.8, 14.0, 14.0 Hz), 2.11 (1H, dddd, J=16.8, 6.8, 1.6, 1.6 Hz), 1.48 (1H, ddd, J=13.2, 11.6, 9.2 Hz), 1.35 (1H, d, J=7.2 Hz), 1.08 (3H, s), 1.07 (3H, s) ppm; ¹³C NMR (75 MHz, CDCl₃) δ 160.4, 139.0, 134.3, 128.4, 127.6, 127.5, 117.4, 99.5, 76.9, 74.3, 73.3, 64.2, 39.8, 39.6, 37.4, 23.4 ppm.

To a solution of glycal 73 (790 mg, 2.61 mmol) in CH₂Cl₂ (45 mL) and MeOH (15 mL) at 0° C. was added NaHCO₃, followed by m-CPBA (541 mg, 3.135 mmol) in two portions over 10 min. The resultant suspension was stirred at 0° C. for 40 min, followed by the addition of Et₃N (6 mL). After stirring for 15 min, the mixture was poured into hexanes (100 mL), and passed through a pad of celite (Et₂O rince). After concentration of the filtrate, the residue was purified by FC (EtOAc/Hexane/Et₃N, 30:70:0.2) to give 690 mg product 74 (57.4%) and 30 mg of a diastereoisomer (3.3%). 74: [α]_{D}²³=−20.0 (c 0.8, CHCl₃); IR 3412, 2925, 1642, 1454, 1380, 1064, 919, 737, 698 cm⁻¹; ¹H NMR (400 MHz, CDCl₃) δ 7.33 (5H, m), 5.87 (1H, dddd, J=6.8, 6.8, 10.8, 16.4 Hz), 5.10 (1H, d, J=16.4 Hz), 5.05 (1H, d, J=10.8 Hz), 4.55 (1H, d, J=11.6 Hz), 4.41 (1H, d, J=11.6 Hz), 3.90 (1H, dddd, J=4.0, 4.0, 11.0, 11.0 Hz), 3.72 (1H, dd, J=4.0, 4.0 Hz), 3.58 (1H, dddd, J=3.2, 5.6, 8.0, 12.0 Hz), 3.44 (1H, d, J=9.2 Hz), 3.37 (1H, d, J=9.2 Hz), 3.31 (3H, s), 2.55 (1H, d, J=11.0 Hz), 2.29 (1H, ddd, J=6.8, 13.6, 13.6 Hz), 2.25 (1H, ddd, J=6.8, 13.6, 13.6 Hz), 1.65 (1H, ddd, J=3.0, 4.2, 12.0 Hz), 1.55 (1H, dd, J=12.0, 24.0 Hz), 1.11 (3H, s), 1.03 (3H, s) ppm; ¹³C NMR (75 MHz, CDCl₃) δ 137.3, 134.6, 128.7, 128.1, 127.9, 117.2, 103.5, 77.1, 73.8, 70.9, 69.5, 67.1, 52.0, 45.5, 40.3, 33.5, 22.8, 22.2 ppm.

To a solution of diol 74 (5.55 g, 15.8 mmol) in dry THF at 0° C. was added KO^{t}Bu (3.0 g, 25.4 mmol). After stirring for 15 min, CH₃I (23 mL, 369 mmol) was added dropwise and the reaction mixture was stirred for 30 min at 0° C., followed by quenching with saturated aqueous NH₄Cl. The phases were separated and the aqueous layer was extracted with ether. The combined organic phases were dried and concentrated in vacuo to afford 5.73 g crude product which was used in the next step without further purification.

To a solution of alcohol (5.73 g, 15.7 mmol) in CH₂Cl₂ (60 mL) at −78° C. was add lutidine (5 mL, 42.5 mmol) and TESOTf (6.4 mL, 27.8 mmol). After 2 h, the reaction mixture was diluted with Et₂O (300 mL) and NH₄Cl (100 mL). The organic layer was washed with 100 mL saturated aq. NH₄Cl, and dried over MgSO₄. Concentration provided a residue that was purified by FC (1% EtOAc in hexanes) to give product 75 (4.5 g, 81% for two steps). 75: [═]_{D}²³=−11.0 (c 0.8, CHCl₃); IR 2953, 2876, 1454, 1380, 1139, 1098, 1040, 1004, 734, 696 cm⁻¹; ¹H NMR (400 MHz, CDCl₃) δ 7.30 (5H, m), 5.88 (1H, dddd, J=6.8, 6.8, 10.0, 16.8 Hz), 5.05 (2H, m),4.52(1H, d, J=12 Hz), 4.44 (1H, d, J=12), 3.93 (1H, d, J=2 Hz), 3.79 (1H, d, J=8.8 Hz), 3.54 (1H, m), 3.47 (1H, m), 3.32 (3H, s), 3,31 (3H, s), 2.28 (2H, m), 1.59 (2H, m), 1.11 (6H, s), 0.94 (9H, t, J=8.4 Hz), 0.63 (6H, q, J=8.4 Hz) ppm; ¹³C NMR (75 MHz, CDCl₃) δ139.8, 134.8, 128.3, 127.2, 117.1, 104.4, 77.1, 75.8, 73.3, 71.5, 71.2, 55.8, 51.7, 46.1, 40.5, 28.8, 21.5, 21.0, 7.4 (3C), 5.6 (3C) ppm; HRMS Calcd for C₂₇H₄₆O₅SiLi ([M+Li]⁺): 485.3275. Found: 485.3262.

To a solution of olefin 75 (1 g, 2.1 mmol) and 4-methylmorpholine N-oxide (520 μL, 2.5 mmol) in acetone at 0° C. was added OSO₄ (420 μL, 0.1 M solution in t-BuOH). The mixture was stirred for 16 h at RT, followed by the addition of aq. Na₂SO₃ at 0° C. After 1 h, EtOAc was added and the aqueous phase was extracted with EtOAc. The combined organic extracts were dried (Na₂SO₄) concentrated to provide 1.05 g crude diol.

The diol was dissolved in CH₂Cl₂ and cooled to 0° C. Pyridine (639 μL, 7.84 mmol) and Pb(OAc)₄ (1.26 g, 2.24 mmol) were added portion-wise over 5 min, and the mixture was vigorously stirred for 25 min at 0° C. After completion of the reaction, the mixture was filtered through silica gel and washed with ether. The organic extract was washed with brine and dried over Na₂SO₄. Concentration provided a residue that was purified by FC (EtOAc/hexane, 1:8) to give the aldehyde 76 (850 mg, 85% for two steps). 76: [α]_{D}²³=−20.0 (c 0.75, CHCl₃); IR 3954, 2877, 1455, 1384, 1140, 1101, 1039, 735 cm⁻¹; ¹H NMR (400 MHz, CDCl₃) δ 9.85 (1H, t, J=2.0 Hz), 7.32 (5H, m), 4.47 (1H, dd, J=12.4 Hz), 4.08 (1H, dddd, J=4.4, 4.4, 7.2, 12.0 Hz), 3.92 (1H, d, J=3.6 Hz), 3.73 (1H, d, J=8.8 Hz), 3.38 (3H, s), 3.32 (3H, s), 3.53 (1H, m), 3.31 (1H, d, J=8.8 Hz), 2.68 (1H, ddd, J=2.4, 7.6, 16.8 Hz), 2.54 (1H, ddd, J=2.0.4.4, 16.8 Hz), 1.67 (2H, m), 1.10 (3H, s), 1.09 (3H, s), 0.94 (9H, t, J=8.0 Hz), 0.65 (6H, q, J=8.0 Hz) ppm; ¹³C NMR (75 MHz, CDCl₃) δ 201.2, 139.6, 128.4, 127.4, 127.3, 104.6, 76.6, 75.7, 73.3, 70.9, 67.5, 56.0, 52.0, 49.5, 45.8, 29.1, 21.5, 21.2, 7.3 (3C), 5.6 (3C) ppm; MS (ES) m/z (%):503.23 ([M+Na]⁺, 20).

To a solution of 1-(methoxymethyl)oxy-2-propene (7.05 mmol, 1.3 eq) in 45 mL of THF at −78° C. was added sec-BuLi (5.0 mL, 1.3 M solution in cyclohexane, 6.5 mmol, 1.2 eq) over 15 min. The resulting yellow solution was maintained for 30 min at the same temperature, followed by the addition of a solution of (−)-B-methoxydissopinocampheylborane (2.226 g, 7.05 mmol) in THF (6 mL) over 15 min (the yellow solution became colorless). After stirring for 40 min at −78° C., and 2 h at −10° C., the solution was cooled back to −100° C. followed by the drop-wise addition (20 min) of a solution of aldehyde 76 (2.6 g, 5.42 mmol) in THF (1 M). After stirring at −100° C. for 3 h, the mixture was allowed to slowly reach RT. The solvent was removed in vacuo and the residue dissolved in ether, followed by aq. NaOH (2 M, 6 mL) and 30% aq. H₂O₂ (1.6 mL). After stirring for 16 h at RT, ether was added and the organic extract was washed with brine and dried over Na₂SO₄. Concentration provided a residue that was purified by FC (EtOAc/hexane, 1: 10) to provide the product 77 (2.05 g) and a diastereoisomer (525 mg) total yield is 81%. 77: [α]_{D}²³=−30.0 (c 1.0, CHCl₃); IR 3426, 2927, 1456, 1136, 1098, 1038, 733 cm⁻¹; ¹H NMR (400 MHz, CDCl₃) δ 7.31 (5H, m), 5.82 (1H, ddd, J=6.8, 10.4, 17.2 Hz), 5.32 (2H, m,), 4.73 (1H, d, J=6.4 Hz), 4.62 (1H, d, J=6.4 Hz), 4.52 (1H, d, J=12.8 Hz), 4.44 (1H, d, J=12.8 Hz), 4.07 (1H, s), 4.03 (1H, t, J=5.6, 6.8 Hz), 3.89 (1H, d, J=2.4 Hz), 3.88 (1H, m), 3.78 (1H, m), 3.49 (1H, d, J=9.2 Hz), 3.48 (1H, m), 3.39 (3H, s), 3,38 (3H, s), 3.31 (3H, s), 3.29 (1H, d, J=9.2 Hz), 1.75 (2H, m), 1.64 (2H, m), 1.11 (6H, s), 0.92 (9H, t, J=8.4 Hz), 0.63 (6H, q, J=8.4 Hz) ppm; ¹³C NMR (75 MHz, CDCl₃) δ 139.0, 135.0, 128.3, 128.0, 127.5, 119.0, 105.0, 94.4, 80.4, 76.7, 73.3, 73.0, 71.5, 55.8, 55.7, 51.7, 45.1, 38.6, 29.7, 22.0, 21.7, 7.4 (3C), 5.6 (3C). HRMS Calcd for C₃₁H₅₄O₈SiLi ([M+Li]⁺): 589.3748. Found: 589.3765. Isomer: IR 3473, 2940, 2866, 1150, 1093, 1030, 733 cm⁻¹; ¹H NMR (400 MHz, CDCl₃) δ 7.33 (5H, m), 5.67 (1H, ddd, J=8.0, 10.8, 17.2 Hz), 5.32 (2H, m), 4.75 (1H, d, J=6.8 Hz), 4.59 (1H, d, J=6.8 Hz), 4.53 (1H, d, J=12.0 Hz), 4.43 (1H, d, J=12.0 Hz), 3.93 (1H, m), 3.91 (1H, d, J=2.4 Hz), 3.89 (1H, m), 3.85 (1H, d, J=7.2 Hz), 3.72 (1H, d, J=8.8 Hz), 3.50 (1H, ddd, J=2.4, 5.2, 11.6 Hz), 3.40 (3H, s), 3.33 (1H, d, J=8.8 Hz), 3.31 (3H, s), 2.75 (1H, d, J=3.2 Hz), 1.52-1.71 (4H, m), 1.10 (3H, s), 1.09 (3H, s), 0.93 (9H, t, J=8.0 Hz), 0.65 (6H, q, J=8.0 Hz).

To a solution of alcohol 77 (45 mg, 0.077 mmol) in dry THF at 0° C. was added KOBu^{t} (16 mg, 0.135 mmol). After stirring for 15 min, CH₃I (30 μL, 0.477 mmol) was added drop-wise and the reaction mixture was stirred for 30 min at 0° C. and quenched with saturated aqueous NH₄Cl. The phases were separated and the aqueous layer was extracted with ether. The combined organic extracts were dried and concentrated in vacuo. The residue was purified by FC (EtOAc/hexane, 1:5) to provide the product ent-20 (42 mg, 91%). Ent-20: [α]_{D}²³=−24.0 (c 0.5, CHCl₃); IR 2947, 1459, 1099, 1039, 740 cm⁻¹; ¹H NMR (400 MHz, CDCl₃) δ 7.31 (5H, m), 5.83 (1H, dddd, J=7.2, 7.6, 10.0, 17.2 Hz), 5.26 (2H, m), 4.70 (1H, d, J=6.4 Hz), 4.58 (1H, d, J=6.4 Hz), 4.53 (1H, d, J=12.4 Hz), 4.42 (1H, d, J=12.4 Hz), 4.16 (1H. dd, J=4.4, 7.2 Hz), 3.93 (1H, d, J=1.6 Hz), 3.78 (1H, d, J=8.8 Hz), 3.67 (1H, m), 3.47 (1H, m), 3.38 (3H, s), 3.37 (3H, s), 3.34 (1H, d, J=8.8 Hz), 3.33 (3H, s), 3.32 (3H, s), 1.80 (2H, m), 1.65 (2H, m), 1.12 (3H, s), 1.11 (3H, s), 0.94 (9H, t, J=8.0 Hz), 0.66 (6H, q, J=8.0 Hz) ppm; ¹³C NMR (75 MHz, CDCl₃) δ 139.7, 135.3, 128.3, 128.0, 127.4, 118.6, 104.5, 94.4, 80.0, 78.0, 77.2, 75.7, 73.2, 71.2, 69.2, 58.3, 55.9, 55.8, 51.7, 46.0, 36.3, 29.4, 21.5, 21.1, 7.4 (3C), 5.6 (3C) ppm; HRMS Calcd for C₃₂H₅₆O₈SiLi ([M+Li]⁺): 603.3904. Found: 603.3900.

To a solution of olefin 75 (400 mg, 0.83551 mmol) in CH₂Cl₂ (4 mL) at RT was added methyl acrylate (304 μL, 3.34 mmol), followed by the addition of Grubbs's second generation Ru-alkylidene catalyst (71 mg, 0.0836 mmol). The mixture was kept at 38° C. for 15 h, and another portion (71 mg) of catalyst was added. After stirring for 6 h, the solvent was removed and the residue was purified by FC (EtOAc/hexane, 1:10) to give the product 43 (364 mg, 81%). 43: IR 2952, 1731, 1660, 1455, 1435, 1328, 1270, 1038, 805, 736 cm⁻¹; ¹H NMR (400 MHz, CDCl₃) δ 7.34 (4H, m), 7.26 (1H, m), 7.02 (1H, ddd, J=7.2, 7.6, 16.0 Hz), 5.91 (1H, d, J=16.0 Hz), 4.52 (1H, d, J=12.0 Hz), 4.45 (1H, d, J=12.0 Hz), 3.95 (1H, d, J=2.0 Hz), 3.77 (1H, d, J=8.8 Hz), 3.73 (3H, s), 3.66 (1H, m), 3.48 (1H, ddd, J=2.0, 4.4, 11.2 Hz), 3.32 (3H, s), 3.31 (3H, s), 2.44 (2H, m), 1.61 (2H, m), 1.11 (3H, s), 1.10 (3H, s), 0.93 (9H, t, J=8.0 Hz), 0.65 (6H, q, J=8.0 Hz) ppm; ¹³C NMR (75 MHz, CDCl₃) δ 166.9, 145.2, 139.7, 128.3 (2C), 127.4 (2C), 127.3, 123.5, 104.5, 76.8, 75.7, 73.2, 70.9, 70.5, 55.9, 51.8, 51.6, 46.0, 38.7, 28.9, 21.5, 21.1, 7.3 (3C), 5.5 (3C) ppm.

### Example 4

### Synthesis of C2,C3 Peloruside Analogs

Scheme 9 illustrates a strategy for producing peloruside analogs that lack C2,C3 hydroxy functionality. Olefin cross-metathesis of olefin 76 with methyl acrylate produces compound 43 in 81% yield. Conjugate reduction of this material provides the saturated equivalent 44. Both of these materials can be elaborated into peloruside analogs 47 and 48, according to similar procedures as outlined in schemes 6 and 7.

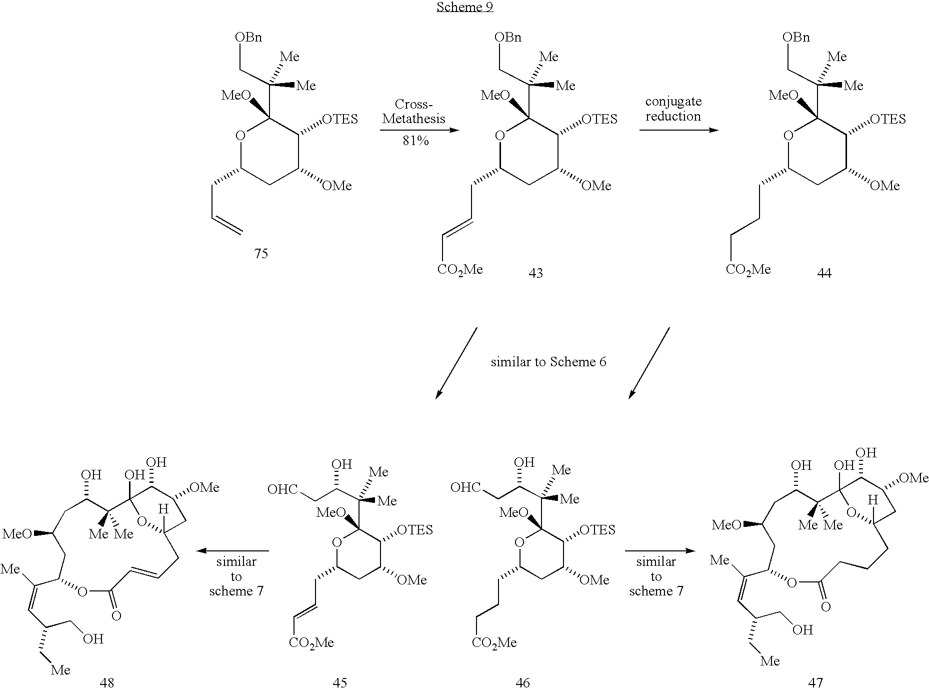

### Example 5

### Synthesis of Peloruside Analogs that Lack C8 Hydroxy Functionality

For producing Peloruside analogs that lack the C8 hydroxyl group, compound 49 is reacted with compound 50 via an aldol reaction (see scheme 10 below). Treatment of the resulting aldol product 51 under acidic conditions in methanol solvent affords tetrahydropyranyl derivative 52. This compound is elaborated into Peloruside analog 53, according to similar procedures as outlined in scheme 7.

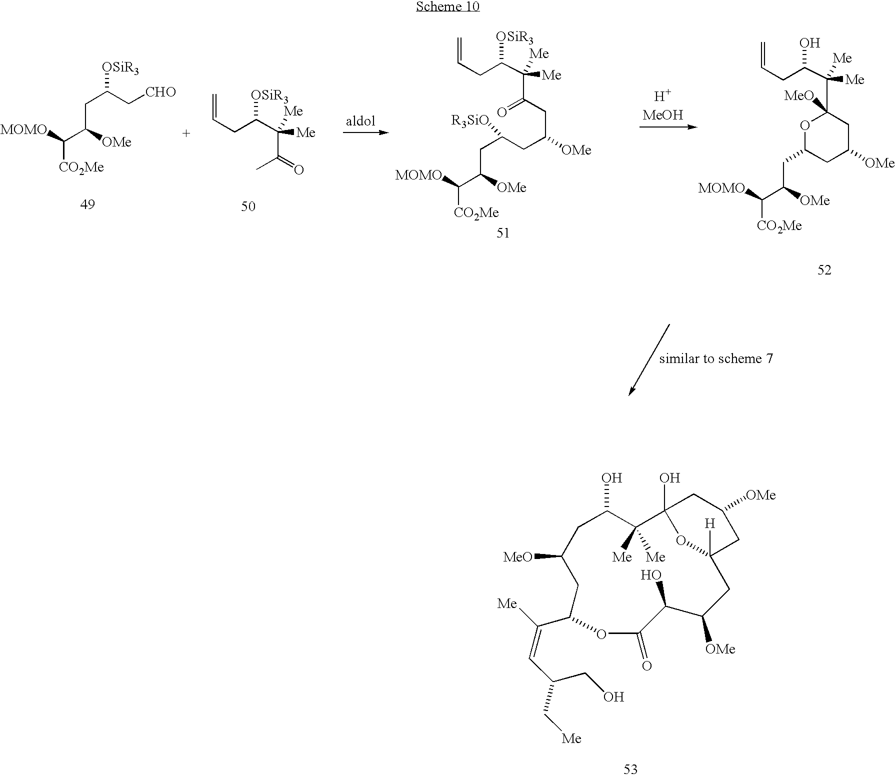

### Example 6

### Cytotoxic Effect of Peloruside A on Various Cancer Cell Lines

The object of the present study is to investigate the effect of a compound of the present invention, Peloruside A, on the proliferation of various tumor cell lines, listed in Table 1. The effects of Peloruside A were compared to the effects of microtubule-stabilizing drug, Taxol®, on cell proliferation. The taxanes, such as paclitaxel or Taxol®, belong to a class of anticancer drugs that stabilize microtubules and regulate tumor cell death. Synthetic (+)-Peloruside A was used for the present example.

TABLE 1
Cell Lines Used in Cytotoxicity Assay

| Cell Lines | Cancer Type | Cells/Well |
|---|---|---|
| MDA-MB-231 | Breast | 125 |
| LoVo | Colon | 250 |
| NCI-H1395 | Lung | 2000 |
| NCI-H2887 | Lung | 500 |
| NCI-H460 | Lung | 100 |
| NCI-H23 | Lung | 250 |
| DU-145 | Prostate | 200 |
| BT-549 | Breast | 250 |
| Capan-1 | Pancreas | 1000 |
| PC-3 | Prostate | 250 |
| Hep-G2 | Liver | 500 |
| SK-HEP-1 | Liver | 250 |
| HCT-116 | Colon | 125 |
| HCT-15 | Colon | 125 |
| SK-MEL-28 | Melanoma | 250 |
| SK-MEL-5 | Melanoma | 1000 |
| Mia PaCa-2 | Pancreas | 250 |
| SK-OV-3 | Ovarian | 250 |
| CAKI-1 | Renal | 500 |
| A498 | Renal | 125 |

Cells were plated on Day 0 in 180 μL of media (RPMI-1640, +10% FBS, +gentamycin) in a 96-well plate as indicated in Table 1. Cells were treated on Days 1, 3, 5, and 7 with 5 μL of media, vehicle, or drug. Peloruside A was resuspended in 0.1% DMSO and added to wells to a final concentration of 0.01, 0.1, 1, 10 or 100 nM. Taxol® (Sigma-Aldrich) was resuspended in 0.1% ethanol and added to cells to a final concentration of 0.1, 1, 10, 100, or 1000 nM.

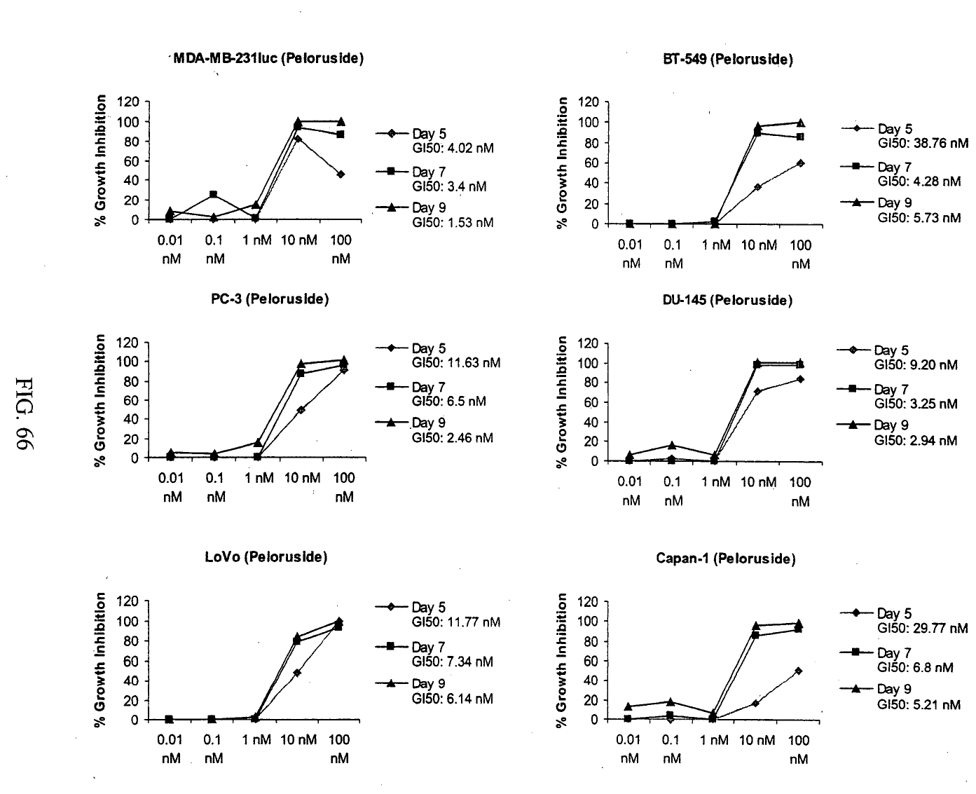

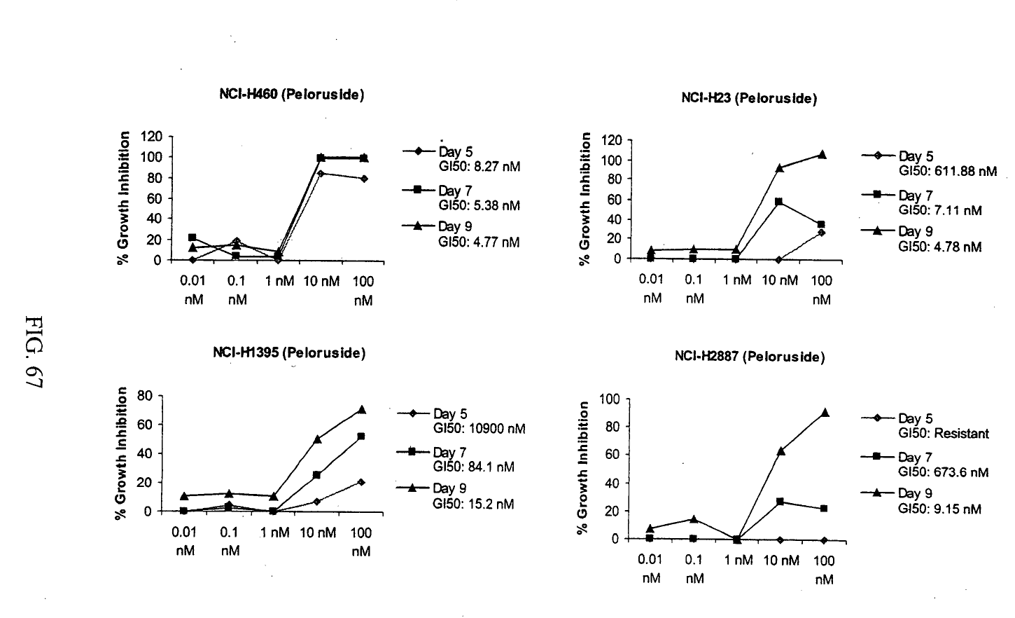

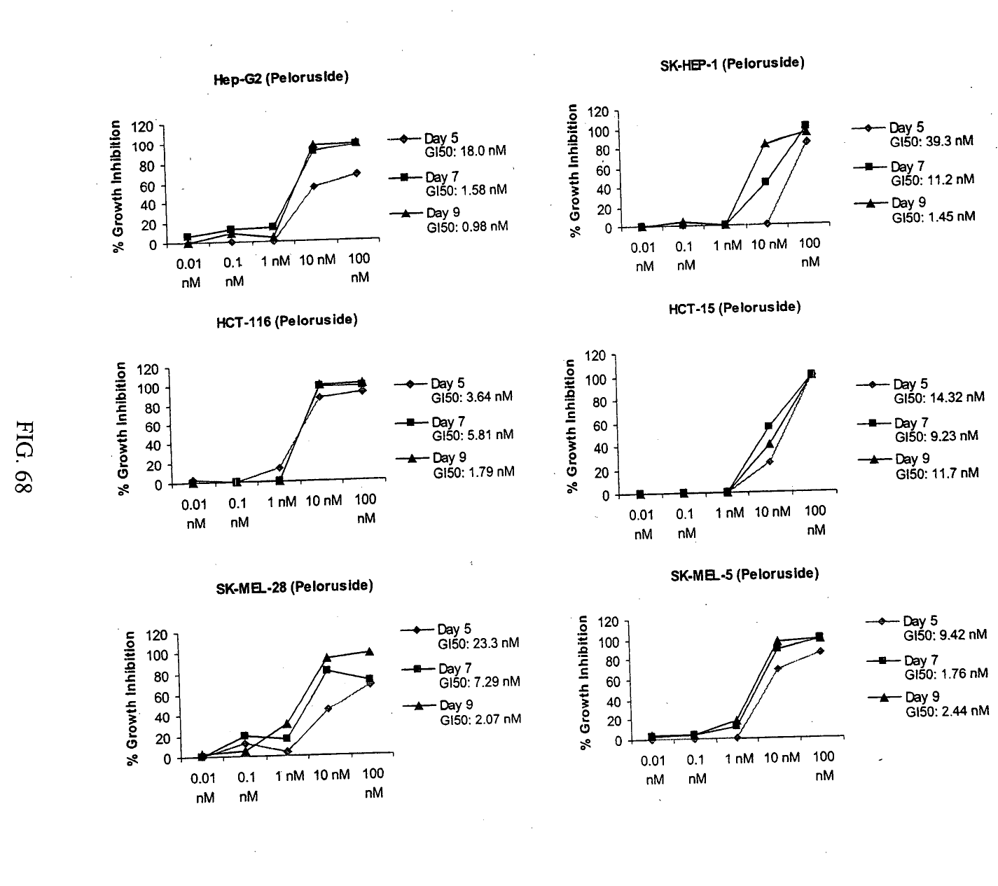

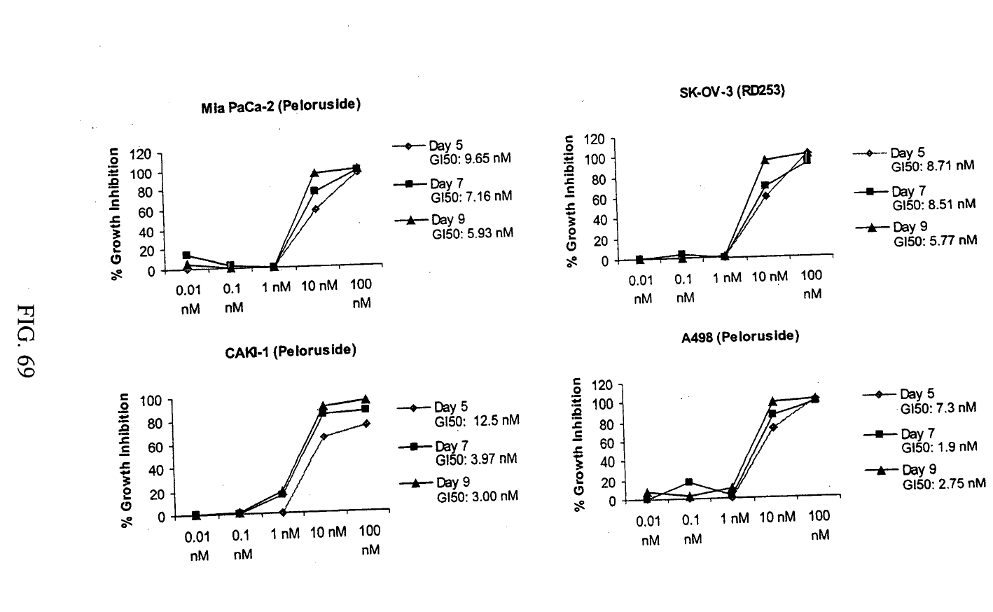

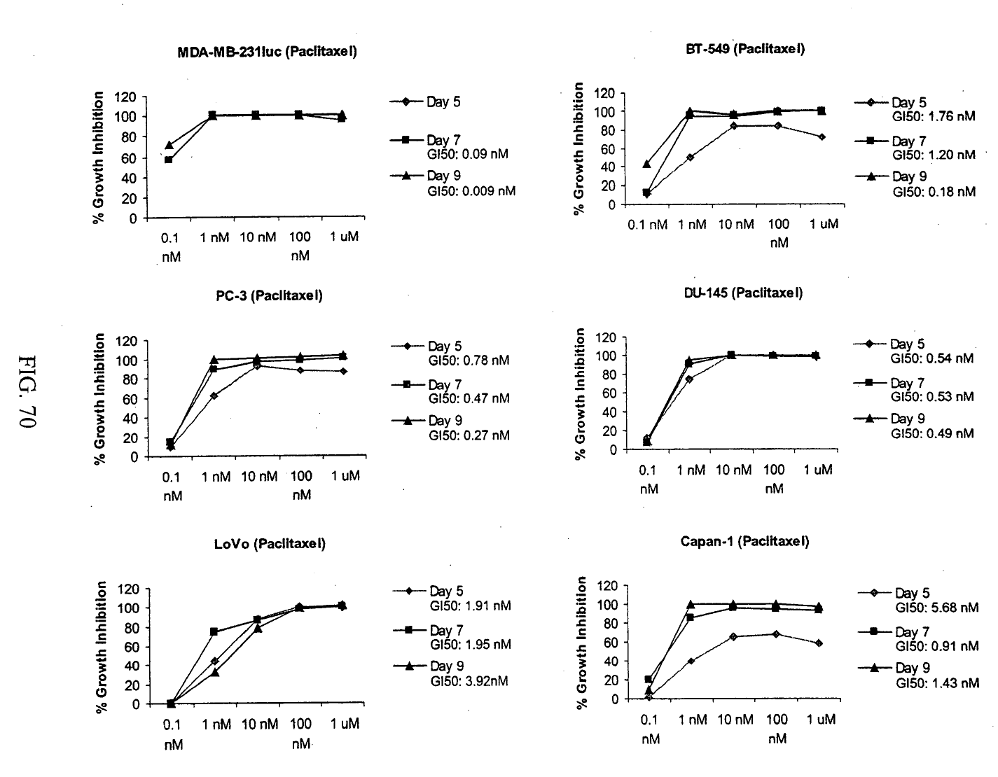

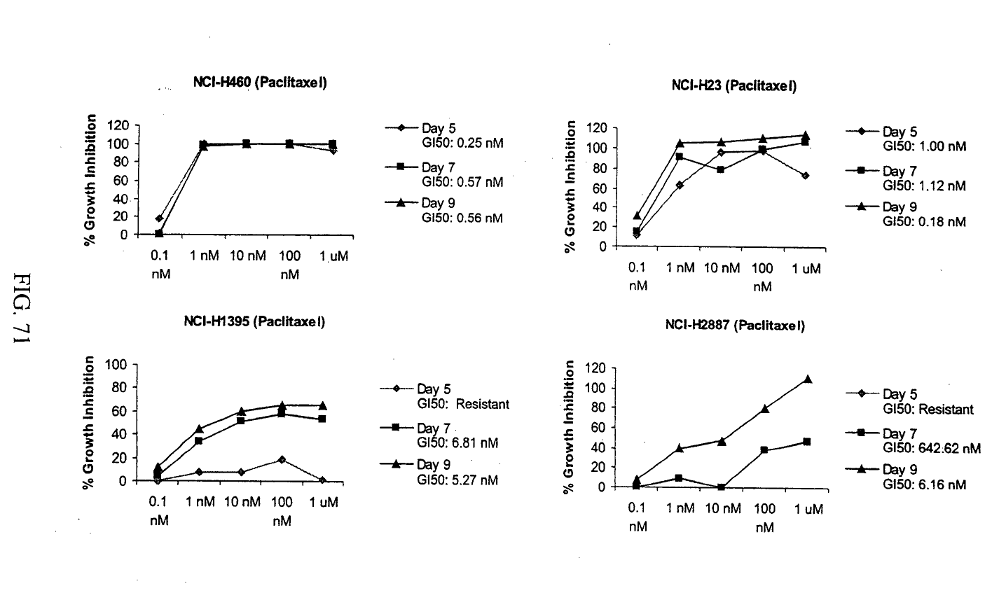

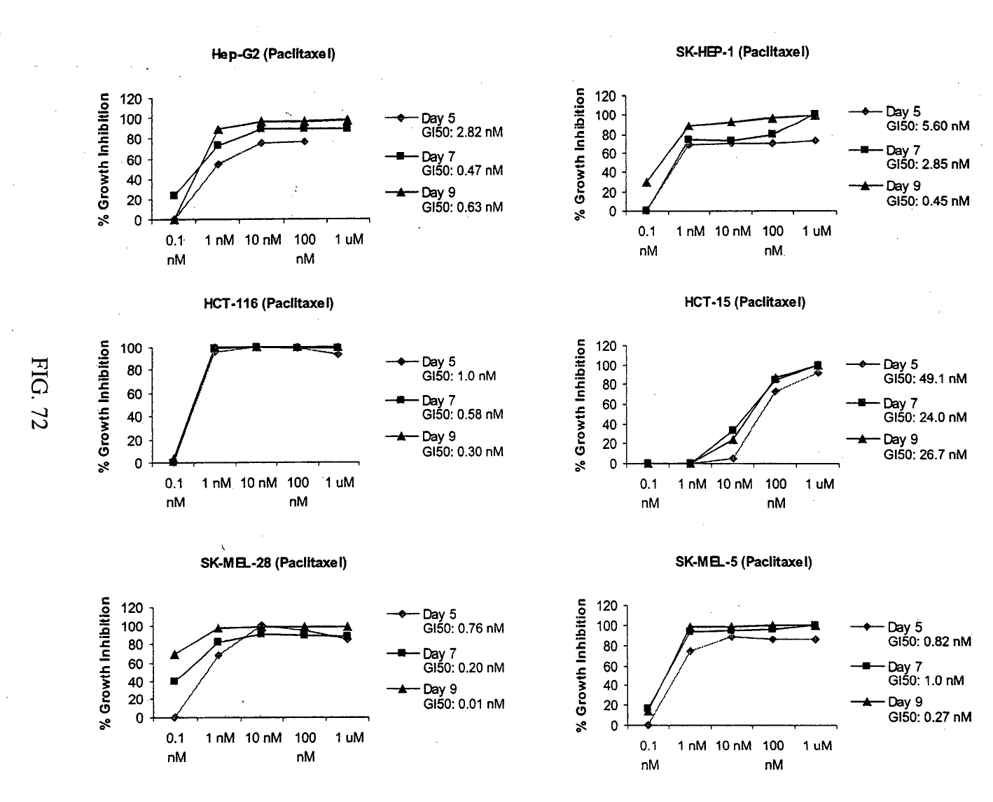

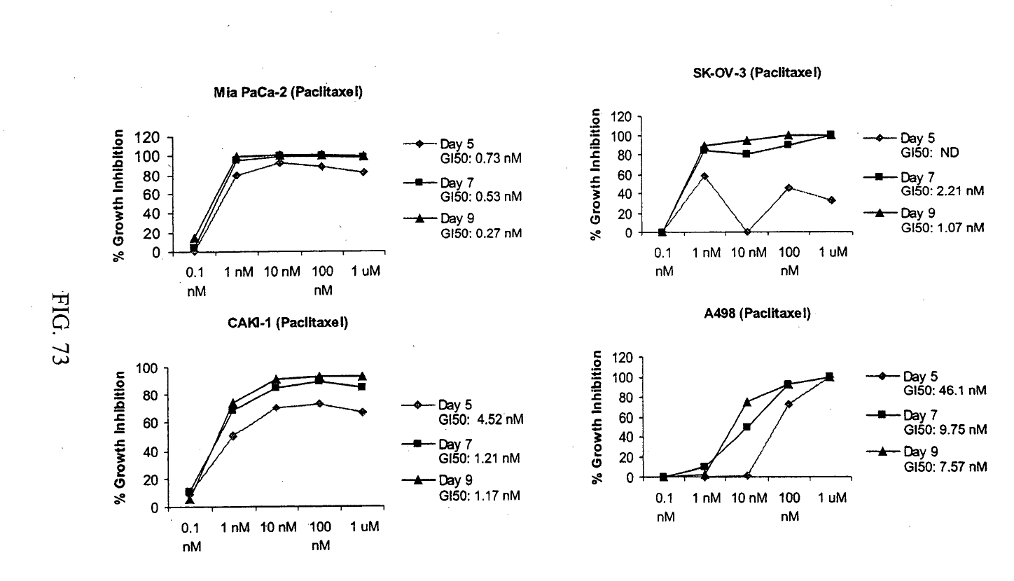

The amount of surviving cells was measured using the sulforhodamine B (SRB) assay as previously described (Skehan et al., New colorimetric cytotoxicity assay for anticancer-drug screening. J. Natl. Cancer Inst., 1990. 82(13): p. 1107-12). Cells were fixed on Days 5, 7, and 9 (representing 4, 6, and 8 days of exposure to drug or control, respectively) with 50% (w/v) trichloroacetic acid for 1 hour at 4° C., rinsed thoroughly with water, and dried overnight. The dried cells were stained with 4% sulforhodamine B in 1% acetic acid, rinsed thoroughly and dried overnight. The bound dye was resuspended in 10 mM Tris and the absorbance was read at 492 nm. Percent growth inhibition (GI) was calculated as follows: %GI=(1−(absorbance sample/absorbance vehicle))×100. The absorbance value for each drug concentration and control represents the average of 6 replicates. The %GI was plotted against the log[drug] to generate the growth curves. The concentration of drug required to inhibit growth by 50% (GI₅₀) was calculated for each time point. The results of the assays are shown in FIGS. 66-73 and Table 2.

Peloruside A inhibited proliferation of a panel of tumor cell lines, including cells derived from colon, pancreas, melanoma, ovarian, renal, liver lung, breast and prostate tissue. Peloruside A treatment created a cytotoxic effect similar to Taxol® treatment of the same cells. In fact, Peloruside A is more effective at regulating cell proliferation in HCT-15 cells in comparison to Taxol® (GI₅₀ of 14.32 nM for Peloruside A in comparison to 49.1 nM for Taxol® after 4 days of treatment with drug). A similar effect was observed with A498 cells (GI50 of 7.3 nM for Peloruside A in comparison to 46.1 nM for Taxol® after 4 days of treatment with drug). These studies suggest that the Peloruside A functions through disruption of microtubule dynamics in a manner similar to Taxol®.

TABLE 2
GI₅₀ of Peloruside A and Taxol ® on Cancer Cell Lines

| Cell Line | Type of Cancer | Paclitaxel G150 | Peloruside G150 |
|---|---|---|---|
| LoVo | Colon | 4 Days: 1.91 nM | 4 Days: 11.77 nM |
| | | 6 Days: 1.95 nM | 6 Days: 7.34 nM |
| | | 8 Days: 3.92 nM | 8 Days: 6.14 nM |
| HCT-15* | Colon | 4 Days: 49.1 nM | 4 Days: 14.32 nM |
| | | 6 Days: 24.0 nM | 6 Days: 9.23 nM |
| | | 8 Days: 26.7 nM | 8 Days: 11.7 nM |
| HCT-116 | Colon | 4 Days: 1.0 nM | 4 Days: 3.64 nM |
| | | 6 Days: 0.58 nM | 6 Days: 5.81 nM |
| | | 8 Days: 0.30 nM | 8 Days: 1.79 nM |
| Capan-1 | Pancreas | 4 Days: 5.68 nM | 4 Days: 29.77 nM |
| | | 6 Days: 0.91 nM | 6 Days: 6.8 nM |
| | | 8 Days: 1.43 nM | 8 Days: 5.21 nM |
| Mia PaCa-2 | Pancreas | 4 Days: 0.73 nM | 4 Days: 9.65 nM |
| | | 6 Days: 0.53 nM | 6 Days: 1.76 nM |
| | | 8 Days: 0.27 nM | 8 Days: 2.44 nM |
| SK-MEL-5 | Melanoma | 4 Days: 0.82 nM | 4 Days: 9.42 nM |
| | | 6 Days: 1.0 nM | 6 Days: 1.76 nM |
| | | 8 Days: 0.27 nM | 8 Days: 2.44 nM |
| SK-MEL-28 | Melanoma | 4 Days: 0.76 nM | 4 Days: 23.3 nM |
| | | 6 Days: 0.20 nM | 6 Days: 7.29 nM |
| | | 8 Days: 0.01 nM | 8 Days: 2.07 nM |
| SK-OV-3 | Ovarian | 4 Days: ND | 4 Days: 8.81 nM |
| | | 6 Days: 2.21 nM | 6 Days: 8.51 nM |
| | | 8 Days: 1.07 nM | 8 Days: 5.77 nM |
| CAK1-1 | Renal | 4 Days: 4.52 nM | 4 Days: 12.5 nM |
| | | 6 Days: 1.21 nM | 6 Days: 3.97 nM |
| | | 8 Days: 1.17 nM | 8 Days: 3.00 nM |
| A498 | Renal | 4 Days: 46.1 nM | 4 Days: 7.3 nM |
| | | 6 Days: 9.75 nM | 6 Days: 1.9 nM |
| | | 8 Days: 7.57 nM | 8 Days: 2.75 nM |
| PC-3 | Prostate | 4 Days: 0.78 nM | 4 Days: 11.63 nM |
| | | 6 Days: 0.47 nM | 6 Days: 6.50 nM |
| | | 8 Days: 0.27 nM | 8 Days: 2.46 nM |
| DU-145 | Prostate | 4 Days: 0.54 nM | 4 Days: 9.20 nM |
| | | 6 Days: 0.53 nM | 6 Days: 3.25 nM |
| | | 8 Days: 0.49 nM | 8 Days: 2.94 nM |
| BT-549 | Breast | 4 Days: 1.76 nM | 4 Days: 38.76 nM |
| | | 6 Days: 1.2 nM | 6 Days: 4.28 nM |
| | | 8 Days: 0.18 nM | 8 Days: 5.73 nM |
| MB-MDA-231 | Breast | 4 Days: ND | 4 Days: 4.02 nM |
| | | 6 Days: 0.09 nM | 6 Days: 3.40 nM |
| | | 8 Days: 0.01 nM | 8 Days: 1.53 nM |
| NCl-H460 | Lung | 4 Days: 0.25 nM | 4 Days: 8.27 nM |
| | | 6 Days: 0.57 nM | 6 Days: 5.38 nM |
| | | 8 Days: 0.56 nM | 8 Days: 4.77 nM |
| NCl-H23 | Lung | 4 Days: 1.00 nM | 4 Days: ND |
| | | 6 Days: 1.12 nM | 6 Days: 7.11 nM |
| | | 8 Days: 0.18 nM | 8 Days: 4.78 nM |
| NCl-H1395 | Lung | 4 Days: Resistant | 4 Days: 1009 nM |
| | | 6 Days: 6.81 nM | 6 Days: 84.1 nM |
| | | 8 Days: 5.27 nM | 8 Days: 15.2 nM |
| NCl-H2887 | Lung | 4 Days: Resistant | 4 Days: Resistant |
| | | 6 Days: 642.62 nM | 6 Days: 673.6 nM |
| | | 8 Days: 6.16 nM | 8 Days: 9.15 nM |
| Hep G2 | Liver | 4 Days: 2.82 nM | 4 Days: 18.0 nM |
| | | 6 Days: 0.47 nM | 6 Days: 1.58 nM |
| | | 8 Days: 0.63 nM | 8 Days: 0.98 nM |
| SK-HEP-1 | Liver | 4 Days: 5.60 nM | 4 Days: 39.3 nM |
| | | 6 Days: 2.85 nM | 6 Days: 11.2 nM |
| | | 8 Days: 0.45 nM | 8 Days: 1.45 nM |

*Peloruside A is more effective at inhibiting cell proliferation then Paclitaxel in HTC-15 cells.

### Example 7

### Peloruside A Effect on Tubulin Polymerization

Taxol® functions to inhibit cell proliferation through the stabilization of microtubules, reducing the pool of cellular free tubulin. As a result, dividing tumor cells become arrested in mitosis. The present example investigates the capability of Peloruside A to polymerize free tubulin in vitro. Synthetic (+)-Peloruside A was used for the present example.

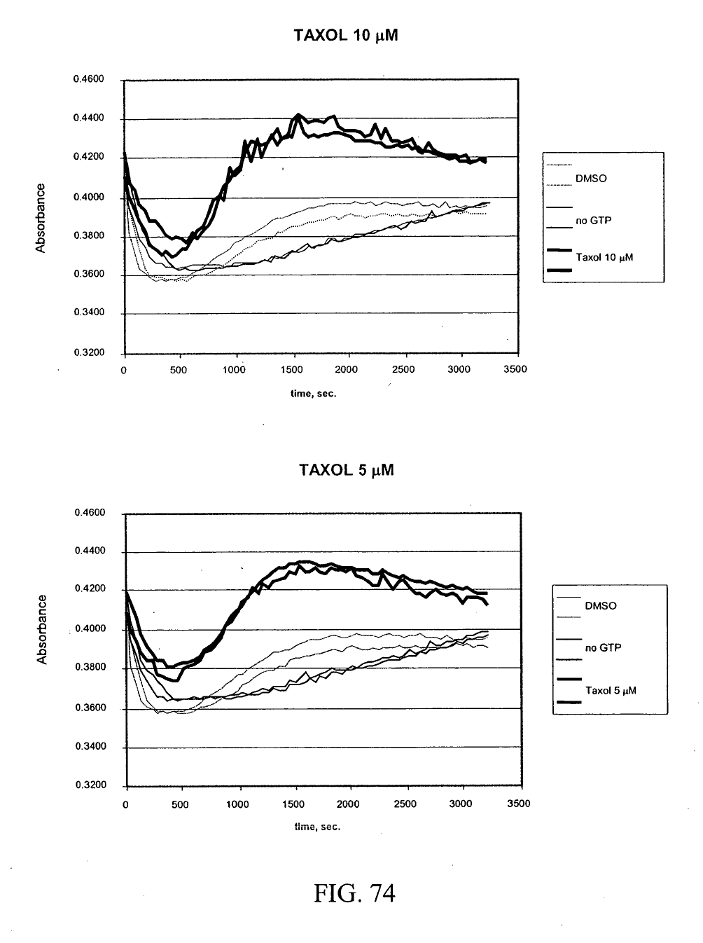

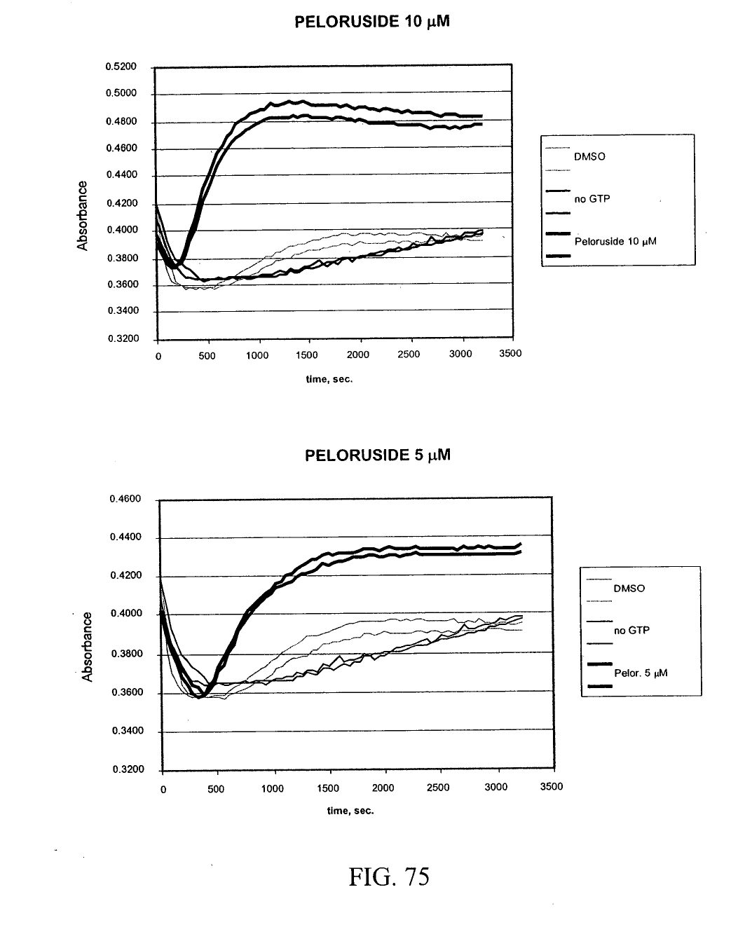

Tubulin, purified from bovine brains and prepared in PEM-buffer (80 mM PIPES, 0.5 mM MgCl₂, 1 mM EGTA, pH 6.9) was thawed under vacuum on ice. Tubulin was incubated at 37° C. until almost melted and resuspended to a final concentration of 2 mg/ml and placed on ice. Peloruside A or Taxol® was added into transparent 96-well plates (Coming Incorp. Costar) and placed on ice. GTP (Sigma) was added to a final concentration of 1 mM. The 96 well plate was warmed to 37° C. and 120 μl of tubulin solution was added into each well. The absorbance of the reaction at 340 nm over time (each 40-50 sec.) was measured on the spectrophotometer TECAN SPECTRAFluor Plus. Peloruside A or Taxol® was added to the tubulin at a final concentration of 5 or 10 μM. The tubulin polymerization curves are shown in FIGS. 74 and 75.

Peloruside A demonstrates a potent tubulin stabilization activity (FIG. 75). The rate of polymerization is similar to the rate of polymerization exhibited by Taxol® at both 5 or 10 μM of drug (FIG. 74). Therefore, Peloruside A may function to inhibit tumor cell growth by disrupting tubulin dynamics.

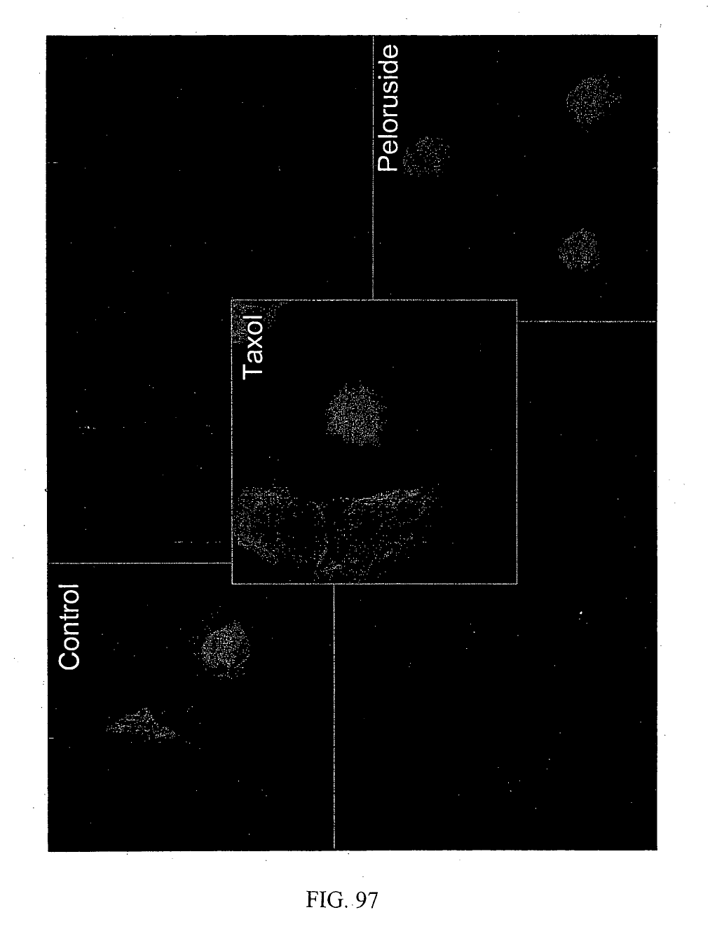

In addition, immunostained tubulin in BSC-1 cells show that Peloruside A inhibits the formation of a bipolar spindle (FIG. 97). Control untreated cells exhibit chromosomes lined at the mitotic plate. Similar to Taxol® treatment, the bipolar spindle does not form when BSC-1 cells are treated with Peloruside A. Instead the chromosomes surround the monoaster that forms in the cell.

### Example 8

### Cytotoxic Effect of Peloruside Analogs

The cytotoxic effect of synthetic Peloruside A analogs, LX3111 and LX3136, on HeLa and SK-MEL-5 cells was investigated. LX3 111 is a compound of the formula II of the present invention and LX3136 is a compound of the formula III of the present invention. Synthetic (+)-Peloruside A analogs were prepared for the present example.

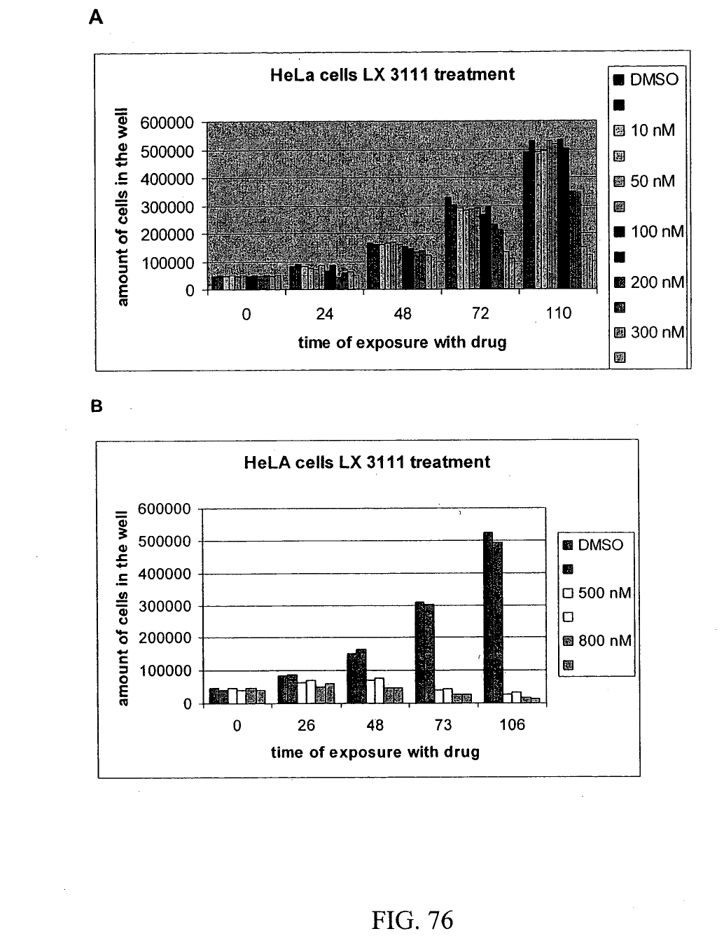

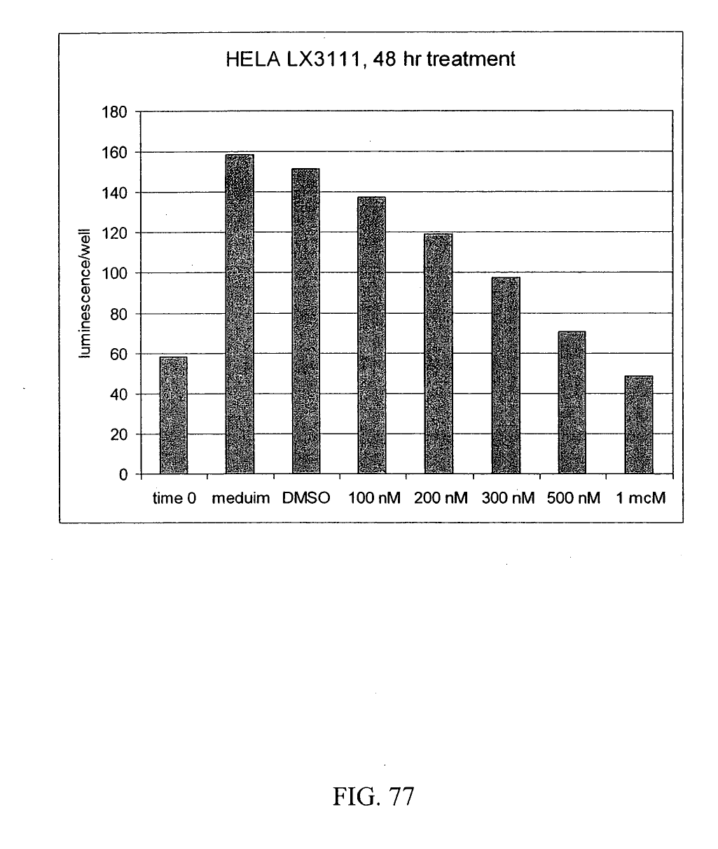

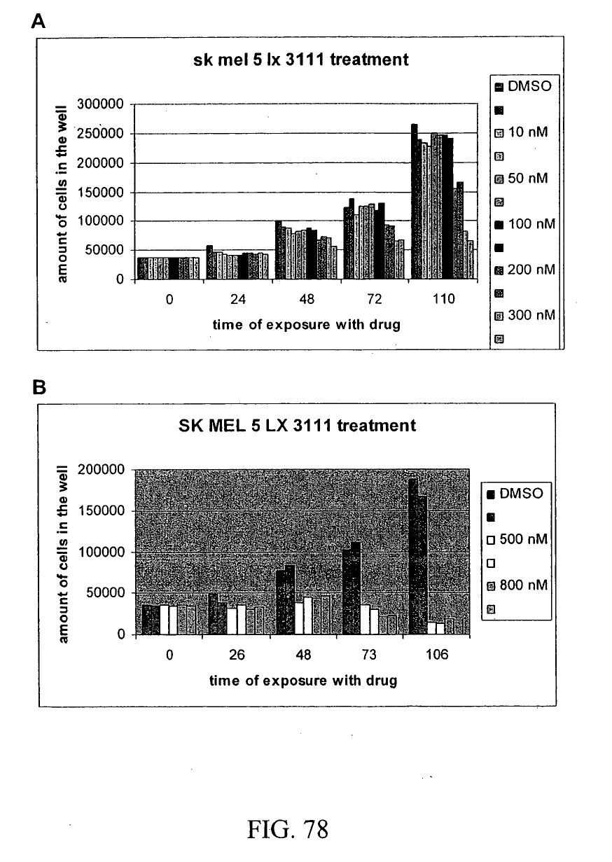

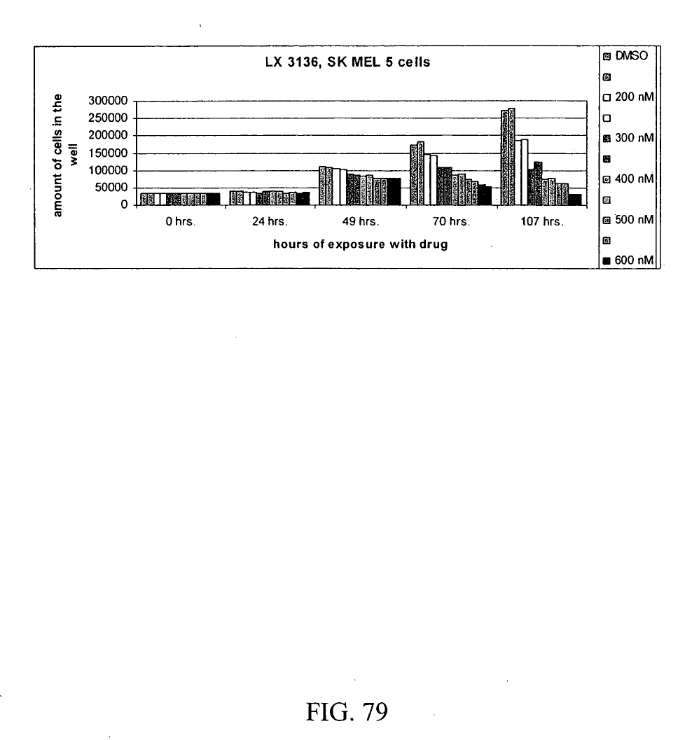

Cells were treated with medium, DMSO, LX3111 or LX3136 at a final concentration of 10, 50, 100, 200, 300, 400, 500, 600, 800 or 1000 nM. Cell viability was measured by counting surviving cells or measuring luminescence at 48 hours after drug treatment using the Cell Titer-Glo Luminescent Cell Viability Assay (Promega). FIGS. 76-79 shows bar graphs of the inhibition of cell growth exhibited by the Peloruside analogs.

Both LX3111 and LX3136 exhibited potent antimitotic activity in inhibiting cell growth of tumor cells, HeLa and SK-MEL-5.

### Example 9

### Peloruside A Treatment of Taxol®-Resistant Cell Lines

Taxol®-resistant ovarian cancer cell lines, Ptx-10 and Ptx-22, isolated from the parent 1A9 cell line, contain point mutations in the beta-tubulin gene (Giannakakou et al., Paclitaxel-resistant human ovarian cancer cell have mutant beta-tubulins that exhibit impaired paclitaxel-driven polymerization, J. Biol. Chem., 272, 17118-17125, 1997). These cells were generated by exposure to Taxol® and verapamil and exhibit defective tubulin polymerization. These cell lines have specific amino acid mutations in tubulin that prevent Taxol®-binding. The present example demonstrates the cytotoxtic effect of Peloruside A treatment on these Taxol)-resistant tumor cell lines in comparison to Taxol® treatment. Synthetic (+)-Peloruside A was used for the present example.

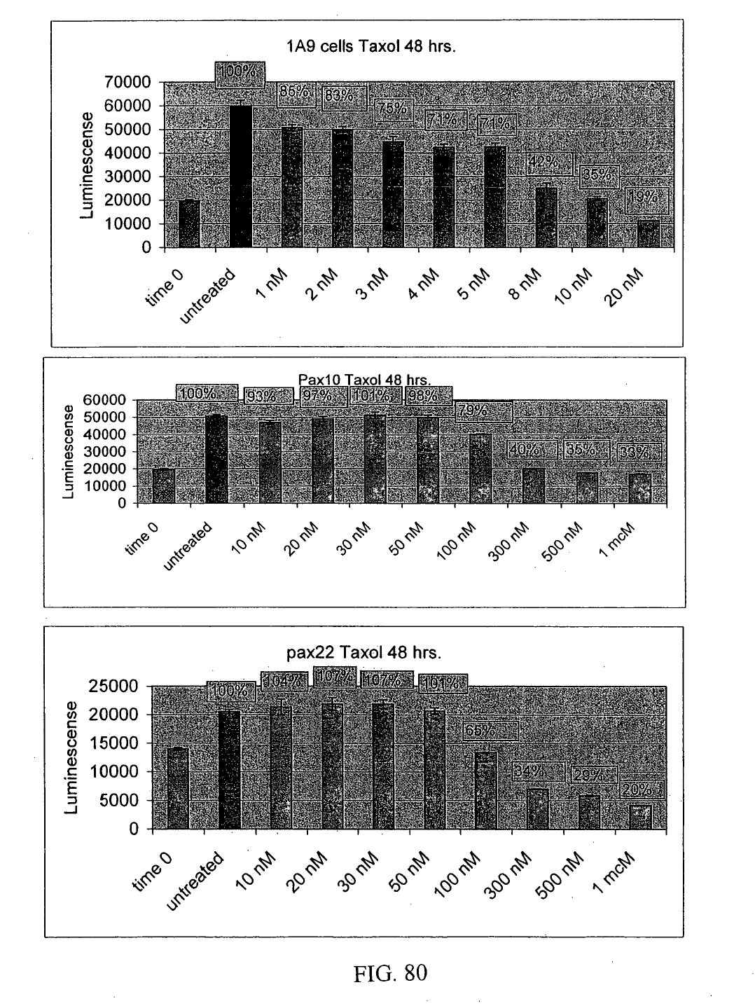

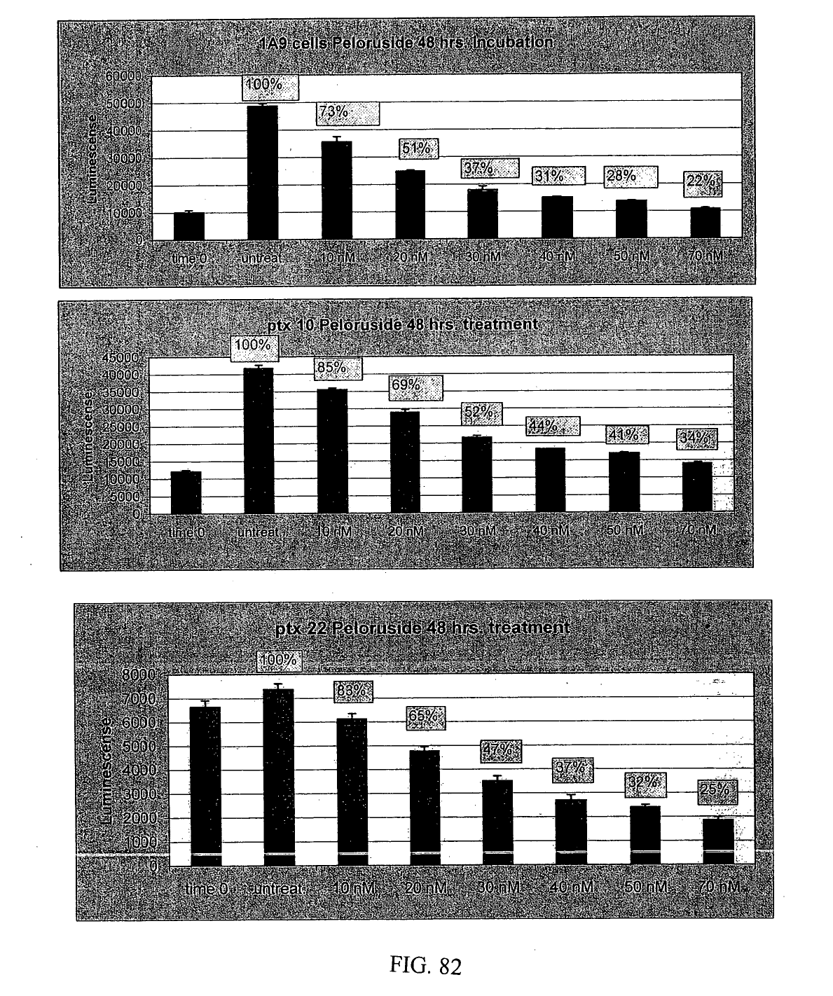

1A9, ptx10 and ptx22 cells were plated in 96-well plates to approximately 50% confluency. On the next day, Taxol® or Peloruside A was added to each well and incubated for 48 hours. A final concentration of 1, 2, 3, 4, 5, 8, 10, 20, 30, 50, 100, 300, 500, or 1000 nM of Taxol® was used. A final concentration of 10, 20, 30, 40, 50 or 70 nM of Peloruside A was used. Cell viability was measured using the Cell Titer-Glo Luminescent Cell Viability Assay (Promega). FIGS. 80-82 show bar graphs of cell viability at various concentrations of Peloruside A and Taxol®.

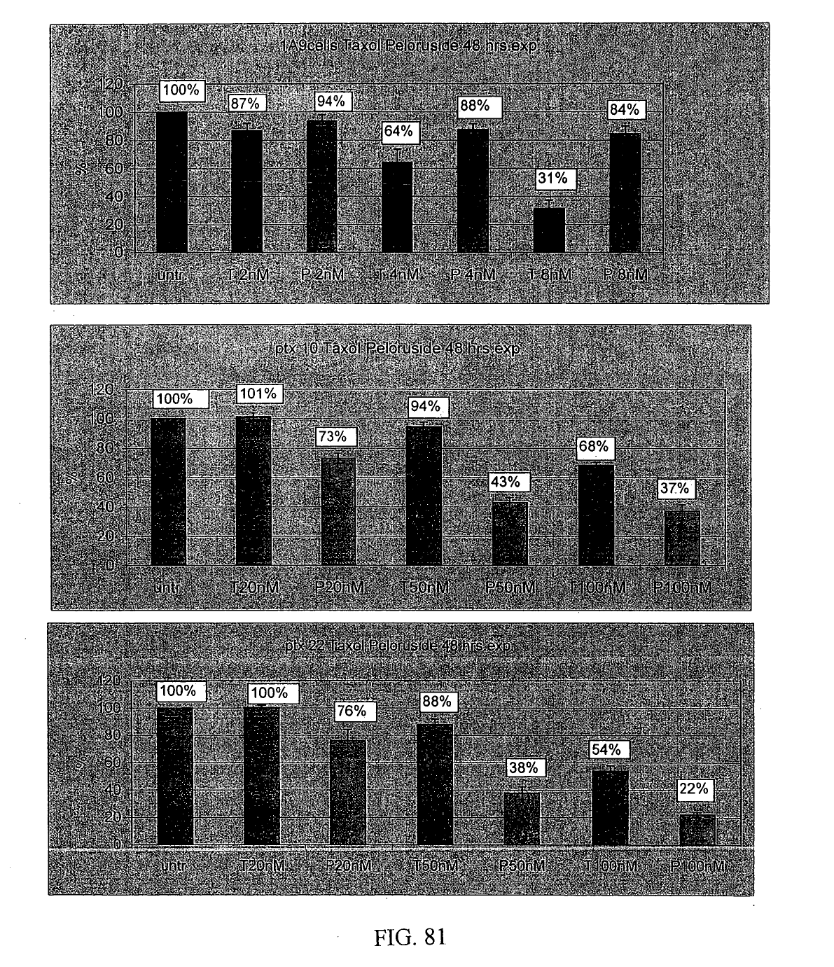

Peloruside A demonstrate potent antimitotic activity by inhibiting cell growth of both the parental and Taxol®-resistant tumor cell lines (FIGS. 80 and 81). Peloruside A reduces cell viability to approximately 50% at 30 nM of drug in ptx10 and ptx22 cells, whereas treatment of these cells must exceed 100 nM to achieve a similar effect with Taxol® treatment. The relative magnitude of the effect caused by Peloruside A treatment suggests a different mechanism of action. Peloruside A may bind either tubulin at the Taxol®-binding site in a manner unaffected by the mutations found in ptx10 and ptx22 or Peloruside A binds tubulin at another site. In any case, the experiments demonstrate the utility of Peloruside A in Taxol®-resistant tumor cells.

All of the compositions and/or methods disclosed and claimed herein can be made and executed without undue experimentation in light of the present disclosure. While the compositions and methods of the invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Patents, publications, product descriptions and protocols are cited throughout this application, the disclosures of which are incorporated herein by reference in their entireties for all purposes.

What is claimed:

1. A synthetic compound of formula:

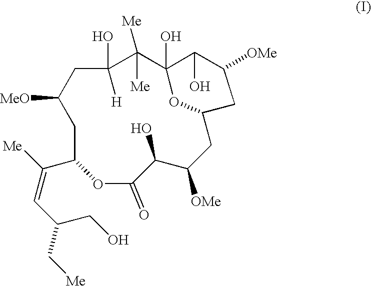

2. A synthetic compound of formula:

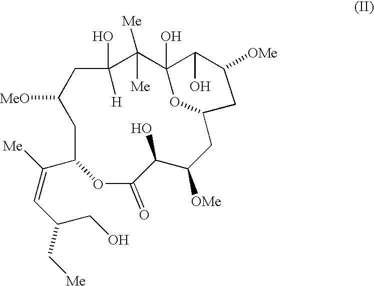

3. A compound of formula:

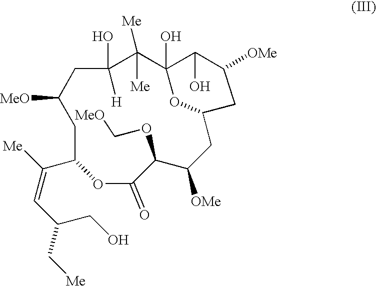

4. A compound of formula: 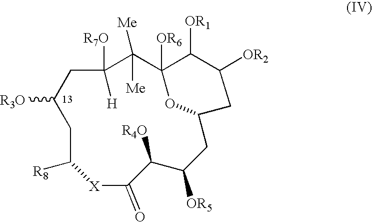
where R₁, R₂, R₃, R₄, R₅ and R₆ can be the same or different and are selected from the group consisting of H, Me, alkyl, and functionalized alkyl, and where R₈ is selected from the group consisting of H, aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl.

5. A compound of formula: 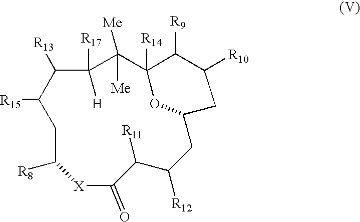
where R₁₃ is H or Me, where R₁₄, R₁₇ can be the same or different and are selected from the group consisting of H, OH, and OR, where R₉, R₁₀, R₁₁, R₁₅ can be the same or different and are selected from the group consisting of H, Me, and OR, where R and R₅ can be the same or different and are selected from the group consisting of H, Me, alkyl, and functionalized alkyl, where R₈ is selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

6. A composition comprising a compound of formula: 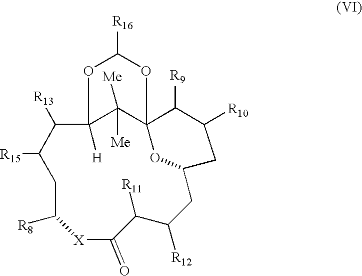
where R₁₃ is H or Me, where R₉, R₁₀, R₁₁, R₁₅ can be the same or different and are selected from the group consisting of H, Me, and OR, where R and R₅ can be the same or different and are selected from the group consisting of H, Me, alkyl, and functionalized alkyl, where R₈, R₁₆ can be the same or different and is selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

7. A composition comprising a compound of formula: 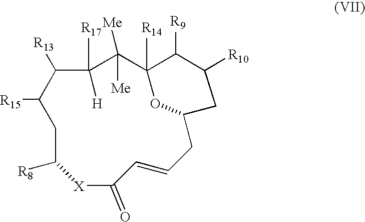
where R₁₃ is H or Me, where R₁₄, R₁₇ can be the same or different and is selected from the group consisting of H, OH, and OR, where R₉, R₁₀, R₁₅ can be the same or different and is selected from the group consisting of H, Me, OR, where R is selected from the group consisting of H, Me, alkyl, and functionalized alkyl, where R₈ is selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

8. A composition comprising a compound of formula: 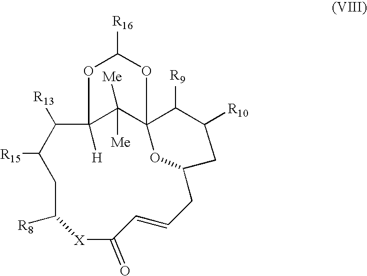
where R₁₃ is H or Me, where R₉, R₁₀, R₁₅ can be the same or different and are selected from the group consisting of H, Me, and OR, where R is selected from the group consisting of H, Me, alkyl, and functionalized alkyl, where R₈, R₁₆ can be the same or different and are selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

9. A composition comprising a compound of formula: 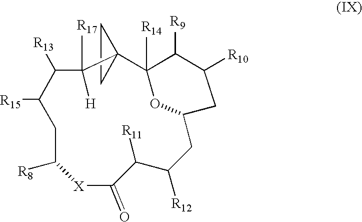
where R₁₃ is H or Me, where R₁₄, R₁₇ can be the same or different and are selected from the group consisting of H, OH, and OR, where R₉, R₁₀, R₁₁, R₁₅ can be the same or different and are selected from the group consisting of H, Me, and OR, where R and R₅ can be the same or different and are selected from the group consisting of H, Me, alkyl, and functionalized alkyl, where R₈ is selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

10. A composition comprising a compound of formula: 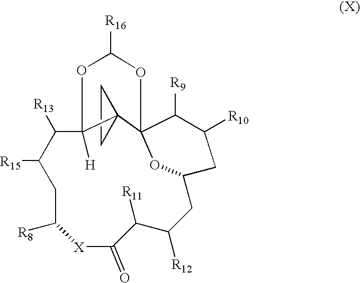
where R₁₃ is H or Me, where R₉, R₁₀, R₁₁, R₁₅ can be the same or different and are selected from the group consisting of H, Me, and OR, where R and R₅ can be the same or different and are selected from the group consisting of H, Me, alkyl, and functionalized alkyl, where R₈, R₁₆ can be the same or different and are selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

11. A composition comprising a compound of formula: 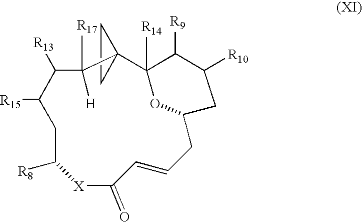
where R₁₃ is H or Me, where R₁₄, R₁₇ can be the same or different and are selected from the group consisting of H, OH, and OR, where R₉, R₁₀, R₁₅ can be the same or different and are selected from the group consisting of H, Me, OR, where R includes H, Me, alkyl, and functionalized alkyl, where R₈ is selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

12. A composition comprising a compound of formula: 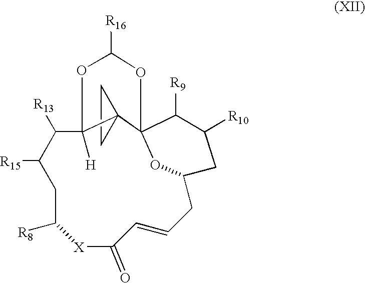
where R₁₃ is H or Me, where R₉, R₁₀, R₁₅ can be the same or different and are selected from the group consisting of H, Me, and OR, where R is selected from the group consisting of H, Me, alkyl, and functionalized alkyl, where R₈, R₁₆ can be the same or different and are selected from the group consisting of H, aryl, heteroaryl, alkyl, functionalized alkyl, alkenyl, functionalized alkenyl, alkynyl, and functionalized alkynyl, where X is O or NH, and wherein the functional group is a heteroatom, a halide, an aryl, or a heteroaryl.

13. A synthetic compound having the ¹³C NMR signature of FIG. 4 and the ¹H NMR signature of FIG. 5, wherein the compound is dextrarotary, and wherein the compound comprises microtubule-stabilizing activity.

14. A method for treating cancer comprising contacting a tumor cell within a subject with a compound of any one of claims 1 through 13 for a period of time and in an amount sufficient to inhibit growth of the tumor cell.

15. A method of suppressing growth of a tumor cell comprising contacting said cell with a compound of any one of claims 1 through 13 for a period of time and in an amount sufficient to suppress growth of the tumor cell.

16. A method of inhibiting cell proliferation comprising contacting the proliferating cells with a compound of any one of claims 1 through 13 for a period of time and in an amount sufficient to inhibit proliferation of the cells.

17. A method of stabilizing microtubule formation in a cell comprising administering to the cell the compound of any one of claims 1 through 13 for a period of time and in an amount sufficient to stabilize microtubule formation.

18. A method of producing macrolactone comprising:
a) synthesizing a pyran containing a first substituent having a carboxylic acid group and a second substituent having a hydroxyl group; and
b) reacting the carboxylic acid group with the hydroxyl group to form a macrolactone.

19. A compound selected from the group consisting of: 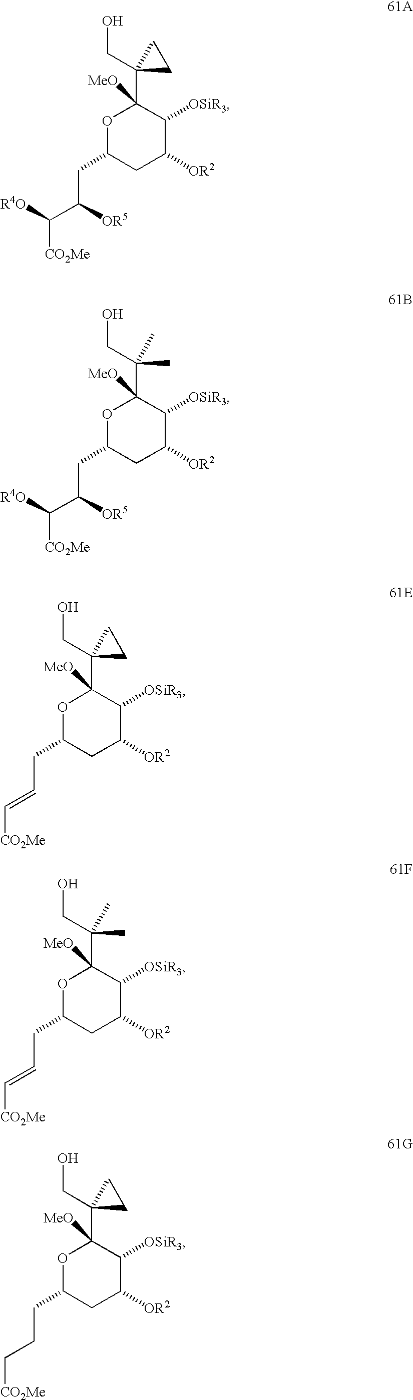 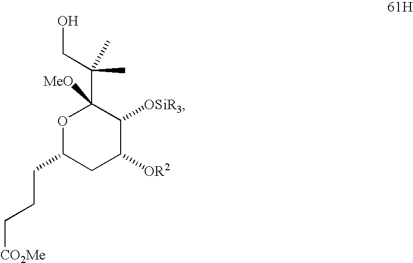
wherein R², R⁴ and R⁵ is the same or different and are selected from the group consisting of alkyl, and functionalized alkyl, wherein the functional group is a heteroatom, a halide, an aryl, or a heteroaryl.

20. A compound selected from the group consisting of: 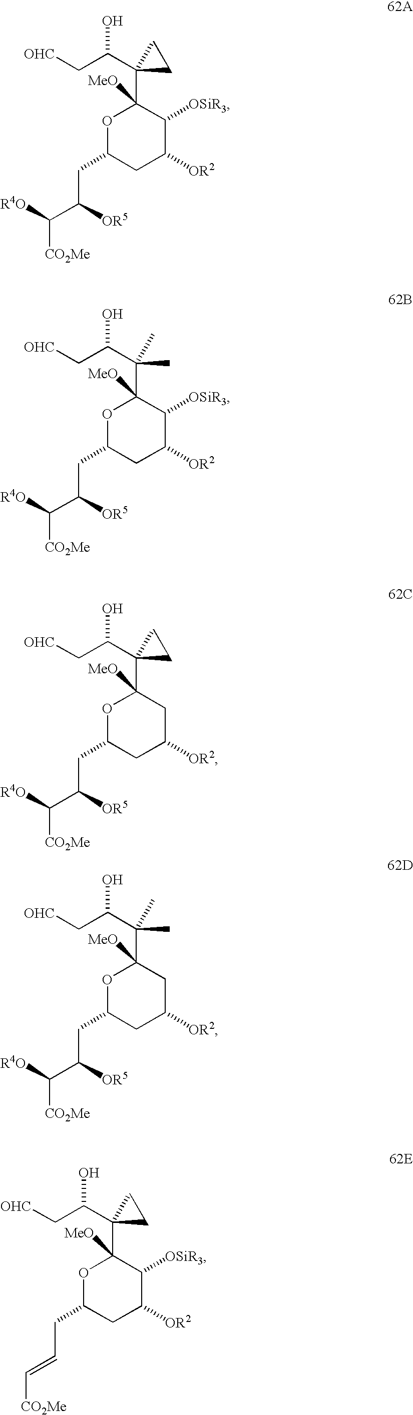 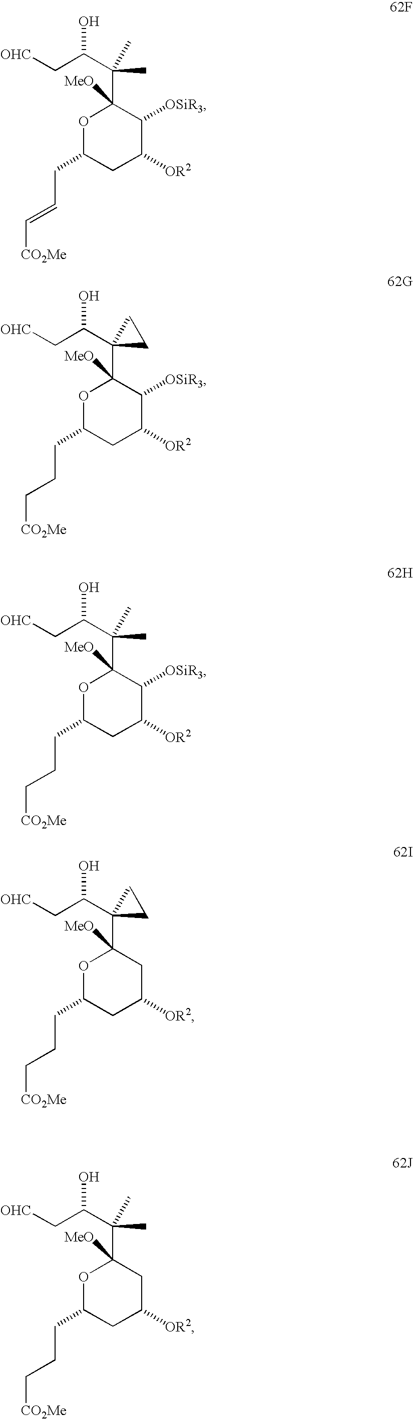
wherein R², R⁴ and R⁵ is the same or different and are selected from the group consisting of alkyl, and functionalized alkyl, wherein the functional group is a heteroatom, a halide, an aryl, or a heteroaryl.

21. A compound of selected from the group consisting of: 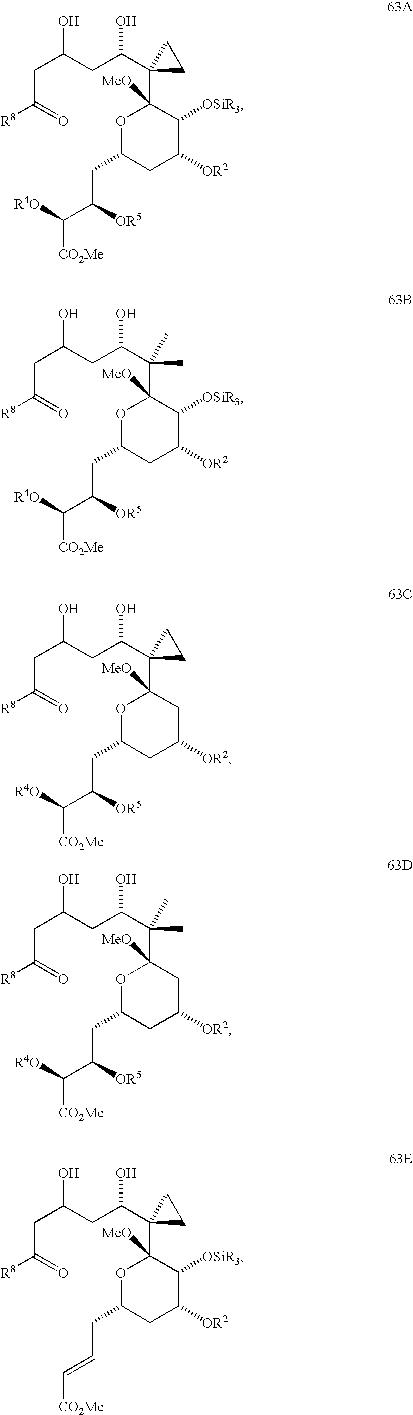 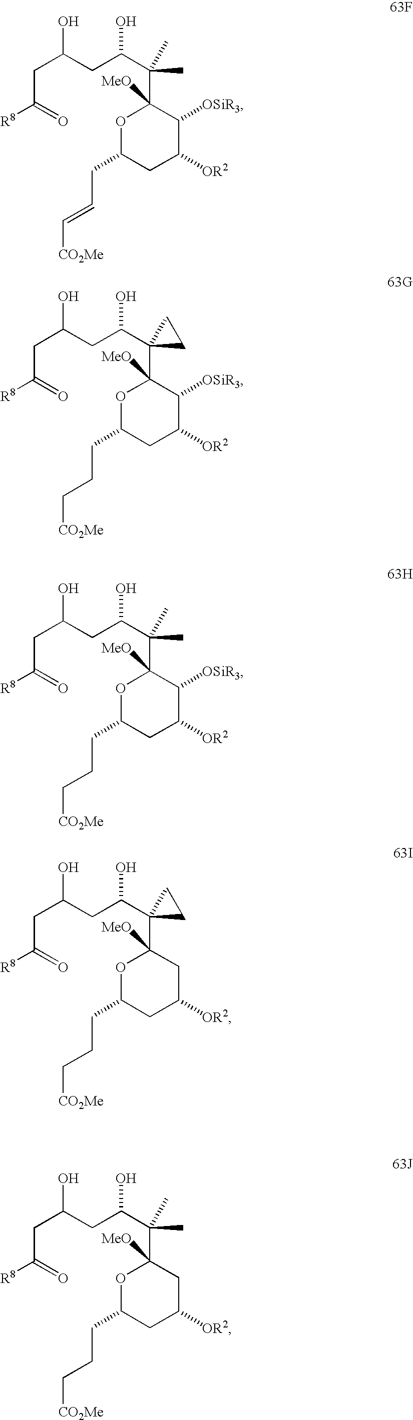
wherein R², R⁴ and R⁵ can be the same or different and are selected from the group consisting of alkyl and functionalized alkyl, and where R⁸ is selected from the group consisting of aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl.

22. A compound of selected from the group consisting of: 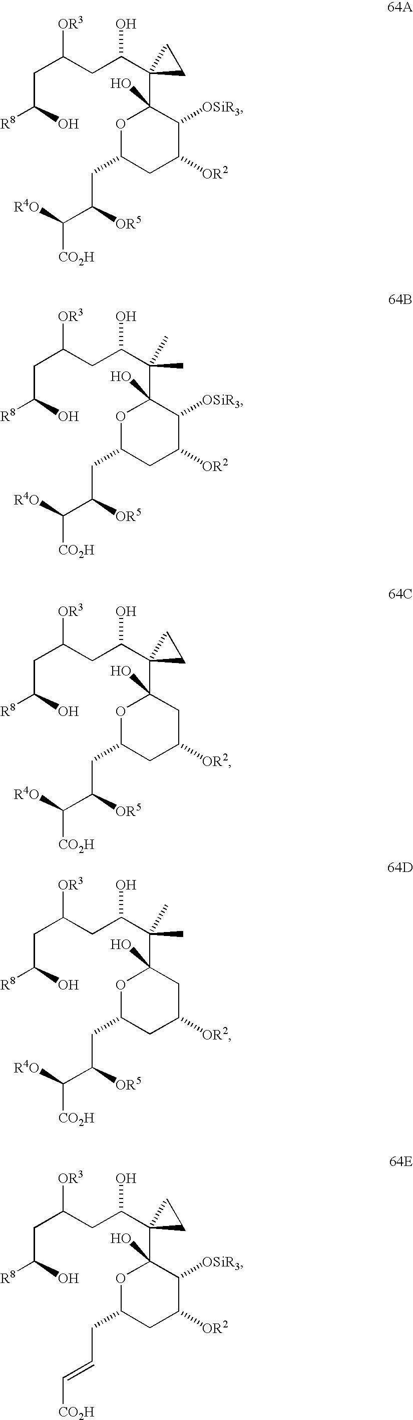 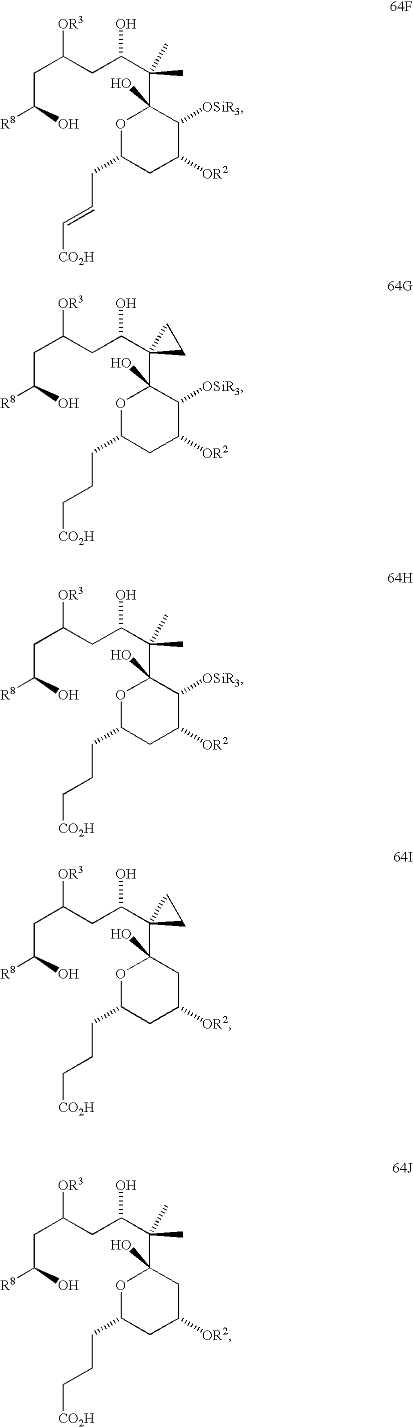
wherein R², R³, R⁴, R⁵ can be the same or different and are selected from the group consisting of alkyl, and functionalized alkyl, and where R⁸ is selected from the group consisting of aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl.

23. A compound selected from the group consisting of: 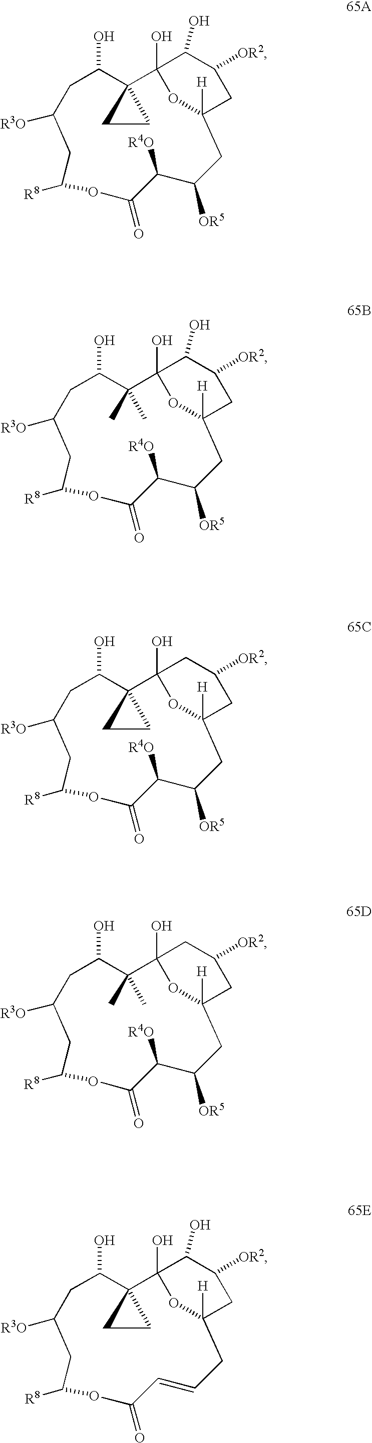 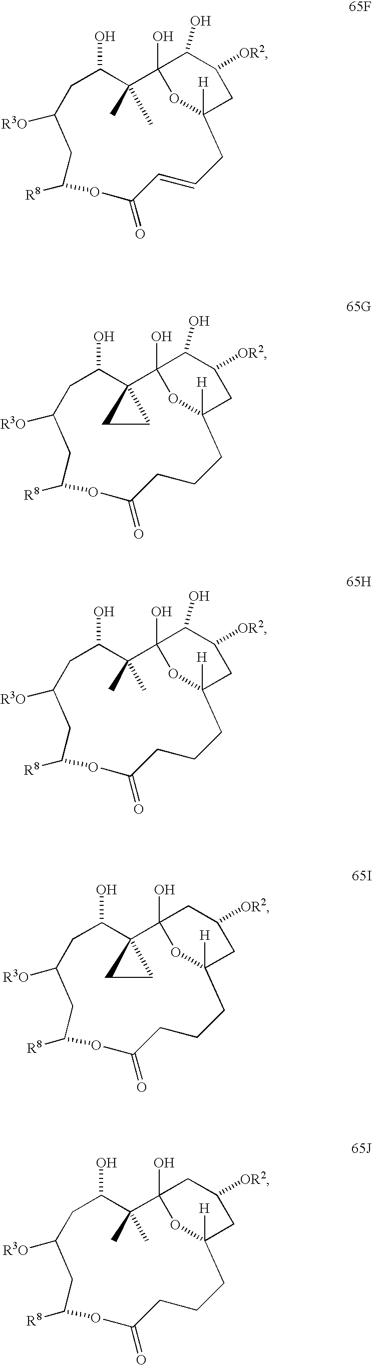
wherein R², R³, R⁴, R⁵ can be the same or different and are selected from the group consisting of alkyl, and functionalized alkyl, and where R⁸ is selected from the group consisting of aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl.

24. A method for producing Peloruside A comprising:
a) oxidizing an alcohol function in a compound selected from the group consisting of: 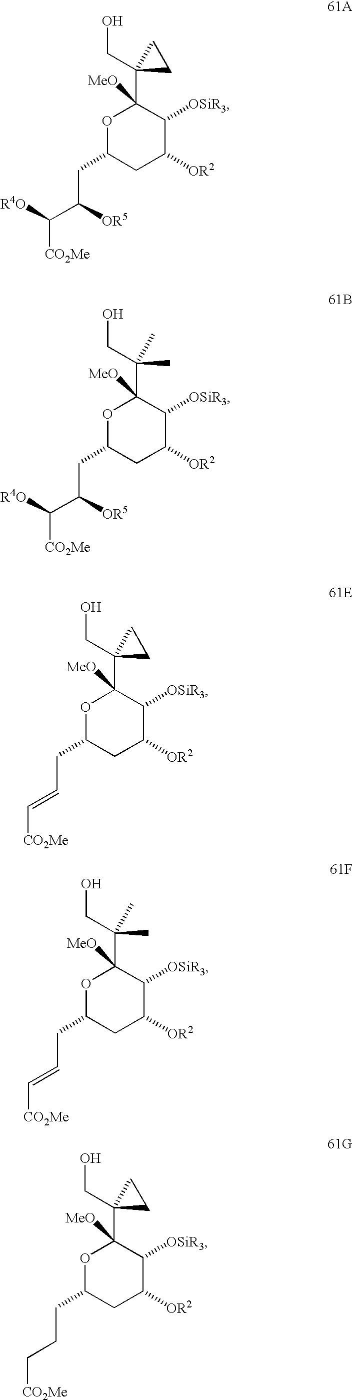 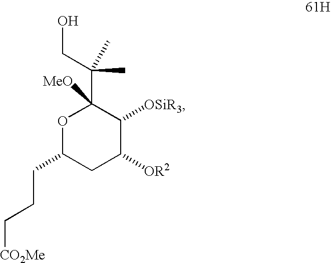
wherein R², R⁴ and R⁵ are the same or different and are selected from the group consisting of alkyl, and functionalized alkyl, wherein the functional group is a heteroatom, a halide, an aryl, or a heteroaryl, to obtain a compound having an aldehyde function;
(b) reacting the compound obtained in (a) with an allylating agent;
(c) subjecting the reaction product of (b) to oxidative cleavage of the aldehyde to obtain a compound selected from the group consisting of: 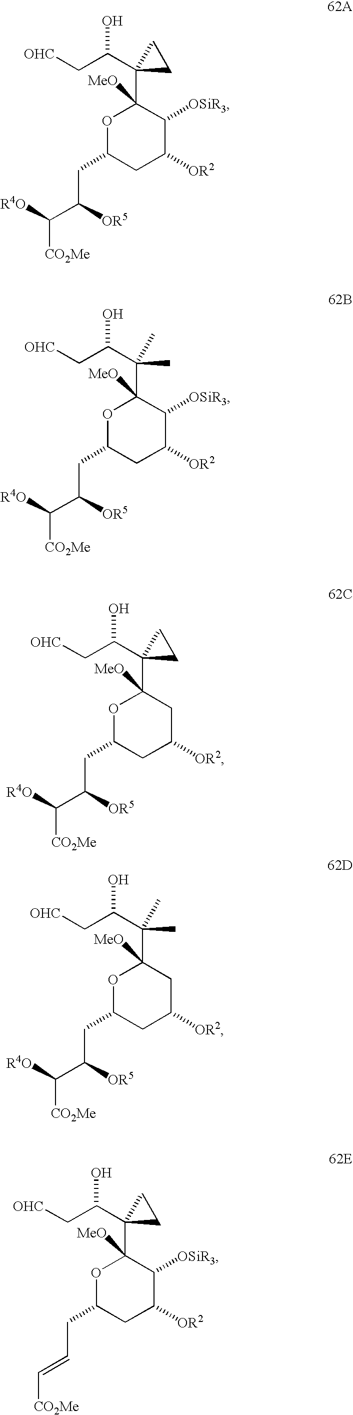 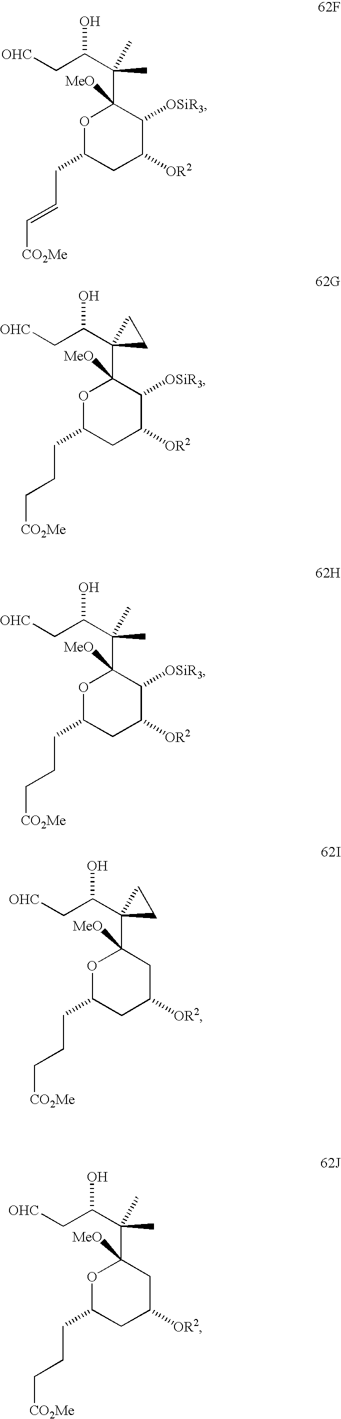
wherein R², R⁴ and R⁵ are the same or different and are selected from the group consisting of alkyl, and functionalized alkyl, wherein the functional group is a heteroatom, a halide, an aryl, or a heteroaryl;
(d) reacting the compound obtained in (c) with an enolate derived from a methyl or ethyl ketone to obtain a compound selected from the group consisting of: 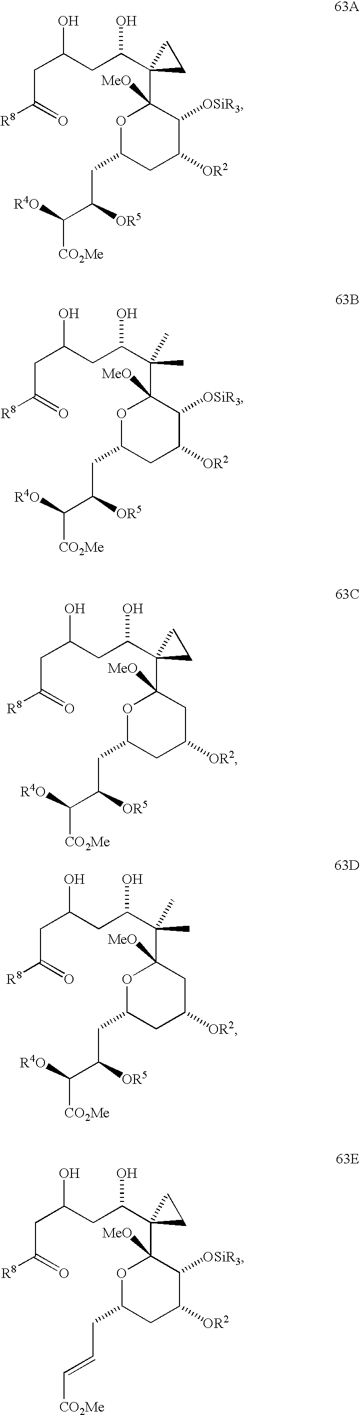 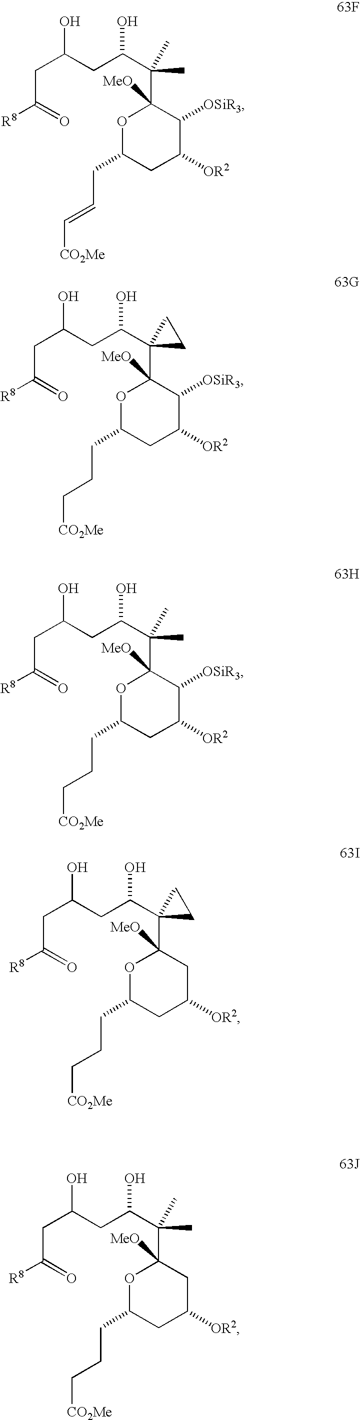
wherein R², R⁴ and R⁵ can be the same or different and are selected from the group consisting of alkyl and functionalized alkyl, and where R⁸ is selected from the group consisting of aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl;
(e) reacting the compound obtained in (d) with an alkylating agent to introduce an R³ group;
(f) subjecting the compound obtained in (e) with a reducing agent to reduce a ketone group in the compound obtained in (e) to an alcohol;
(g) converting the alcohol group of the compound obtained in (f) to an ester group;
(h) subjecting the compound obtained in (g) to an agent that hydrolyzes the ester group of the compound produced in (f) to a carboxylic acid group to obtain a seco-acid compound selected from the group consisting of: 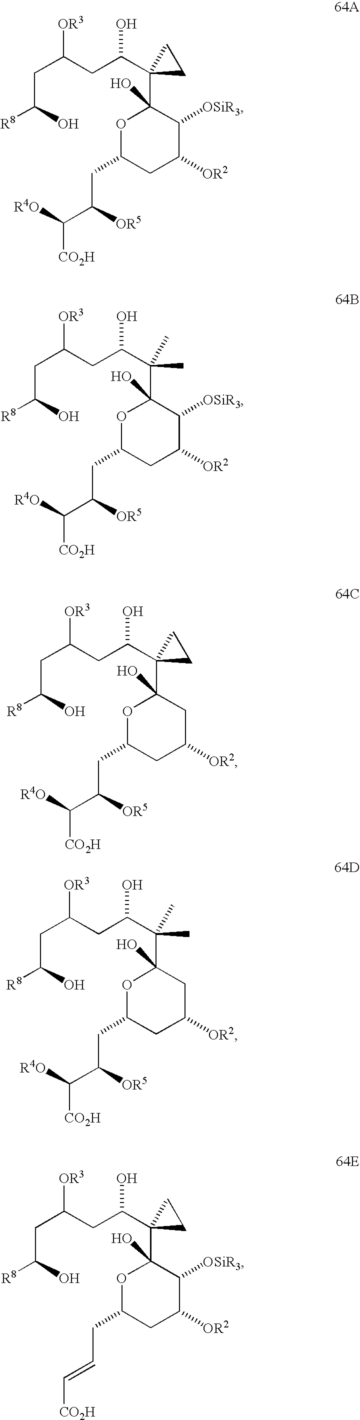 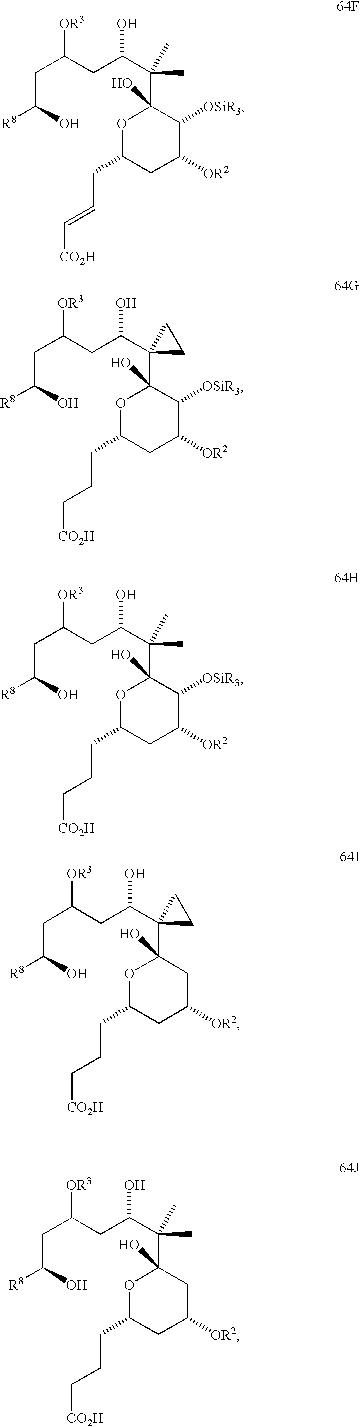
wherein R², R³, R⁴, R⁵ can be the same or different and are selected from the group consisting of alkyl, and functionalized alkyl, and where R⁸ is selected from the group consisting of aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl; and
(i) reacting the carboxylic acid group and a hydroxyl group of the compound produced in (h) to obtain a macrolactone selected from the group consisting of: 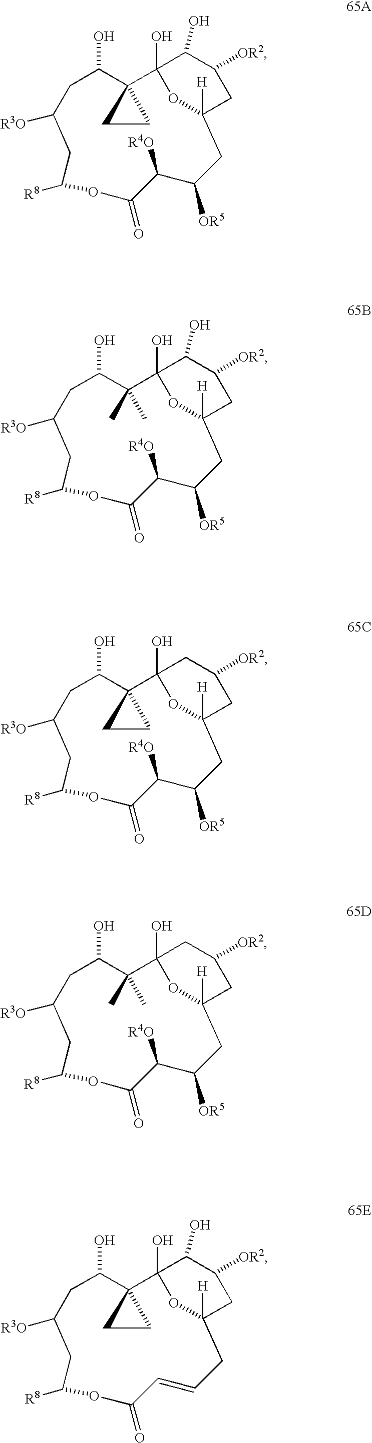 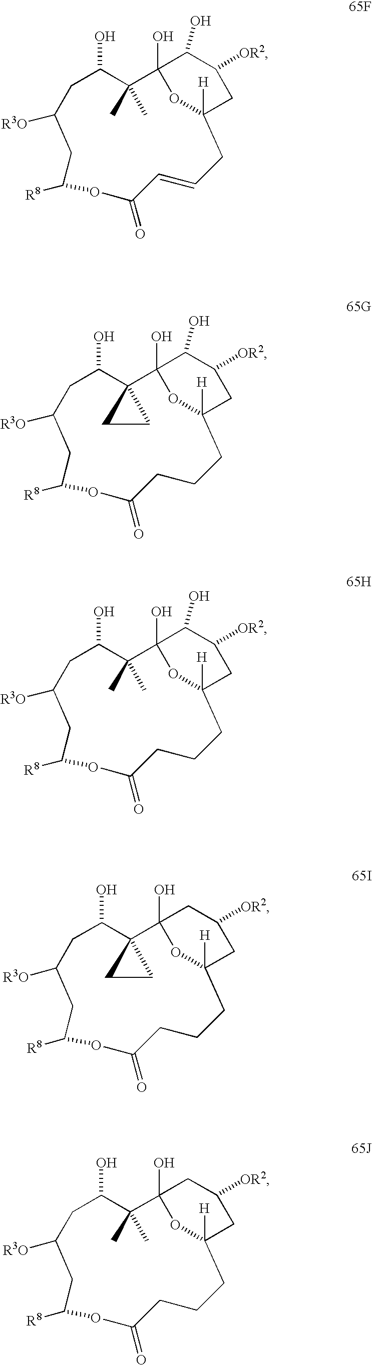
wherein R², R³, R⁴, R⁵ can be the same or different and are selected from the group consisting of alkyl, and functionalized alkyl, and where R⁸ is selected from the group consisting of aryl, heteroaryl, alkyl, functionalized alkyl, alkenyl, functionalized alkenyl, alkynyl, and functionalized alkynyl, wherein the functional group is a heteroatom, a halide, an aryl, or a heteroaryl.

25. The method of claim 24 wherein the macrolactone is Peloruside A.

26. The method of claim 24 wherein the macrolactone has the formula:

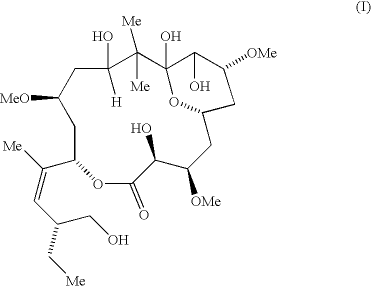

27. The method of claim 24 wherein the macrolactone has the formula: [chemical structure]
